(19) United States
(12) Patent Application Publication
(10) Pub. No.: US 2025/0318429 A1
(43) Pub. Date: Oct. 9, 2025

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si, (KR)

(72) Inventors: Wook KIM, suwon-s i (KR); Hyung Sun KIM, suwon-si (KR); Dalho HUH, suwon-si (KR); Hanill LEE, suwon-si (KR); Jiyun KWON, Suwon-si (KR); Jonghoon KIM, Suwon-si (KR); Seungjae LEE, Suwon-si (KR); Jiah YOON, Suwon-si (KR); Seonyeong GWAK, suwon-si (KR); Suheon IM, suwon-si (KR); Minjj PARK, suwon-si (KR)

(21) Appl. No.: 19/078,707

(22) Filed: Mar. 13, 2025

(30) Foreign Application Priority Data
Apr. 5, 2024 (KR) 10-2024-0046880

(51) Int. Cl. H10K 85/60; C07D 403/14; C07F 7/08; C09K 11/02; H10K 50/11

(52) U.S. Cl. CPC H10K 85/654; C07D 403/14; C07F 7/0812; C09K 11/02; H10K 85/6572; H10K 50/11

(57) ABSTRACT

A compound for an organic optoelectronic device, a composition for an organic optoelectronic device including the compound, an organic optoelectronic device including the compound or the composition for an organic optoelectronic device, and a display device including the organic optoelectronic device, the compound being represented by Chemical Formula 1:

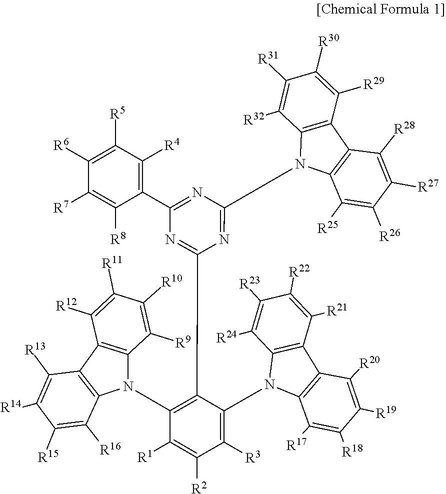

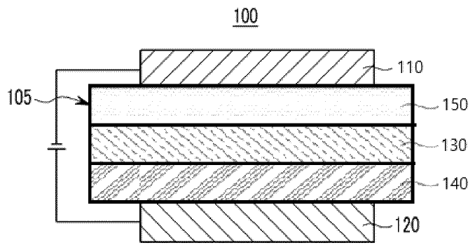

### CROSS-REFERENCE TO RELATED APPLICATION

This application claims priority to and the benefit of Korean Patent Application No. 10-2024-0046880 filed in the Korean Intellectual Property Office on Apr. 5, 2024, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device may include an organic photoelectric device, an organic light emitting diode, an organic solar cell, or an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### SUMMARY

The embodiments may be realized by providing a compound for an organic optoelectronic device, the compound being represented by Chemical Formula 1:

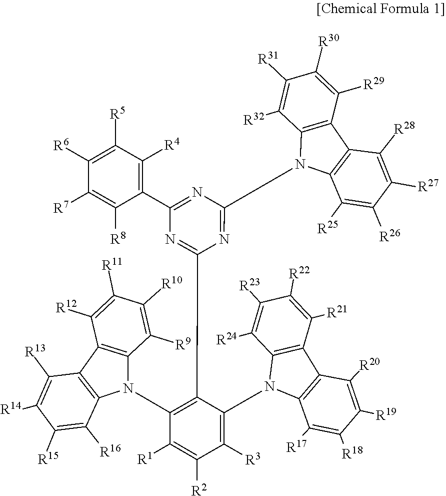

wherein, in Chemical Formula 1, R¹ to R³ are each independently hydrogen, deuterium, or a substituted or unsubstituted phenyl group, and R⁴ to R³² are each independently hydrogen, deuterium, a cyano group, a substituted or unsubstituted C1 to C20 alkyl group, a substituted or unsubstituted C1 to C20 alkylsilyl group, or a substituted or unsubstituted C6 to C30 aryl group.

The embodiments may be realized by providing a composition for an organic optoelectronic device, the composition including a first compound; and a second compound, wherein the first compound is the compound for an organic optoelectronic device according to an embodiment, and the second compound is represented by Chemical Formula 2; a combination of Chemical Formula 3 and Chemical Formula 4; or Chemical Formula 5,

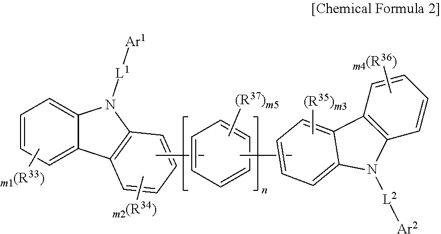

in Chemical Formula 2, R³³ to R³⁷ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar¹ and Ar² are each independently a substituted or unsubstituted C6 to C20 aryl group or a substituted or unsubstituted C2 to C30 heterocyclic group, L¹ and L² are each independently a single bond or a substituted or unsubstituted C6 to C20 arylene group, m1, m4, and m5 are each independently an integer of 1 to 4, m2 and m3 are each independently an integer of 1 to 3, when m1 is 2, 3, or 4, each R³³ is the same or different from each other, when m2 is 2 or 3, each R³⁴ is the same or different from each other, when m3 is 2 or 3, each R³⁵ is the same or different from each other, when m4 is 2, 3, or 4, each R³⁶ is the same or different from each other, when m5 is 2, 3, or 4, each R³⁷ is the same or different from each other, and n is an integer of 0 to 2;

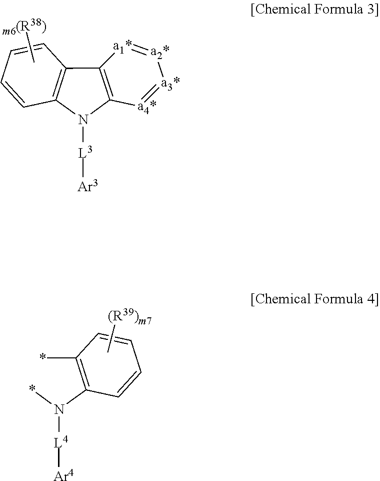

in Chemical Formula 3 and Chemical Formula 4, two adjacent ones of a₁* to a₄* in Chemical Formula 3 are linking carbons linked at * in Chemical Formula 4, and the remaining two of a₁* to a₄* in Chemical Formula 3, not linked at * of Chemical Formula 4, are C-L^{a}-R^{a}, L^{a}, L³, and L⁴ are each independently a single bond or a substituted or unsubstituted C6 to C20 arylene group, R^{a}, R³⁸ and R³⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar³ and Ar⁴ are each independently a substituted or unsubstituted C6 to C20 aryl group or a substituted or unsubstituted C2 to C30 heterocyclic group, m6 and m7 are each independently an integer of 1 to 4, when m6 is 2, 3, or 4, each R³⁸ is the same or different from each other, and when m7 is 2, 3, or 4, each R³⁹ is the same or different from each other;

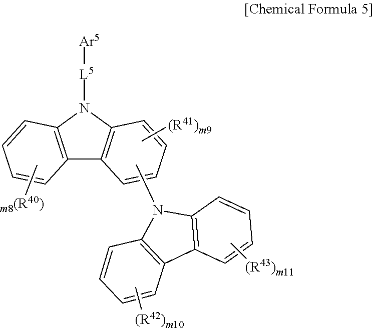

in Chemical Formula 5, L⁵ is a single bond or a substituted or unsubstituted C6 to C20 arylene group, R⁴⁰ to R⁴³ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar⁵ is a substituted or unsubstituted C6 to C20 aryl group or a substituted or unsubstituted C2 to C30 heterocyclic group, m8, m10 and m11 are each independently an integer of 1 to 4, m9 is an integer of 1 to 3, when m8 is 2, 3, or 4, each R⁴⁰ is the same or different from each other, when m9 is 2 or 3, each R⁴¹ is the same or different from each other, when m10 is 2, 3, or 4, each R⁴² is the same or different from each other, and when m11 is 2, 3, or 4, each R⁴³ is the same or different from each other.

The embodiments may be realized by providing an organic optoelectronic device including an anode and a cathode facing each other, and at least one organic layer between the anode and the cathode, and the at least one organic layer including the compound for an organic optoelectronic device according to an embodiment.

The embodiments may be realized by providing an organic optoelectronic device including an anode and a cathode facing each other, and at least one organic layer between the anode and the cathode, and the at least one organic layer including the composition for an organic optoelectronic device according to an embodiment.

The embodiments may be realized by providing a display device including the organic optoelectronic device.

### BRIEF DESCRIPTION OF THE DRAWING

Features will become apparent to those of skill in the art by describing in detail exemplary embodiments with reference to the attached drawing in which:

The FIGURE is a cross-sectional view showing an organic light emitting diode according to some embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. As used herein, the term “or” is not necessarily an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present embodiments, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present embodiments, the “substituted” refers to replacement of at least one hydrogen of a substituent or a compound by deuterium, a C1 to C20 alkyl group, a C1 to C5 alkylsilyl group, a C6 to C20 aryl group, a C2 to C20 heteroaryl group, or a cyano group. In specific example of the present embodiments, the “substituted” refers to replacement of at least one hydrogen of a substituent or a compound by deuterium, a C1 to C5 alkyl group, a C1 to C5 alkylsilyl group, a C6 to C18 aryl group, a C2 to C18 heteroaryl group, or a cyano group. In specific example of the present embodiments, the “substituted” refers to replacement of at least one hydrogen of a substituent or a compound by deuterium, a cyano group, a methyl group, an ethyl group, an n-propyl group, an iso-propyl group, an n-butyl group, an iso-butyl group, a sec-butyl group, a tert-butyl group, a trimethylsilyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).” For example, any hydrogen in any compound described herein may be protium, deuterium, or tritium (e.g., based on natural or artificial substitution).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. In an implementation, the heterocyclic group may be a fused ring, and the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to some embodiments is described.

The compound for an organic optoelectronic device according to some embodiments may be represented by Chemical Formula 1.

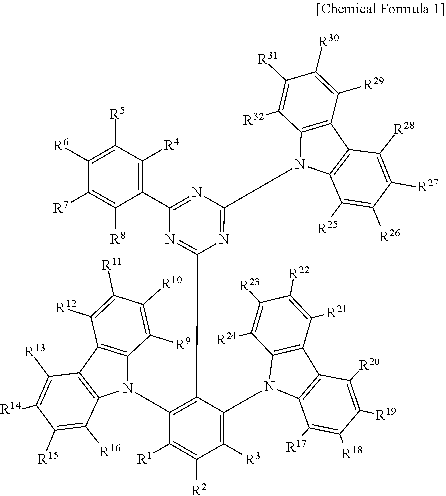

In Chemical Formula 1, R¹ to R³ may each independently be or include, e.g., hydrogen, deuterium, or a substituted or unsubstituted phenyl group.

R⁴ to R³² may each independently be or include, e.g., hydrogen, deuterium, a cyano group, a substituted or unsubstituted C1 to C20 alkyl group, a substituted or unsubstituted C1 to C20 alkylsilyl group, or a substituted or unsubstituted C6 to C30 aryl group.

The compound represented by Chemical Formula 1 may have a structure in which two N-carbazoles are substituted at the ortho positions (2 and 6) of phenylene linked to the triazine, and another N-carbazole is substituted for the triazine. As the energy transfer efficiency to the phosphorescent dopant may be particularly improved thereby, it may be a desirable material as a phosphorescent host.

In an implementation, a dihedral angle may increase due to steric hindrance between the N-carbazole additionally substituted on the triazine and the two N-carbazoles substituted via ortho-phenylene with respect to the triazine. The two N-carbazoles substituted for the triazine via ortho-phenylene may be twisted together to increase the dihedral angle. This may mean that electron clouds of a HOMO energy level and a LUMO energy level are mostly separated without overlap, and because it has a small ΔEst, fast energy transfer may be possible, showing high efficiency characteristics, especially when applied as a phosphorescent host. In addition, a side reaction path in the excited state may be reduced, resulting in an additional life-span increase.

In an implementation, by substituting at least one aryl group on the triazine, electron mobility may be increased and stability may be increased, thereby improving driving and life-span.

In an implementation, R⁴ to R⁸ may each independently be, e.g., hydrogen, deuterium, or a substituted or unsubstituted C6 to C20 aryl group.

R⁹ to R³² may each independently be, e.g., hydrogen, deuterium, a cyano group, a substituted or unsubstituted C1 to C10 alkyl group, a substituted or unsubstituted C1 to C10 alkylsilyl group, or a substituted or unsubstituted C6 to C20 aryl group.

In an implementation, R⁴ to R⁸ may each independently be, e.g., hydrogen, deuterium, or a substituted or unsubstituted C6 to C12 aryl group.

R⁹ to R³² may each independently be, e.g., hydrogen, deuterium, a cyano group, a substituted or unsubstituted C1 to C10 alkyl group, a substituted or unsubstituted C1 to C10 alkylsilyl group, or a substituted or unsubstituted C6 to C12 aryl group.

In an implementation, R⁴ to R⁸ may each independently be, e.g., hydrogen, deuterium, a substituted or unsubstituted phenyl group, or a substituted or unsubstituted biphenyl group.

R⁹ to R³² may each independently be, e.g., hydrogen, deuterium, a cyano group, a substituted or unsubstituted methyl group, a substituted or unsubstituted ethyl group, a substituted or unsubstituted propyl group, a substituted or unsubstituted trimethylsilyl group, a substituted or unsubstituted phenyl group, or a substituted or unsubstituted biphenyl group.

In an implementation, the compound represented by Chemical Formula 1 may be a compound of Group 1.

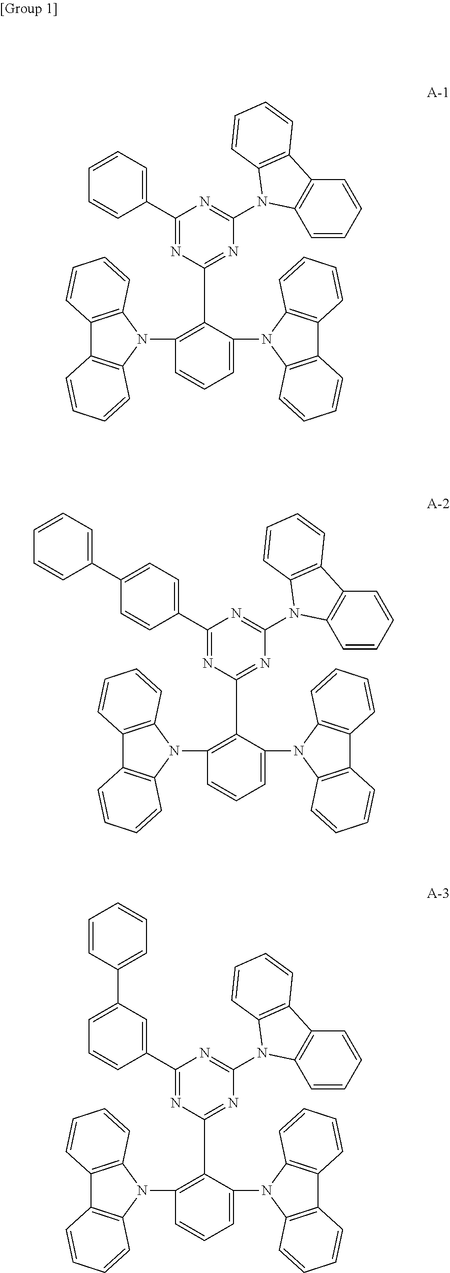 [SMILES: c1ccc(-c2nc(-c3c(-n4c5ccccc5c5ccccc54)cccc3-n3c4ccccc4c4ccccc43)nc(-n3c4ccccc4c4ccccc43)n2)cc1 ; c1ccc(-c2ccc(-c3nc(-c4c(-n5c6ccccc6c6ccccc65)cccc4-n4c5ccccc5c5ccccc54)nc(-n4c5ccccc5c5ccccc54)n3)cc2)cc1 ; c1ccc(-c2cccc(-c3nc(-c4c(-n5c6ccccc6c6ccccc65)cccc4-n4c5ccccc5c5ccccc54)nc(-n4c5ccccc5c5ccccc54)n3)c2)cc1]

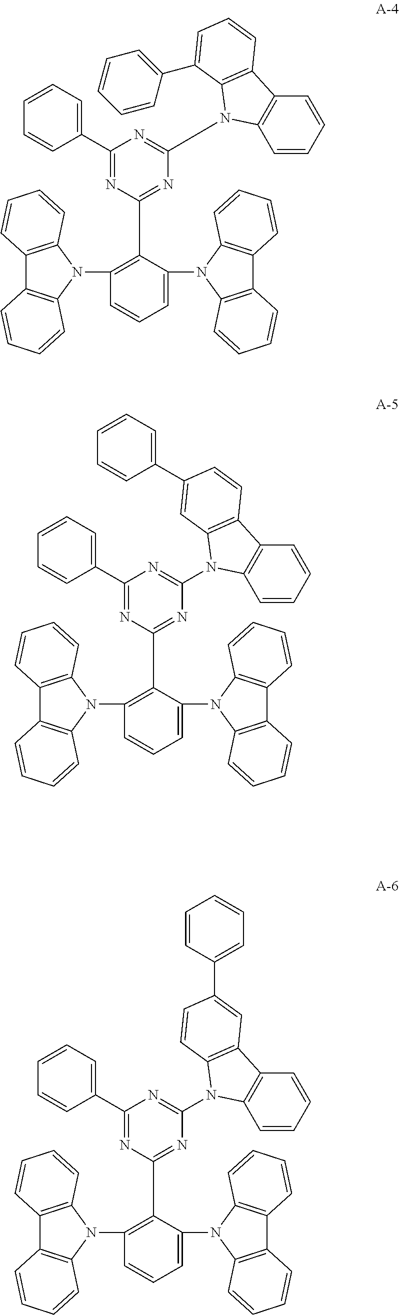 [SMILES: c1ccc(-c2nc(-c3c(-n4c5ccccc5c5ccccc54)cccc3-n3c4ccccc4c4ccccc43)nc(-n3c4ccccc4c4cccc(-c5ccccc5)c43)n2)cc1 ; c1ccc(-c2ccc3c4ccccc4n(-c4nc(-c5ccccc5)nc(-c5c(-n6c7ccccc7c7ccccc76)cccc5-n5c6ccccc6c6ccccc65)n4)c3c2)cc1 ; c1ccc(-c2ccc3c(c2)c2ccccc2n3-c2nc(-c3ccccc3)nc(-c3c(-n4c5ccccc5c5ccccc54)cccc3-n3c4ccccc4c4ccccc43)n2)cc1]

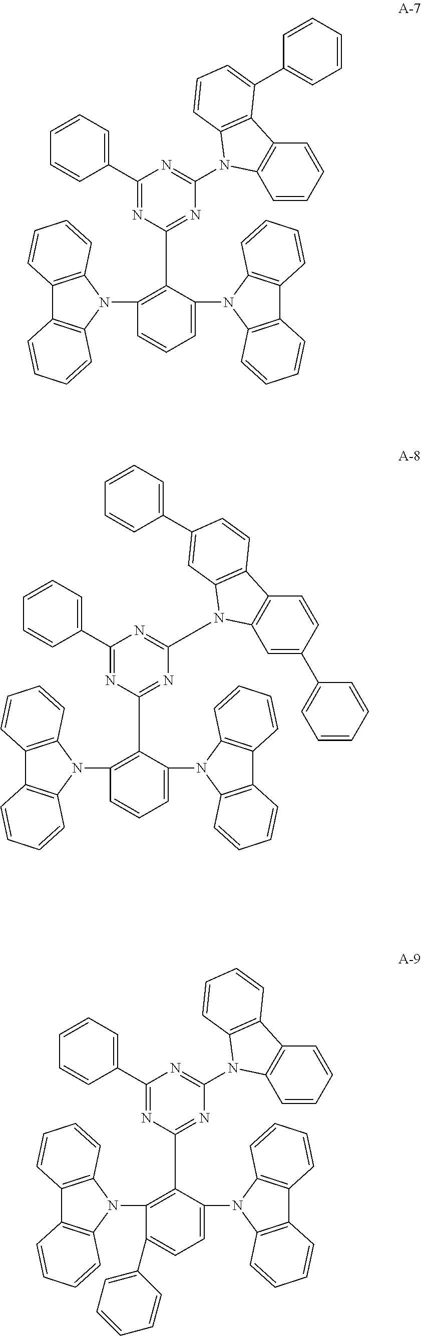 [SMILES: c1ccc(-c2nc(-c3c(-n4c5ccccc5c5ccccc54)cccc3-n3c4ccccc4c4ccccc43)nc(-n3c4ccccc4c4c(-c5ccccc5)cccc43)n2)cc1 ; c1ccc(-c2ccc3c4ccc(-c5ccccc5)cc4n(-c4nc(-c5ccccc5)nc(-c5c(-n6c7ccccc7c7ccccc76)cccc5-n5c6ccccc6c6ccccc65)n4)c3c2)cc1 ; c1ccc(-c2nc(-c3c(-n4c5ccccc5c5ccccc54)ccc(-c4ccccc4)c3-n3c4ccccc4c4ccccc43)nc(-n3c4ccccc4c4ccccc43)n2)cc1]

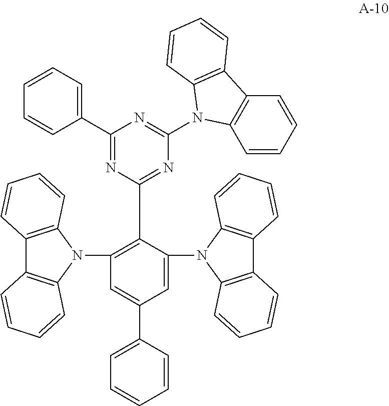 [SMILES: c1ccc(-c2cc(-n3c4ccccc4c4ccccc43)c(-c3nc(-c4ccccc4)nc(-n4c5ccccc5c5ccccc54)n3)c(-n3c4ccccc4c4ccccc43)c2)cc1]

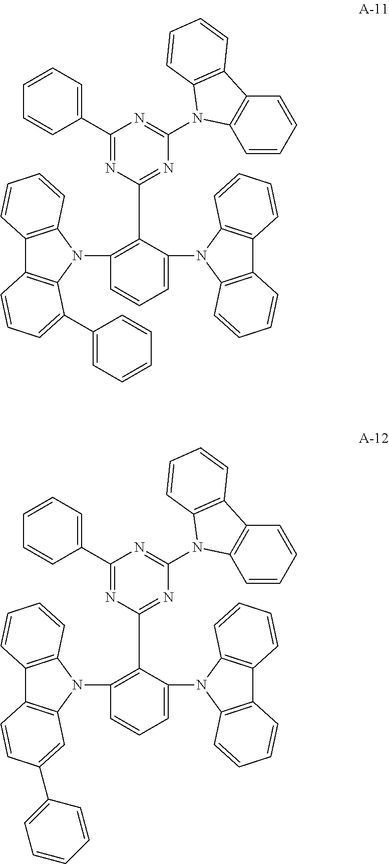 [SMILES: c1ccc(-c2nc(-c3c(-n4c5ccccc5c5ccccc54)cccc3-n3c4ccccc4c4cccc(-c5ccccc5)c43)nc(-n3c4ccccc4c4ccccc43)n2)cc1 ; c1ccc(-c2ccc3c4ccccc4n(-c4cccc(-n5c6ccccc6c6ccccc65)c4-c4nc(-c5ccccc5)nc(-n5c6ccccc6c6ccccc65)n4)c3c2)cc1]

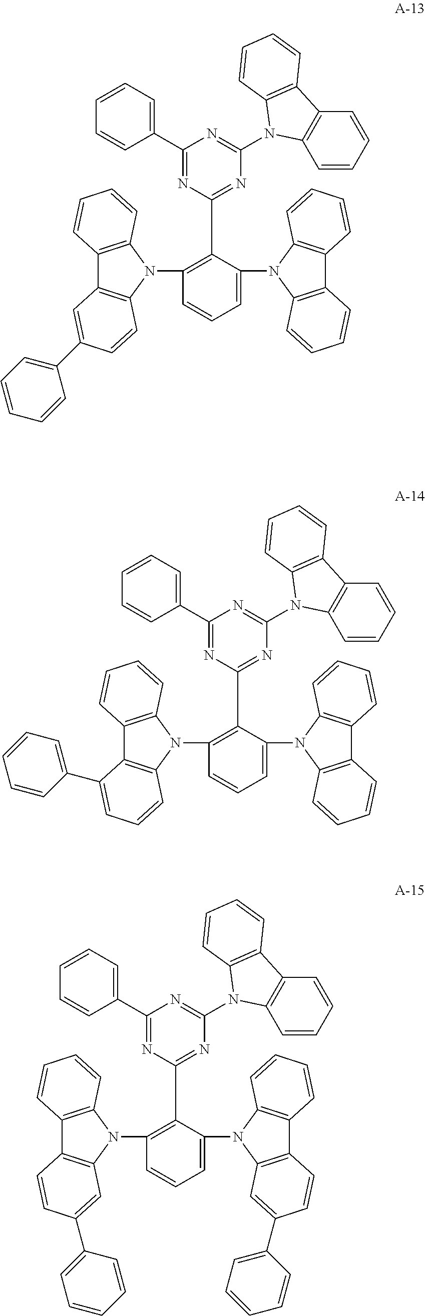 [SMILES: c1ccc(-c2ccc3c(c2)c2ccccc2n3-c2cccc(-n3c4ccccc4c4ccccc43)c2-c2nc(-c3ccccc3)nc(-n3c4ccccc4c4ccccc43)n2)cc1 ; c1ccc(-c2nc(-c3c(-n4c5ccccc5c5ccccc54)cccc3-n3c4ccccc4c4c(-c5ccccc5)cccc43)nc(-n3c4ccccc4c4ccccc43)n2)cc1 ; c1ccc(-c2ccc3c4ccccc4n(-c4cccc(-n5c6ccccc6c6ccc(-c7ccccc7)cc65)c4-c4nc(-c5ccccc5)nc(-n5c6ccccc6c6ccccc65)n4)c3c2)cc1]

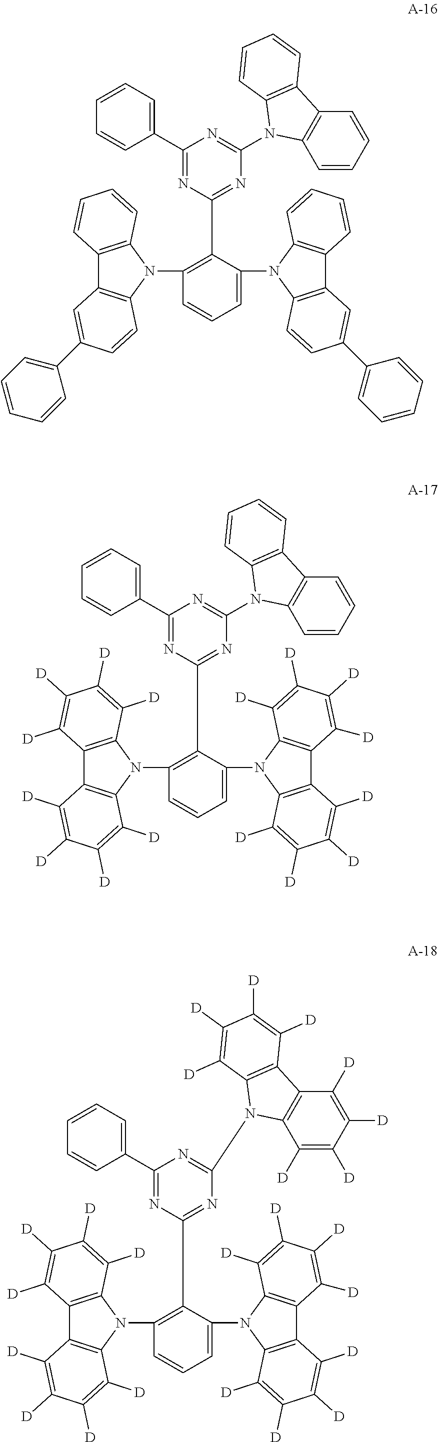

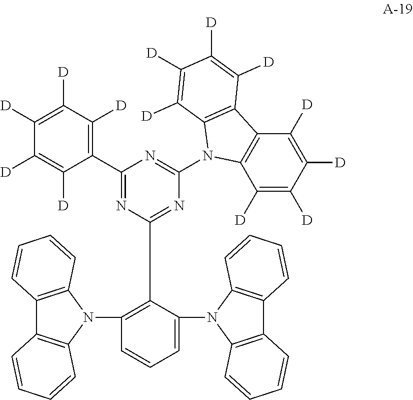 [SMILES: [2H]c1c([2H])c([2H])c(-c2nc(-c3c(-n4c5ccccc5c5ccccc54)cccc3-n3c4ccccc4c4ccccc43)nc(-n3c4c([2H])c([2H])c([2H])c([2H])c4c4c([2H])c([2H])c([2H])c([2H])c43)n2)c([2H])c1[2H]]

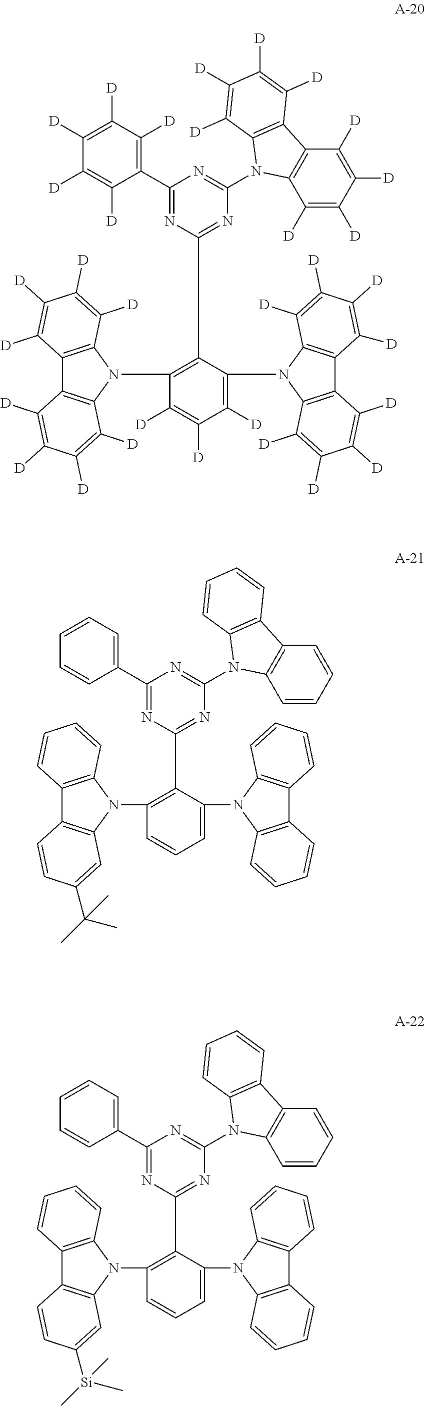 [SMILES: [2H]c1c([2H])c([2H])c(-c2nc(-c3c(-n4c5c([2H])c([2H])c([2H])c([2H])c5c5c([2H])c([2H])c([2H])c([2H])c54)c([2H])c([2H])c([2H])c3-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c([2H])c1[2H] ; CC(C)(C)c1ccc2c3ccccc3n(-c3cccc(-n4c5ccccc5c5ccccc54)c3-c3nc(-c4ccccc4)nc(-n4c5ccccc5c5ccccc54)n3)c2c1 ; C[Si](C)(C)c1ccc2c3ccccc3n(-c3cccc(-n4c5ccccc5c5ccccc54)c3-c3nc(-c4ccccc4)nc(-n4c5ccccc5c5ccccc54)n3)c2c1]

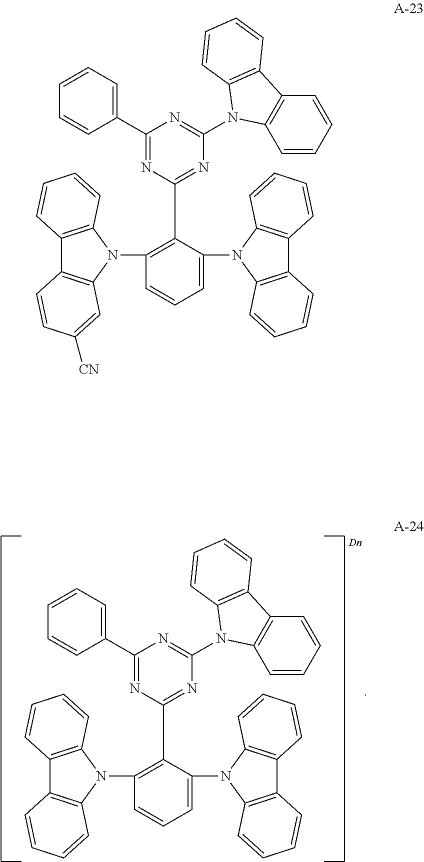

A composition for an organic optoelectronic device according to some embodiments may include a first compound, and a second compound. In an implementation, the first compound may be the aforementioned compound for an organic optoelectronic device and the second compound may be represented by, e.g., Chemical Formula 2; a combination of Chemical Formula 3 and Chemical Formula 4; or Chemical Formula 5.

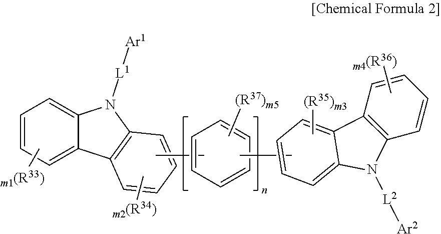

In Chemical Formula 2, R³³ to R³⁷ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar¹ and Ar² may each independently be or include, e.g., a substituted or unsubstituted C6 to C20 aryl group or a substituted or unsubstituted C2 to C30 heterocyclic group.

L¹ and L² may each independently be, e.g., a single bond or a substituted or unsubstituted C6 to C20 arylene group.

m1, m4, and m5 may each independently be, e.g., an integer of 1 to 4.

m2 and m3 may each independently be, e.g., an integer of 1 to 3.

In an implementation, m1 to m5 may each be 2 or more, and each R³³ to R³⁷ may be the same or different from each other.

n may be, e.g., an integer of 0 to 2;

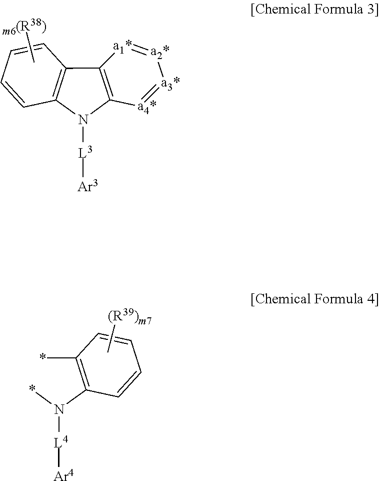

In Chemical Formula 3 and Chemical Formula 4, two adjacent ones among a₁* to a₄* in Chemical Formula 3 may be linking carbons linked at * of Chemical Formula 4. The remaining two of a₁* to a₄* in Chemical Formula 3, not linked at * of Chemical Formula 4, may each independently be, e.g., C-L^{a}-R^{a}. As used herein, the term “linking carbon” refers to a shared carbon at which fused rings are linked.

L^{a}, L³, and L⁴ may each independently be, e.g., a single bond or a substituted or unsubstituted C6 to C20 arylene group.

R^{a}, R³⁸ and R³⁹ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar³ and Ar⁴ may each independently be, e.g., a substituted or unsubstituted C6 to C20 aryl group or a substituted or unsubstituted C2 to C30 heterocyclic group.

m6 and m7 may each independently be, e.g., an integer of 1 to 4.

In an implementation, m6 and m7 may each be 2 or more, and each R³⁸ and R³⁹ may be the same or different from each other.

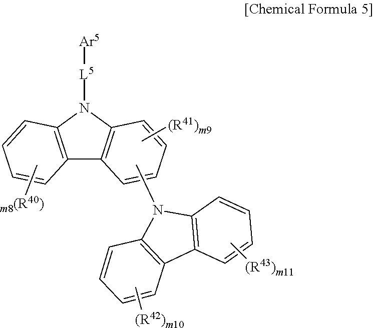

In Chemical Formula 5, L⁵ may be, e.g., a single bond or a substituted or unsubstituted C6 to C20 arylene group.

R⁴⁰ to R⁴³ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar⁵ may be, e.g., a substituted or unsubstituted C6 to C20 aryl group or a substituted or unsubstituted C2 to C30 heterocyclic group.

m8, m10 and m11 may each independently be, e.g., an integer of 1 to 4.

m9 may be, e.g., an integer of 1 to 3.

The second compound may be used in the light emitting layer together with the first compound to improve luminous efficiency and life-span characteristics by helping increase charge mobility and stability.

In an implementation, in Chemical Formula 2, m1 may be, e.g., 2, 3, or 4, and each R³³ may be the same or different from each other.

In an implementation, in Chemical Formula 2, m2 may be, e.g., 2 or 3, and each R³⁴ may be the same or different from each other.

In an implementation, in Chemical Formula 2, m3 may be, e.g., 2 or 3, and each R³⁵ may be the same or different from each other.

In an implementation, in Chemical Formula 2, m4 may be, e.g., 2, 3, or 4, and each R³⁶ may be the same or different from each other.

In an implementation, in Chemical Formula 2, m5 may be, e.g., 2, 3, or 4, and each R³⁷ may be the same or different from each other.

In an implementation, in Chemical Formula 3 and Chemical Formula 4, m6 may be, e.g., 2, 3, or 4, and each R³⁸ may be the same or different from each other.

In an implementation, in Chemical Formula 3 and Chemical Formula 4, m7 may be, e.g., 2, 3, or 4, and each R³⁹ may be the same or different from each other.

In an implementation, in Chemical Formula 5, m8 may be, e.g., 2, 3, or 4, and each R⁴⁰ may be the same or different from each other.

In an implementation, in Chemical Formula 5, m9 may be, e.g., 2 or 3, and each R⁴¹ may be the same or different from each other.

In an implementation, in Chemical Formula 5, m10 may be, e.g., 2, 3, or 4, and each R⁴² may be the same or different from each other.

In an implementation, in Chemical Formula 5, m11 may be, e.g., 2, 3, or 4, and each R⁴³ may be the same or different from each other.

In an implementation, in Chemical Formula 2, Ar¹ and Ar² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, in Chemical Formula 2, L¹ and L² may each independently be, e.g., a single bond, a substituted or unsubstituted phenylene group, or a substituted or unsubstituted biphenylene group.

In an implementation, in Chemical Formula 2, R³³ to R³⁷ may each independently be, e.g., hydrogen, deuterium, or a substituted or unsubstituted C6 to C12 aryl group.

In an implementation, n may be, e.g., 0 or 1.

In an implementation, in Chemical Formula 2, “substituted” refers to replacement of at least one hydrogen by deuterium, a C1 to C4 alkyl group, a C6 to C18 aryl group, or a C2 to C30 heteroaryl group.

In an implementation, in Chemical Formula 2, Ar¹ and Ar² may each independently be, e.g., a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, Chemical Formula 2 may be represented by one of Chemical Formula 2-1 to Chemical Formula 2-15.

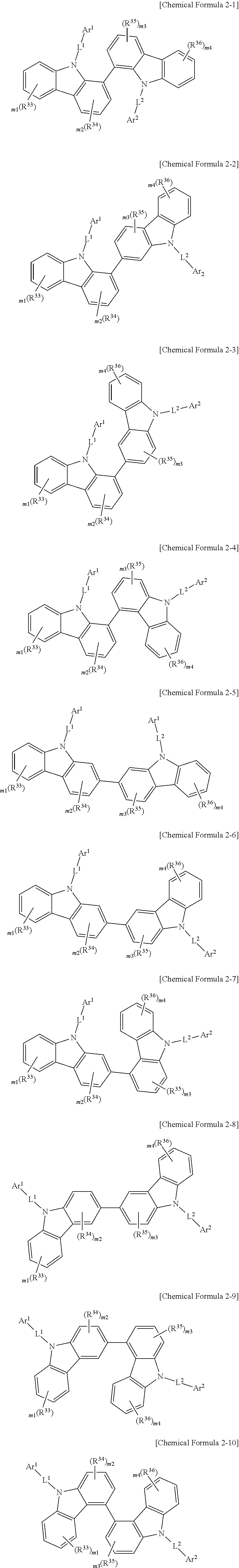

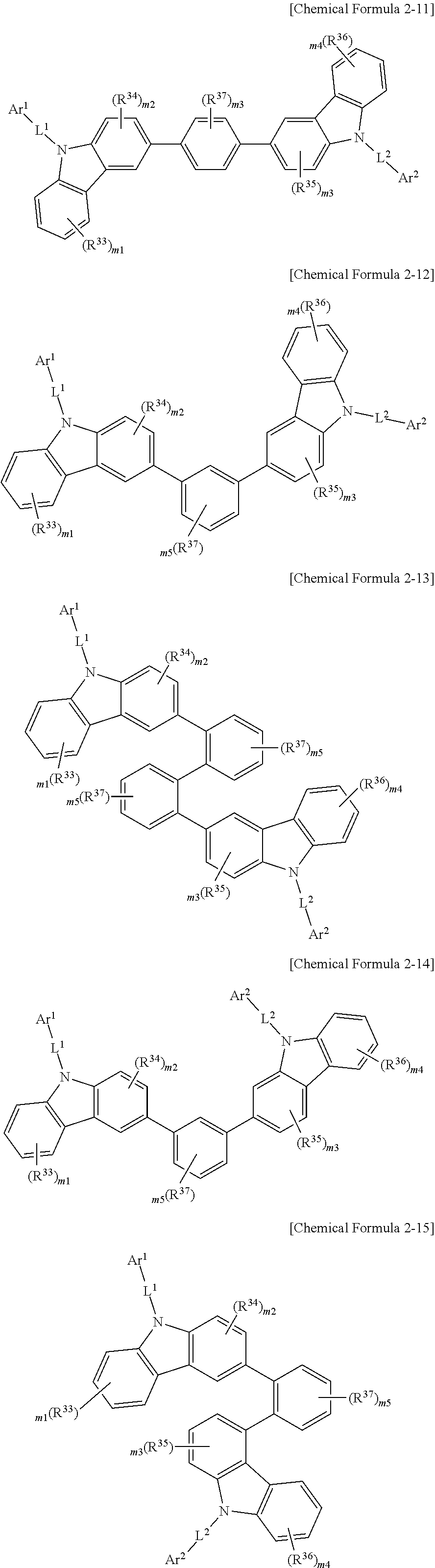

In Chemical Formula 2-1 to Chemical Formula 2-15, R³³ to R³⁷ may each independently be, e.g., hydrogen, deuterium or a substituted or unsubstituted C6 to C12 aryl group, and moieties -L¹-Ar¹ and -L²-Ar² may each independently be a moiety of Group I.

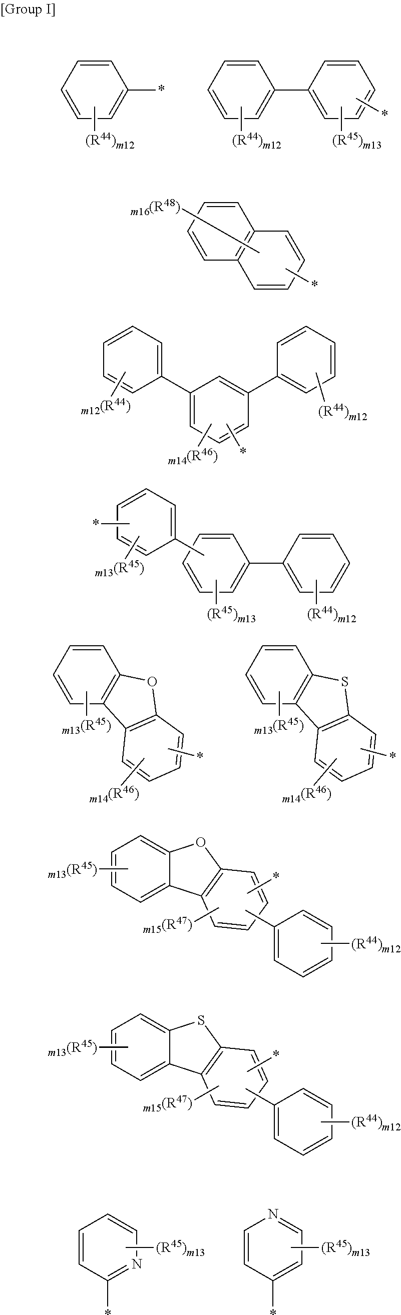

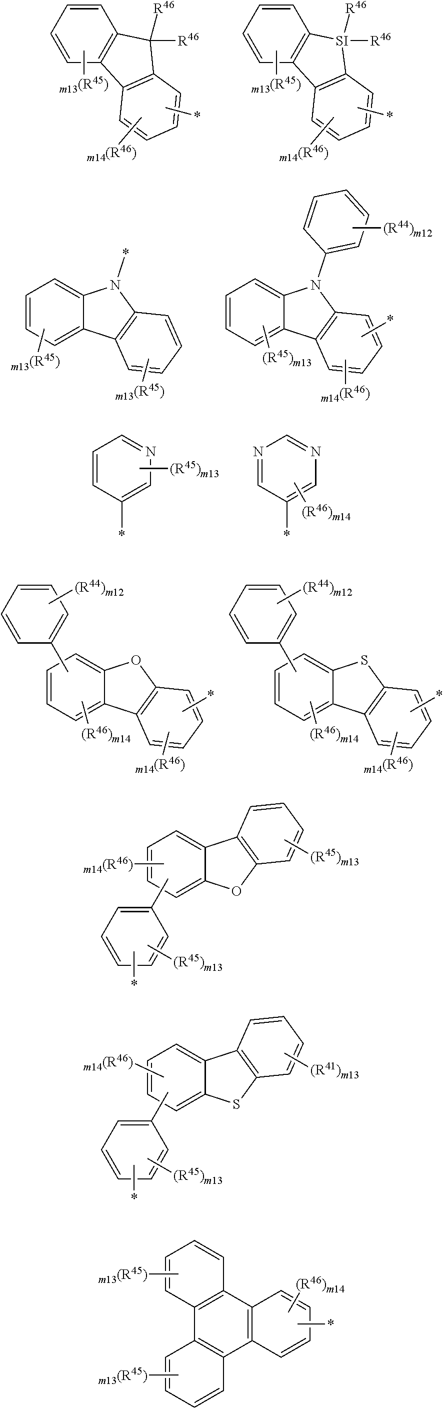

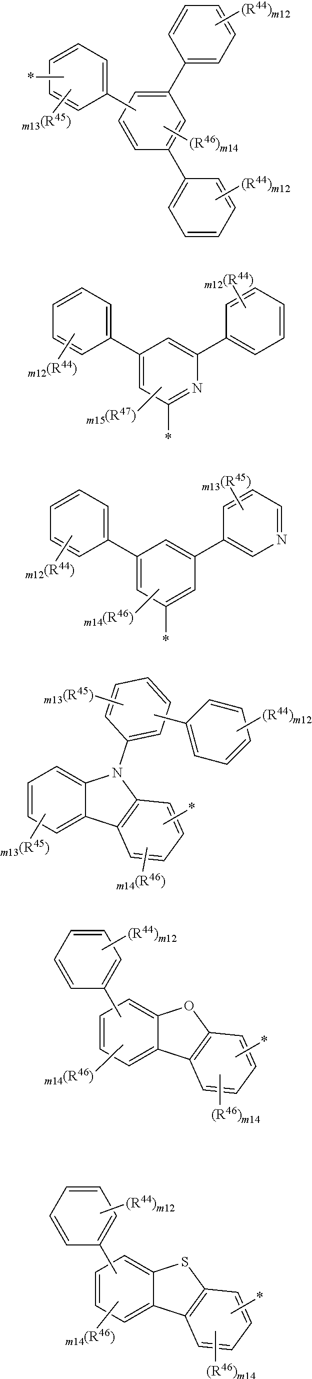

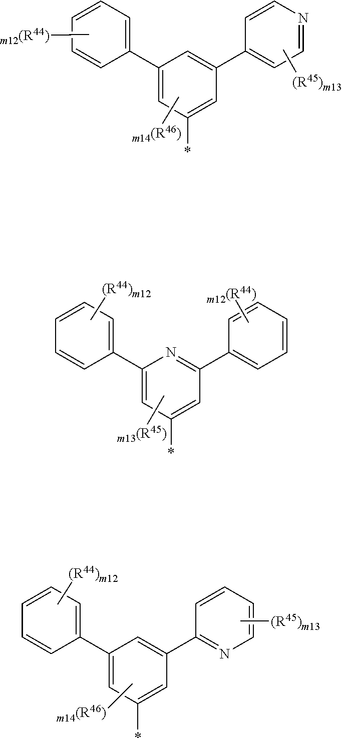

In Group I, R⁴⁴ to R⁴⁸ may each independently be or include, e.g., hydrogen, deuterium, a cyano group, a substituted or unsubstituted C1 to C10 alkyl group, or a substituted or unsubstituted C6 to C12 aryl group.

m12 may be, e.g., an integer of 1 to 5.

m13 may be, e.g., an integer of 1 to 4.

m14 may be, e.g., an integer of 1 to 3.

m15 may be, e.g., an integer of 1 or 2.

m16 may be, e.g., an integer of 1 to 7.

* is a linking point.

In an implementation, in Group I, m12 may be, e.g., 2, 3, 4, or 5, and each R⁴⁴ may be the same or different from each other.

In an implementation, in Group I, m13 may be, e.g., 2, 3, or 4, and each R⁴⁵ may be the same or different from each other.

In an implementation, in Group I, m14 may be, e.g., 2 or 3, and each R⁴⁶ may be the same or different from each other.

In an implementation, in Group I, m15 may be, e.g., 2, and each R⁴⁷ may be the same or different from each other.

In an implementation, in Group I, m16 may be, e.g., 2, 3, 4, 5, 6, or 7, and each R⁴⁸ may be the same or different from each other.

The combination of Chemical Formula 3 and Chemical Formula 4 may be represented, e.g., by one of Chemical Formula 3A, Chemical Formula 3B, Chemical Formula 3C, Chemical Formula 3D, and Chemical Formula 3E.

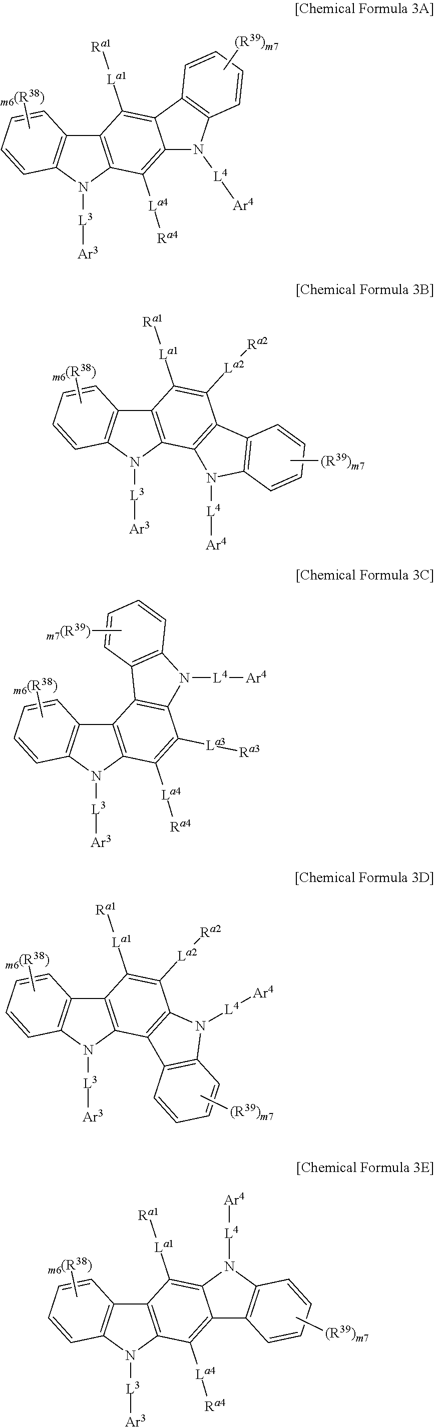

In Chemical Formula 3A to Chemical Formula 3E, L³, L⁴, Ar³, Ar⁴, R³⁸, R³⁹, m6 and m7 may be defined the same as those described above.

L^{a1} to L^{a4} may be defined the same as L³ and L⁴ described above.

R^{a1} to R^{a4} may be defined the same as R³⁸ and R³⁹ described above.

In an implementation, in Chemical Formula 3 and Chemical Formula 4, Ar³ and Ar⁴ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, R^{a1} to R^{a4}, R³⁸ and R³⁹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formulas 3 and 4, moieties -L³-Ar³ and -L⁴-Ar⁴ may each independently be a moiety of Group I.

In an implementation, R^{a1} to R^{a4}, R³⁸, and R³⁹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4}, R³⁸ and R³⁹ may each independently be, e.g., hydrogen, deuterium, a cyano group, or a substituted or unsubstituted phenyl group.

In an implementation, R^{a1} to R^{a4}, R³⁸ and R³⁹ may each independently be, e.g., hydrogen, deuterium or a substituted or unsubstituted phenyl group.

In an implementation, the second compound may be represented by Chemical Formula 2-8, and in Chemical Formula 2-8, Ar¹ and Ar² may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L¹ and L² may each independently be, e.g., a single bond, or a substituted or unsubstituted C6 to C20 arylene group, and R³³ to R³⁶ may each independently be, e.g., a hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 2-8, R³³ to R³⁶ may each independently be, e.g., hydrogen, deuterium or a substituted or unsubstituted C6 to C12 aryl group, and moieties -L¹-Ar¹ and -L²-Ar² may each independently be, e.g., a moiety of Group I.

In an implementation, the second compound may be represented by Chemical Formula 3C, and in Chemical Formula 3C, L^{a3} and L^{a4} may each be, e.g., a single bond, L³ and L⁴ may each independently be, e.g., a single bond or a substituted or unsubstituted C6 to C12 arylene group, R³⁸, R³⁹, R^{a3}, and R^{a4} may each independently be, e.g., hydrogen, deuterium, or a substituted or unsubstituted phenyl group, and Ar³ and Ar⁴ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 3C, L^{a3} and L^{a4} may be, e.g., a single bond, R³⁸, R³⁹, R^{a3}, and R^{a4} may each independently be, e.g., hydrogen, deuterium, or a substituted or unsubstituted C6 to C12 aryl group, and moieties -L³-Ar³ and -L⁴-Ar⁴ may each independently be a moiety of Group I.

In an implementation, Chemical Formula 5 may be, e.g., represented by one of Chemical Formula 5-1 to Chemical Formula 5-4.

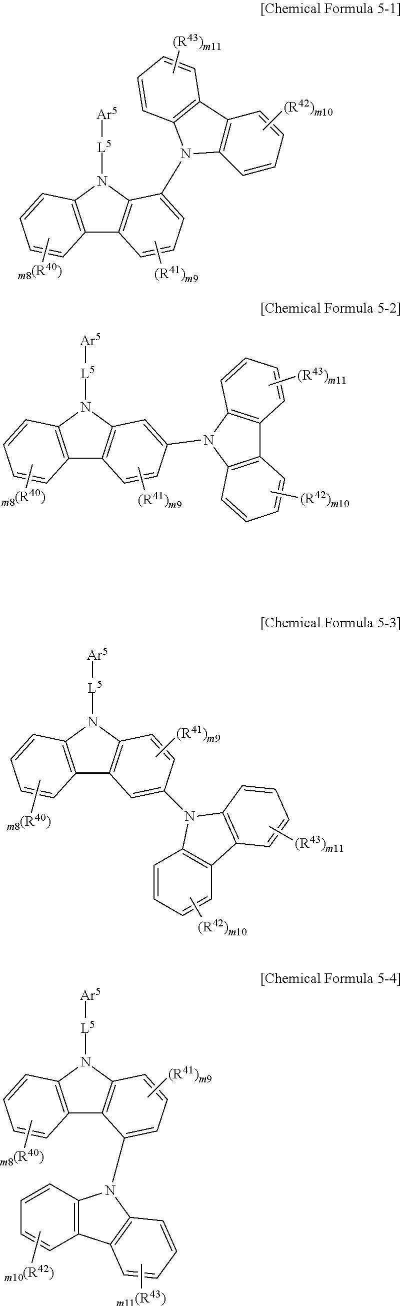

In Chemical Formula 5-1 to Chemical Formula 5-4, L⁵, Ar⁵, R⁴⁰ to R⁴³, and m8 to m11 may be defined the same as those described above.

In an implementation, in Chemical Formula 5, Ar⁵ may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, R⁴⁰ to R⁴³ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 5, the moiety -L⁵-Ar⁵ may be a moiety of Group I.

In an implementation, R⁴⁰ to R⁴³ may each independently be, e.g., hydrogen, deuterium, a cyano group, or a substituted or unsubstituted phenyl group.

In an implementation, the second compound for organic optoelectronic device compound may be, e.g., a compound of Group 2.

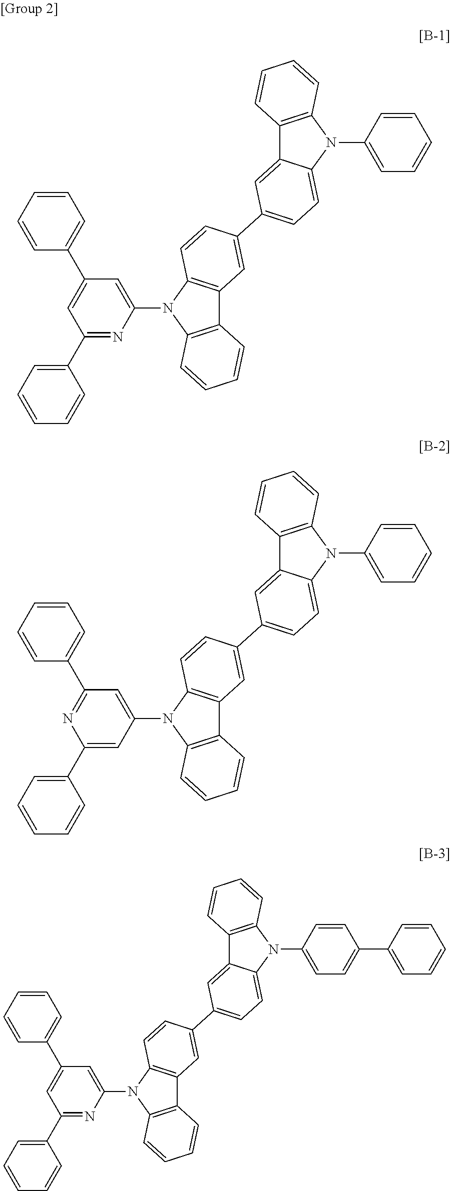 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

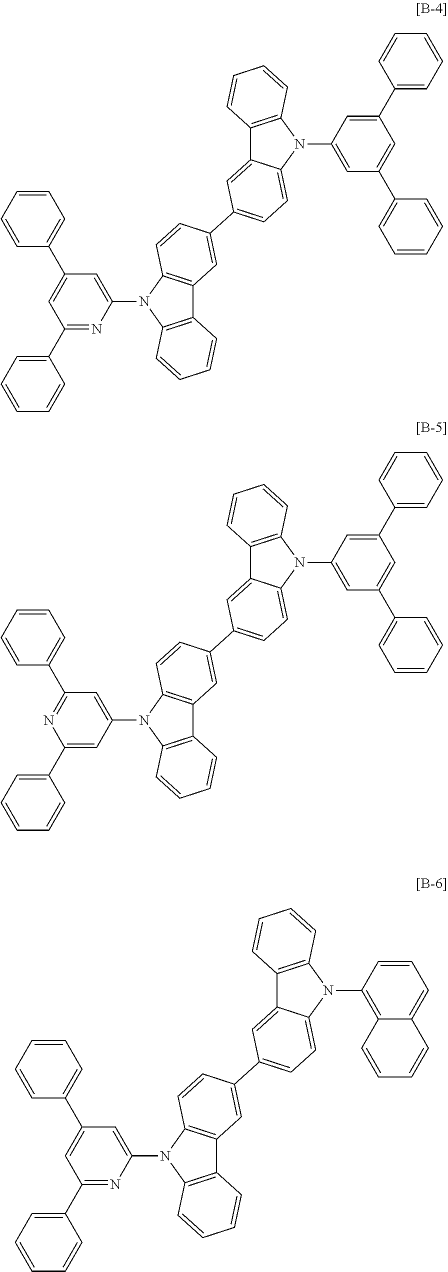 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1]

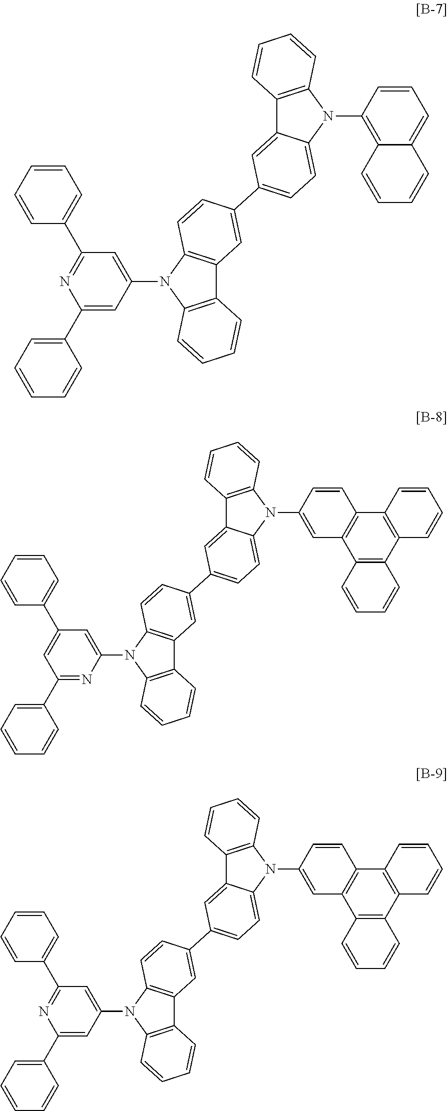 [SMILES: c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-n3c4ccccc4c4cc(-c5ccc6c(c5)c5ccccc5n6-c5ccc6c7ccccc7c7ccccc7c6c5)ccc43)cc(-c3ccccc3)n2)cc1]

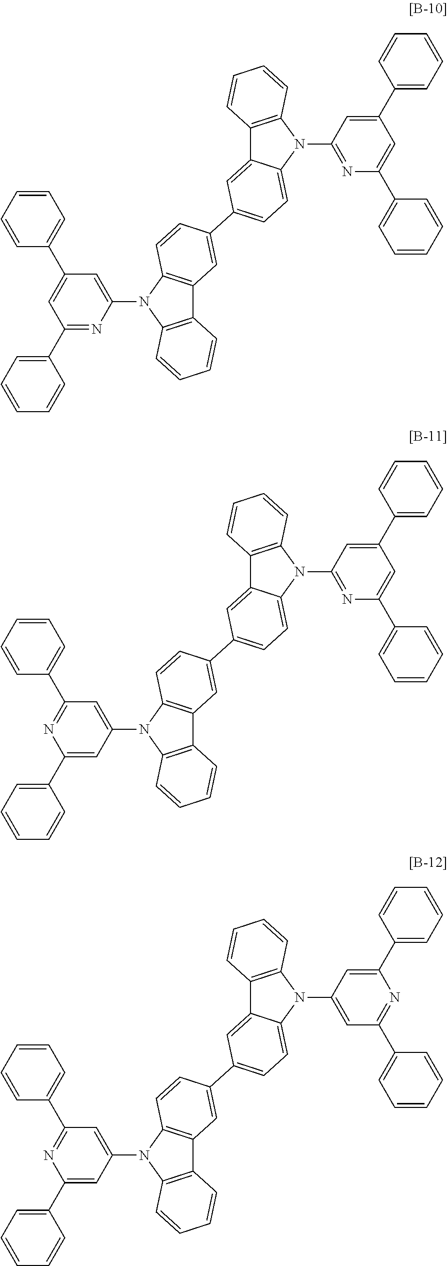 [SMILES: c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1]

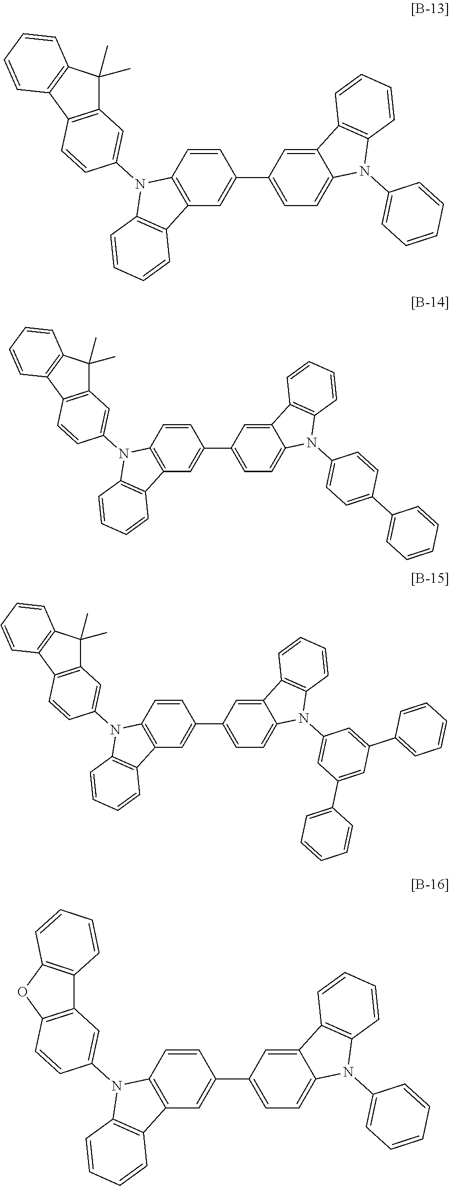 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-n2c3ccccc3c3cc(-c4ccc5c(c4)c4ccccc4n5-c4ccc5oc6ccccc6c5c4)ccc32)cc1]

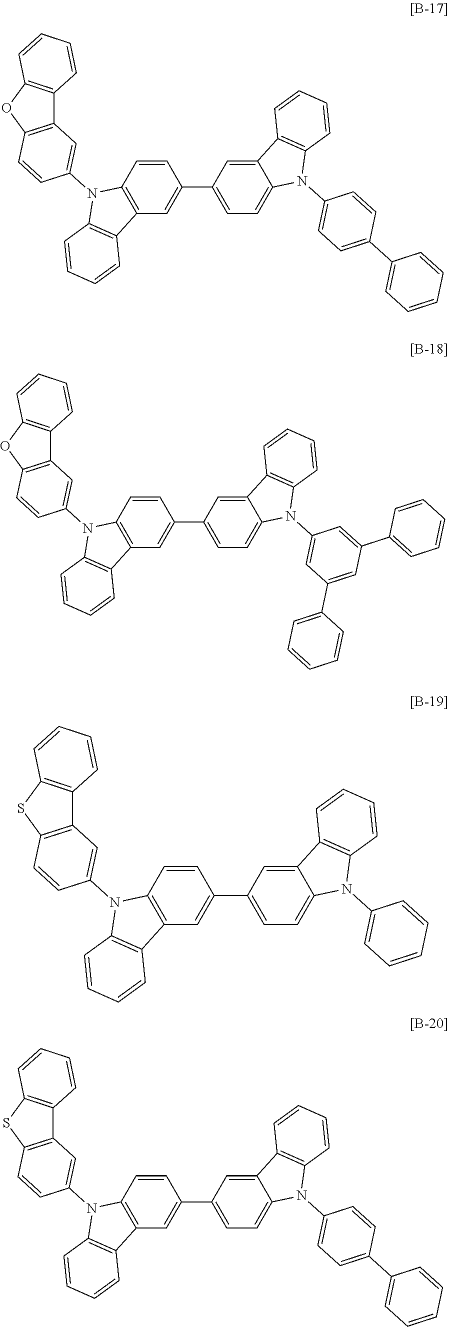 [SMILES: c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

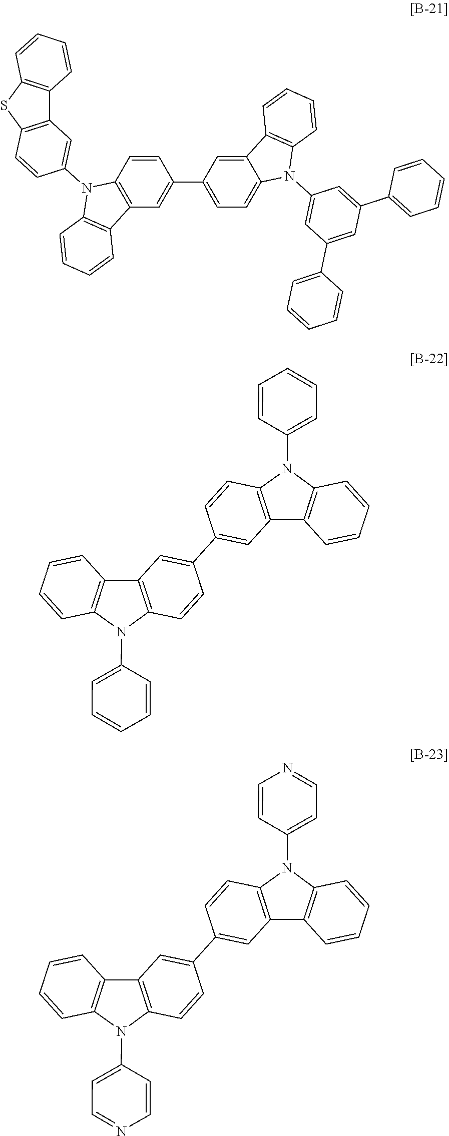

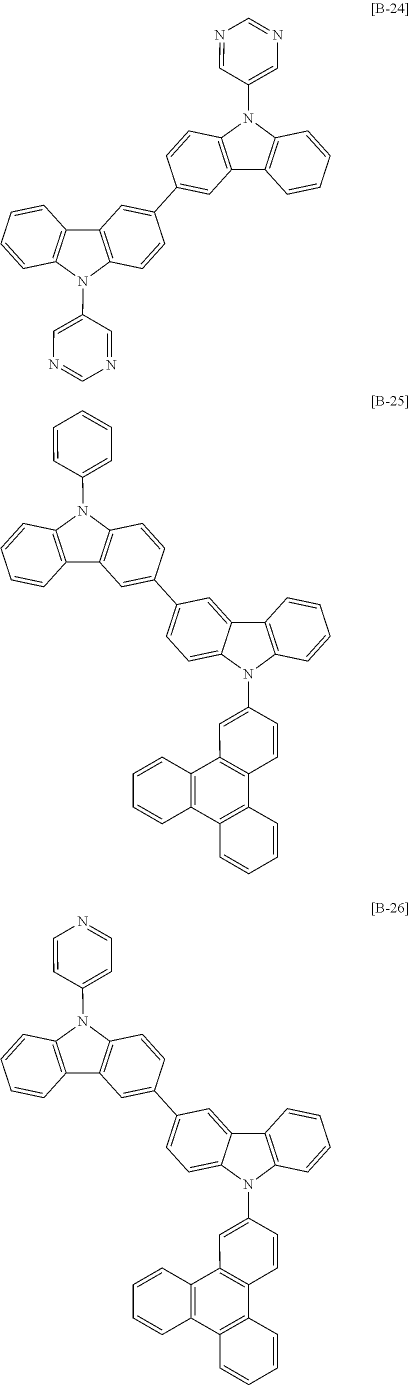

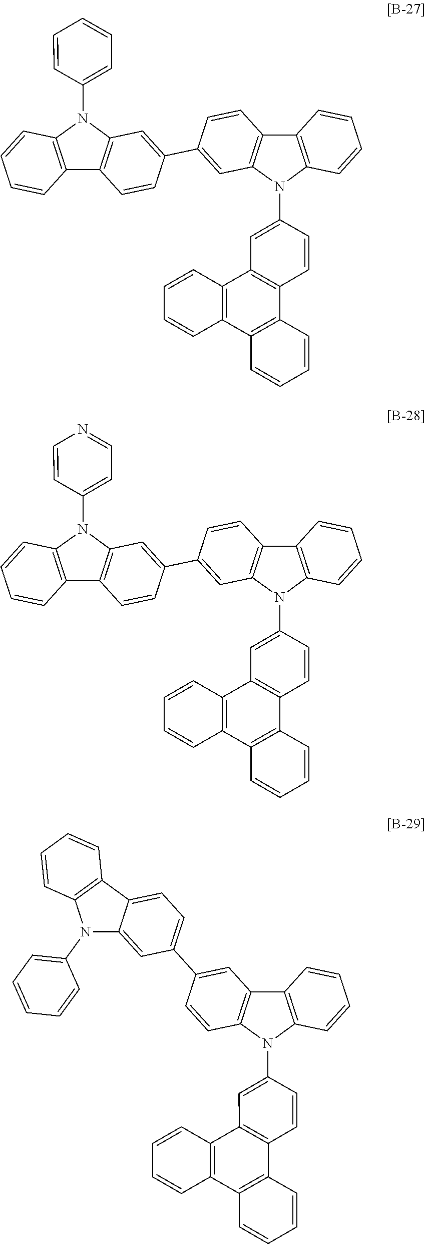 [SMILES: c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21 ; c1ccc(-n2c3ccccc3c3ccc(-c4ccc5c(c4)c4ccccc4n5-c4ccc5c6ccccc6c6ccccc6c5c4)cc32)cc1]

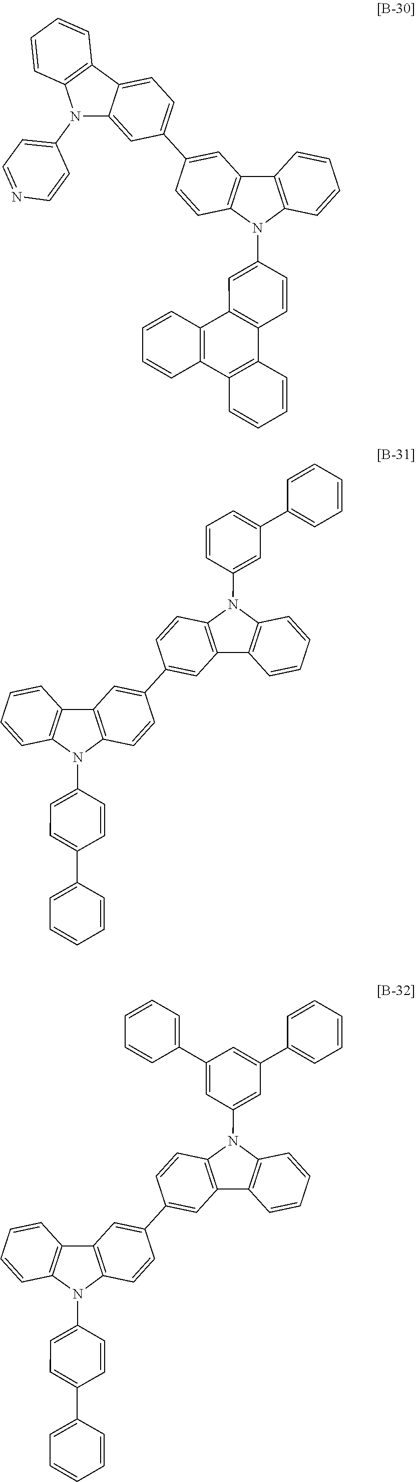 [SMILES: c1ccc2c(c1)c1ccccc1c1cc(-n3c4ccccc4c4cc(-c5ccc6c7ccccc7n(-c7ccncc7)c6c5)ccc43)ccc21 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

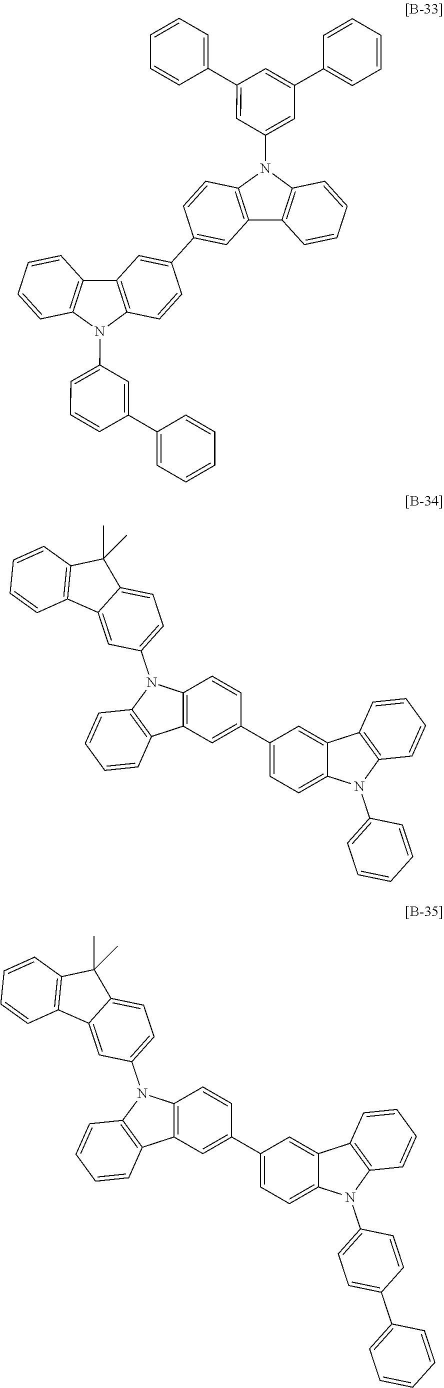 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21]

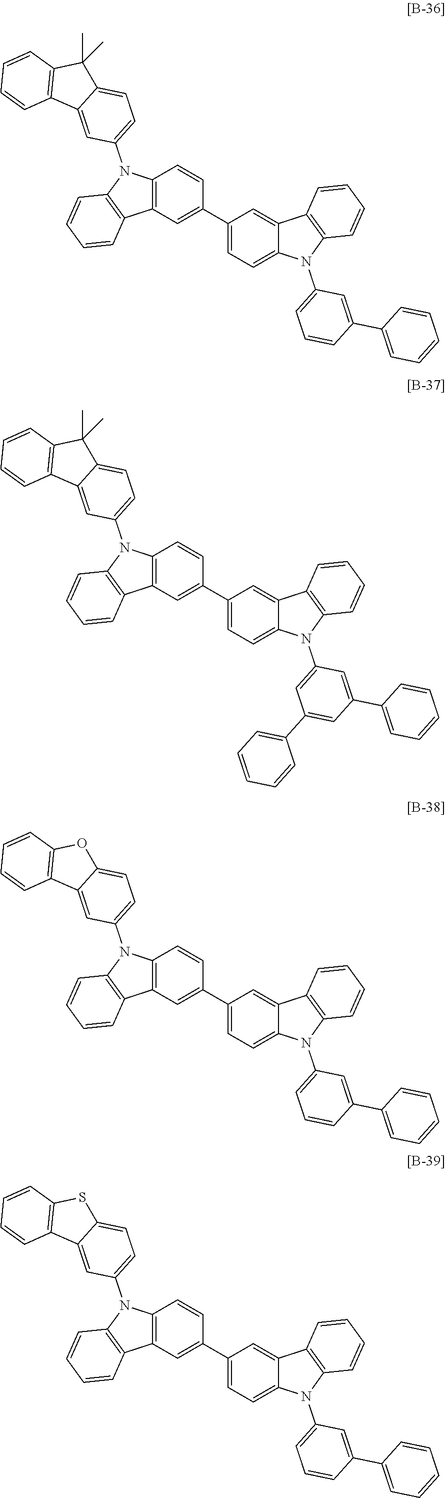 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

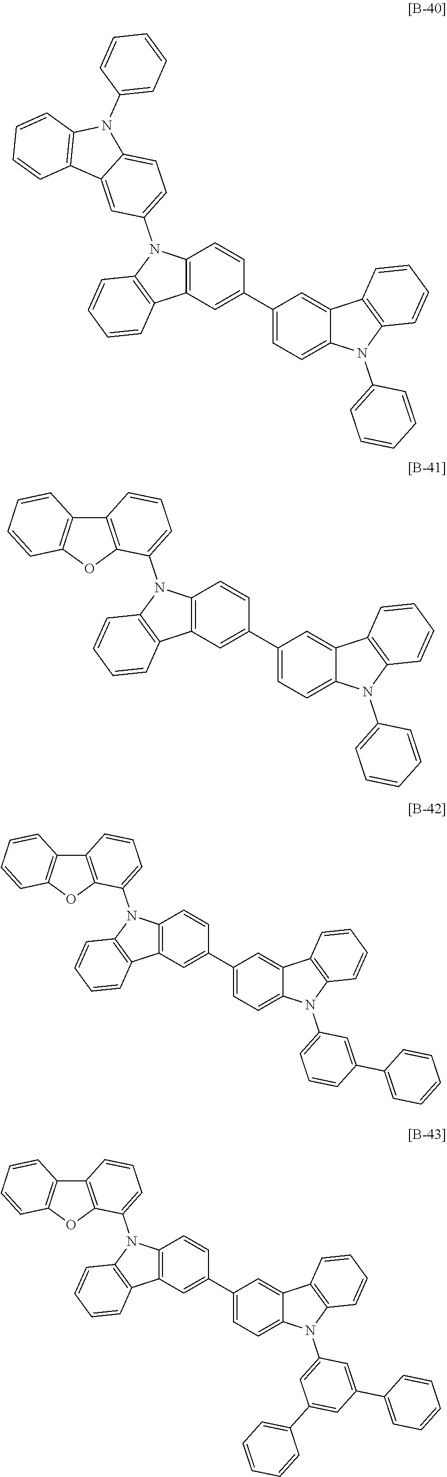 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1]

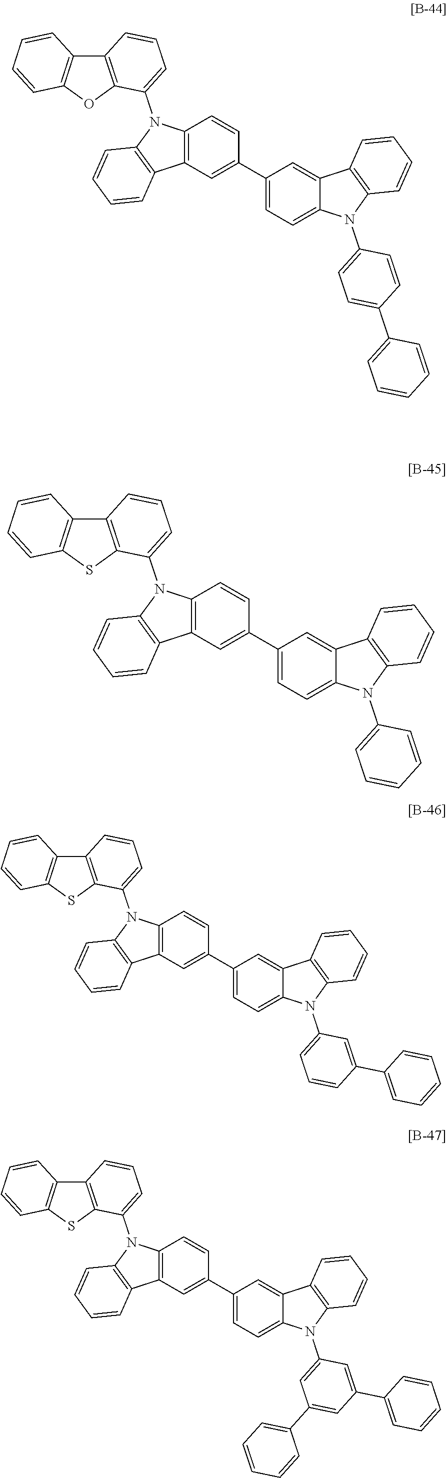 [SMILES: c1ccc(-c2ccc(-n3c4ccccc4c4cc(-c5ccc6c(c5)c5ccccc5n6-c5cccc6c5oc5ccccc56)ccc43)cc2)cc1 ; c1ccc(-n2c3ccccc3c3cc(-c4ccc5c(c4)c4ccccc4n5-c4cccc5c4sc4ccccc45)ccc32)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1]

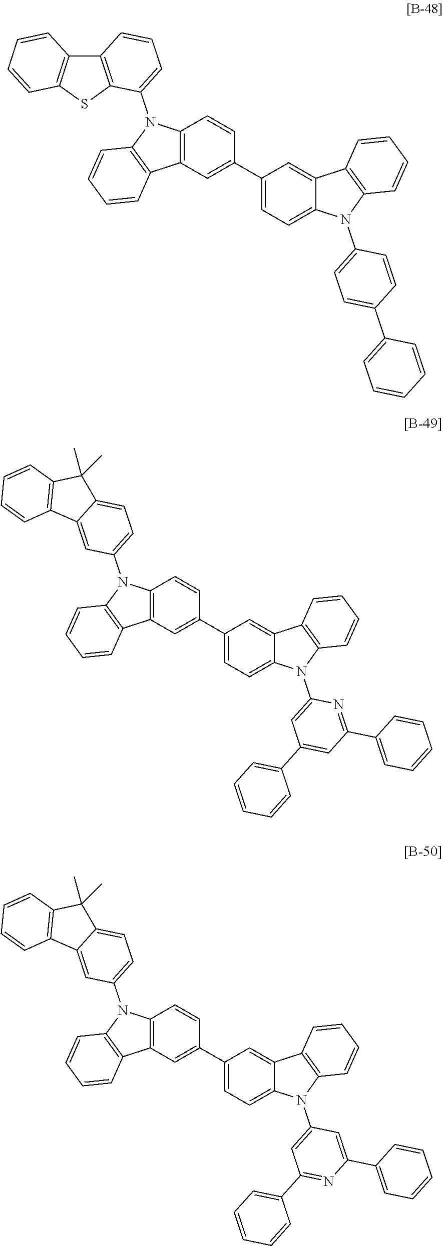 [SMILES: c1ccc(-c2ccc(-n3c4ccccc4c4cc(-c5ccc6c(c5)c5ccccc5n6-c5cccc6c5sc5ccccc56)ccc43)cc2)cc1 ; CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

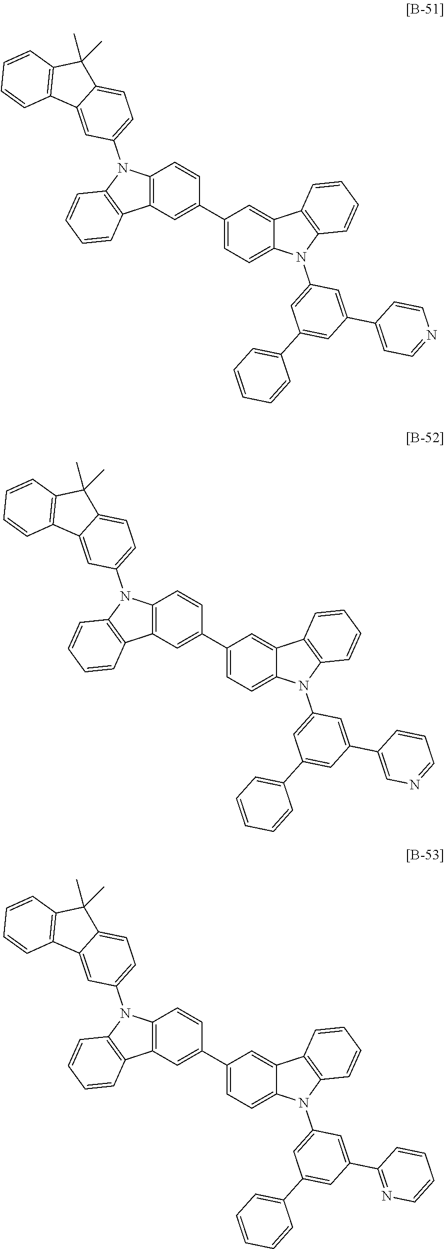 [SMILES: CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21]

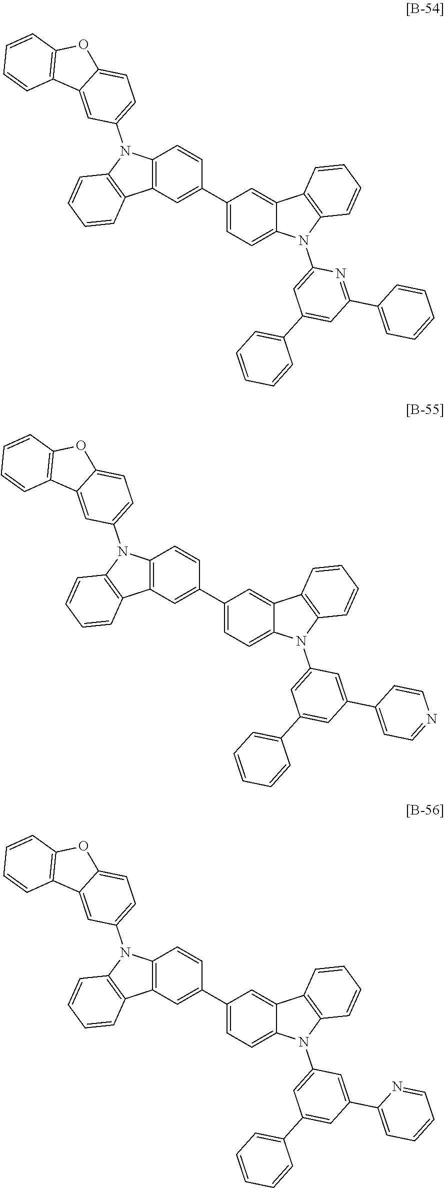 [SMILES: c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1]

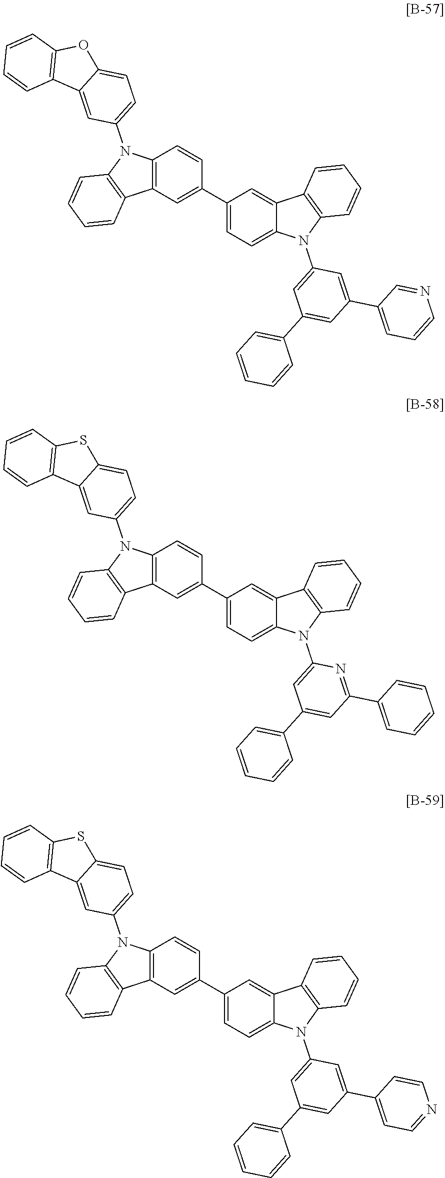 [SMILES: c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1]

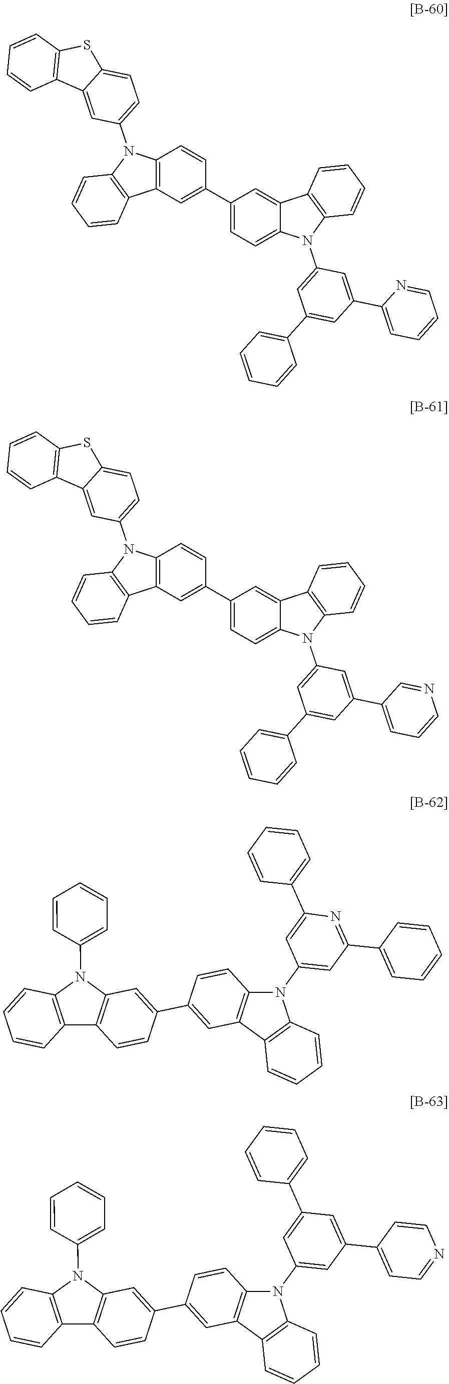 [SMILES: c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1]

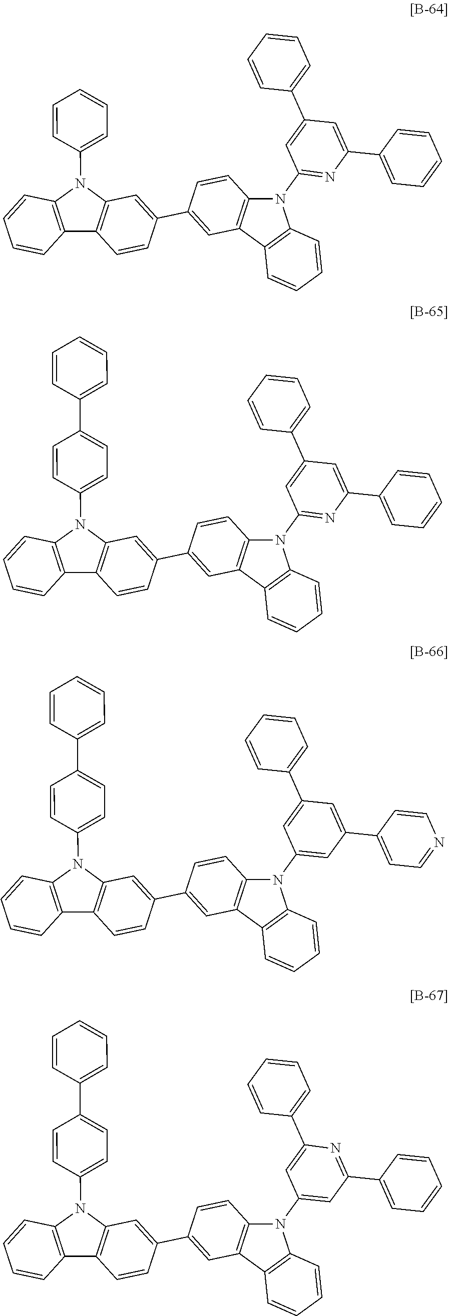 [SMILES: c1ccc(-c2cc(-c3ccccc3)nc(-n3c4ccccc4c4cc(-c5ccc6c7ccccc7n(-c7ccccc7)c6c5)ccc43)c2)cc1 ; c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1 ; c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1]

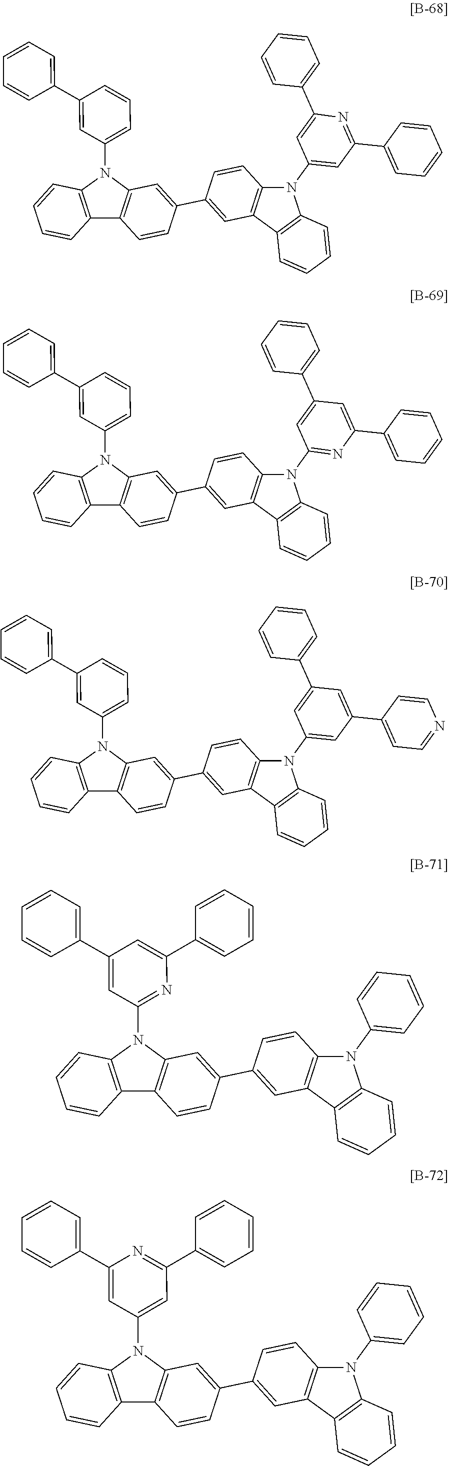 [SMILES: c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1 ; c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1]

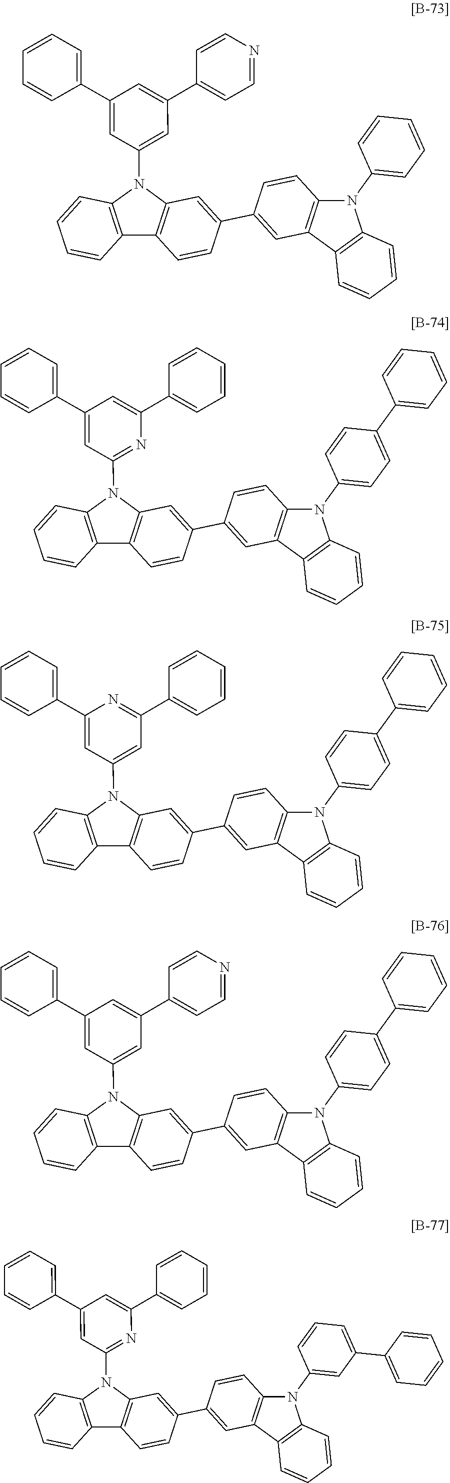 [SMILES: c1ccc(-c2cc(-c3ccncc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1 ; c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1]

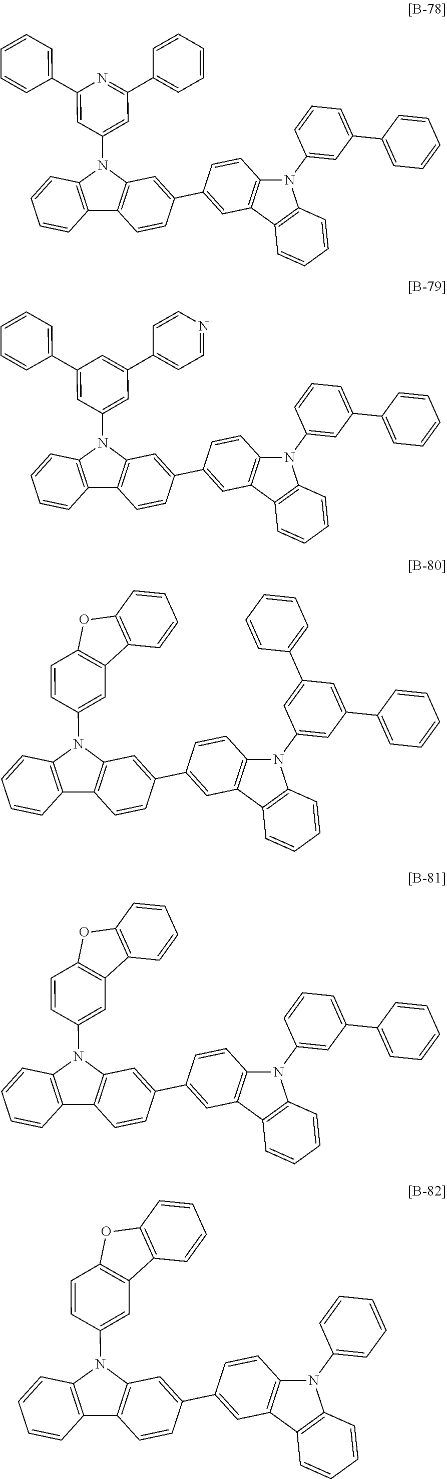 [SMILES: c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1 ; c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1]

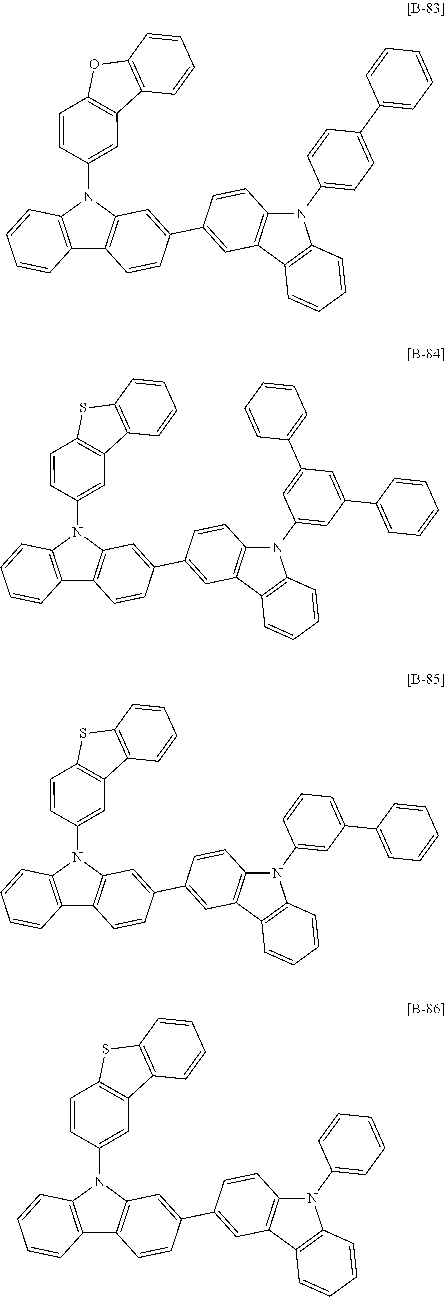 [SMILES: c1ccc(-c2ccc(-n3c4ccccc4c4cc(-c5ccc6c7ccccc7n(-c7ccc8oc9ccccc9c8c7)c6c5)ccc43)cc2)cc1 ; c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1 ; c1ccc(-n2c3ccccc3c3cc(-c4ccc5c6ccccc6n(-c6ccc7sc8ccccc8c7c6)c5c4)ccc32)cc1]

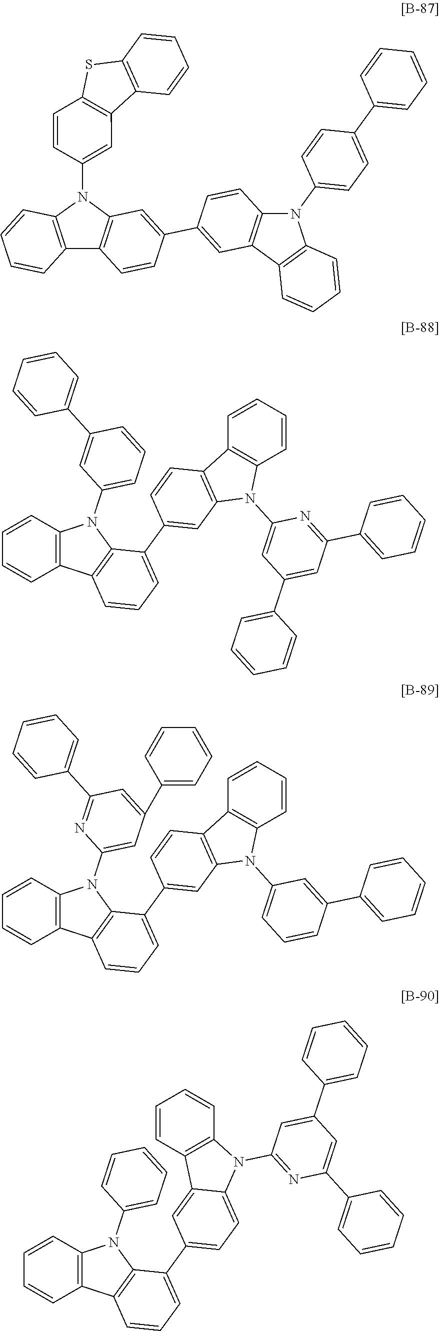 [SMILES: c1ccc(-c2ccc(-n3c4ccccc4c4cc(-c5ccc6c7ccccc7n(-c7ccc8sc9ccccc9c8c7)c6c5)ccc43)cc2)cc1 ; c1ccc(-c2cccc(-n3c4ccccc4c4cccc(-c5ccc6c7ccccc7n(-c7cc(-c8ccccc8)cc(-c8ccccc8)n7)c6c5)c43)c2)cc1 ; c1ccc(-c2cccc(-n3c4ccccc4c4ccc(-c5cccc6c7ccccc7n(-c7cc(-c8ccccc8)cc(-c8ccccc8)n7)c56)cc43)c2)cc1 ; c1ccc(-c2cc(-c3ccccc3)nc(-n3c4ccccc4c4cc(-c5cccc6c7ccccc7n(-c7ccccc7)c56)ccc43)c2)cc1]

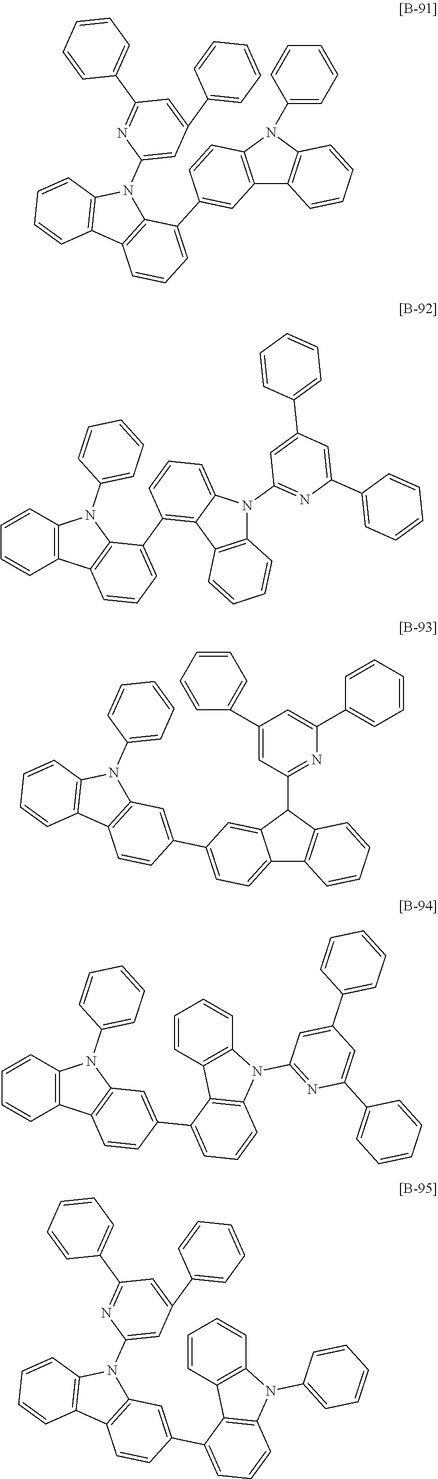 [SMILES: c1ccc(-c2cc(-c3ccccc3)nc(-n3c4ccccc4c4cccc(-c5ccc6c(c5)c5ccccc5n6-c5ccccc5)c43)c2)cc1 ; c1ccc(-c2cc(-c3ccccc3)nc(-n3c4ccccc4c4c(-c5cccc6c7ccccc7n(-c7ccccc7)c56)cccc43)c2)cc1 ; c1ccc(-c2cc(-c3ccccc3)nc(C3c4ccccc4-c4ccc(-c5ccc6c7ccccc7n(-c7ccccc7)c6c5)cc43)c2)cc1 ; c1ccc(-c2cc(-c3ccccc3)nc(-n3c4ccccc4c4c(-c5ccc6c7ccccc7n(-c7ccccc7)c6c5)cccc43)c2)cc1 ; c1ccc(-c2cc(-c3ccccc3)nc(-n3c4ccccc4c4ccc(-c5cccc6c5c5ccccc5n6-c5ccccc5)cc43)c2)cc1]

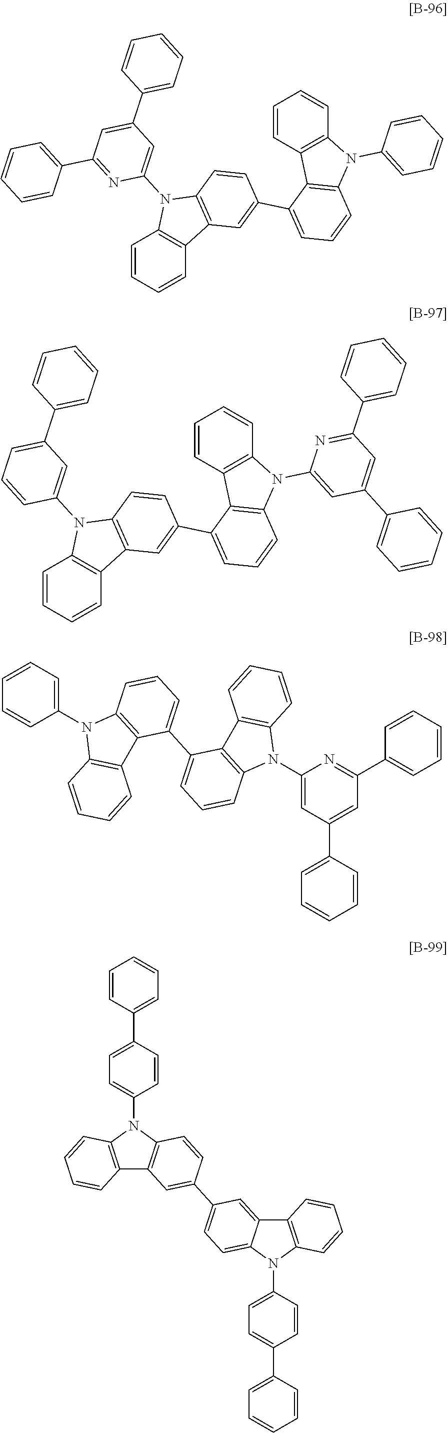 [SMILES: c1ccc(-c2cc(-c3ccccc3)nc(-n3c4ccccc4c4cc(-c5cccc6c5c5ccccc5n6-c5ccccc5)ccc43)c2)cc1 ; c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

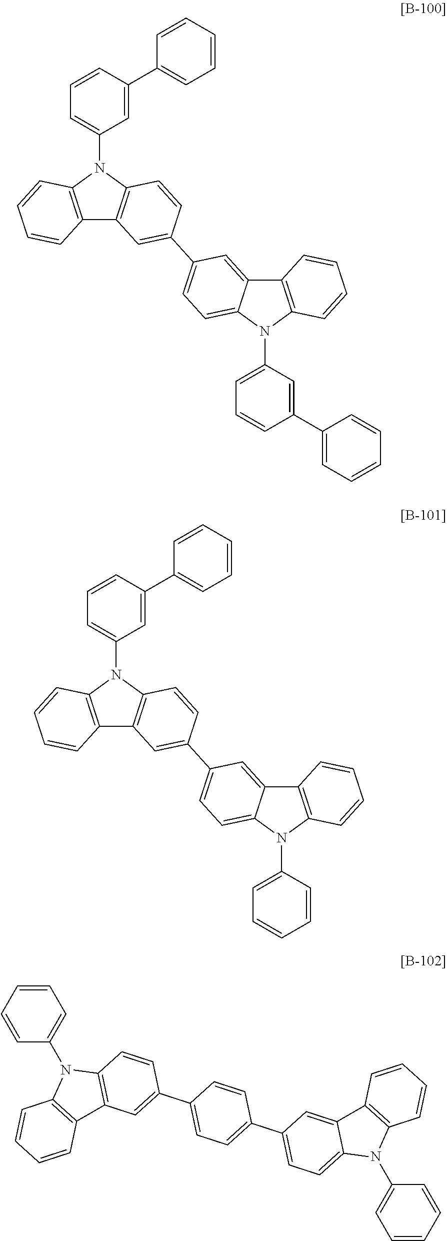 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1]

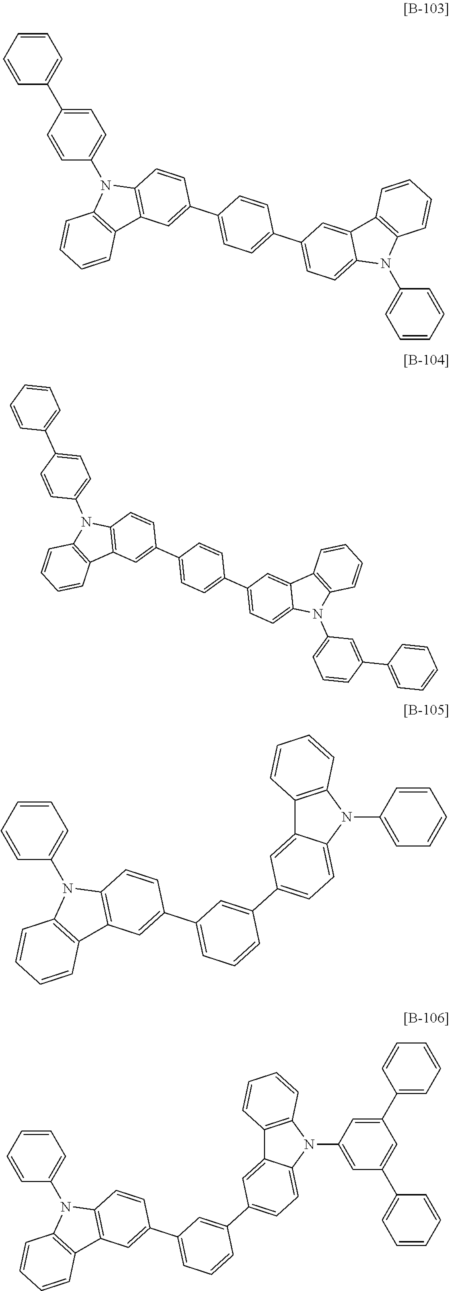 [SMILES: c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1 ; c1ccc(-n2c3ccccc3c3cc(-c4cccc(-c5ccc6c(c5)c5ccccc5n6-c5ccccc5)c4)ccc32)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1]

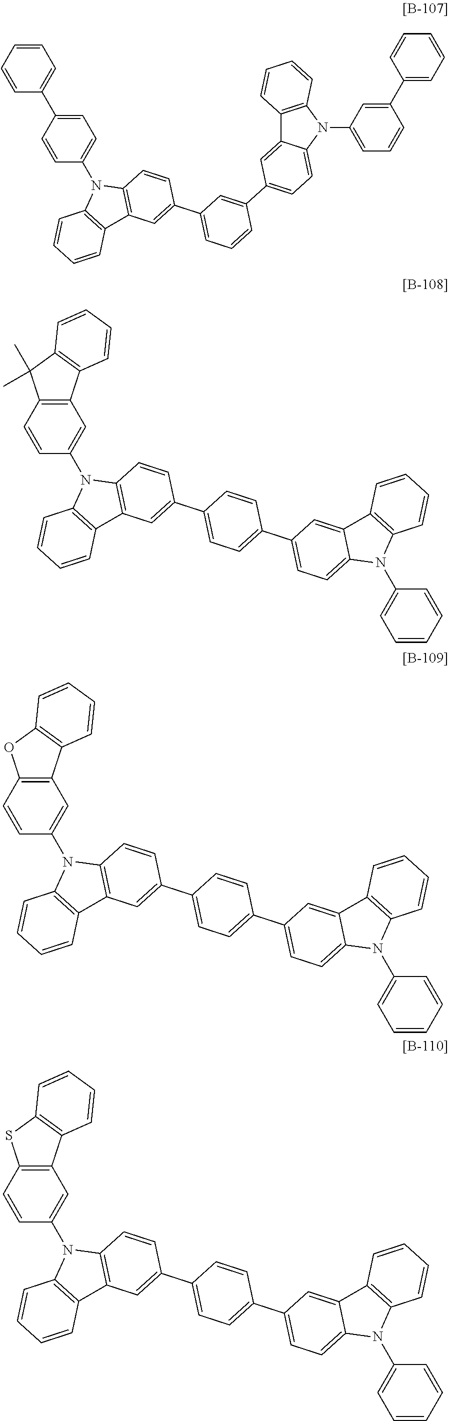 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccc(-c6ccc7c(c6)c6ccccc6n7-c6ccccc6)cc5)ccc43)ccc21 ; c1ccc(-n2c3ccccc3c3cc(-c4ccc(-c5ccc6c(c5)c5ccccc5n6-c5ccc6oc7ccccc7c6c5)cc4)ccc32)cc1 ; c1ccc(-n2c3ccccc3c3cc(-c4ccc(-c5ccc6c(c5)c5ccccc5n6-c5ccc6sc7ccccc7c6c5)cc4)ccc32)cc1]

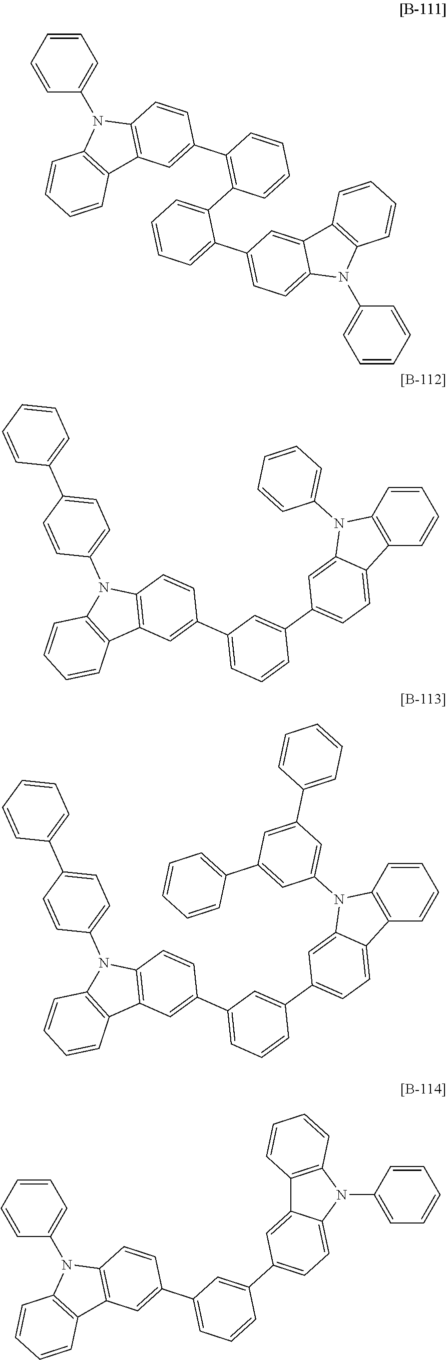 [SMILES: c1ccc(-n2c3ccccc3c3cc(-c4ccccc4-c4ccccc4-c4ccc5c(c4)c4ccccc4n5-c4ccccc4)ccc32)cc1 ; c1ccc(-c2ccc(-n3c4ccccc4c4cc(-c5cccc(-c6ccc7c8ccccc8n(-c8ccccc8)c7c6)c5)ccc43)cc2)cc1 ; c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1 ; c1ccc(-n2c3ccccc3c3cc(-c4cccc(-c5ccc6c(c5)c5ccccc5n6-c5ccccc5)c4)ccc32)cc1]

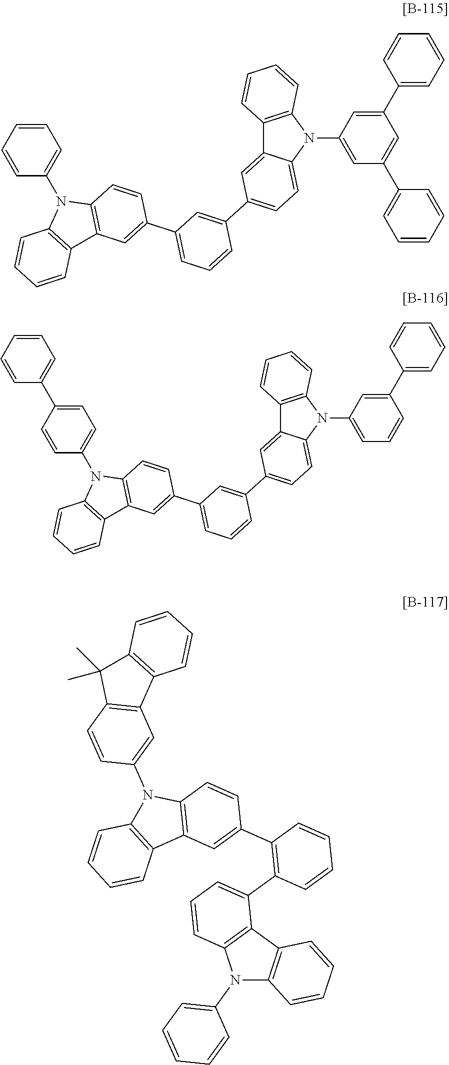 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccccc5-c5cccc6c5c5ccccc5n6-c5ccccc5)ccc43)ccc21]

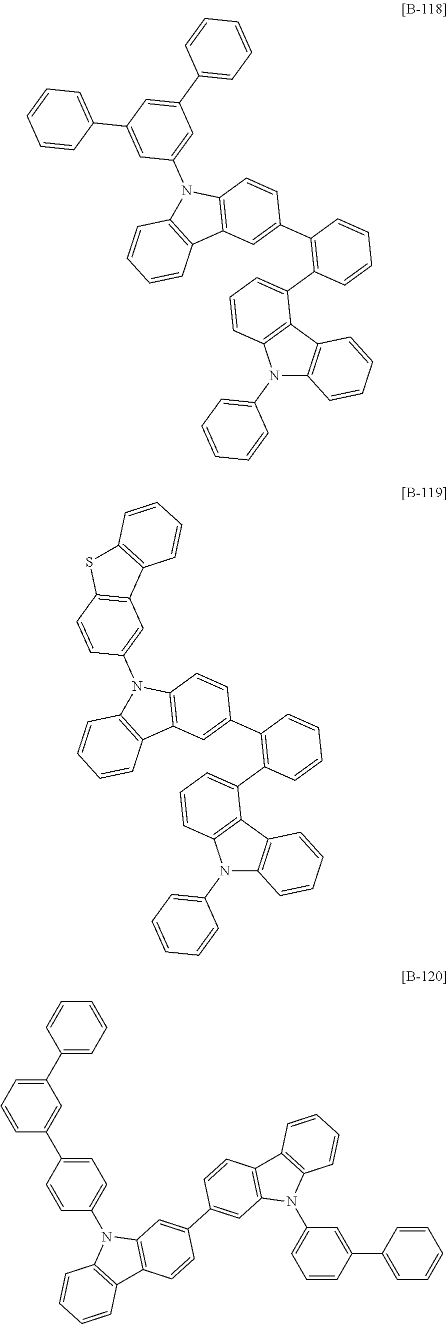 [SMILES: c1ccc(-c2cc(-c3ccccc3)cc(-n3c4ccccc4c4cc(-c5ccccc5-c5cccc6c5c5ccccc5n6-c5ccccc5)ccc43)c2)cc1 ; c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

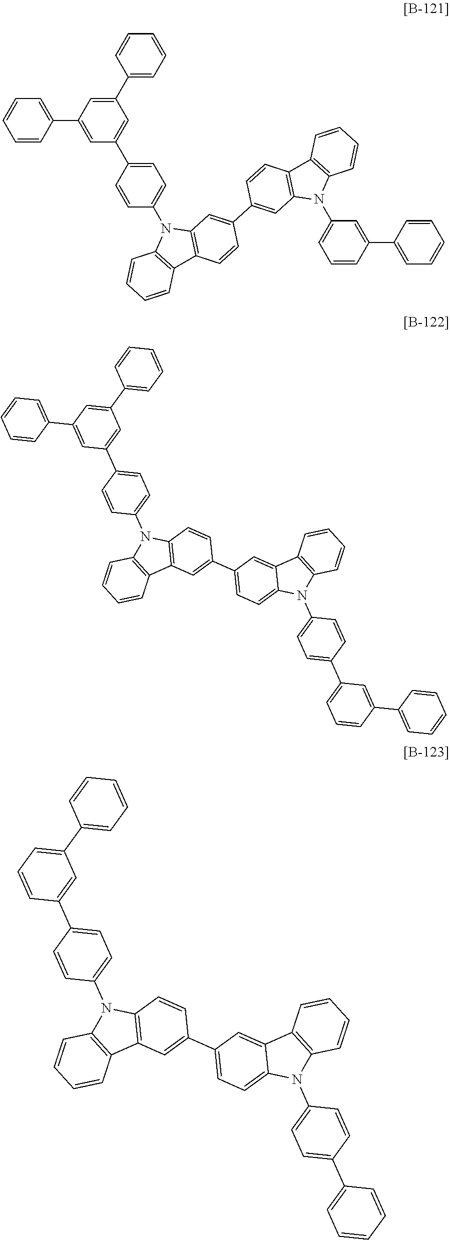 [SMILES: c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

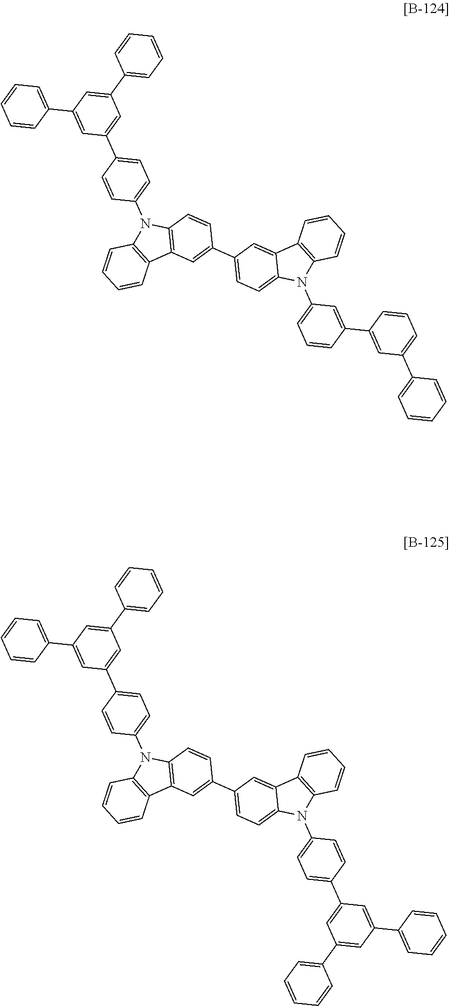 [SMILES: c1ccc(-c2cccc(-c3cccc(-n4c5ccccc5c5cc(-c6ccc7c(c6)c6ccccc6n7-c6ccc(-c7cc(-c8ccccc8)cc(-c8ccccc8)c7)cc6)ccc54)c3)c2)cc1 ; c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

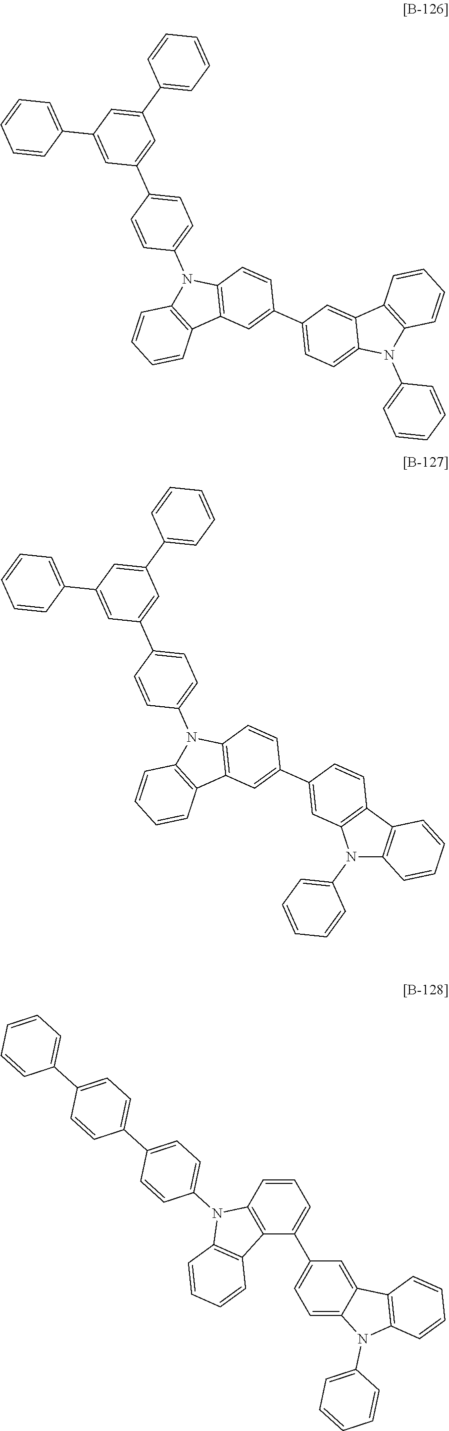 [SMILES: c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1 ; c1ccc(-c2cc(-c3ccccc3)cc(-c3ccc(-n4c5ccccc5c5cc(-c6ccc7c8ccccc8n(-c8ccccc8)c7c6)ccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1]

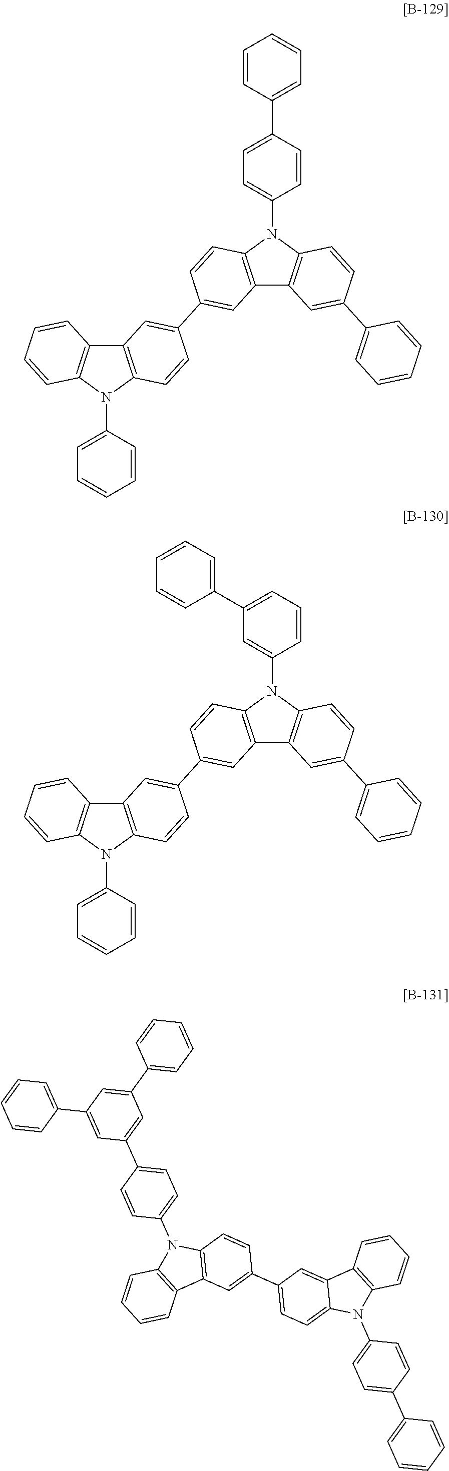 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1]

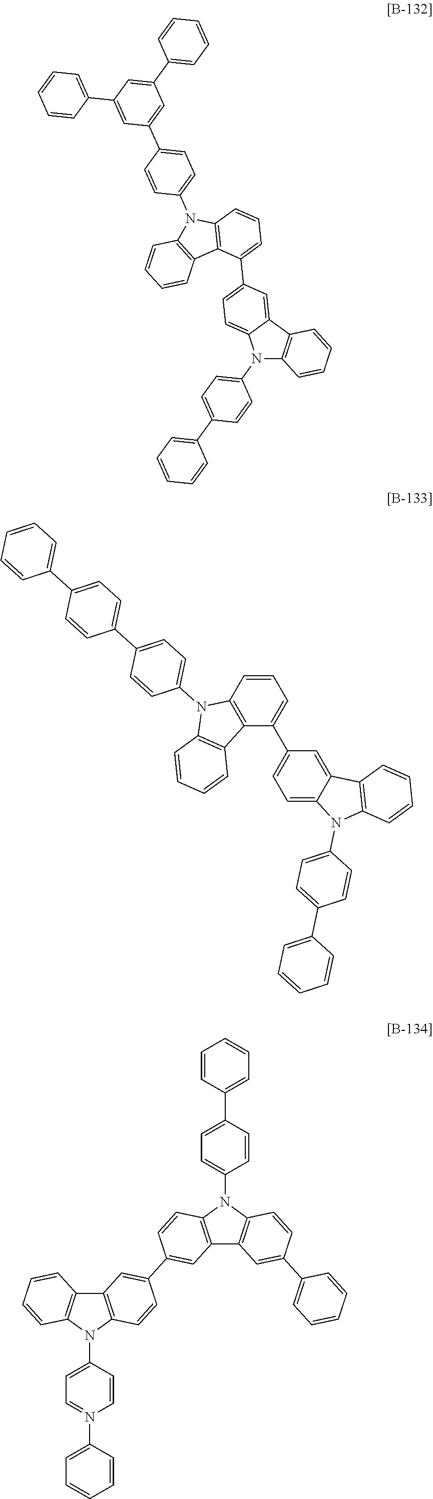

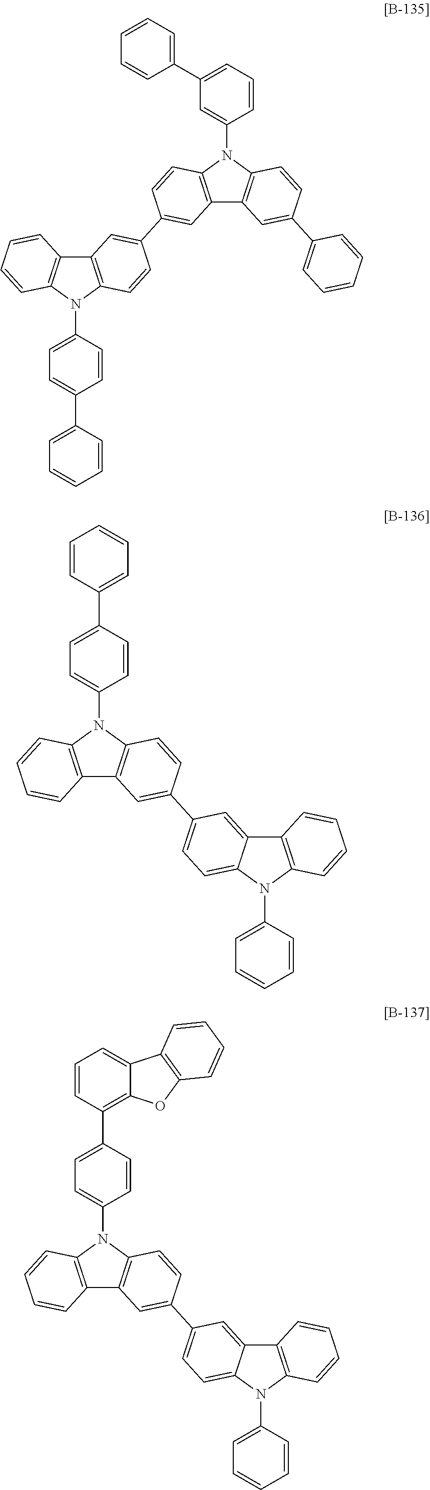 [SMILES: c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1]

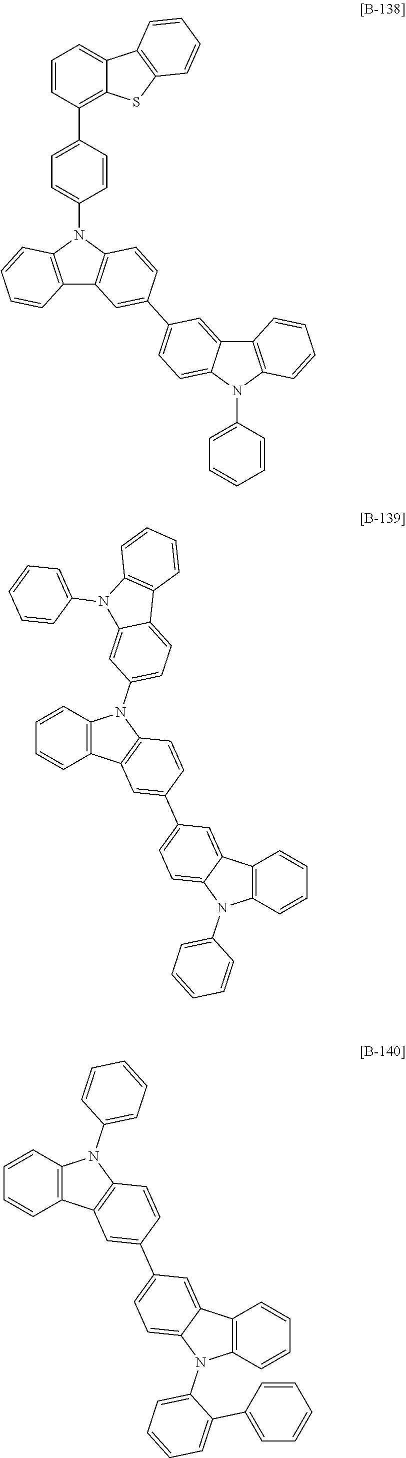 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccccc2-n2c3ccccc3c3cc(-c4ccc5c(c4)c4ccccc4n5-c4ccccc4)ccc32)cc1]

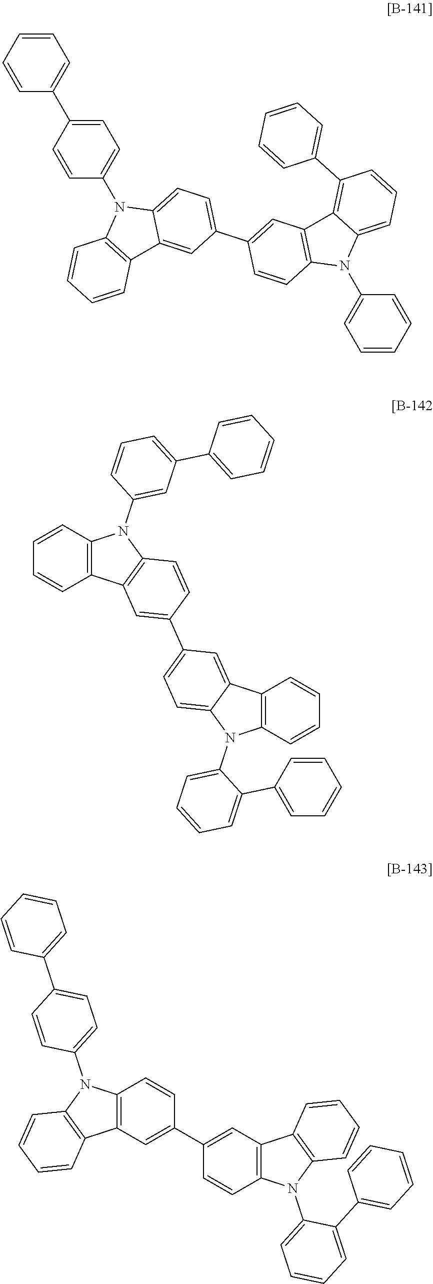 [SMILES: c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

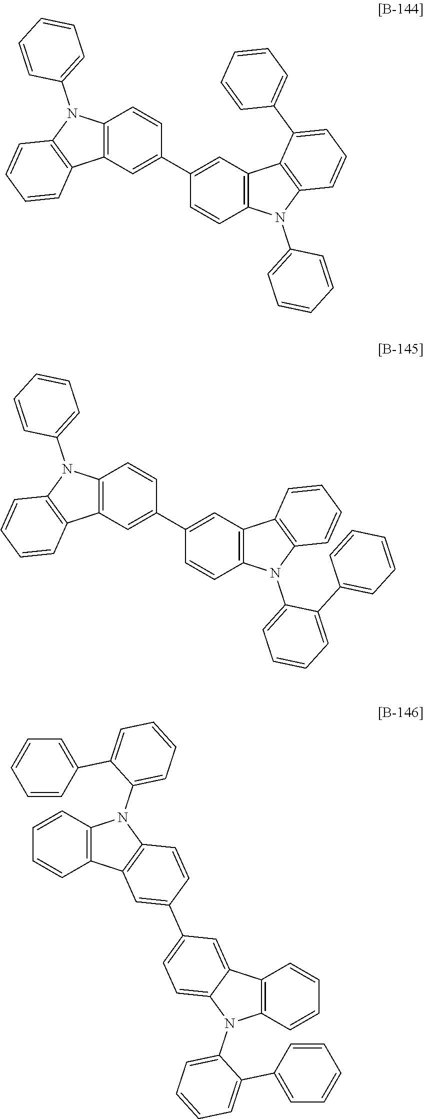 [SMILES: c1ccc(-c2cccc3c2c2cc(-c4ccc5c(c4)c4ccccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4-c4ccccc4)ccc32)cc1]

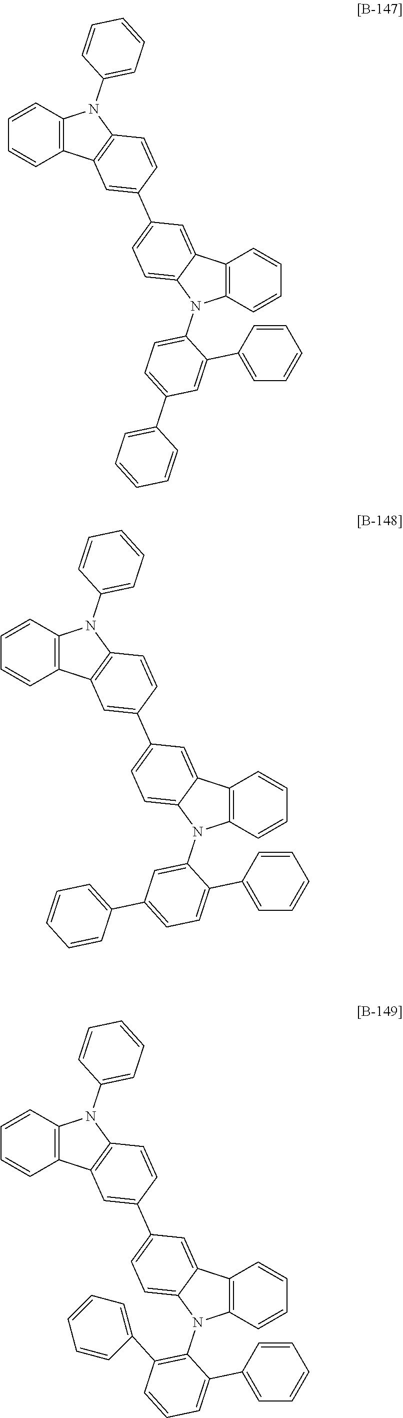

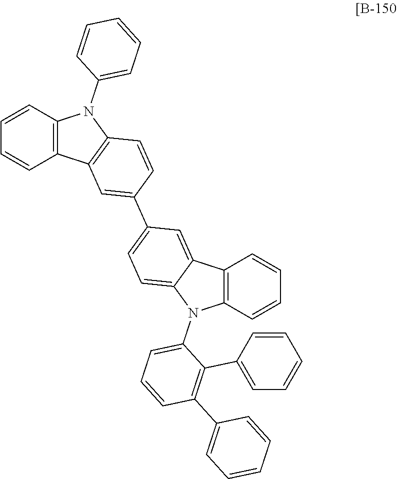 [SMILES: c1ccc(-c2cccc(-n3c4ccccc4c4cc(-c5ccc6c(c5)c5ccccc5n6-c5ccccc5)ccc43)c2-c2ccccc2)cc1]

Examples of Compound B-1 to Compound B-150 from Group 2 in which at least one hydrogen is replaced with deuterium are below.

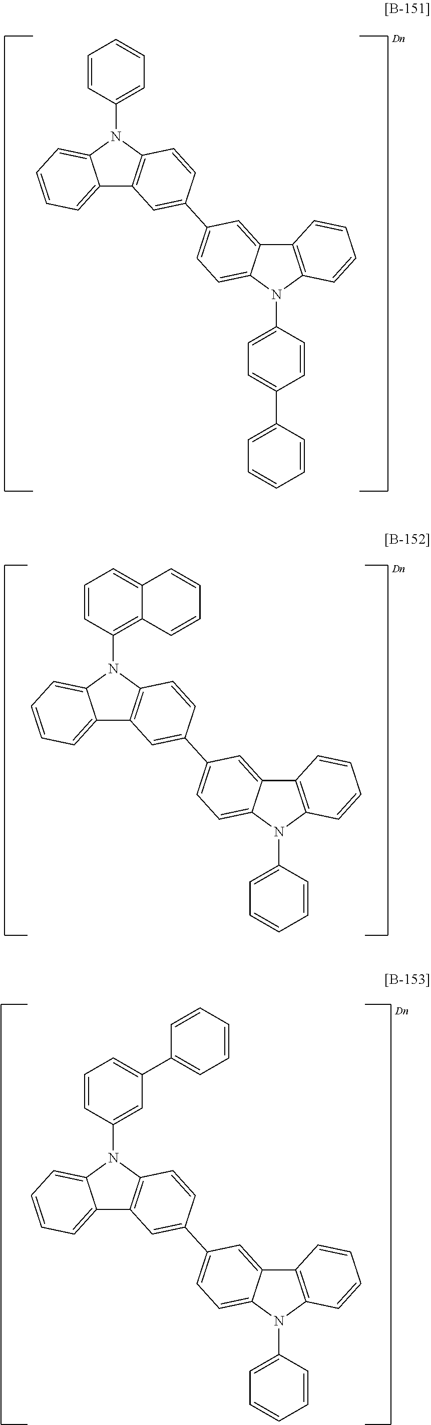

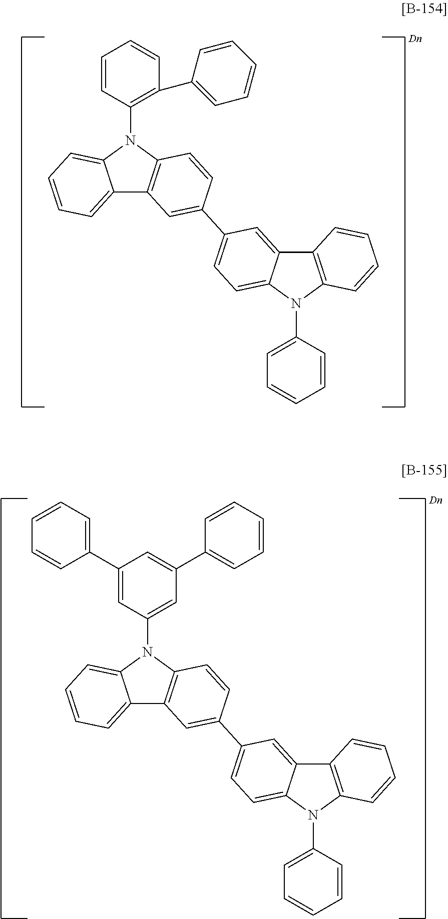

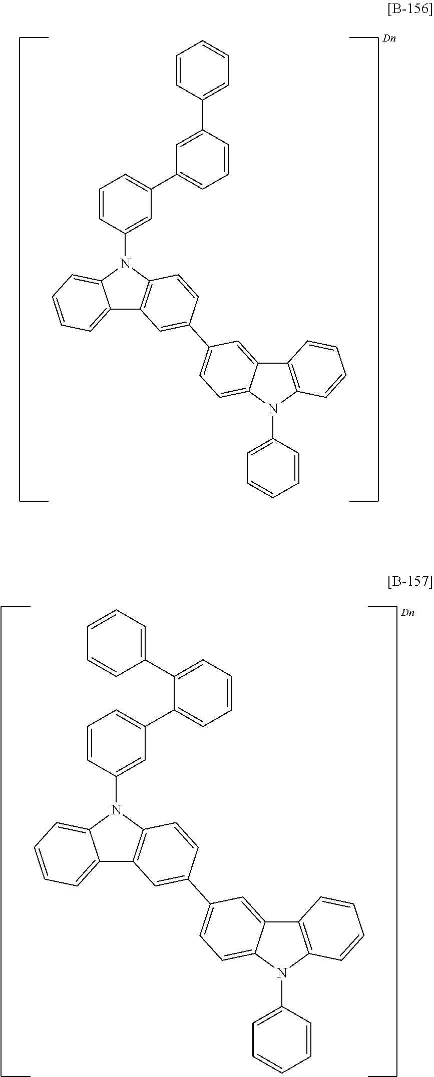

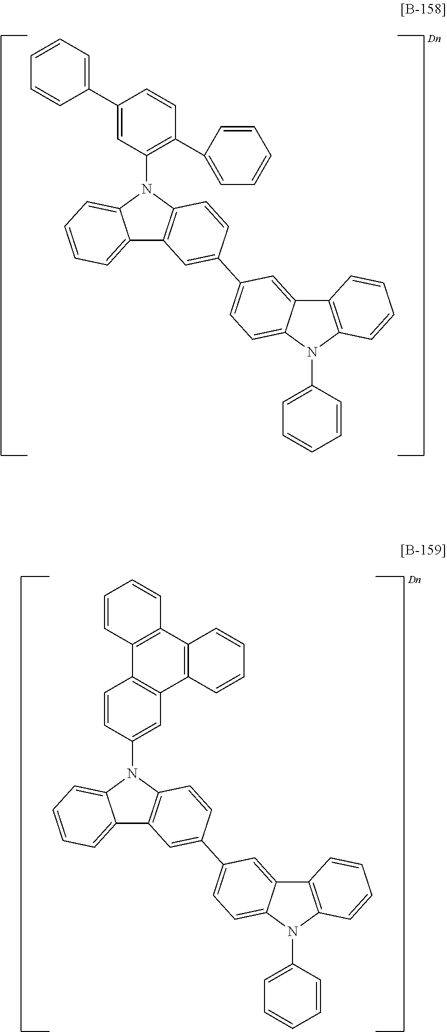

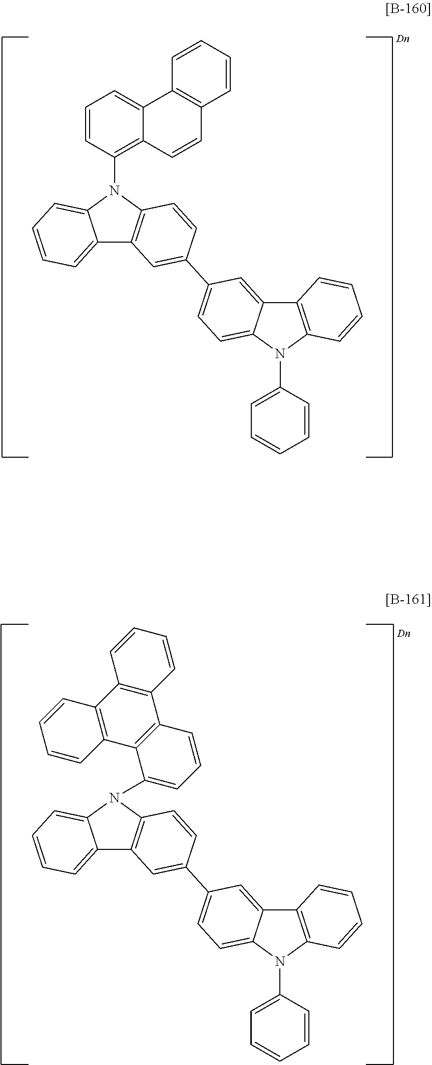

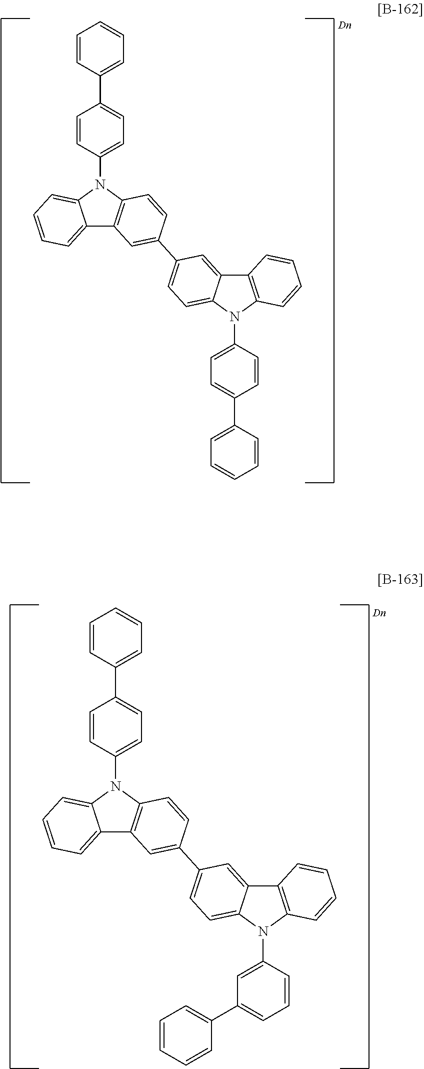

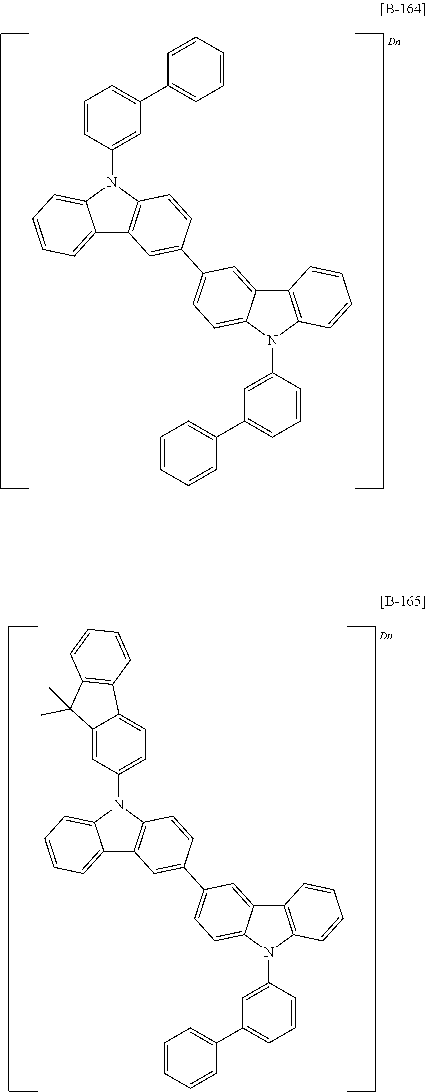

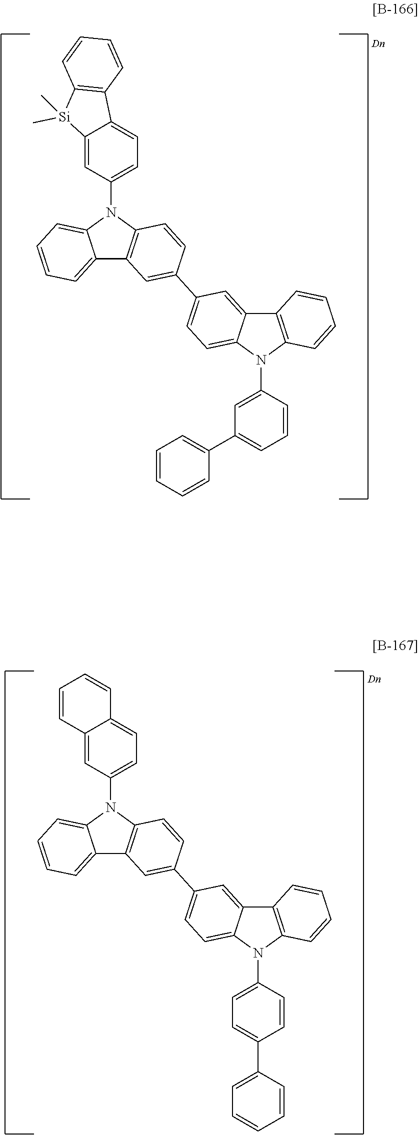

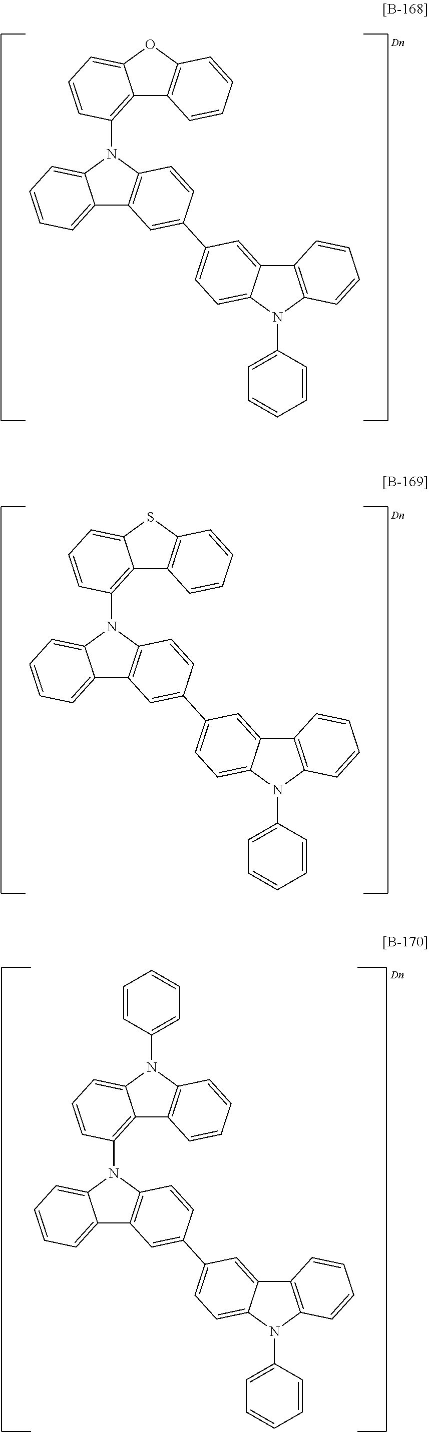

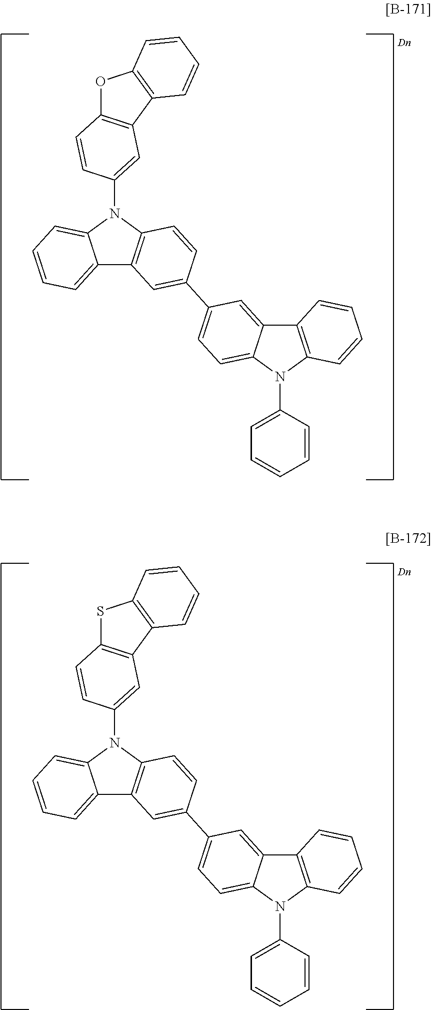

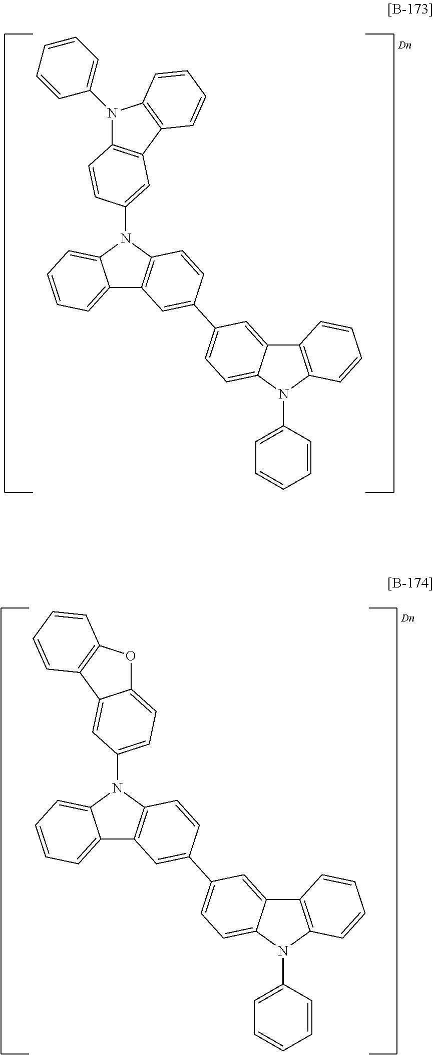

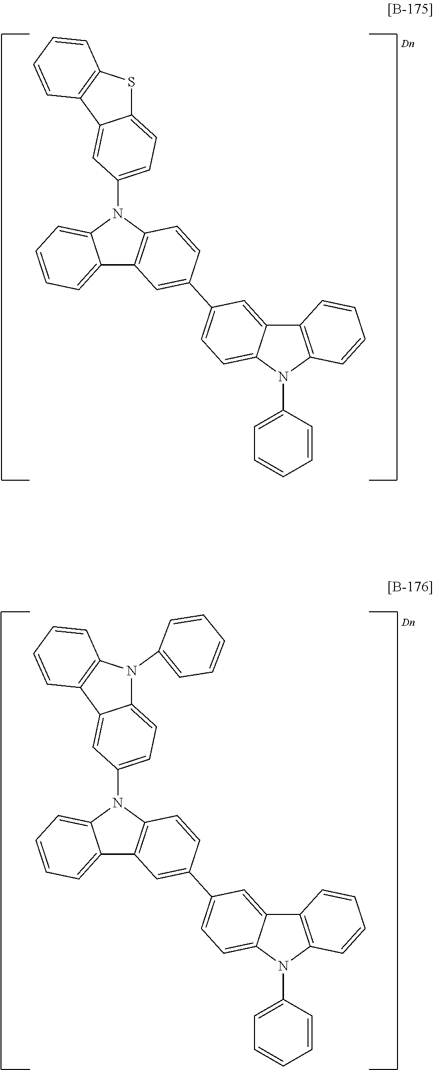

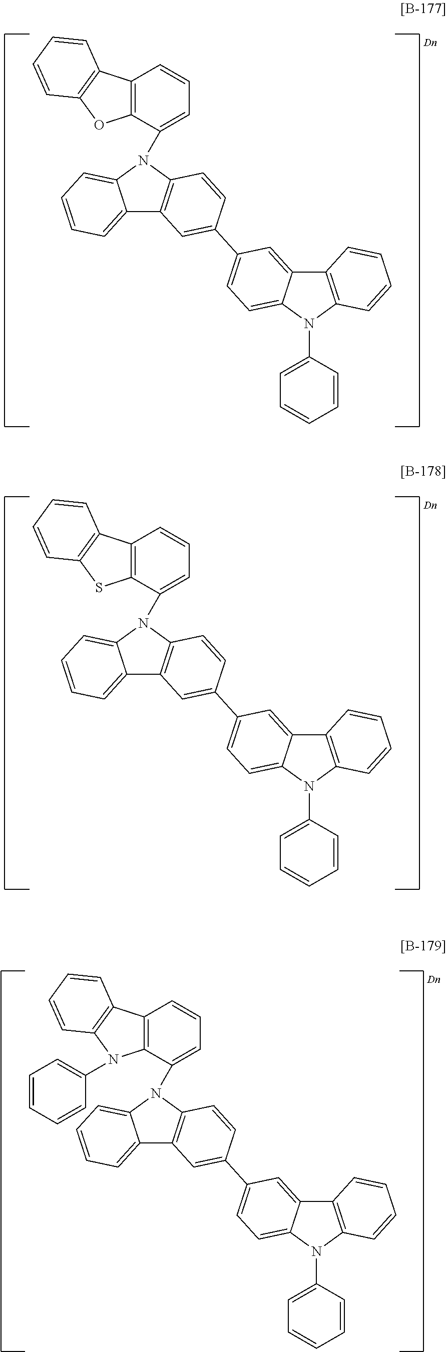

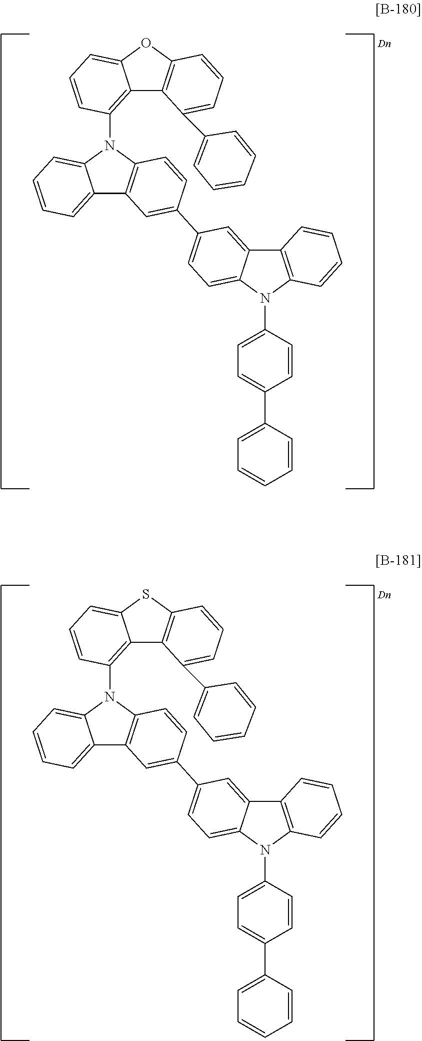

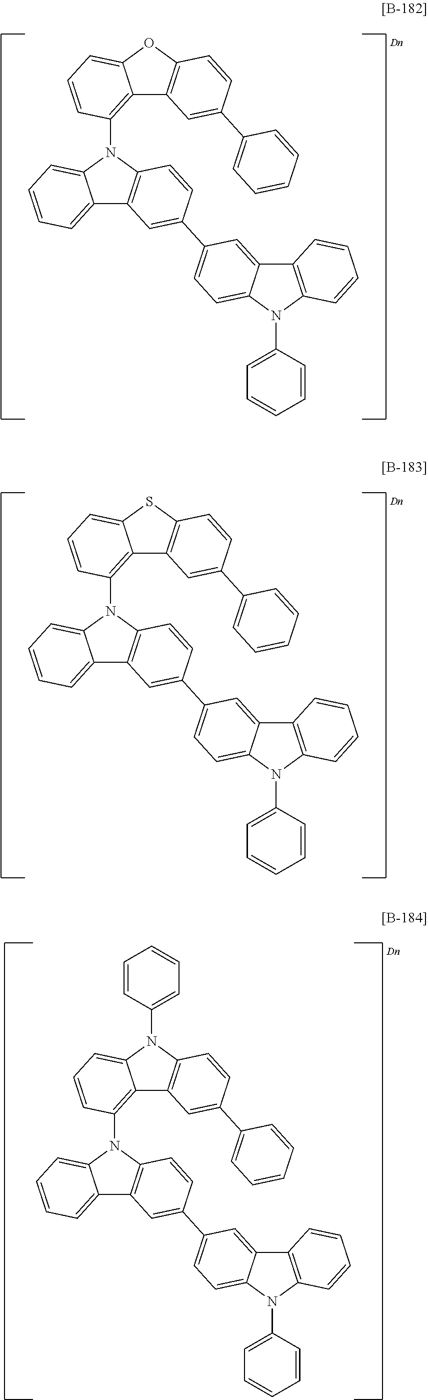

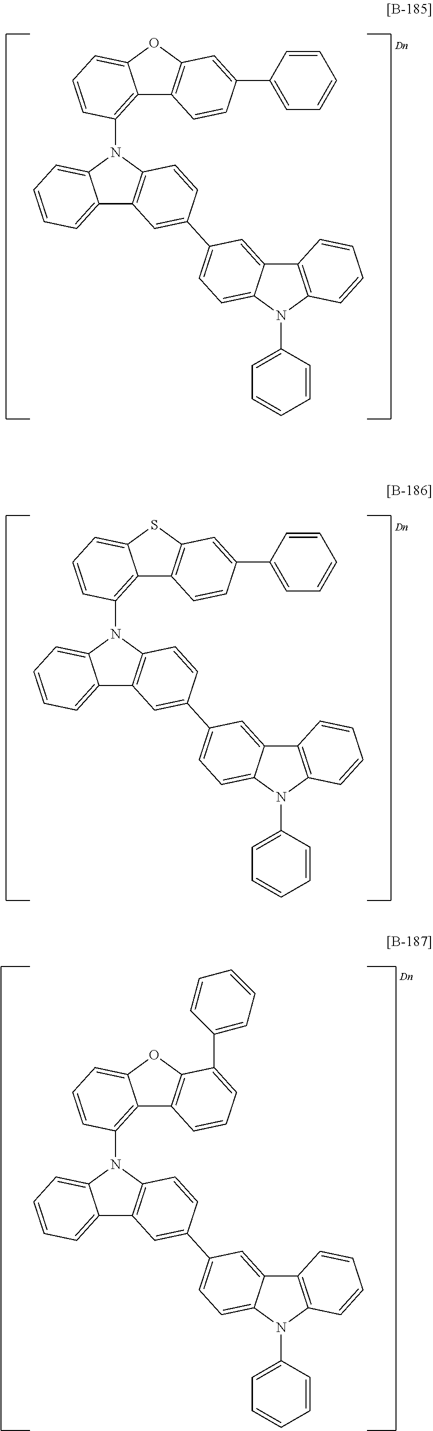

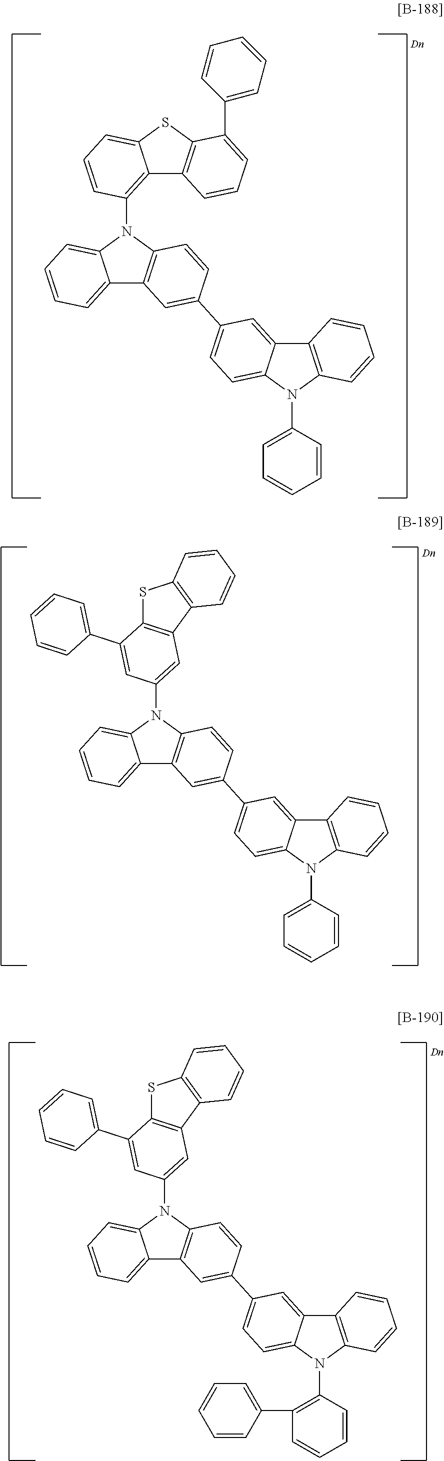

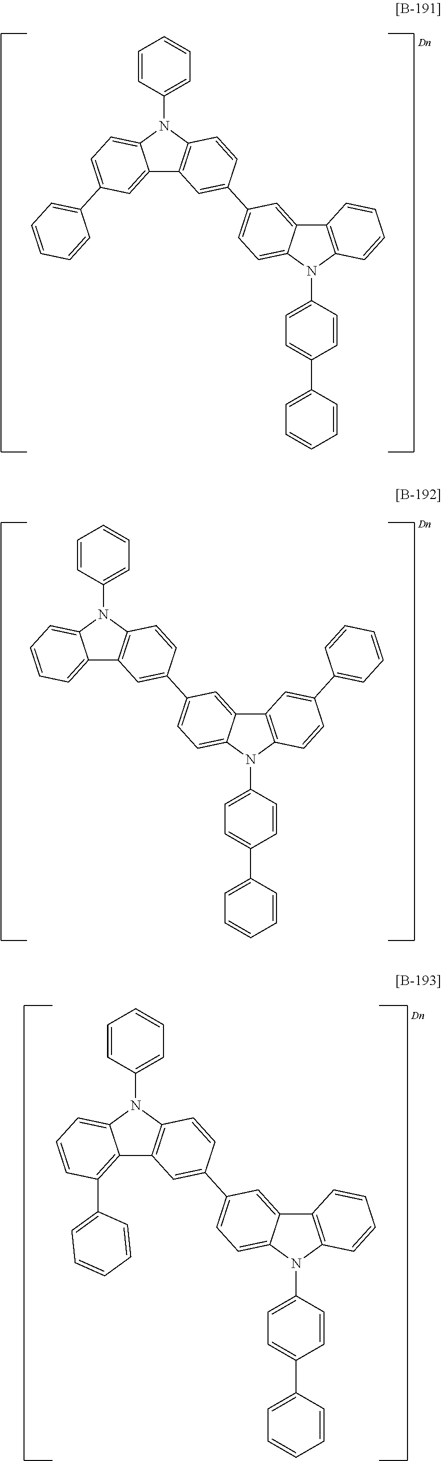

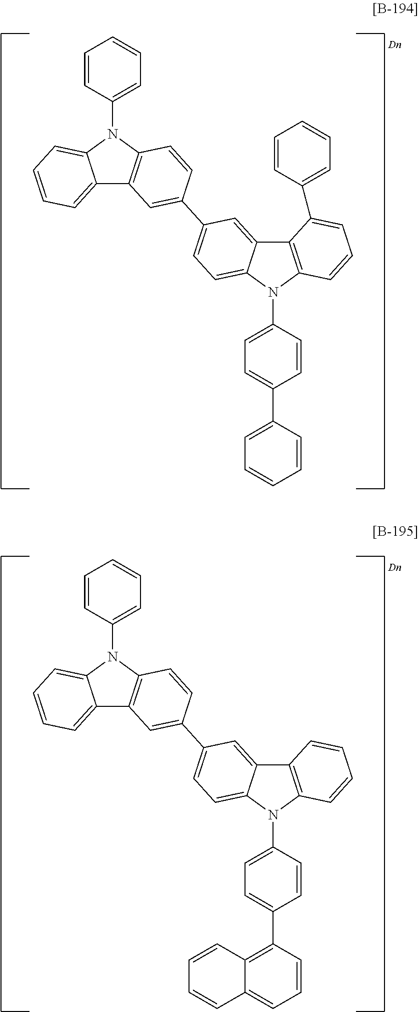

(Dn refers to the number of deuterium substitutions and indicates a structure substituted with one or more deuterium atoms)

The most specific structures for Compound B-151 to Compound B-195 of Group 2 are presented below as examples according to the position and substitution ratio of deuterium substitution.

In an implementation, deuterium may be substituted, and the deuterium substitution position, deuterium substitution ratio, or the like may include all changeable ranges within the range of Compound B-1 to Compound B-195.

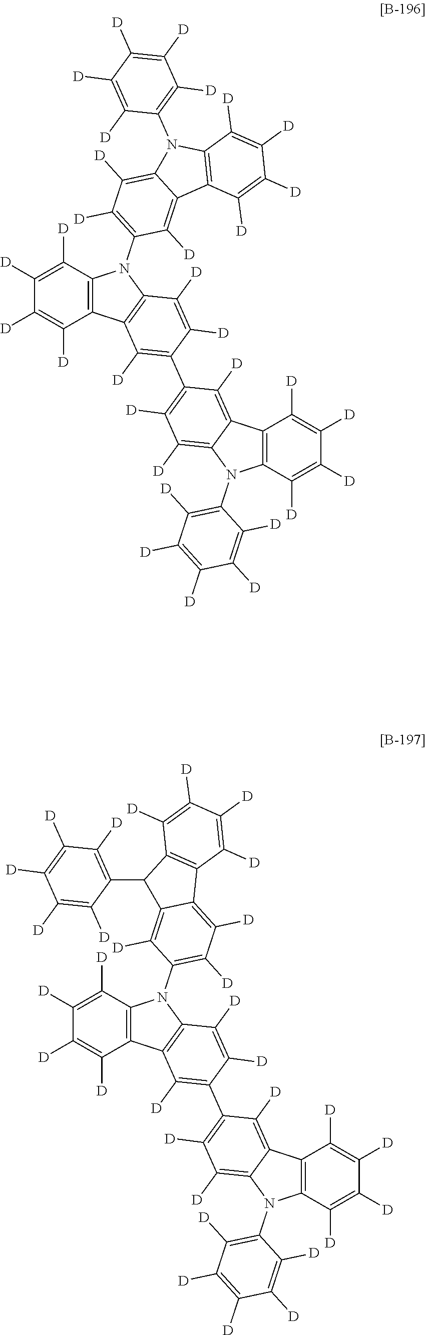

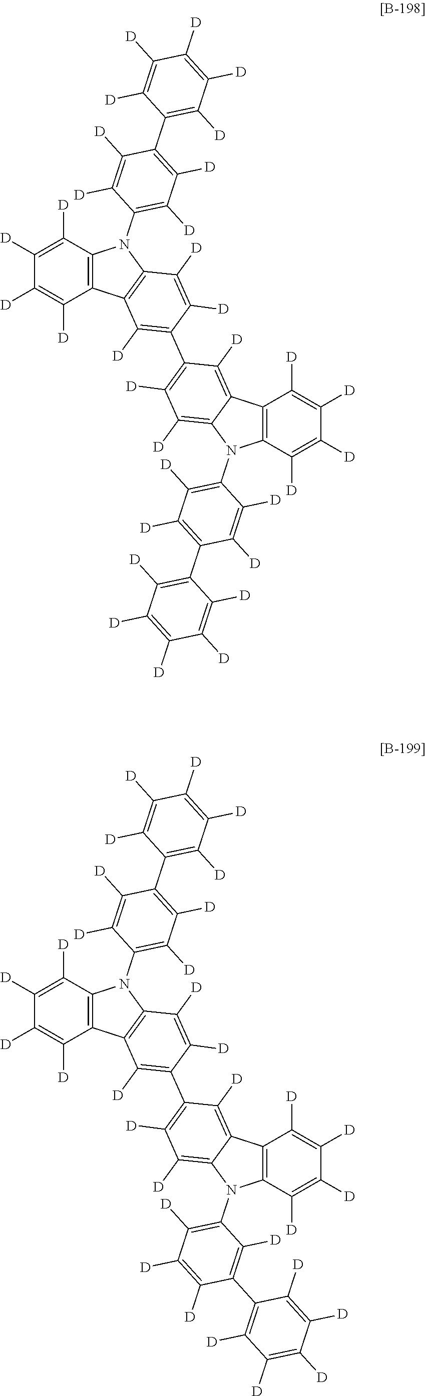

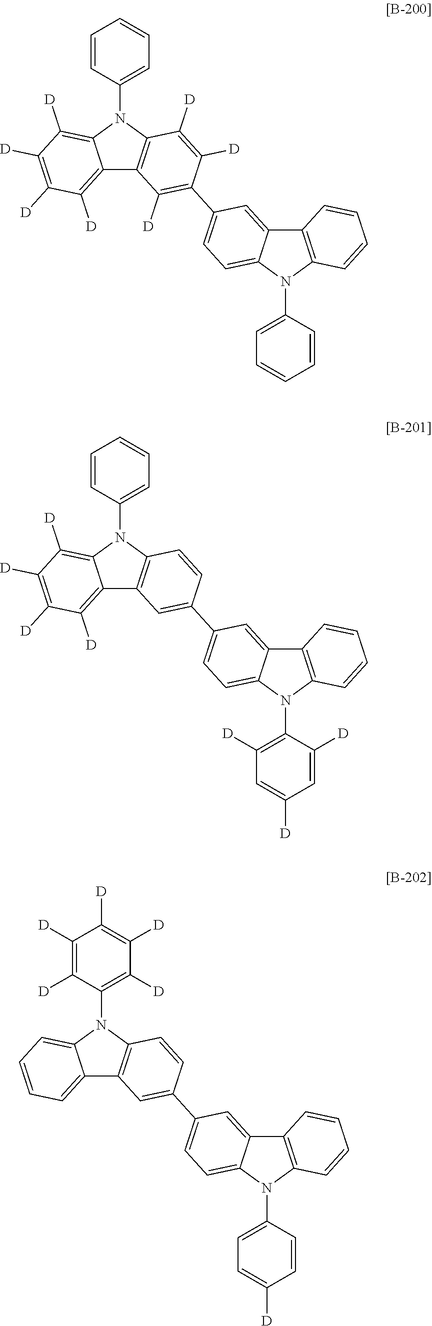 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccccc1 ; [2H]c1cc([2H])c(-n2c3ccccc3c3cc(-c4ccc5c(c4)c4c([2H])c([2H])c([2H])c([2H])c4n5-c4ccccc4)ccc32)c([2H])c1 ; [2H]c1ccc(-n2c3ccccc3c3cc(-c4ccc5c(c4)c4ccccc4n5-c4c([2H])c([2H])c([2H])c([2H])c4[2H])ccc32)cc1]

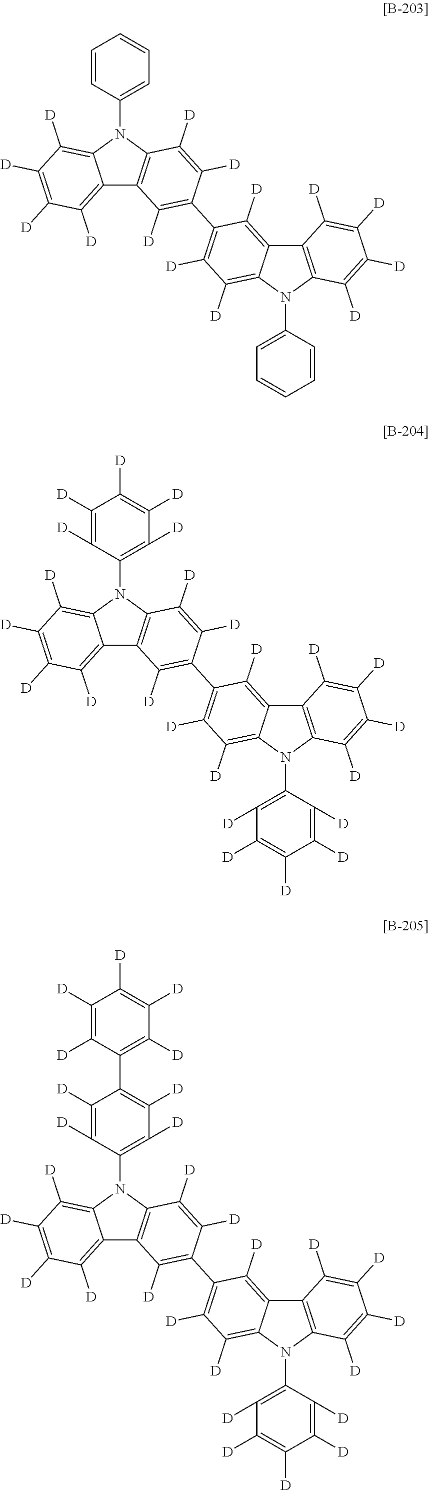

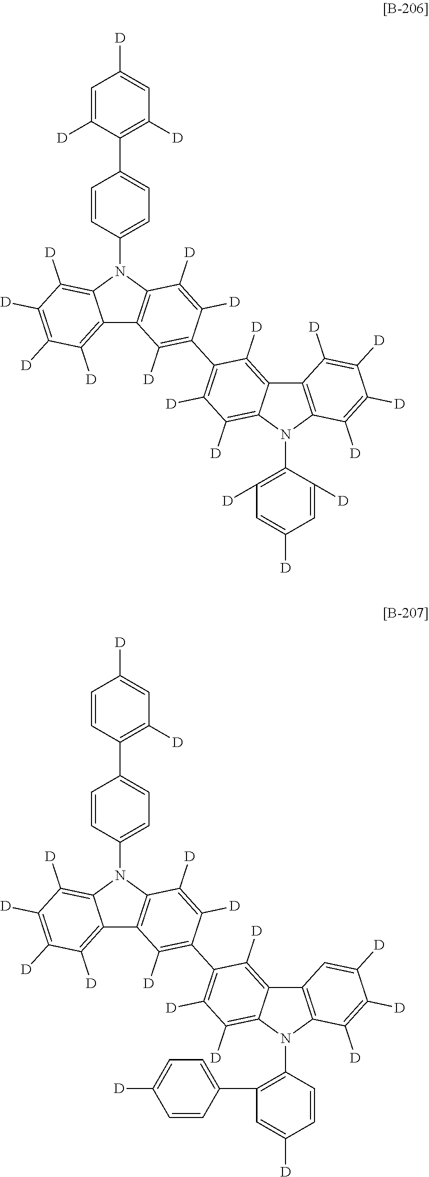 [SMILES: [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1 ; [2H]c1ccc(-c2cc([2H])ccc2-n2c3c([2H])c([2H])c([2H])cc3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1]

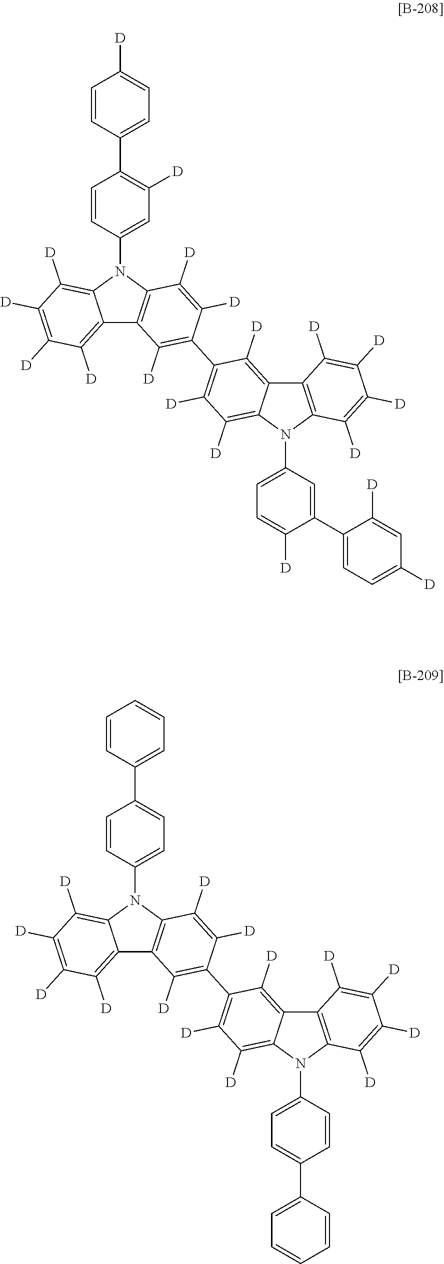 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1]

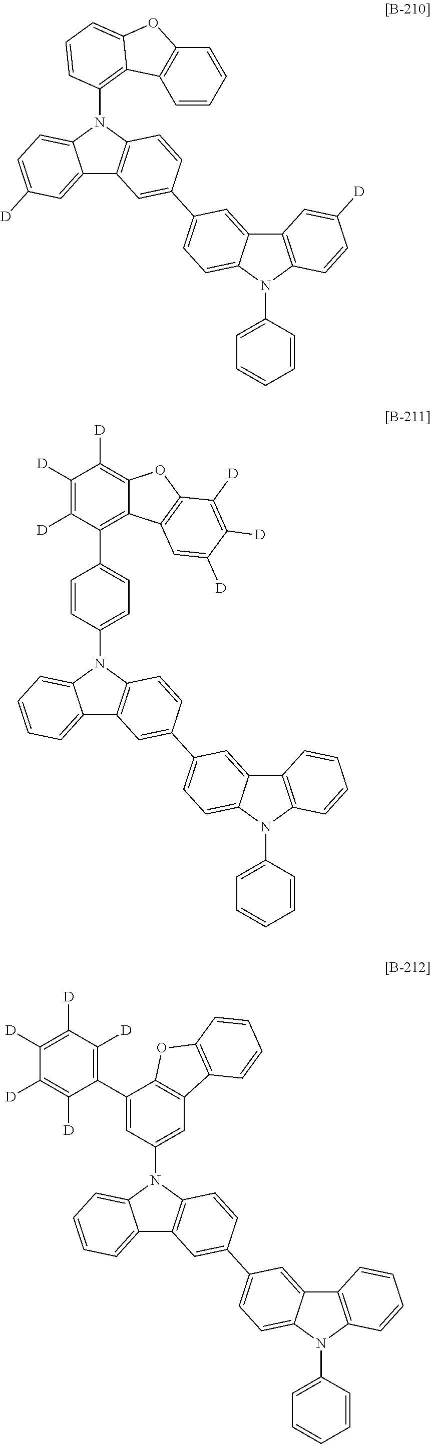 [SMILES: [2H]c1ccc2c(c1)c1cc(-c3ccc4c(c3)c3cc([2H])ccc3n4-c3cccc4oc5ccccc5c34)ccc1n2-c1ccccc1 ; [2H]c1cc2c(oc3c([2H])c([2H])c([2H])c(-c4ccc(-n5c6ccccc6c6cc(-c7ccc8c(c7)c7ccccc7n8-c7ccccc7)ccc65)cc4)c32)c([2H])c1[2H] ; [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H]]

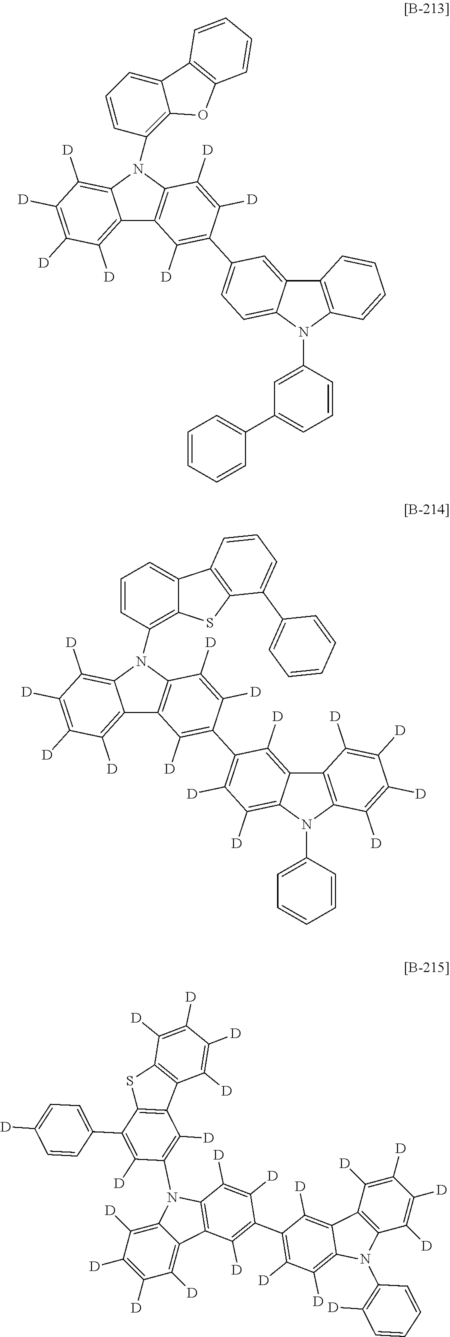

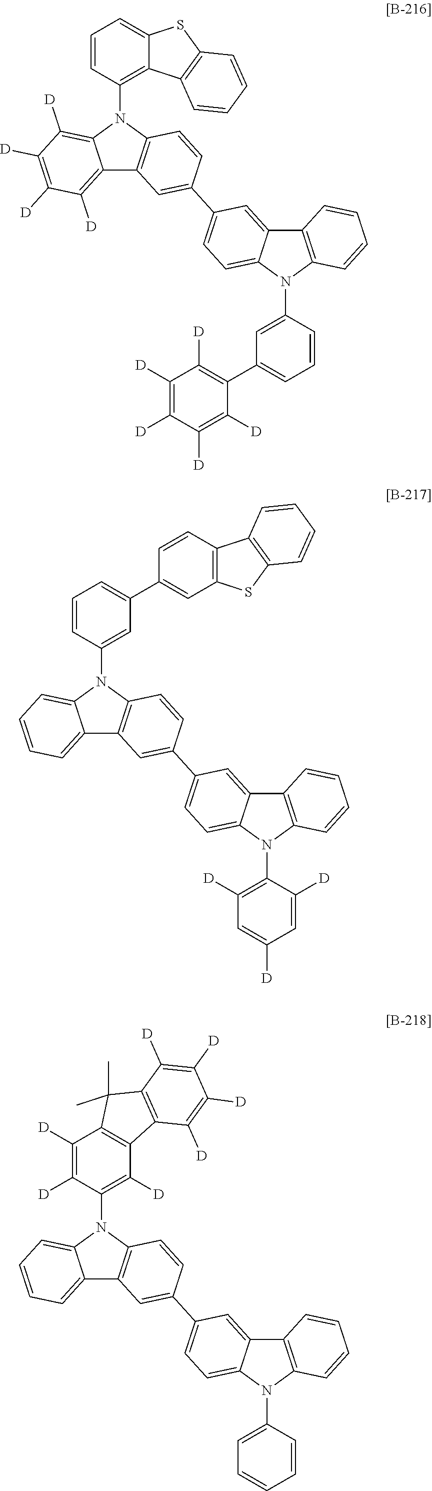 [SMILES: [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1 ; [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C]

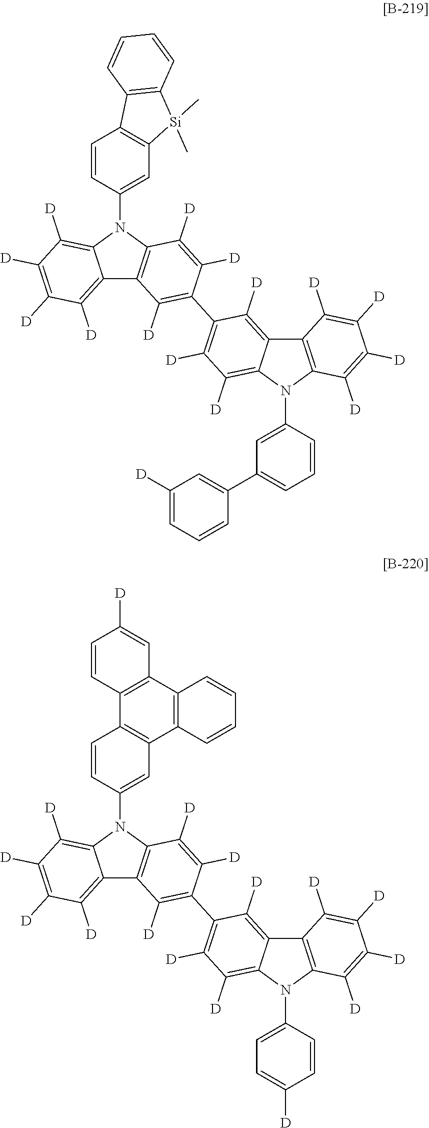 [SMILES: [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1 ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1]

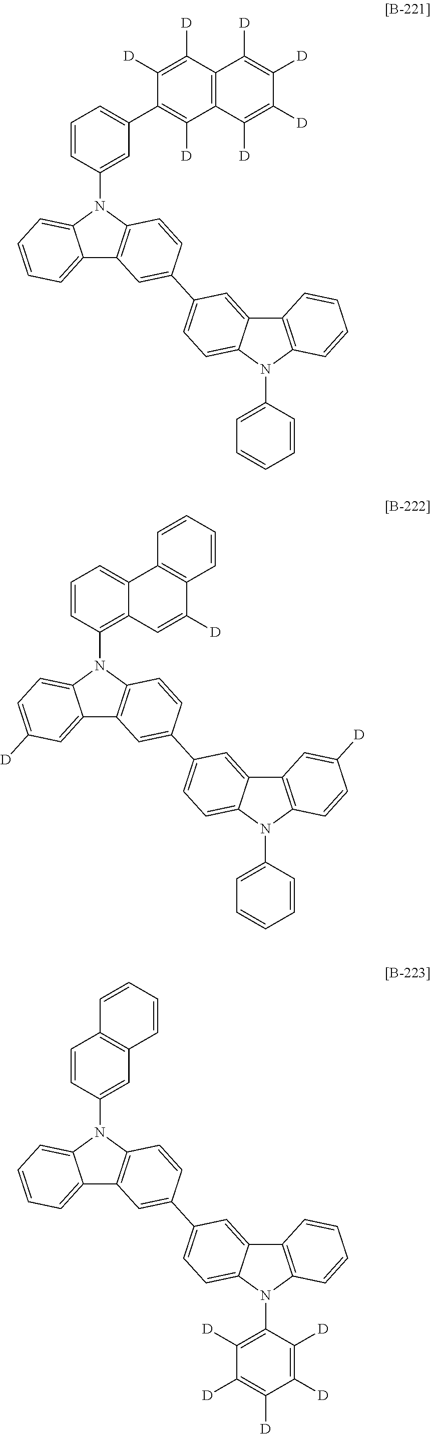 [SMILES: [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H] ; [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H]]

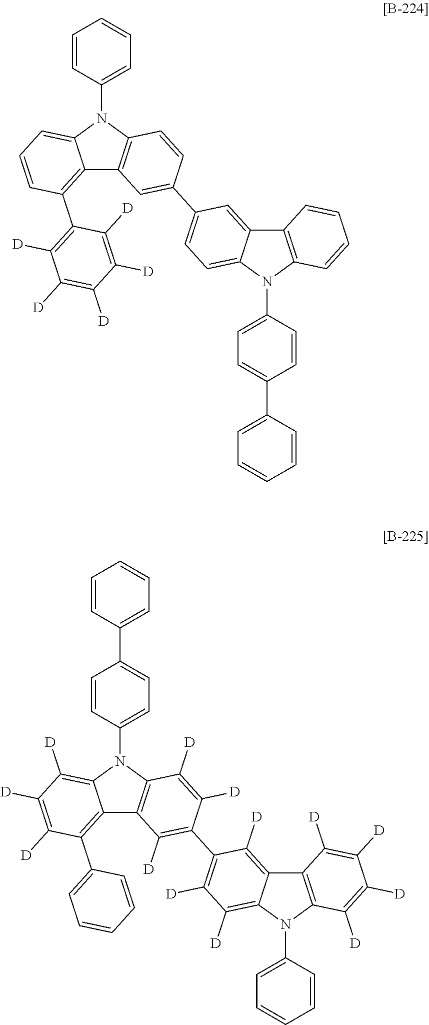 [SMILES: [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1]

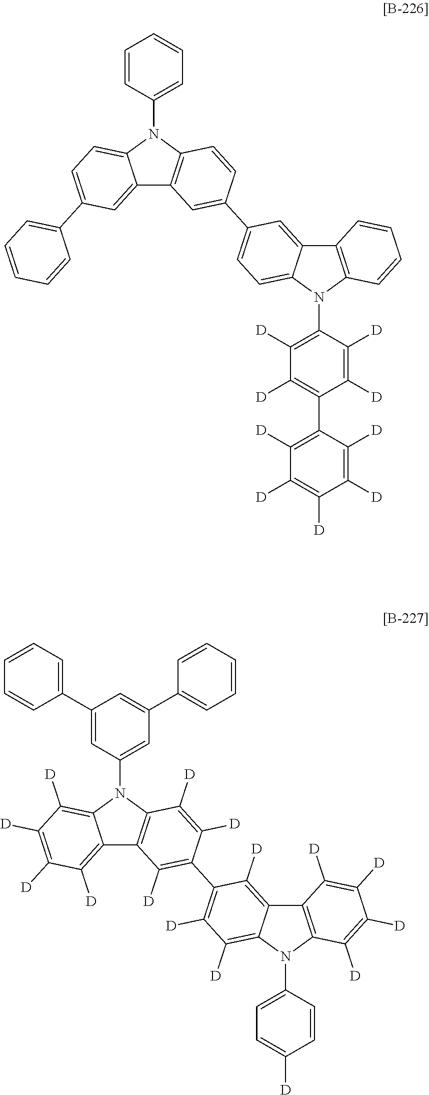 [SMILES: [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H] ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1]

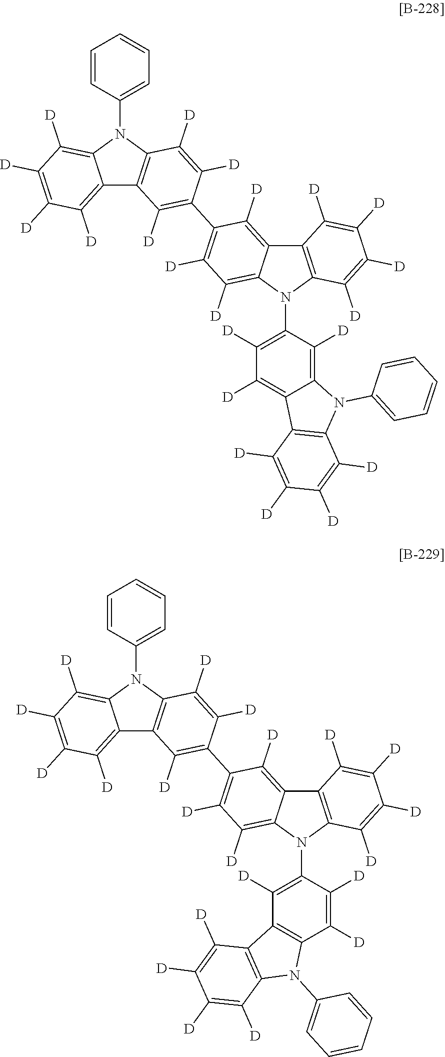 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1]

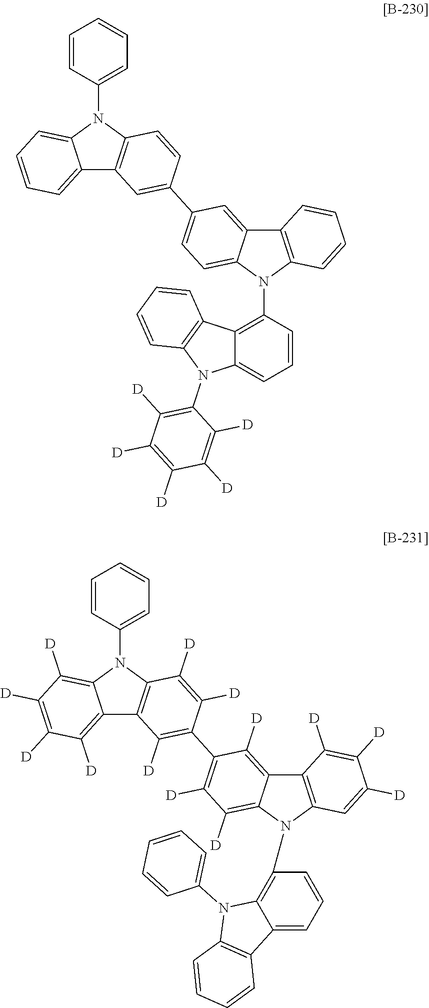 [SMILES: [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H] ; [2H]c1cc2c(c([2H])c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1cccc2c3ccccc3n(-c3ccccc3)c12]

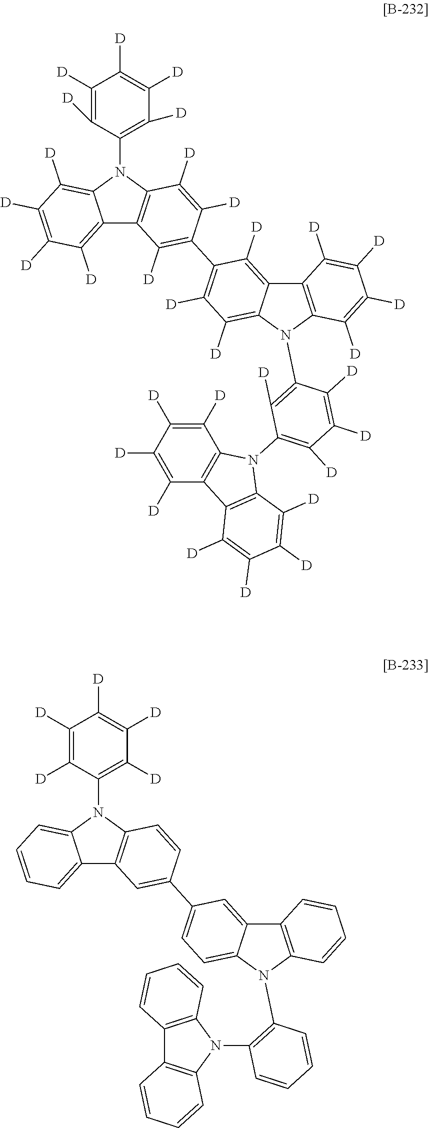 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H] ; [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H]]

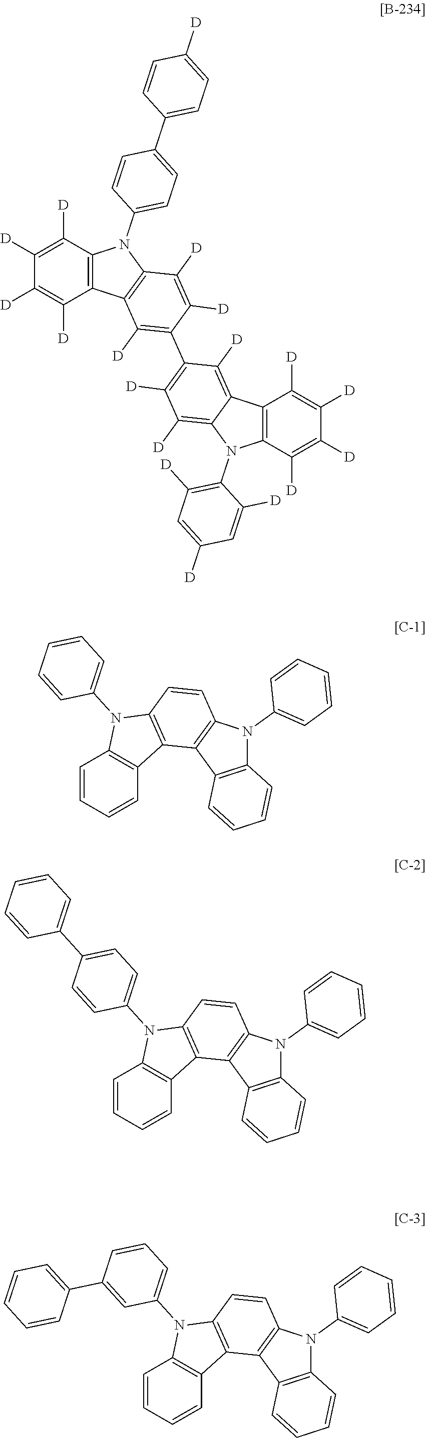 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1 ; c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1]

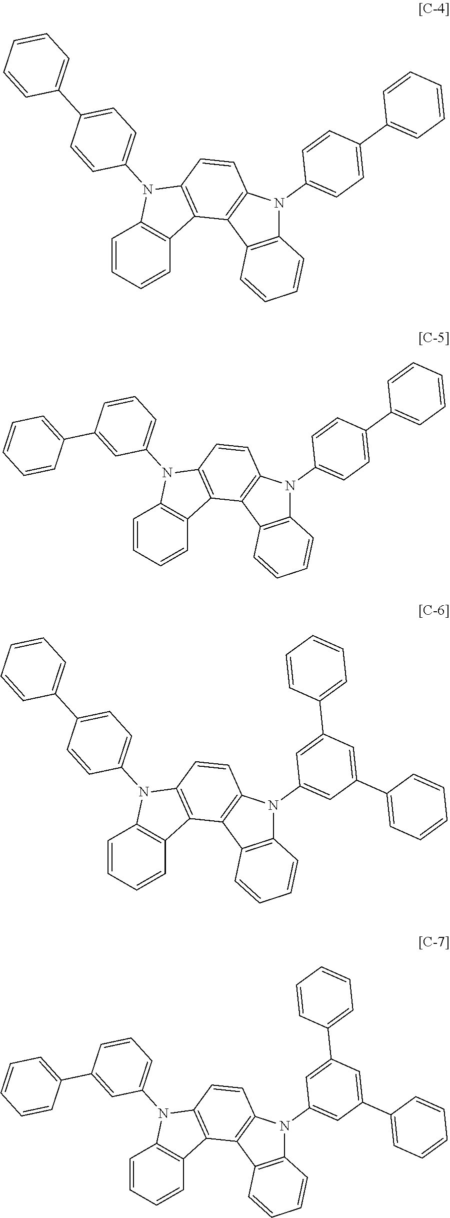 [SMILES: c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1]

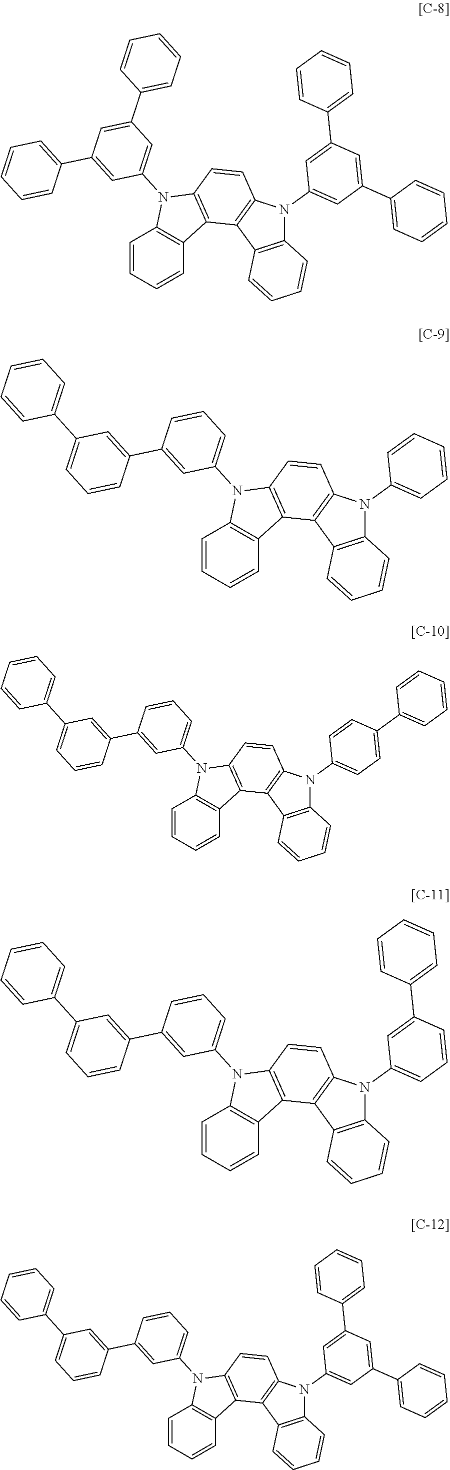 [SMILES: c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1 ; c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

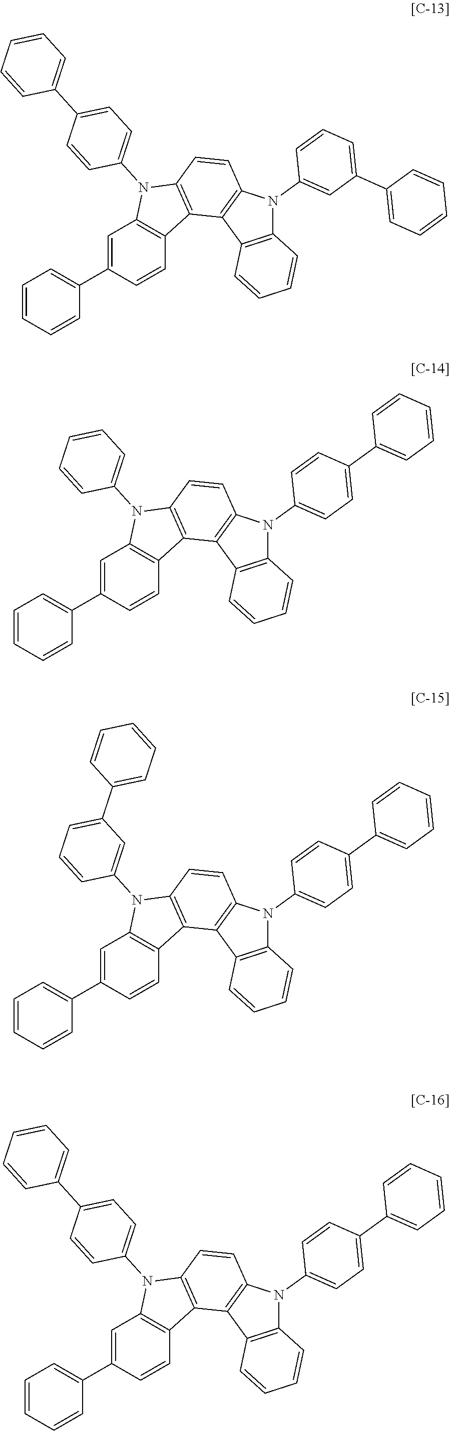 [SMILES: c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

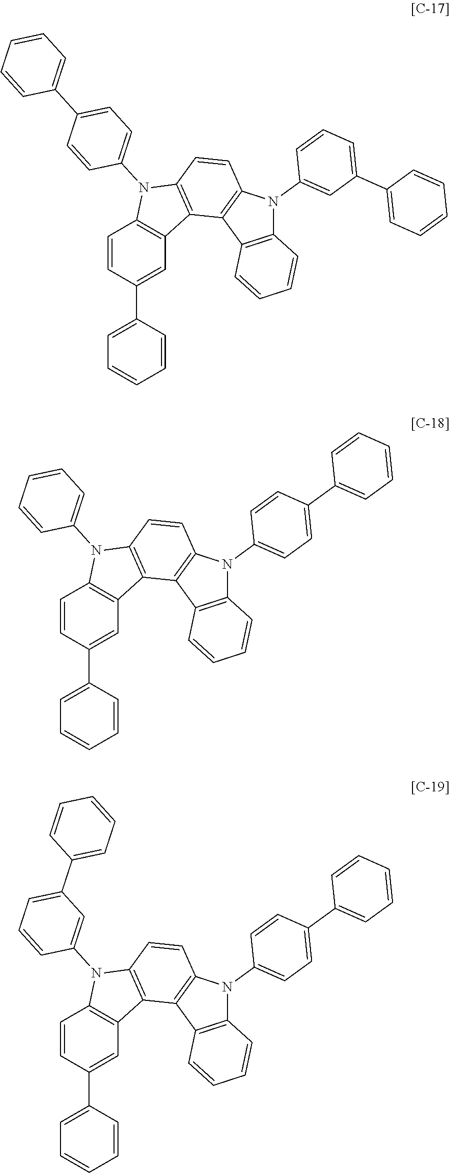 [SMILES: c1ccc(-c2ccc(-n3c4ccc(-c5ccccc5)cc4c4c5c6ccccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1]

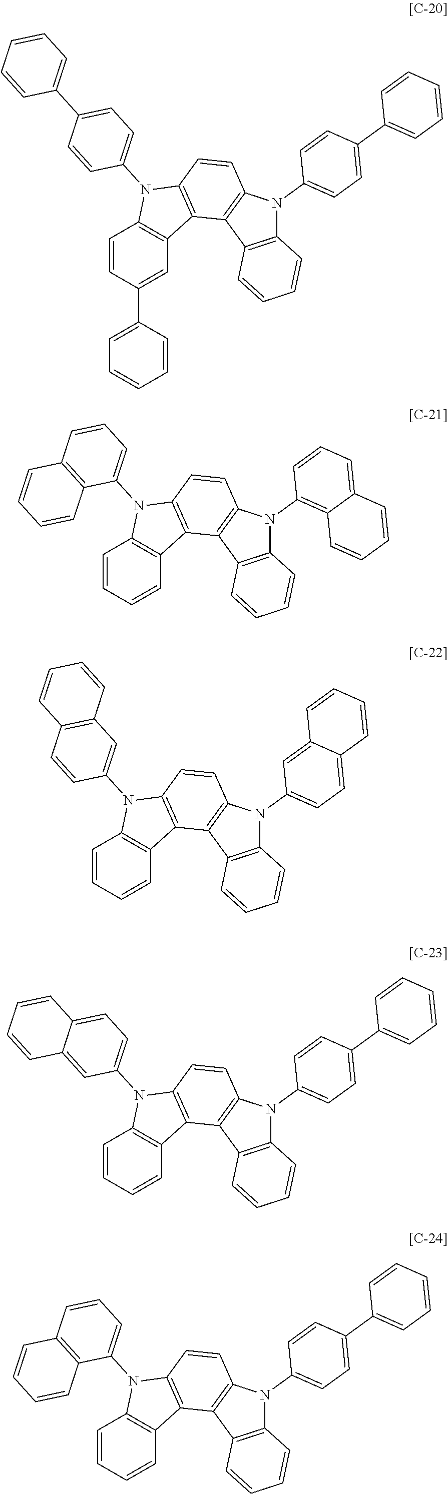 [SMILES: c1ccc(-c2ccc(-n3c4ccccc4c4c5c6cc(-c7ccccc7)ccc6n(-c6ccc(-c7ccccc7)cc6)c5ccc43)cc2)cc1 ; c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1 ; c1ccc(-c2ccc(-n3c4ccccc4c4c5c6ccccc6n(-c6cccc7ccccc67)c5ccc43)cc2)cc1]

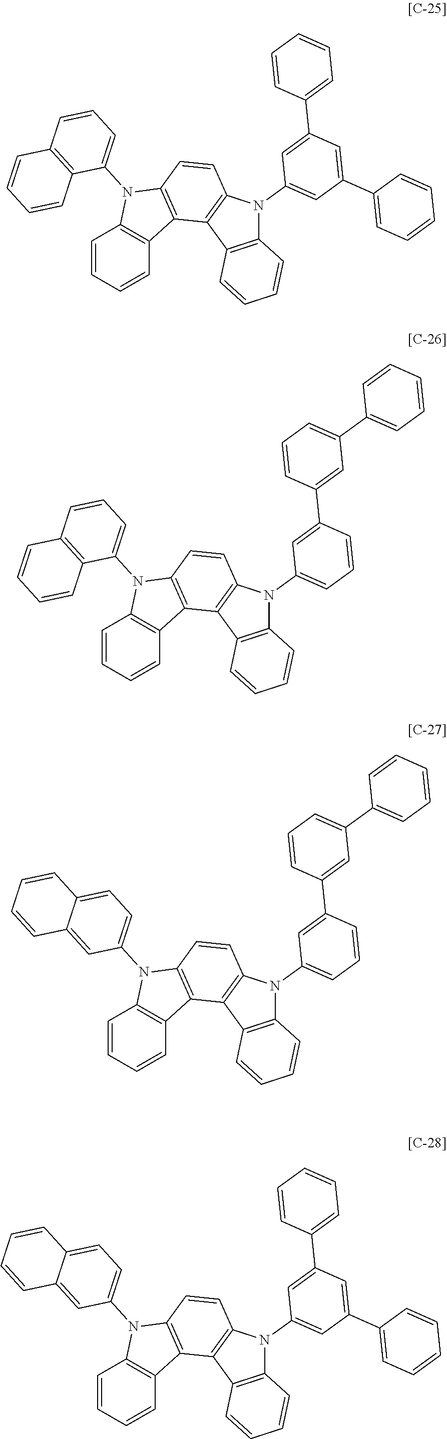 [SMILES: c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1 ; c1ccc(-c2cc(-c3ccccc3)cc(-n3c4ccccc4c4c5c6ccccc6n(-c6ccc7ccccc7c6)c5ccc43)c2)cc1]

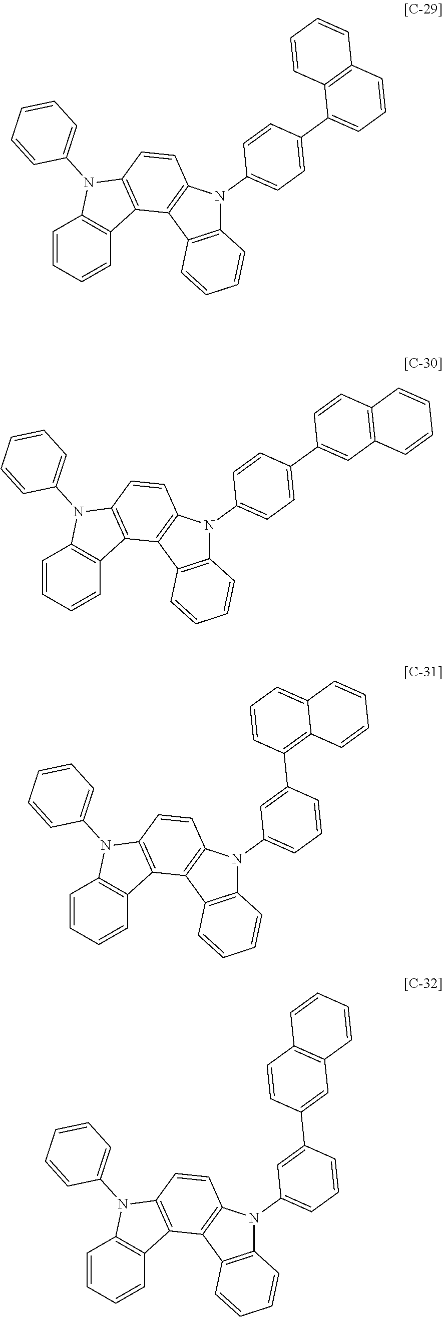 [SMILES: c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1 ; c1ccc(-n2c3ccccc3c3c4c5ccccc5n(-c5cccc(-c6ccc7ccccc7c6)c5)c4ccc32)cc1]

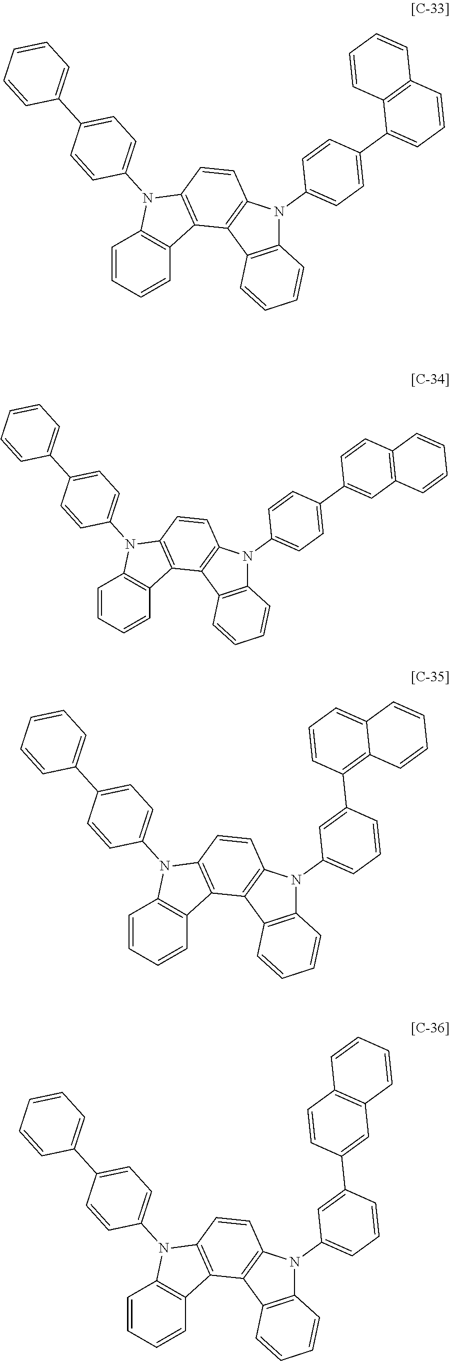 [SMILES: c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1 ; c1ccc(-c2ccc(-n3c4ccccc4c4c5c6ccccc6n(-c6cccc(-c7ccc8ccccc8c7)c6)c5ccc43)cc2)cc1]

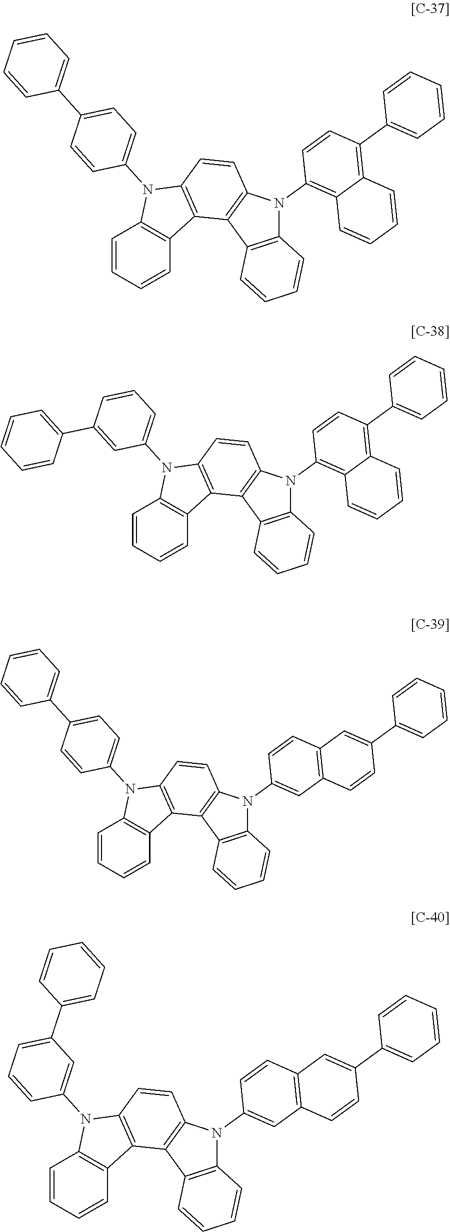 [SMILES: c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1 ; c1ccc(-c2cccc(-n3c4ccccc4c4c5c6ccccc6n(-c6ccc7cc(-c8ccccc8)ccc7c6)c5ccc43)c2)cc1]

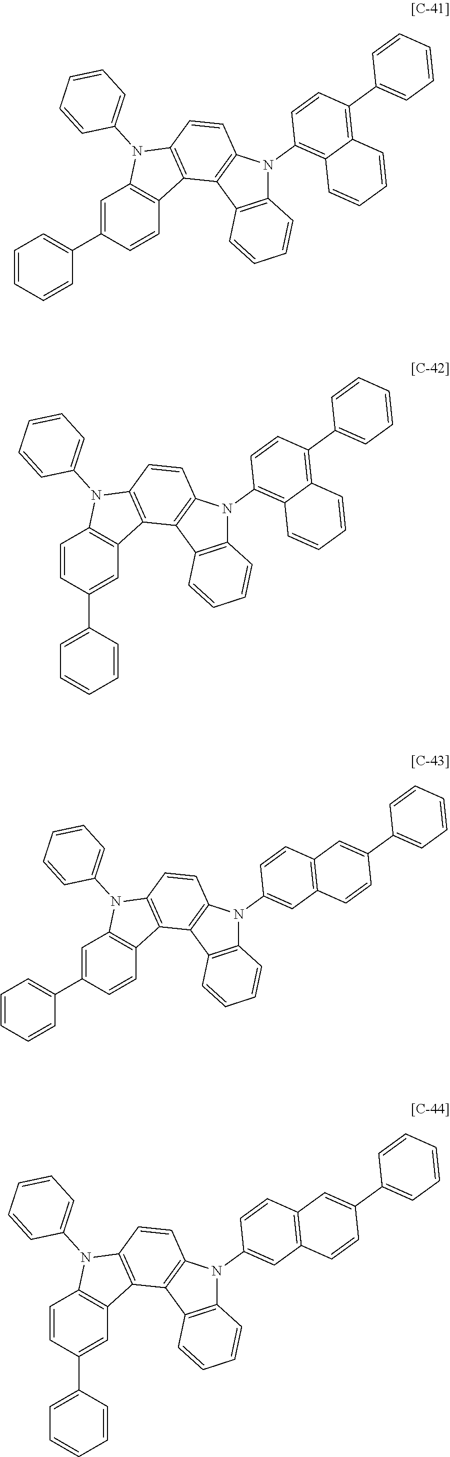 [SMILES: c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1 ; c1ccc(-c2ccc3cc(-n4c5ccccc5c5c6c7cc(-c8ccccc8)ccc7n(-c7ccccc7)c6ccc54)ccc3c2)cc1]

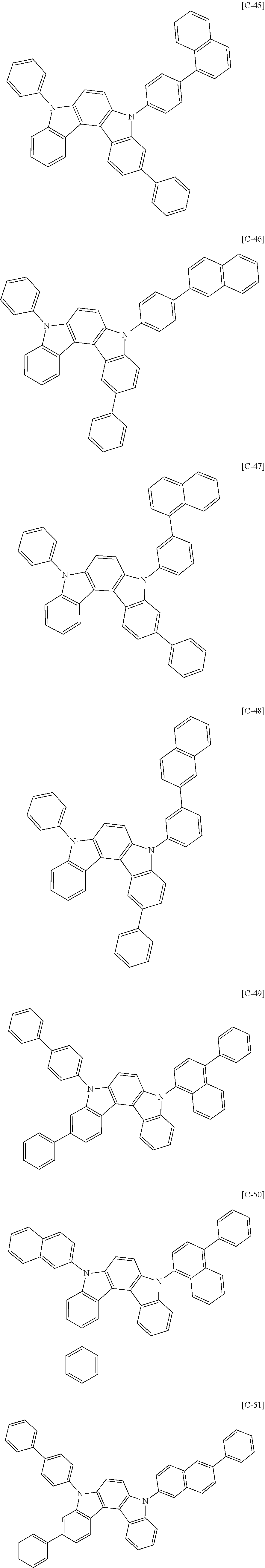

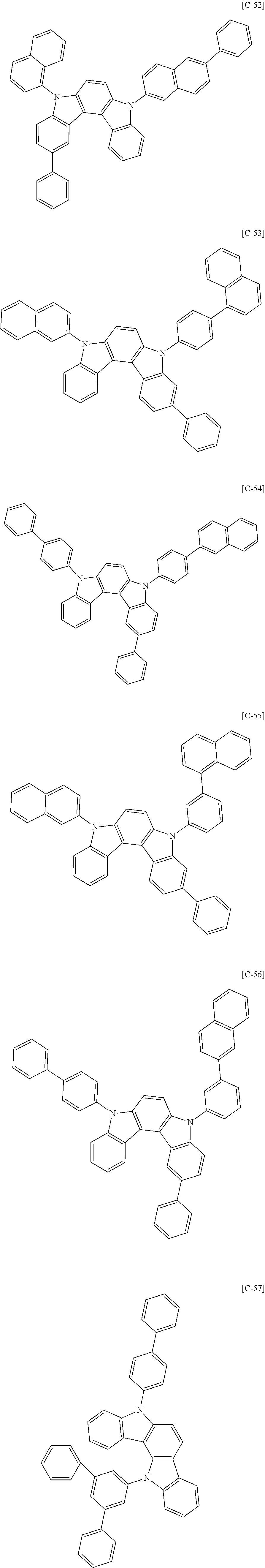

Examples of Compound C-1 to Compound C-57 listed in Group 2 in which at least one hydrogen is replaced with deuterium are shown below.

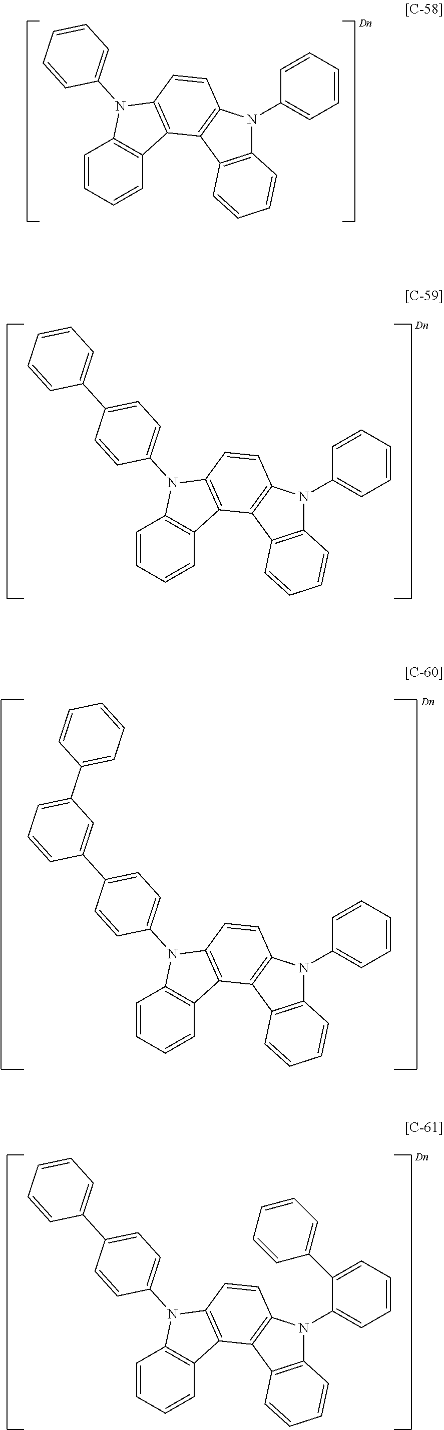

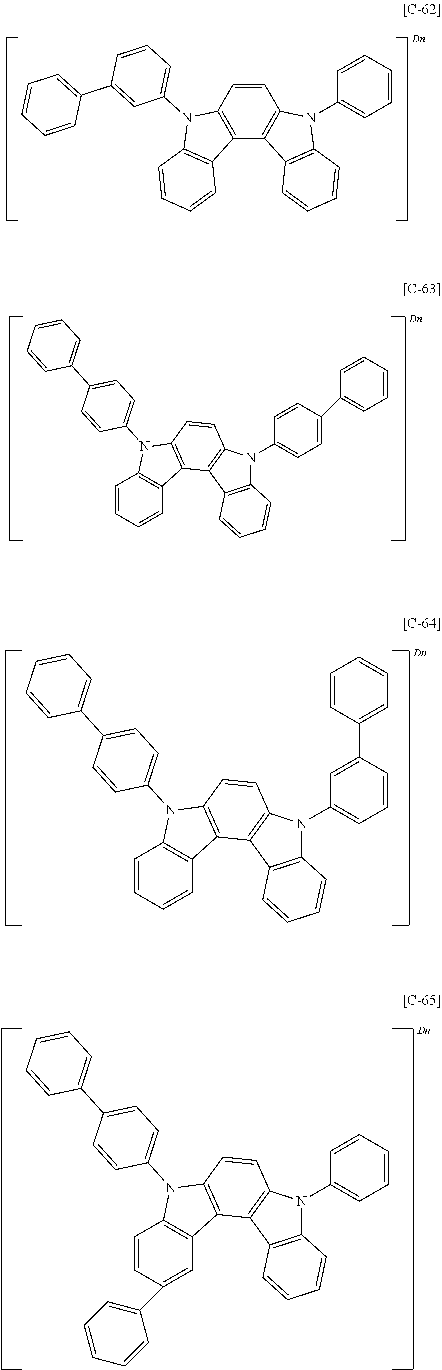

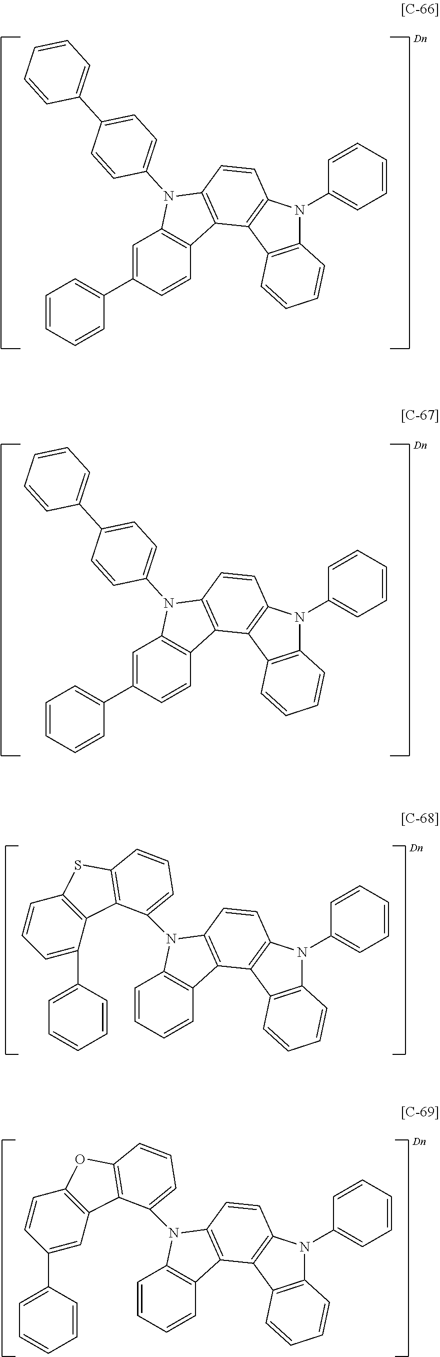

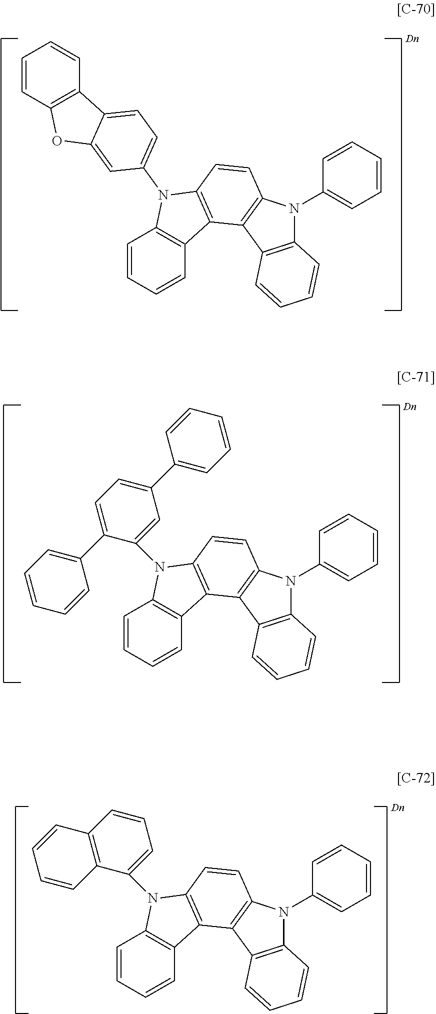

(Dn refers to the number of deuterium substitutions and indicates a structure substituted with one or more deuterium atoms)

The most specific structures for Compound C-58 to Compound C-72 of Group 2 are presented below as examples according to the position and substitution ratio of deuterium.

In an implementation, the deuterium substitution position, deuterium substitution ratio, or the like may include all changeable ranges within the range of Compound C-58 to Compound C-72.

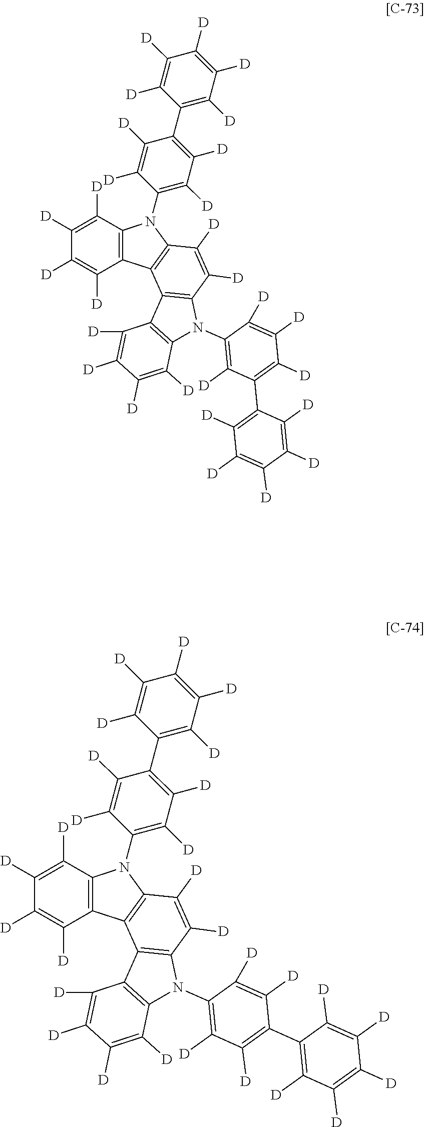 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

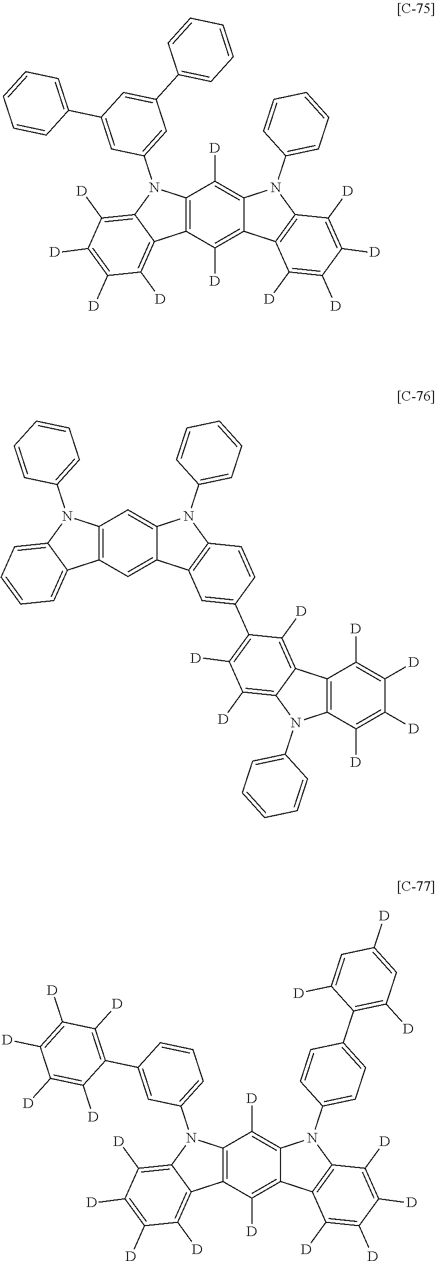 [SMILES: [2H]c1c([2H])c([2H])c2c(c1[2H])c1c([2H])c3c4c([2H])c([2H])c([2H])c([2H])c4n(-c4cc(-c5ccccc5)cc(-c5ccccc5)c4)c3c([2H])c1n2-c1ccccc1 ; [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1]

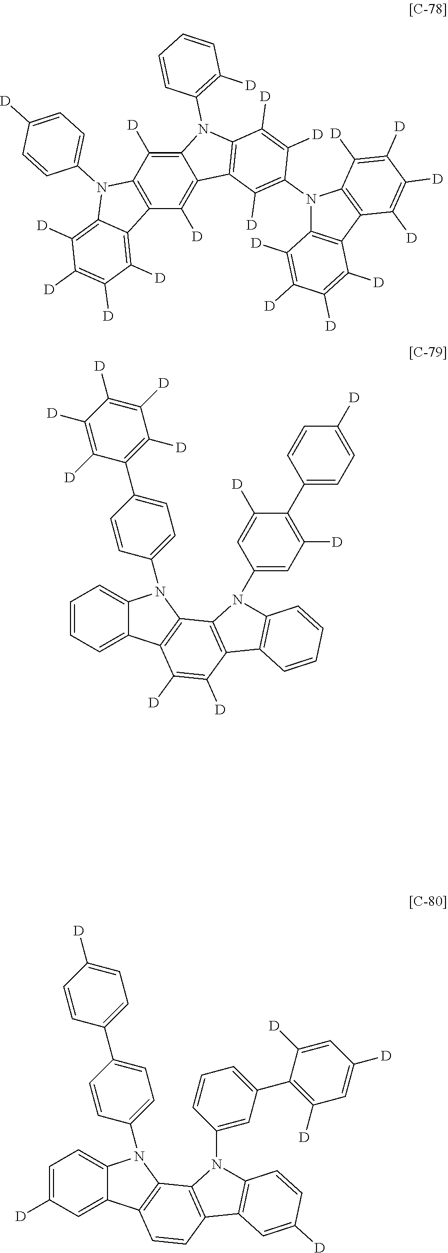 [SMILES: [2H]c1ccc(-n2c3c([2H])c([2H])c([2H])c([2H])c3c3c([2H])c4c5c([2H])c(-n6c7c([2H])c([2H])c([2H])c([2H])c7c7c([2H])c([2H])c([2H])c([2H])c76)c([2H])c([2H])c5n(-c5ccccc5[2H])c4c([2H])c32)cc1 ; [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1 ; [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1]

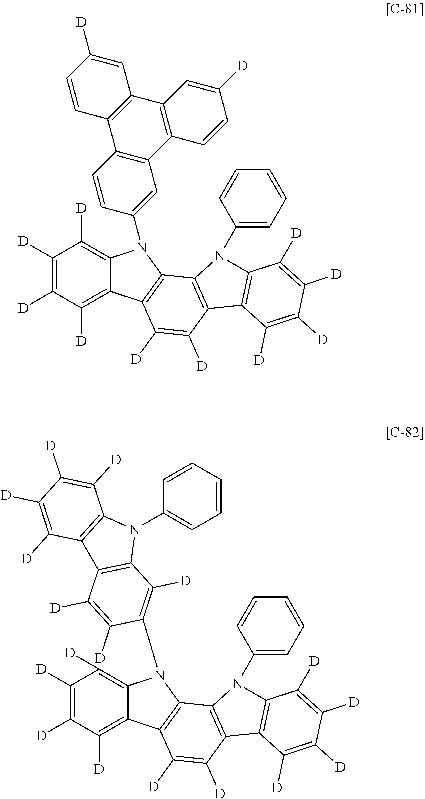 [SMILES: [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1 ; [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1]

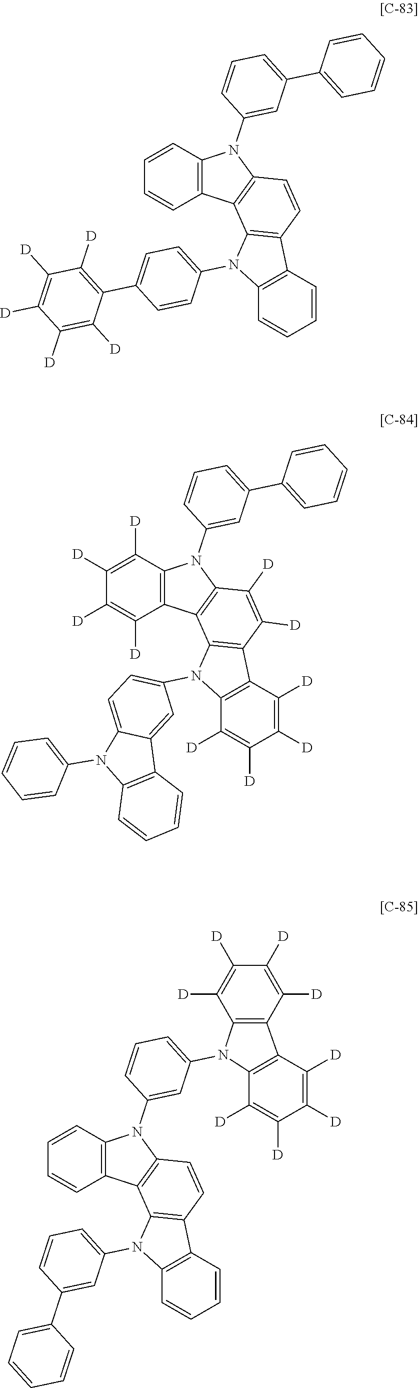 [SMILES: [2H]c1c([2H])c([2H])c(-c2ccc(-n3c4ccccc4c4ccc5c(c6ccccc6n5-c5cccc(-c6ccccc6)c5)c43)cc2)c([2H])c1[2H] ; [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1 ; [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1]

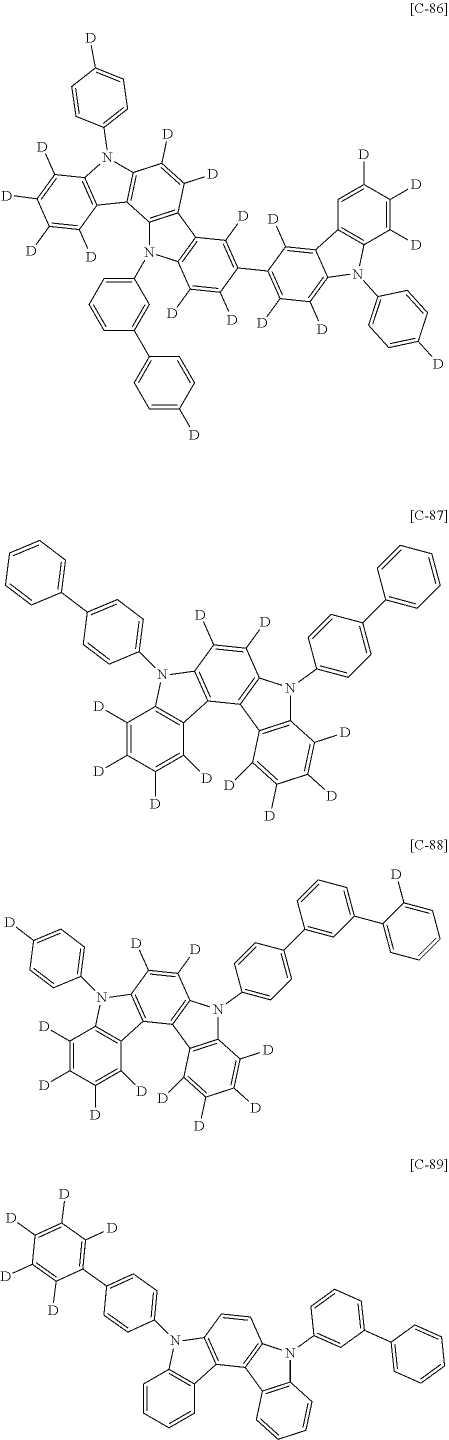

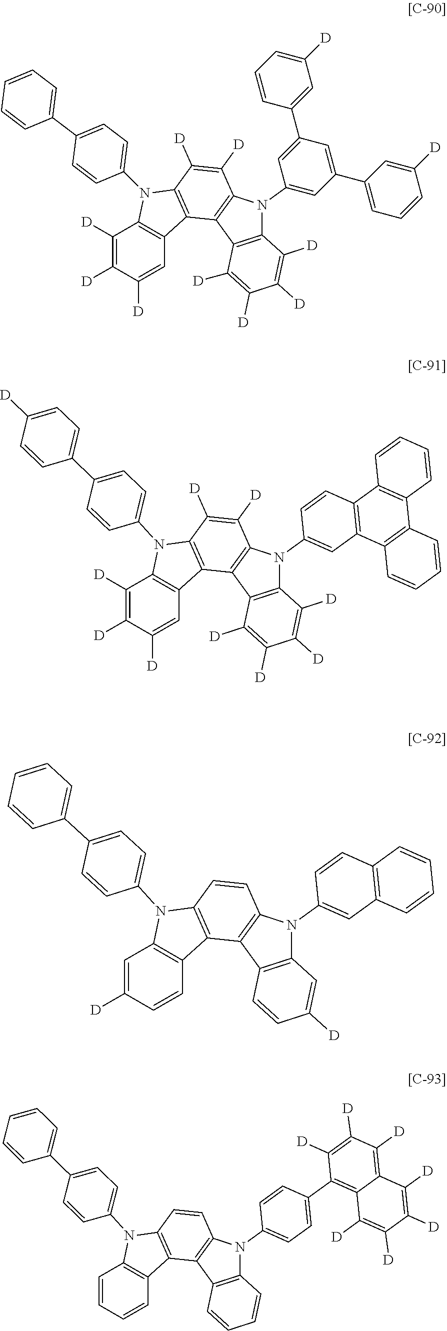 [SMILES: [2H]c1cccc(-c2cc(-c3cccc([2H])c3)cc(-n3c4c([2H])c([2H])c([2H])c([2H])c4c4c5c6cc([2H])c([2H])c([2H])c6n(-c6ccc(-c7ccccc7)cc6)c5c([2H])c([2H])c43)c2)c1 ; [2H]c1ccc(-c2ccc(-n3c4c([2H])c([2H])c([2H])cc4c4c5c6c([2H])c([2H])c([2H])c([2H])c6n(-c6ccc7c8ccccc8c8ccccc8c7c6)c5c([2H])c([2H])c43)cc2)cc1 ; [2H]c1ccc2c3c4c5ccc([2H])cc5n(-c5ccc6ccccc6c5)c4ccc3n(-c3ccc(-c4ccccc4)cc3)c2c1 ; [2H]c1c([2H])c([2H])c2c(-c3ccc(-n4c5ccccc5c5c6c7ccccc7n(-c7ccc(-c8ccccc8)cc7)c6ccc54)cc3)c([2H])c([2H])c([2H])c2c1[2H]]

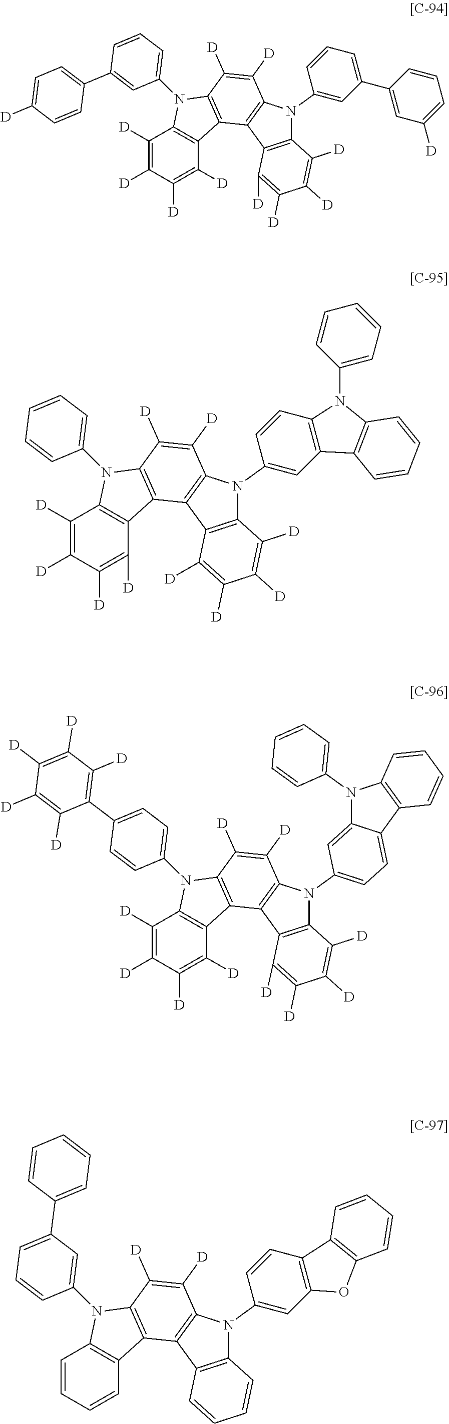

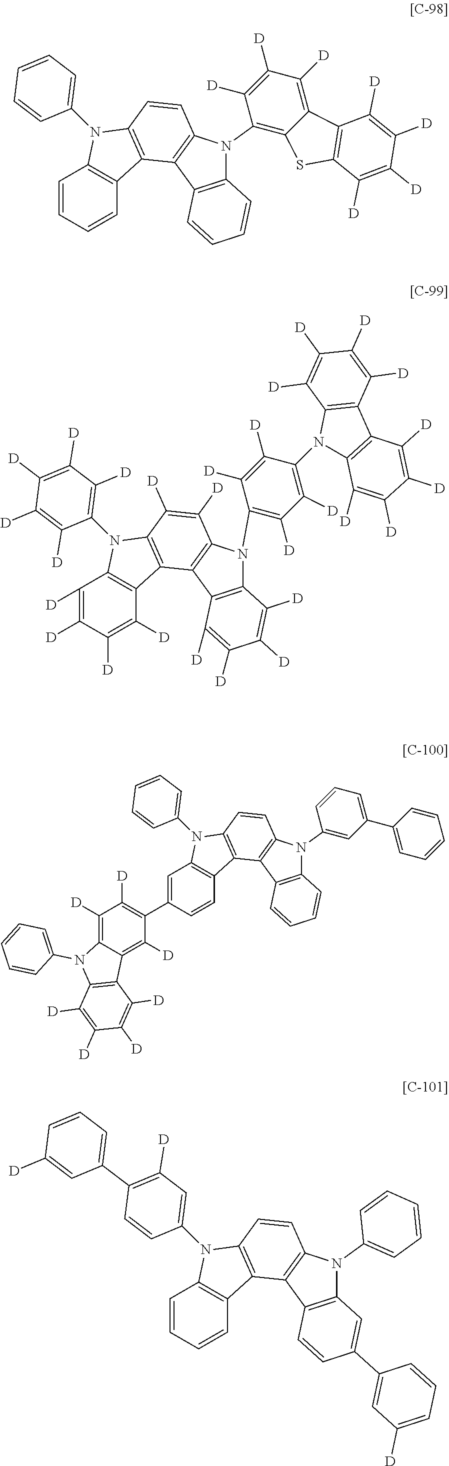

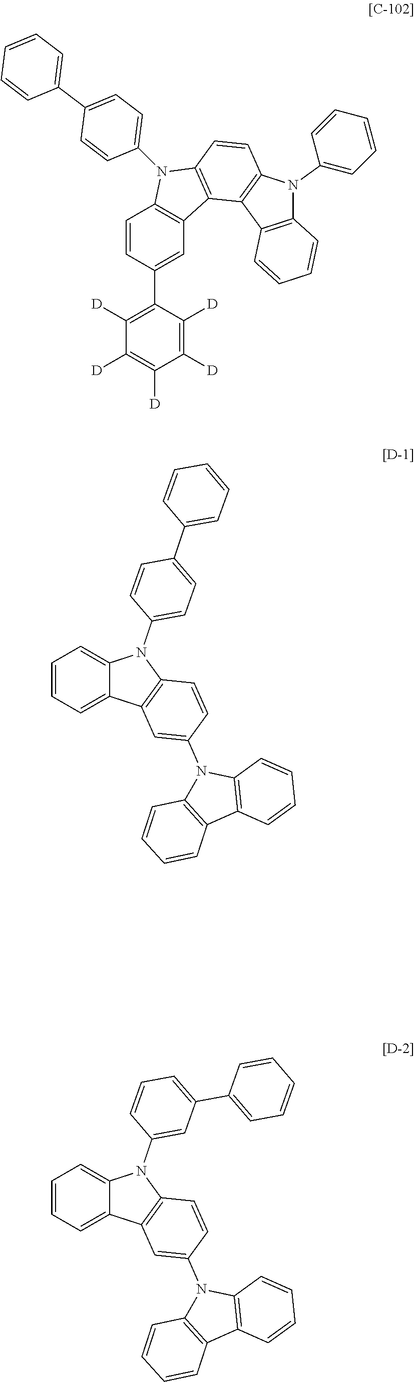 [SMILES: [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H] ; c1ccc(-c2ccc(-n3c4ccccc4c4cc(-n5c6ccccc6c6ccccc65)ccc43)cc2)cc1 ; c1ccc(-c2cccc(-n3c4ccccc4c4cc(-n5c6ccccc6c6ccccc65)ccc43)c2)cc1]

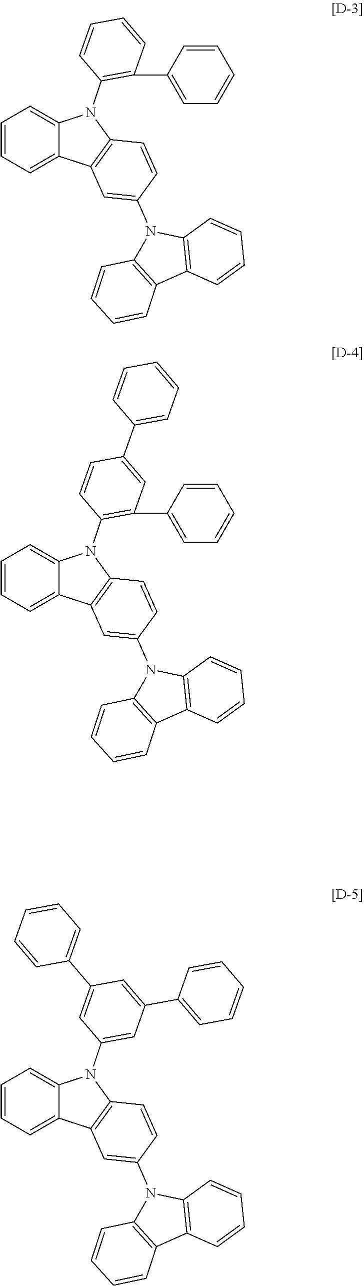

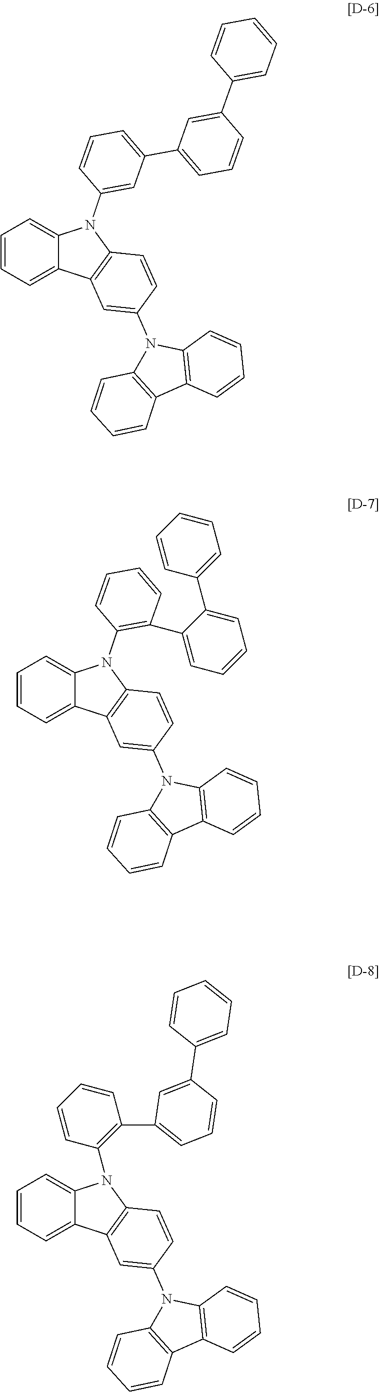 [SMILES: c1ccc(-c2cccc(-c3cccc(-n4c5ccccc5c5cc(-n6c7ccccc7c7ccccc76)ccc54)c3)c2)cc1 ; c1ccc(-c2ccccc2-c2ccccc2-n2c3ccccc3c3cc(-n4c5ccccc5c5ccccc54)ccc32)cc1 ; c1ccc(-c2cccc(-c3ccccc3-n3c4ccccc4c4cc(-n5c6ccccc6c6ccccc65)ccc43)c2)cc1]

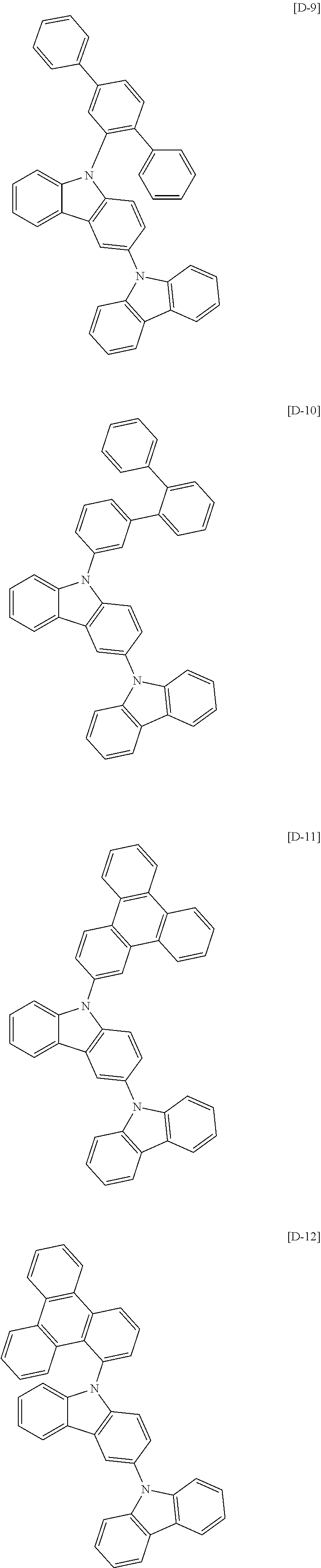 [SMILES: c1ccc(-c2ccc(-c3ccccc3)c(-n3c4ccccc4c4cc(-n5c6ccccc6c6ccccc65)ccc43)c2)cc1 ; c1ccc(-c2ccccc2-c2cccc(-n3c4ccccc4c4cc(-n5c6ccccc6c6ccccc65)ccc43)c2)cc1 ; c1ccc2c(c1)c1ccccc1c1cc(-n3c4ccccc4c4cc(-n5c6ccccc6c6ccccc65)ccc43)ccc21 ; c1ccc2c(c1)c1ccccc1c1c(-n3c4ccccc4c4cc(-n5c6ccccc6c6ccccc65)ccc43)cccc21]

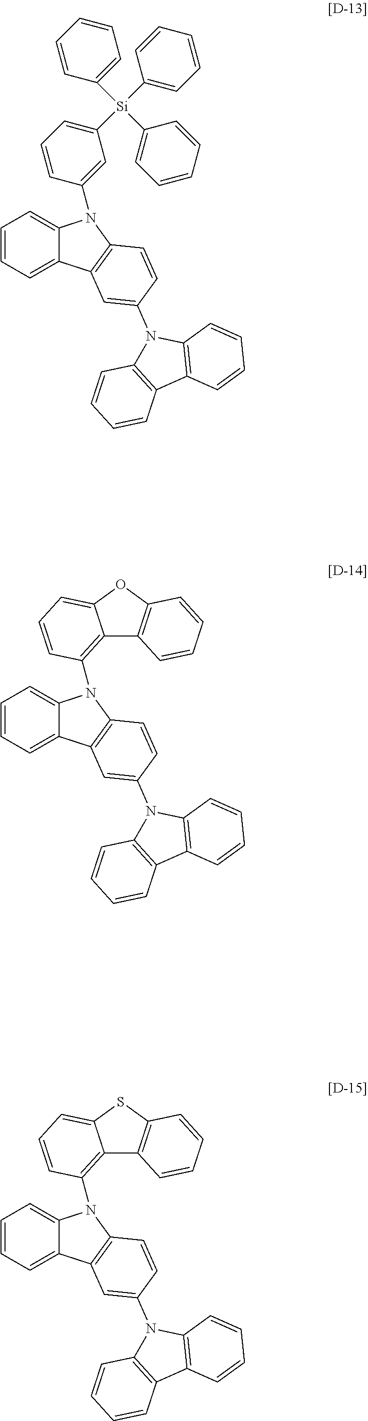 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc2c(c1)oc1cccc(-n3c4ccccc4c4cc(-n5c6ccccc6c6ccccc65)ccc43)c12 ; c1ccc2c(c1)sc1cccc(-n3c4ccccc4c4cc(-n5c6ccccc6c6ccccc65)ccc43)c12]

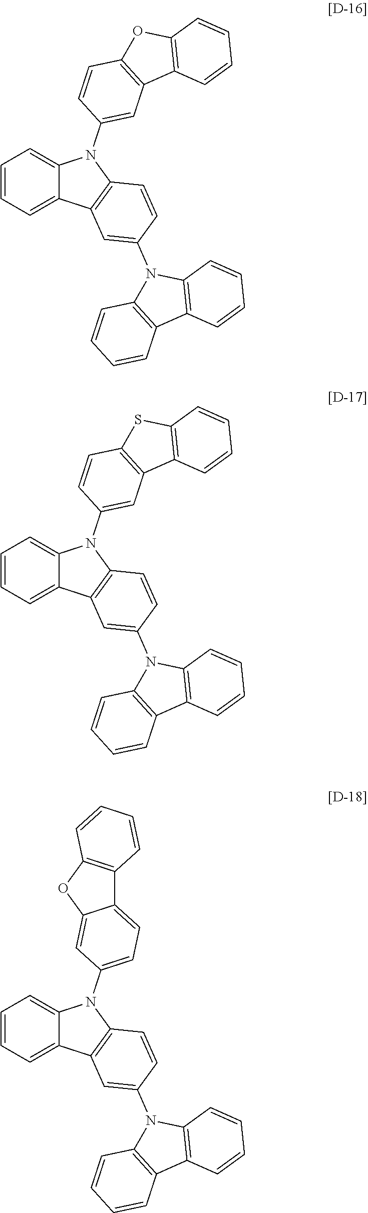 [SMILES: c1ccc2c(c1)oc1ccc(-n3c4ccccc4c4cc(-n5c6ccccc6c6ccccc65)ccc43)cc12 ; c1ccc2c(c1)sc1ccc(-n3c4ccccc4c4cc(-n5c6ccccc6c6ccccc65)ccc43)cc12 ; c1ccc2c(c1)oc1cc(-n3c4ccccc4c4cc(-n5c6ccccc6c6ccccc65)ccc43)ccc12]

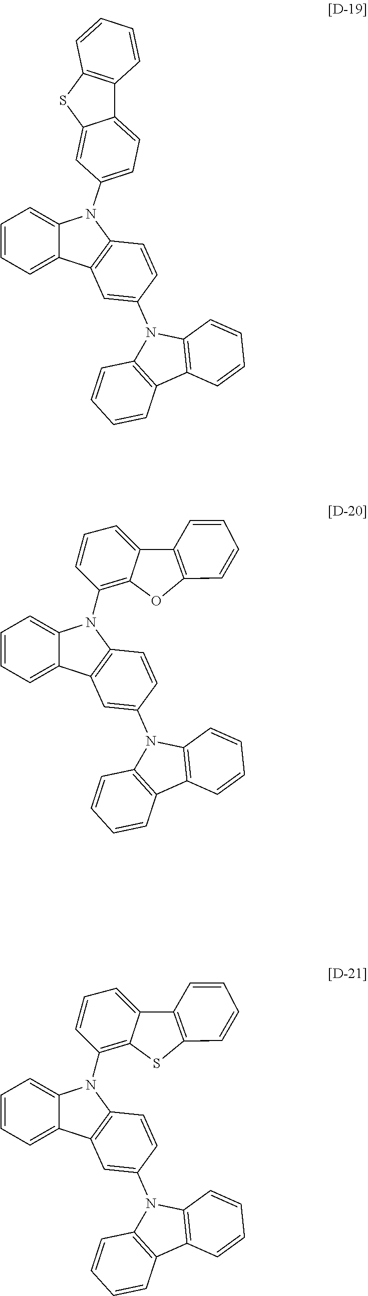 [SMILES: c1ccc2c(c1)sc1cc(-n3c4ccccc4c4cc(-n5c6ccccc6c6ccccc65)ccc43)ccc12 ; c1ccc2c(c1)oc1c(-n3c4ccccc4c4cc(-n5c6ccccc6c6ccccc65)ccc43)cccc12 ; c1ccc2c(c1)sc1c(-n3c4ccccc4c4cc(-n5c6ccccc6c6ccccc65)ccc43)cccc12]

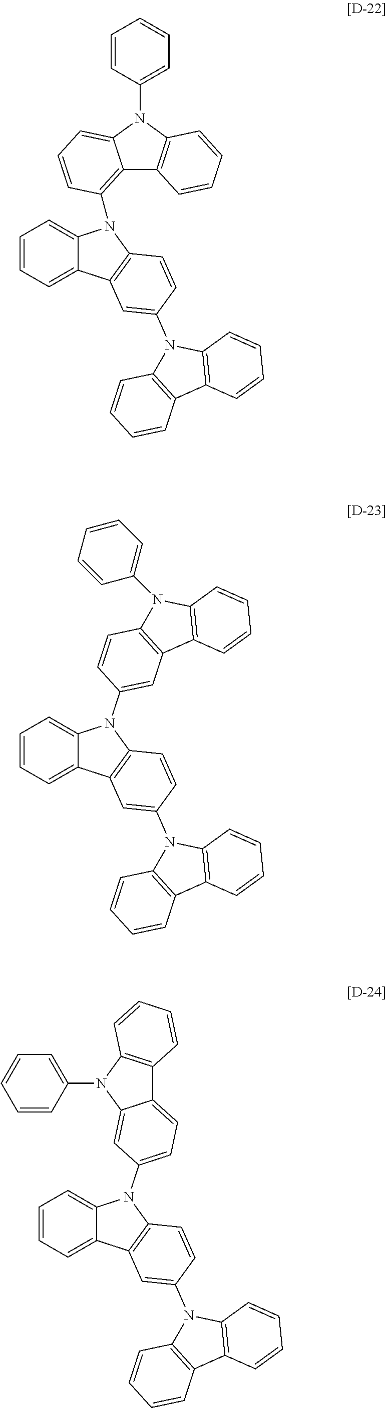 [SMILES: c1ccc(-n2c3ccccc3c3c(-n4c5ccccc5c5cc(-n6c7ccccc7c7ccccc76)ccc54)cccc32)cc1 ; c1ccc(-n2c3ccccc3c3cc(-n4c5ccccc5c5cc(-n6c7ccccc7c7ccccc76)ccc54)ccc32)cc1 ; c1ccc(-n2c3ccccc3c3ccc(-n4c5ccccc5c5cc(-n6c7ccccc7c7ccccc76)ccc54)cc32)cc1]

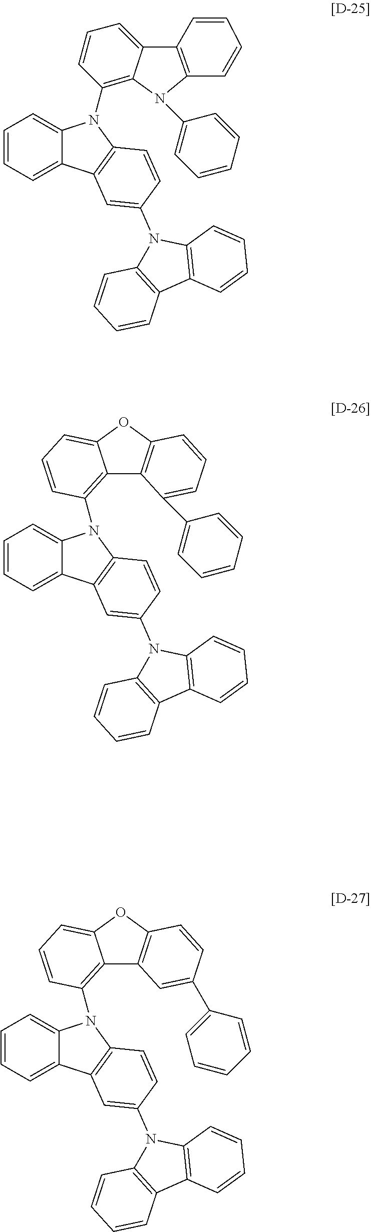 [SMILES: c1ccc(-n2c3ccccc3c3cccc(-n4c5ccccc5c5cc(-n6c7ccccc7c7ccccc76)ccc54)c32)cc1 ; c1ccc(-c2cccc3oc4cccc(-n5c6ccccc6c6cc(-n7c8ccccc8c8ccccc87)ccc65)c4c23)cc1 ; c1ccc(-c2ccc3oc4cccc(-n5c6ccccc6c6cc(-n7c8ccccc8c8ccccc87)ccc65)c4c3c2)cc1]

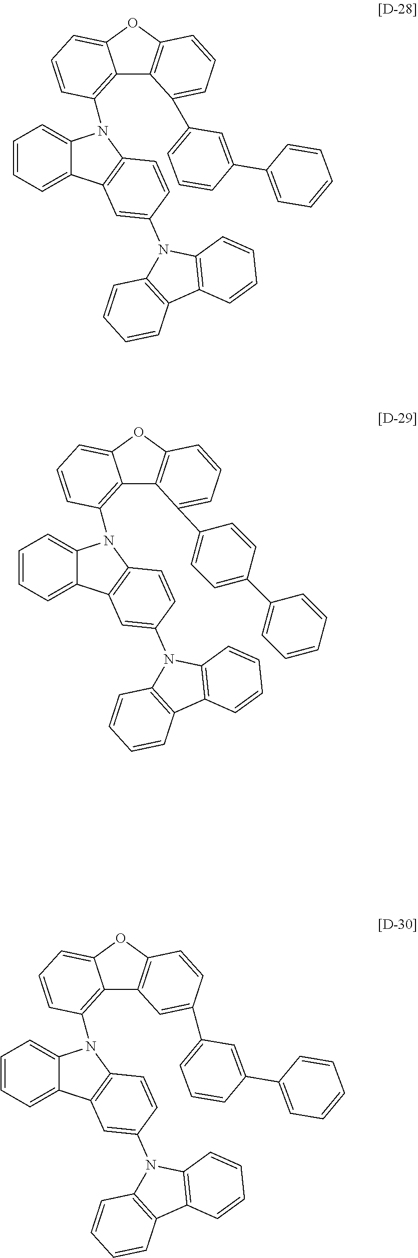 [SMILES: c1ccc(-c2cccc(-c3cccc4oc5cccc(-n6c7ccccc7c7cc(-n8c9ccccc9c9ccccc98)ccc76)c5c34)c2)cc1 ; c1ccc(-c2ccc(-c3cccc4oc5cccc(-n6c7ccccc7c7cc(-n8c9ccccc9c9ccccc98)ccc76)c5c34)cc2)cc1 ; c1ccc(-c2cccc(-c3ccc4oc5cccc(-n6c7ccccc7c7cc(-n8c9ccccc9c9ccccc98)ccc76)c5c4c3)c2)cc1]

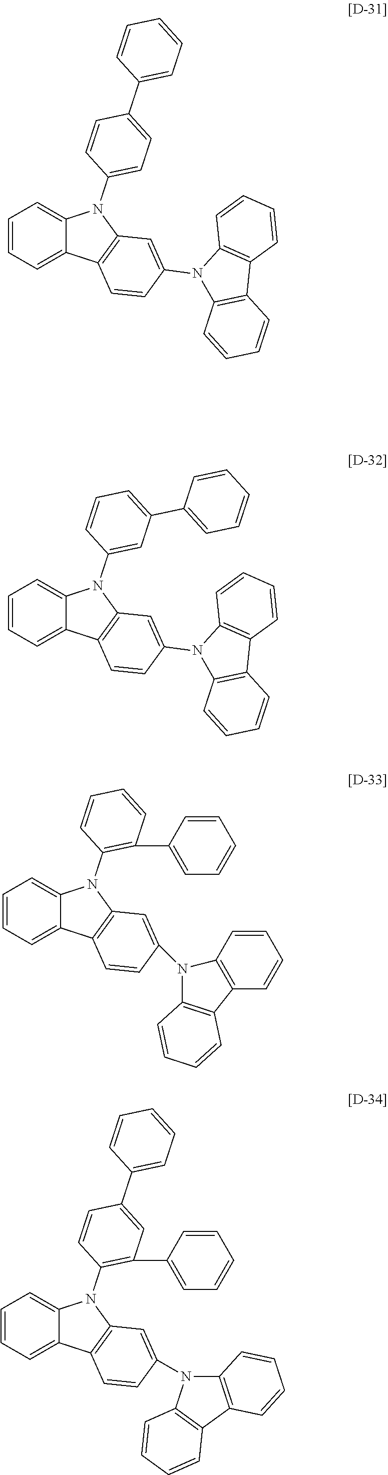 [SMILES: c1ccc(-c2ccc(-n3c4ccccc4c4ccc(-n5c6ccccc6c6ccccc65)cc43)cc2)cc1 ; c1ccc(-c2cccc(-n3c4ccccc4c4ccc(-n5c6ccccc6c6ccccc65)cc43)c2)cc1 ; c1ccc(-c2ccccc2-n2c3ccccc3c3ccc(-n4c5ccccc5c5ccccc54)cc32)cc1 ; c1ccc(-c2ccc(-n3c4ccccc4c4ccc(-n5c6ccccc6c6ccccc65)cc43)c(-c3ccccc3)c2)cc1]

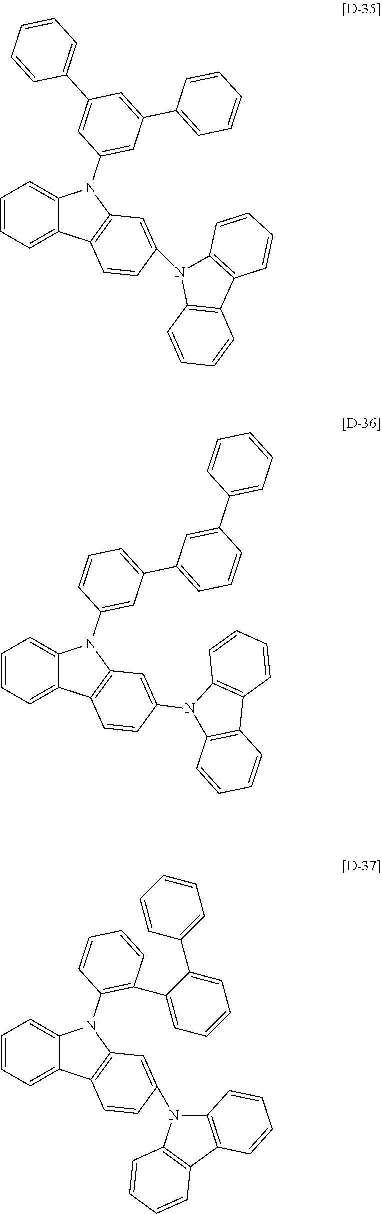 [SMILES: c1ccc(-c2cc(-c3ccccc3)cc(-n3c4ccccc4c4ccc(-n5c6ccccc6c6ccccc65)cc43)c2)cc1 ; c1ccc(-c2cccc(-c3cccc(-n4c5ccccc5c5ccc(-n6c7ccccc7c7ccccc76)cc54)c3)c2)cc1 ; c1ccc(-c2ccccc2-c2ccccc2-n2c3ccccc3c3ccc(-n4c5ccccc5c5ccccc54)cc32)cc1]

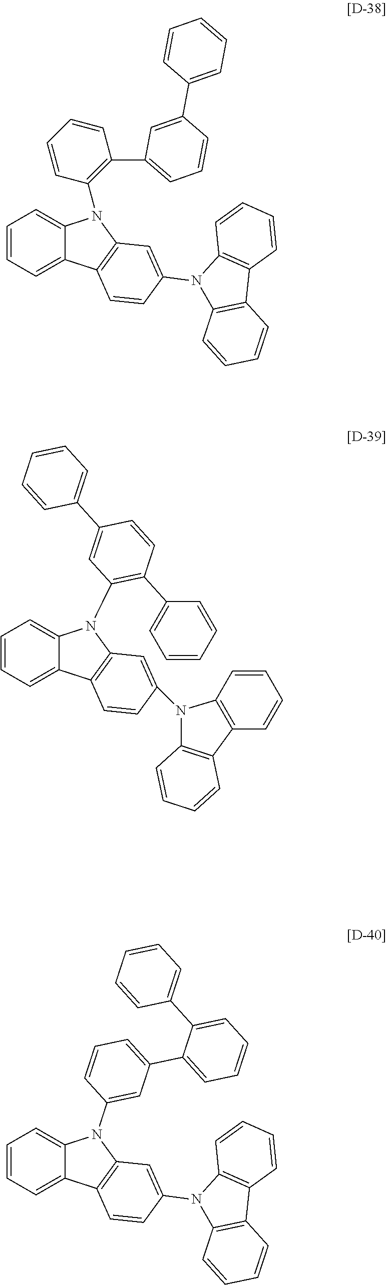 [SMILES: c1ccc(-c2cccc(-c3ccccc3-n3c4ccccc4c4ccc(-n5c6ccccc6c6ccccc65)cc43)c2)cc1 ; c1ccc(-c2ccc(-c3ccccc3)c(-n3c4ccccc4c4ccc(-n5c6ccccc6c6ccccc65)cc43)c2)cc1 ; c1ccc(-c2ccccc2-c2cccc(-n3c4ccccc4c4ccc(-n5c6ccccc6c6ccccc65)cc43)c2)cc1]

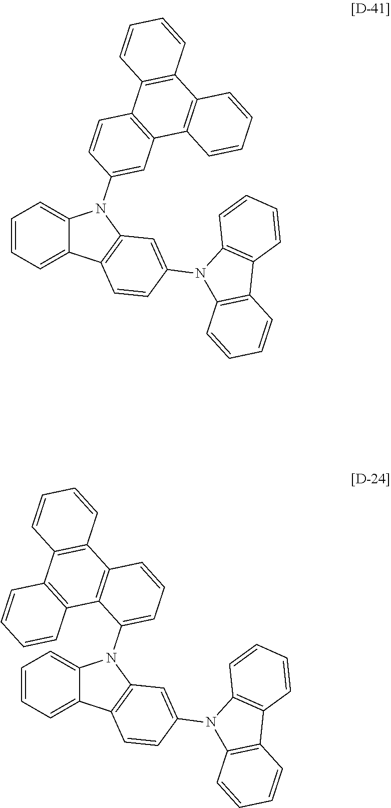 [SMILES: c1ccc2c(c1)c1ccccc1c1cc(-n3c4ccccc4c4ccc(-n5c6ccccc6c6ccccc65)cc43)ccc21 ; c1ccc2c(c1)c1ccccc1c1c(-n3c4ccccc4c4ccc(-n5c6ccccc6c6ccccc65)cc43)cccc21]

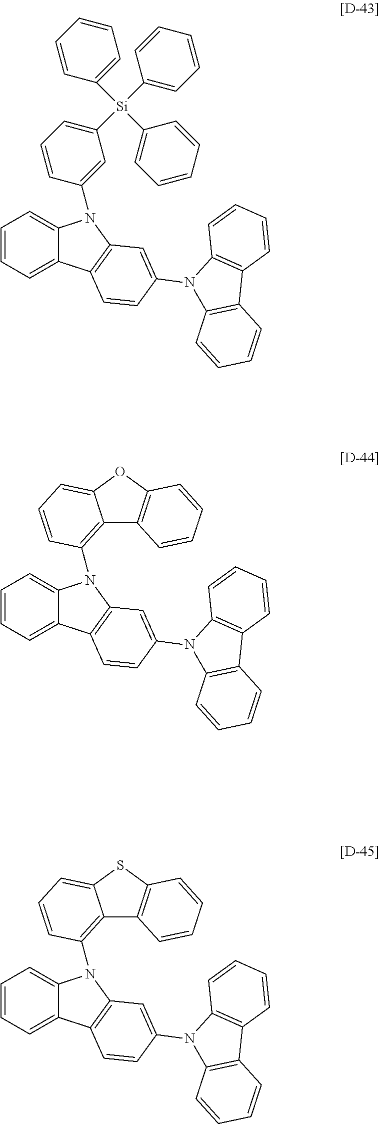 [SMILES: c1ccc([Si](c2ccccc2)(c2ccccc2)c2cccc(-n3c4ccccc4c4ccc(-n5c6ccccc6c6ccccc65)cc43)c2)cc1 ; c1ccc2c(c1)oc1cccc(-n3c4ccccc4c4ccc(-n5c6ccccc6c6ccccc65)cc43)c12 ; c1ccc2c(c1)sc1cccc(-n3c4ccccc4c4ccc(-n5c6ccccc6c6ccccc65)cc43)c12]

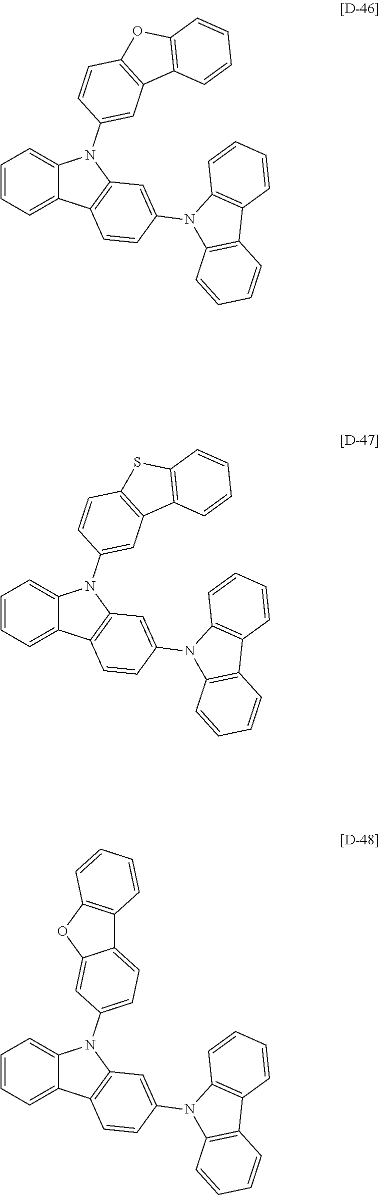 [SMILES: c1ccc2c(c1)oc1ccc(-n3c4ccccc4c4ccc(-n5c6ccccc6c6ccccc65)cc43)cc12 ; c1ccc2c(c1)sc1ccc(-n3c4ccccc4c4ccc(-n5c6ccccc6c6ccccc65)cc43)cc12 ; c1ccc2c(c1)oc1cc(-n3c4ccccc4c4ccc(-n5c6ccccc6c6ccccc65)cc43)ccc12]

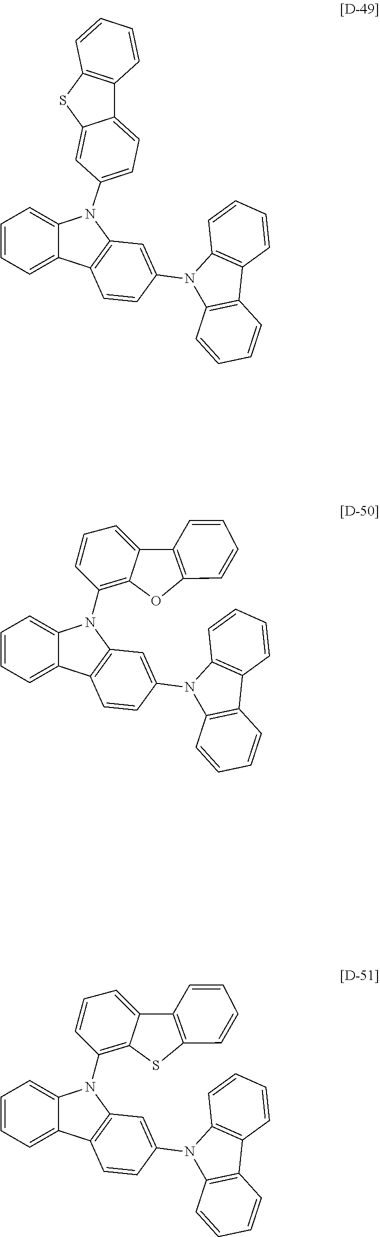 [SMILES: c1ccc2c(c1)sc1cc(-n3c4ccccc4c4ccc(-n5c6ccccc6c6ccccc65)cc43)ccc12 ; c1ccc2c(c1)oc1c(-n3c4ccccc4c4ccc(-n5c6ccccc6c6ccccc65)cc43)cccc12 ; c1ccc2c(c1)sc1c(-n3c4ccccc4c4ccc(-n5c6ccccc6c6ccccc65)cc43)cccc12]

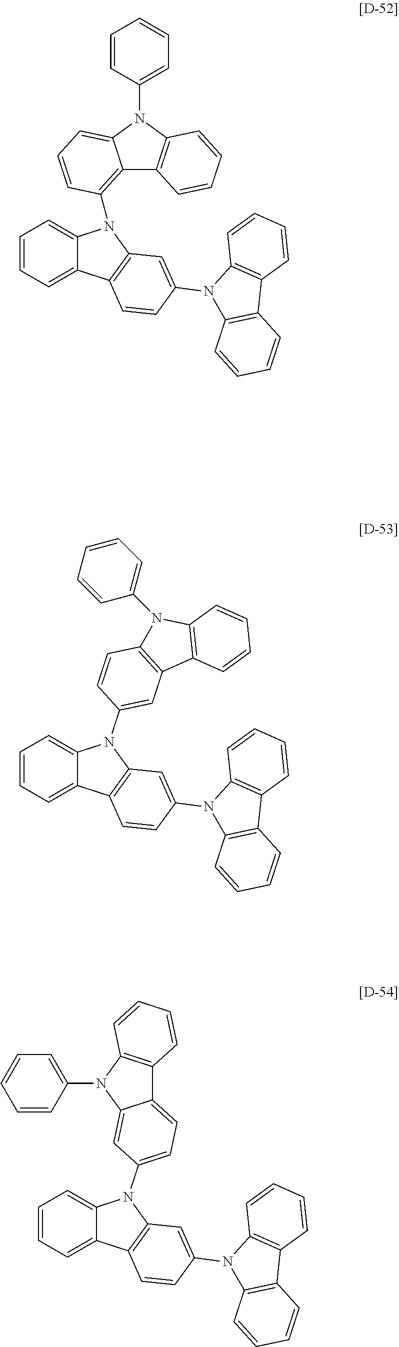 [SMILES: c1ccc(-n2c3ccccc3c3c(-n4c5ccccc5c5ccc(-n6c7ccccc7c7ccccc76)cc54)cccc32)cc1 ; c1ccc(-n2c3ccccc3c3cc(-n4c5ccccc5c5ccc(-n6c7ccccc7c7ccccc76)cc54)ccc32)cc1 ; c1ccc(-n2c3ccccc3c3ccc(-n4c5ccccc5c5ccc(-n6c7ccccc7c7ccccc76)cc54)cc32)cc1]

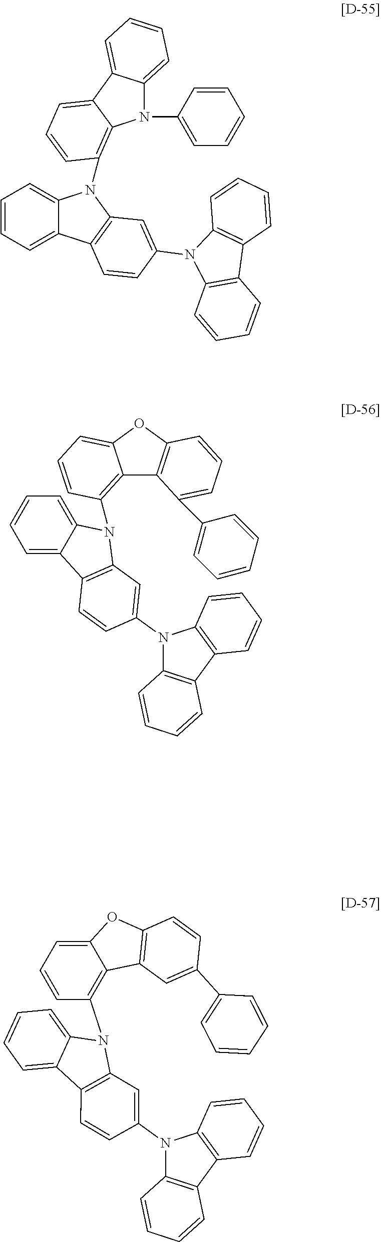 [SMILES: c1ccc(-n2c3ccccc3c3cccc(-n4c5ccccc5c5ccc(-n6c7ccccc7c7ccccc76)cc54)c32)cc1 ; c1ccc(-c2cccc3oc4cccc(-n5c6ccccc6c6ccc(-n7c8ccccc8c8ccccc87)cc65)c4c23)cc1 ; c1ccc(-c2ccc3oc4cccc(-n5c6ccccc6c6ccc(-n7c8ccccc8c8ccccc87)cc65)c4c3c2)cc1]

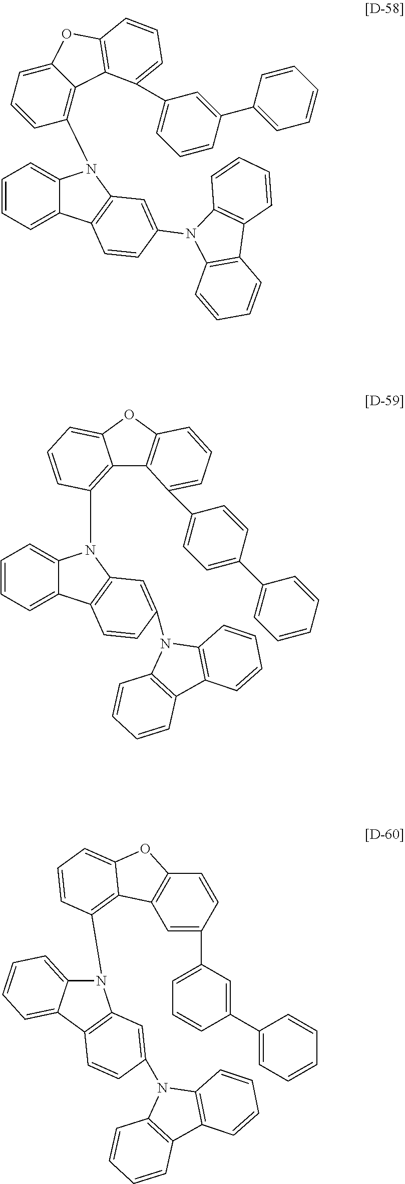 [SMILES: c1ccc(-c2cccc(-c3cccc4oc5cccc(-n6c7ccccc7c7ccc(-n8c9ccccc9c9ccccc98)cc76)c5c34)c2)cc1 ; c1ccc(-c2ccc(-c3cccc4oc5cccc(-n6c7ccccc7c7ccc(-n8c9ccccc9c9ccccc98)cc76)c5c34)cc2)cc1 ; c1ccc(-c2cccc(-c3ccc4oc5cccc(-n6c7ccccc7c7ccc(-n8c9ccccc9c9ccccc98)cc76)c5c4c3)c2)cc1]

Examples of Compound D-1 to Compound D-60 listed in Group 2 in which at least one hydrogen is replaced with deuterium are shown below.

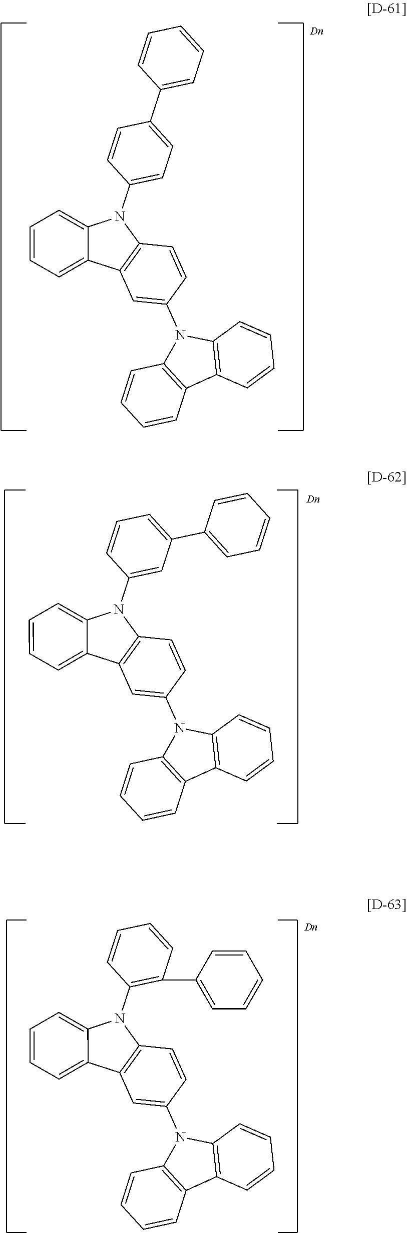

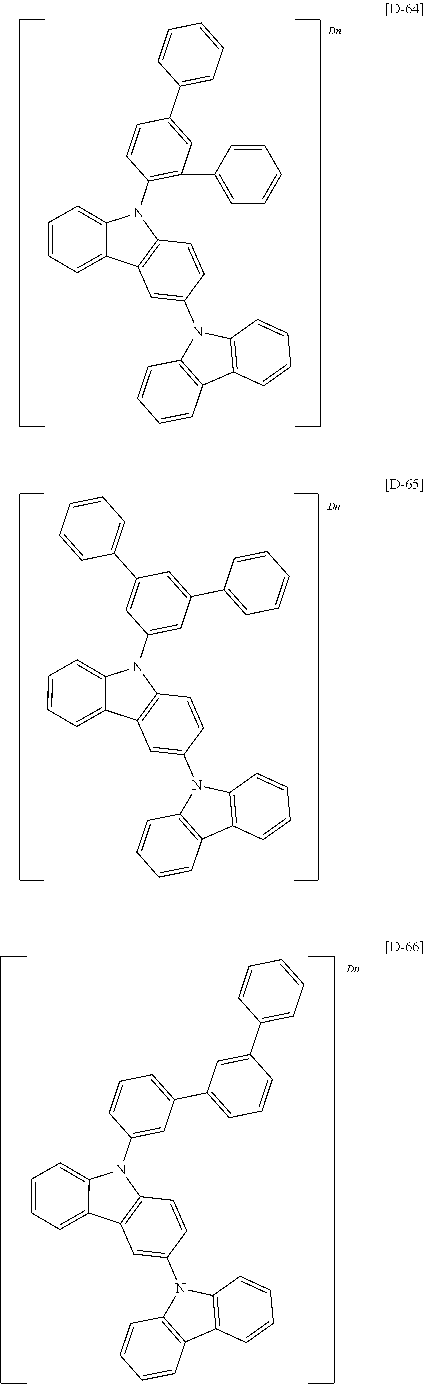

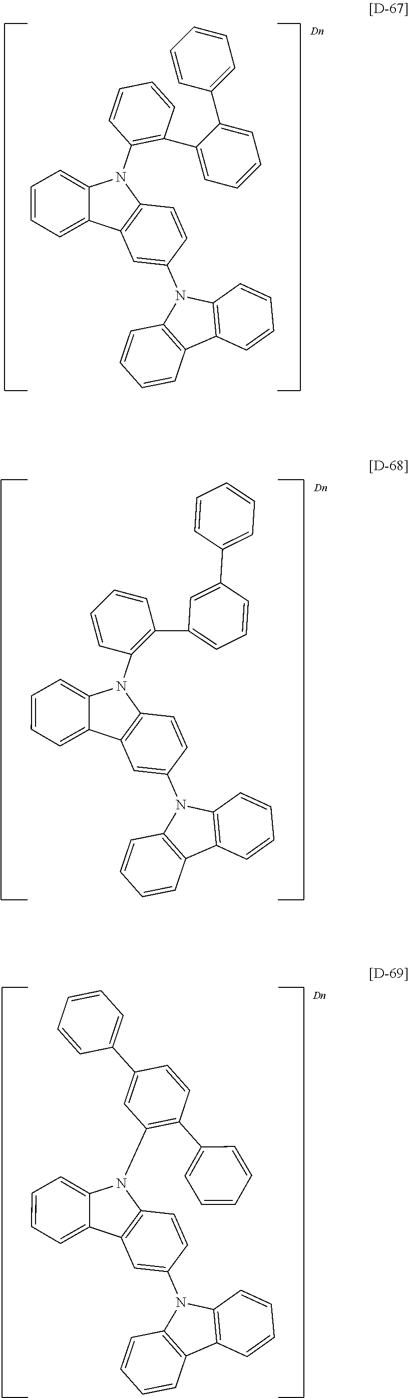

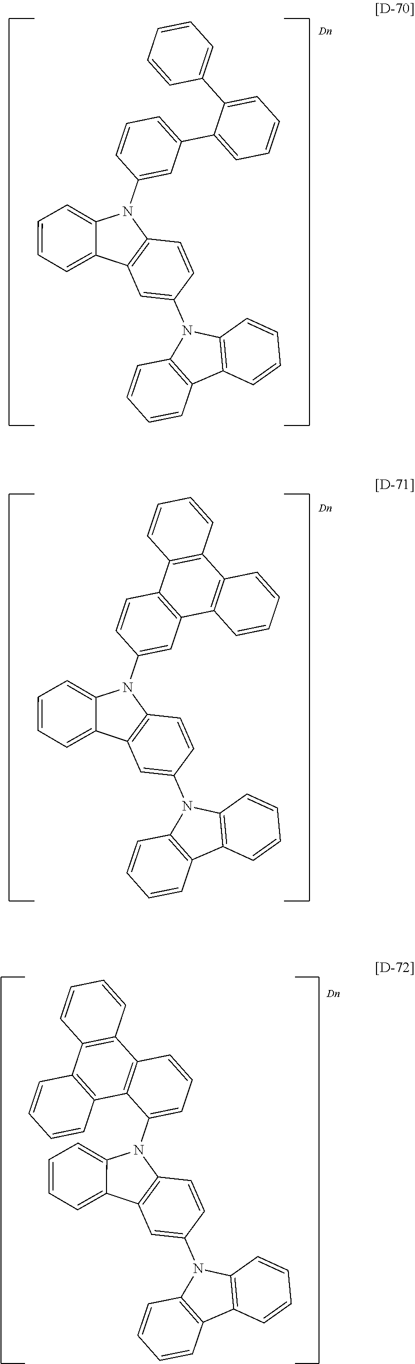

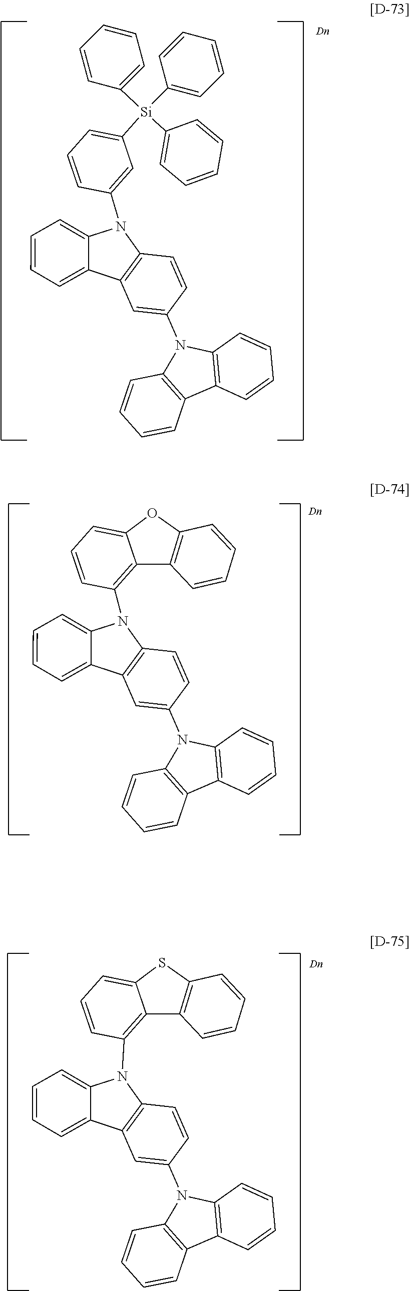

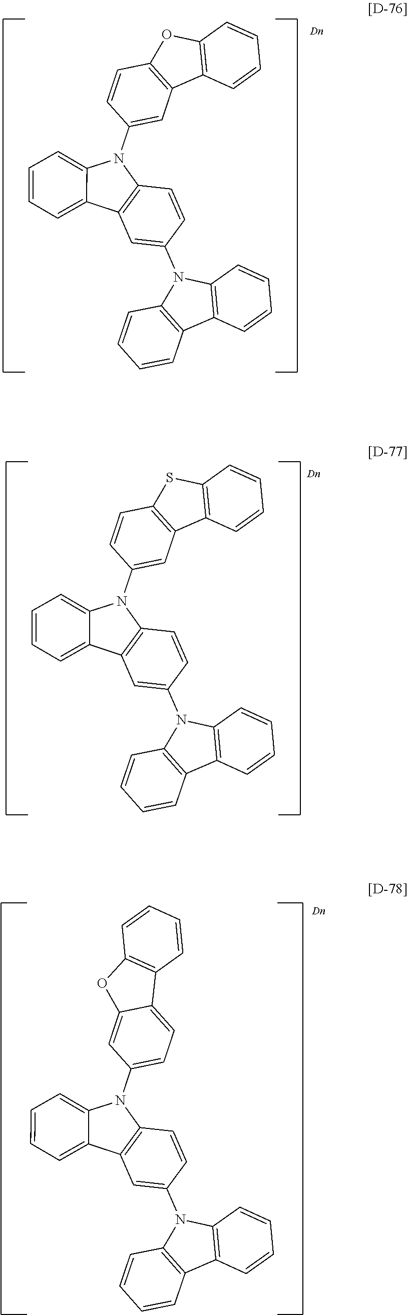

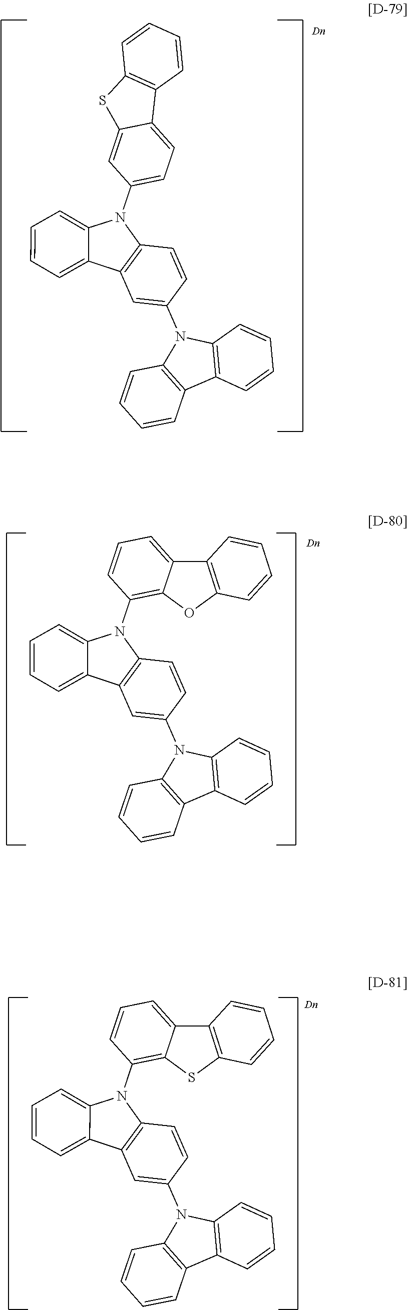

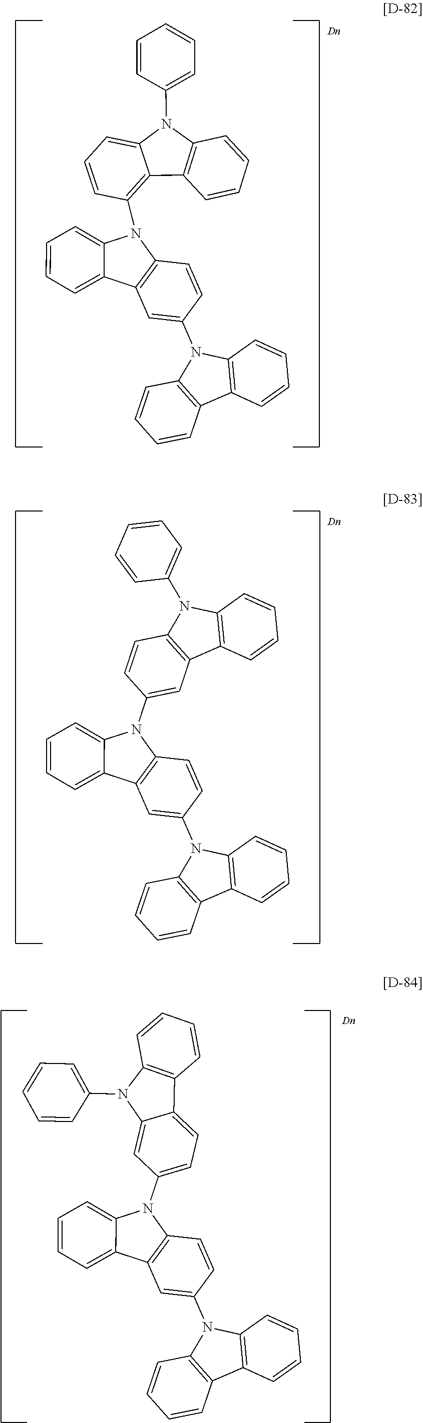

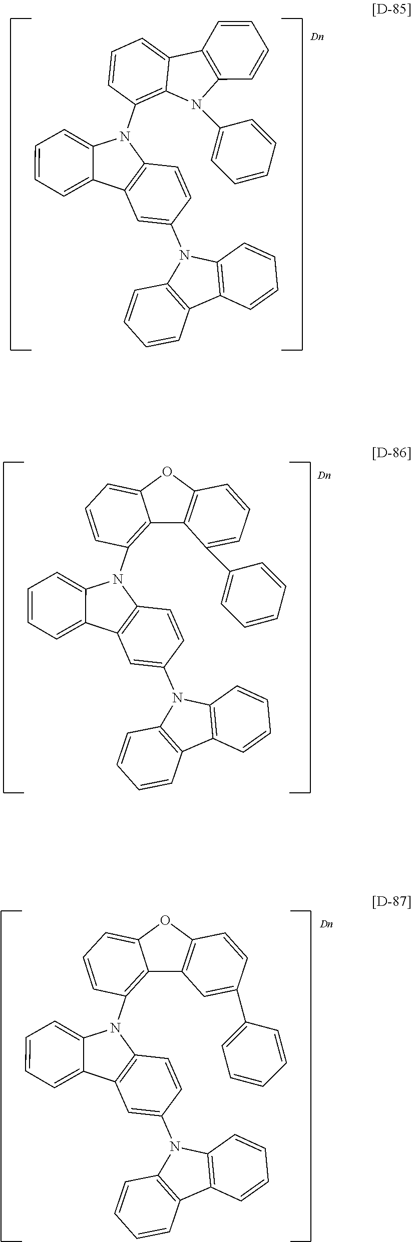

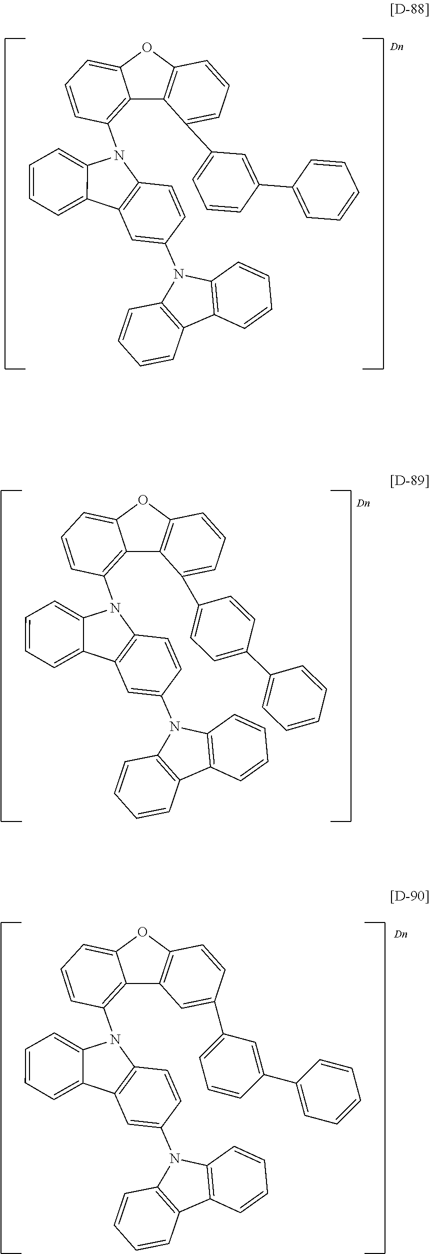

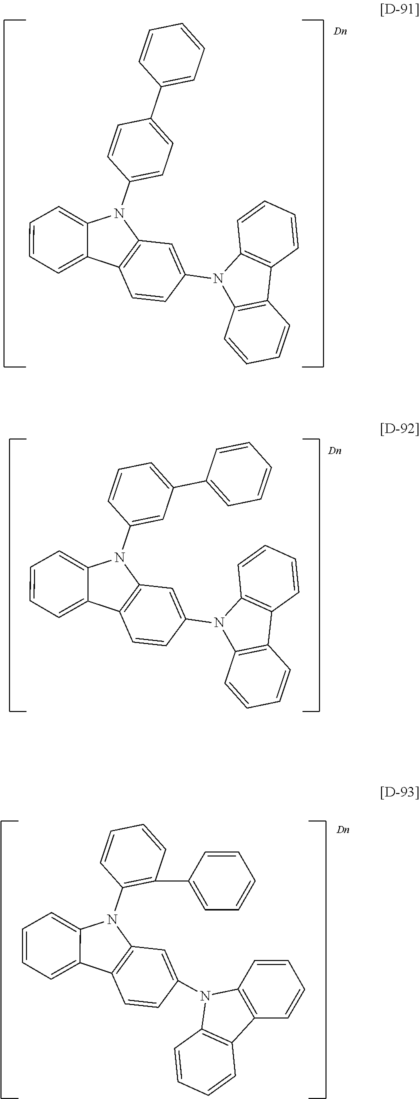

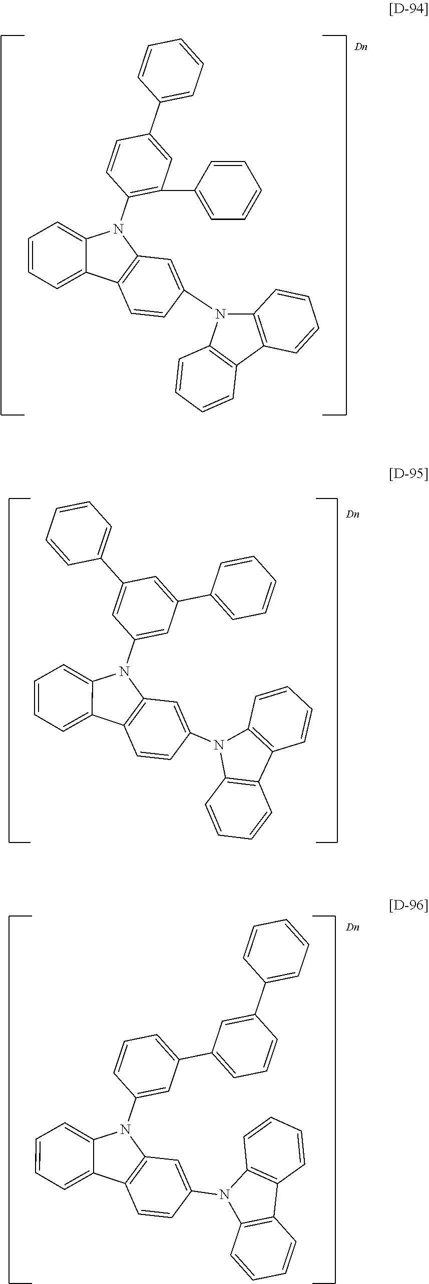

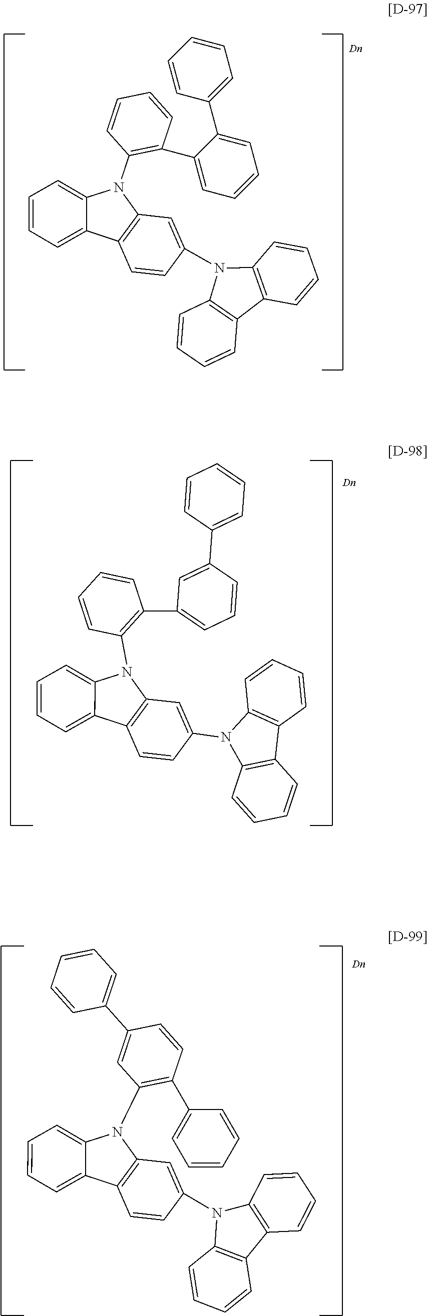

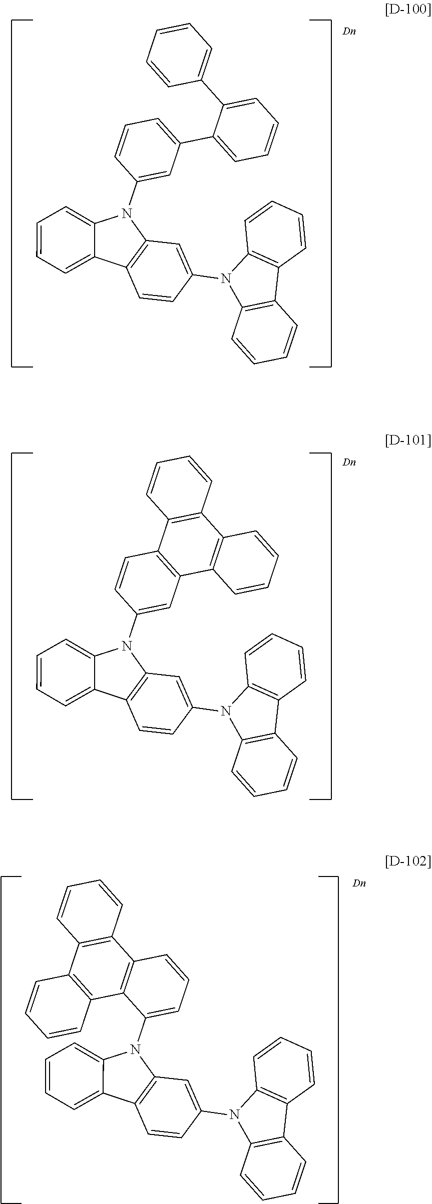

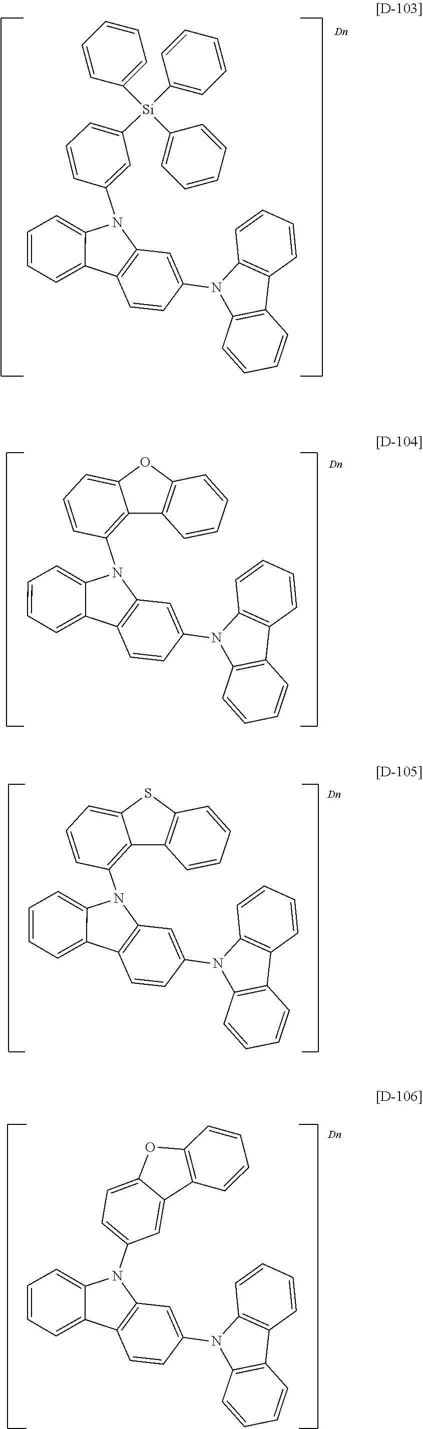

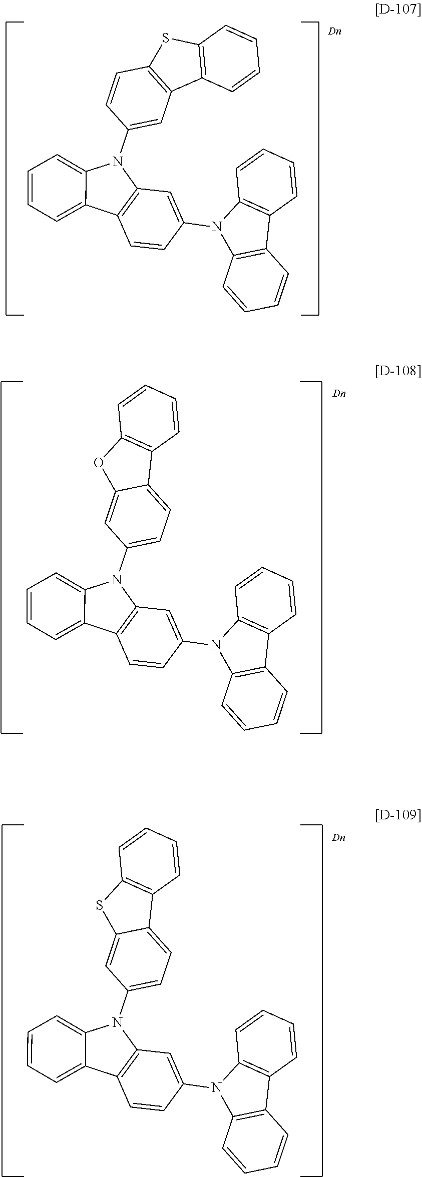

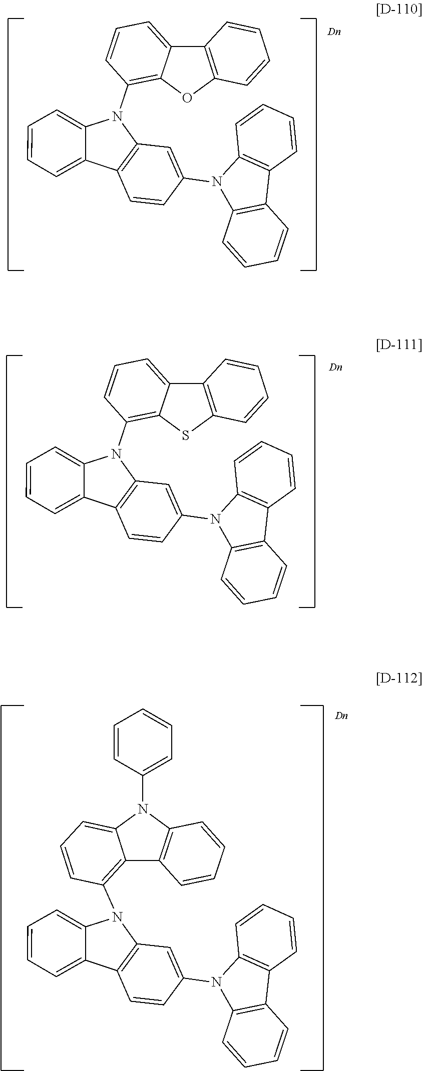

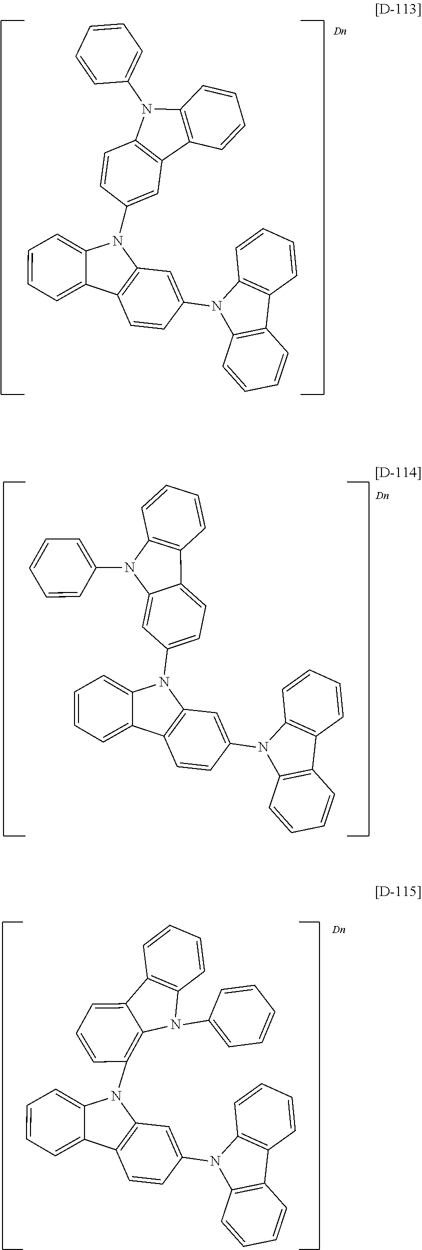

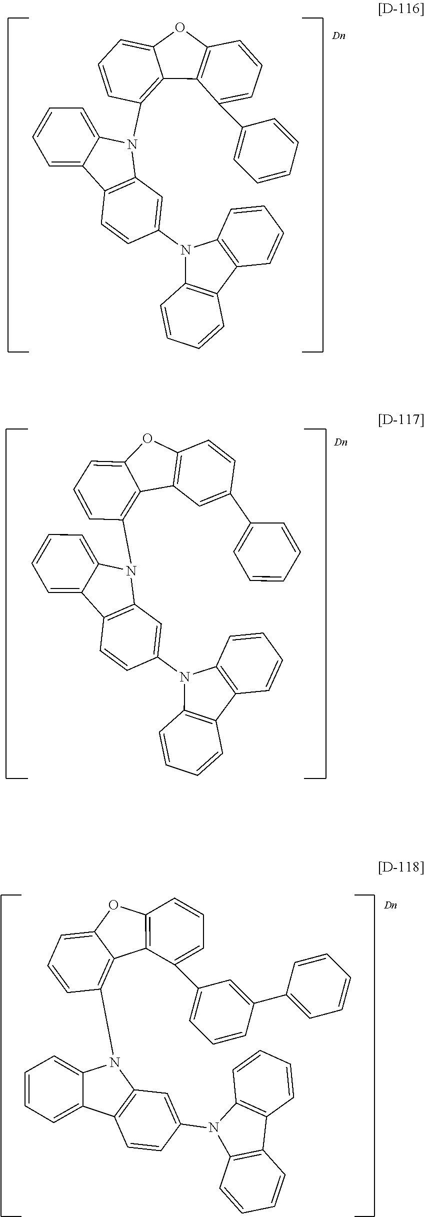

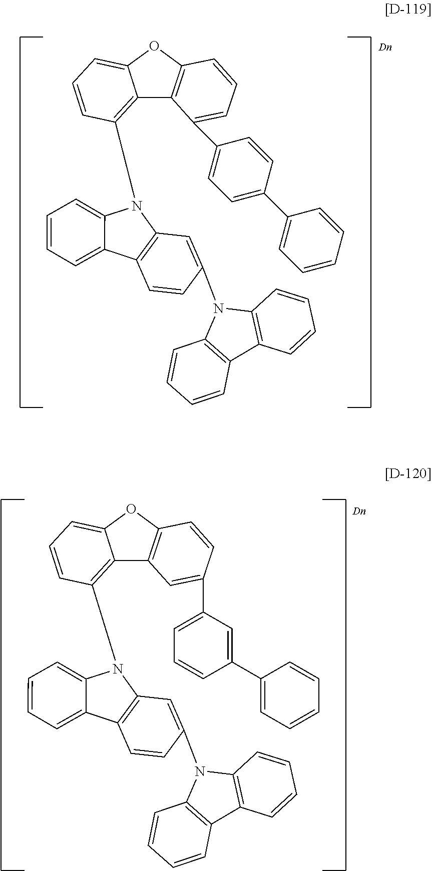

(Dn refers to the number of deuterium substitutions and indicates a structure substituted with one or more deuterium atoms)

In an implementation, the second compound may be represented by Chemical Formula 3C.

The first compound and the second compound may be included (e.g., mixed) in a weight ratio of, e.g., about 1:99 to about 99:1. By being included in the above range, efficiency and life-span can be improved by implementing bipolar characteristics by adjusting the appropriate weight ratio using the electron transport capability of the first compound and the hole transport capability of the second compound. Within the above range, they may be included in a weight ratio of about 10:90 to about 90:10, about 20:80 to about 80:20, e.g., about 20:80 to about 70:30, about 20:80 to about 60:40, and about 30:70 to about 60:40. In an implementation, they may be included in a weight ratio of about 40:60, about 50:50, or about 60:40.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to the drawing.

The FIGURE is a cross-sectional view showing an organic light emitting diode according to some embodiments.

Referring to the FIGURE, an organic light emitting diode 100 according to some embodiments may include, e.g., an anode 120 and a cathode 110 facing each other and an organic layer 105 between the anode 120 and cathode 110.

The anode 120 may be made of a conductor having a large work function to help hole injection, and may be, e.g., a metal, a metal oxide and/or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, and/or a conductive polymer. The cathode 110 may be, e.g., a metal such as magnesium, calcium, sodium, potassium, titanium, indium, yttrium, lithium, gadolinium, aluminum, silver, tin, lead, cesium, barium, or the like, or an alloy thereof; a multi-layer structure material such as LiF/Al, LiO₂/Al, LiF/Ca, LiF/Al, or BaF₂/Ca.

The organic layer 105 may include the aforementioned compound for an organic optoelectronic device or composition for an organic optoelectronic device.

The organic layer 105 may include a light emitting layer 130, and the light emitting layer 130 may include a host and a dopant, the host may include the aforementioned compound for an organic optoelectronic device or composition for an organic optoelectronic device, and the dopant may be, e.g., a phosphorescent dopant, e.g., a red, green or blue phosphorescent dopant, e.g., a red or green phosphorescent dopant.

The dopant may be a material mixed with the compound or composition for an organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may be a phosphorescent dopant and examples of the phosphorescent dopant may include an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L⁶MX¹ [Chemical Formula Z]

In Chemical Formula Z, M may be a metal, and L⁶ and X¹ may each independently be ligands forming a complex compound with M.

M may be, e.g., Ir, Pt, Os, Ti, Zr, Hf, Eu, Tb, Tm, Fe, Co, Ni, Ru, Rh, Pd, or a combination thereof, and L⁶ and X¹ may be, e.g., a bidentate ligand.

In an implementation, the ligands represented by L⁶ and X¹ may be a ligand of Group A.

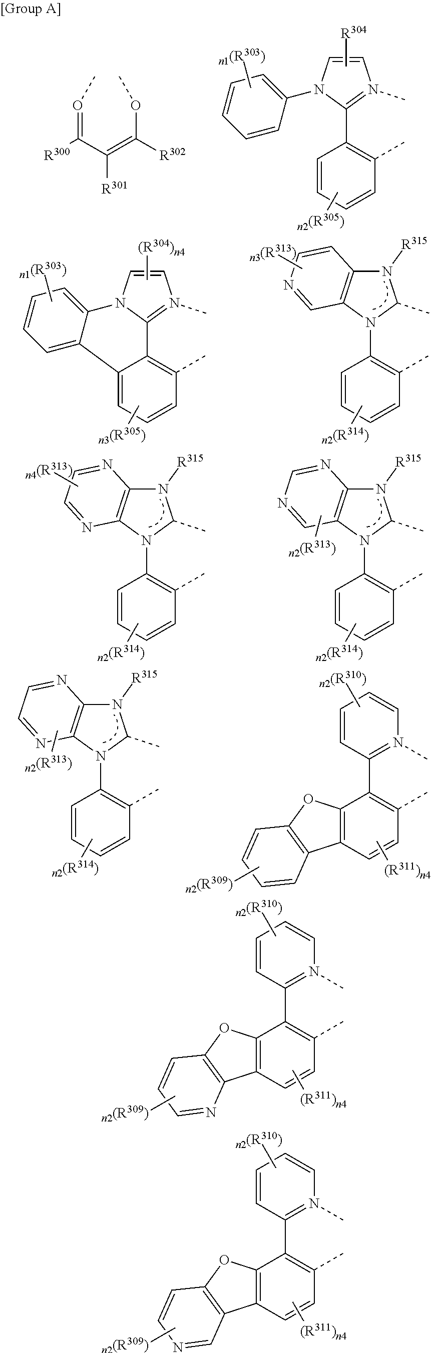

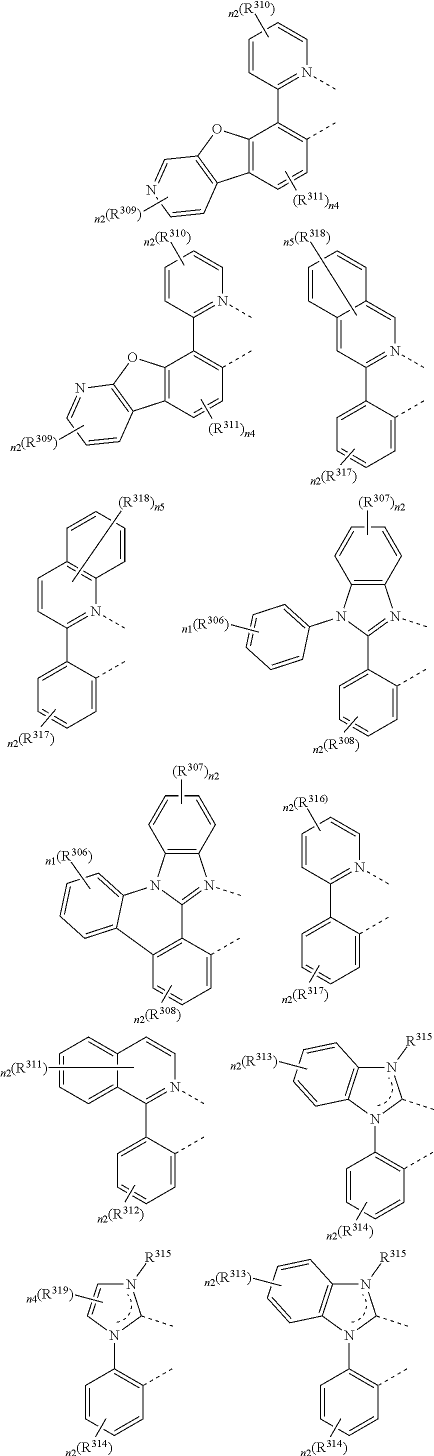

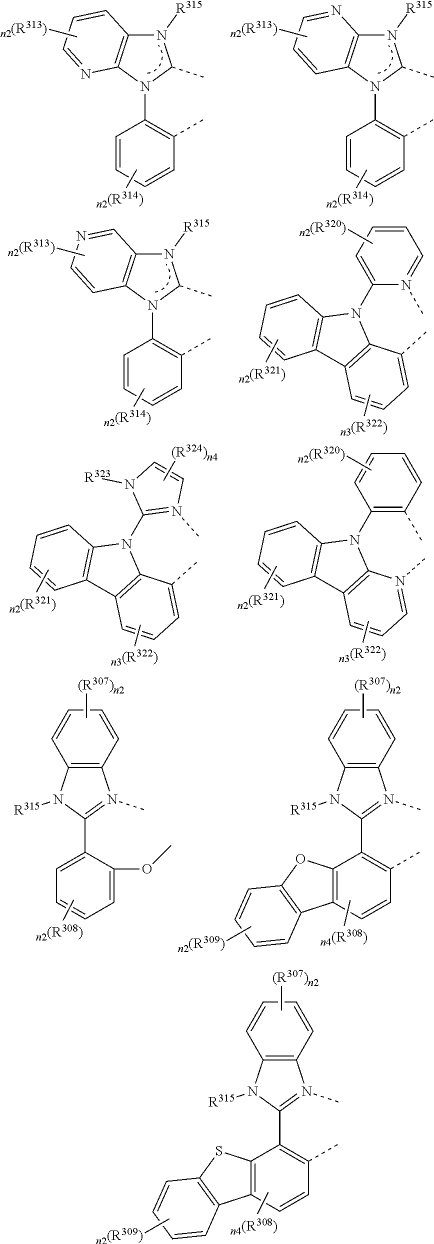

In Group A, R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³⁰³ to R³²⁴ may each independently be, e.g., hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

n1 may be, e.g., an integer of 1 to 5.

n2 may be, e.g., an integer of 1 to 4.

n3 may be, e.g., an integer of 1 to 3.

n4 may be, e.g., an integer of 1 or 2.

n5 may be, e.g., an integer of 1 to 6.

The dopant according to some embodiments may be an iridium complex, and may be represented by, e.g., Chemical Formula 6-1 or Chemical Formula 6-2.

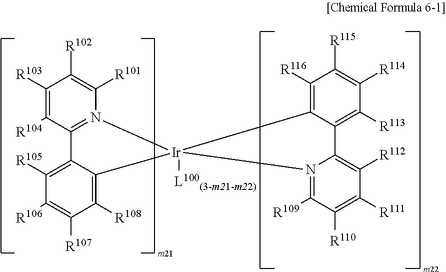

In Chemical Formula 6-1, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

At least one of R¹⁰¹ to R¹¹⁶ may be a functional group represented by Chemical Formula V-1.

L¹⁰⁰ may be a bidentate ligand of a monovalent anion, and may be a ligand that coordinates to iridium through a lone pair of carbons or heteroatoms.

m21 and m22 may each independently be, e.g., an integer of 0 to 3, and m21+m22 may be an integer of 1 to 3.

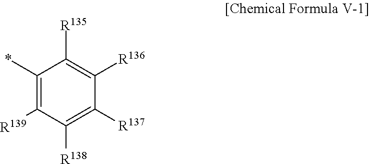

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

* means a portion linked to a carbon atom.

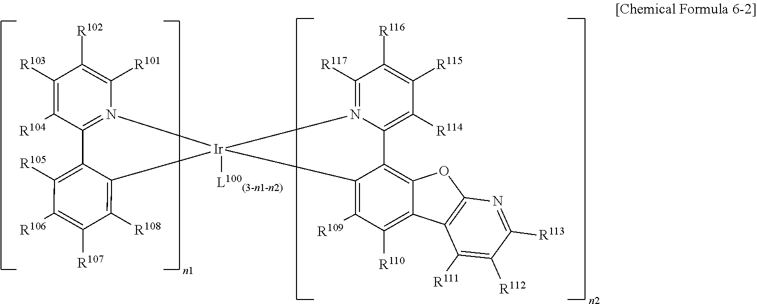

In Chemical Formula 6-2, R¹⁰¹ to R¹¹⁷ may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

L¹⁰⁰ may be a bidentate ligand of a monovalent anion, and may be a ligand that coordinates to iridium through a lone pair of carbons or heteroatoms.

n1 and n2 may each independently be, e.g., an integer of 0 to 3, and n1+n2 may be, e.g., an integer of 1 to 3.

The dopant according to some embodiments may be a platinum complex, and may be represented, e.g., by Chemical Formula Z-1.

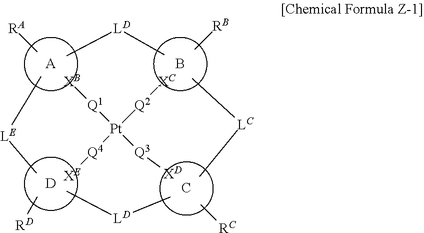

In Chemical Formula Z-1, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution, or unsubstitution.

L^{B}, L^{C}, and L^{D} may each independently be, e.g., a direct bond, BR, NR, PR, O, S, Se, C═O, S═O, SO₂, CRR′, SiRR′, GeRR′, or a combination thereof.

In an implementation, nA may be 1, and L^{E} may be a direct bond, BR, NR, PR, O, S, Se, C═O, S═O, SO₂, CRR′, SiRR′, GeRR′, or a combination thereof. In an implementation, nA may be 0 and L^{E} does not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof. In an implementation, any adjacent ones of R^{A}, R^{B}, R^{C}, R^{D}, R, and R′ may be separate or may be linked to each other to provide a ring; X^{B}, X^{C}, X^{D}, and X^{E} may each independently be, e.g., carbon or nitrogen; and Q¹, Q², Q³, and Q⁴ may each independently be, e.g., oxygen or a direct bond.

The platinum complex may be represented, e.g., by Chemical Formula 7-1 or Chemical Formula 7-2.

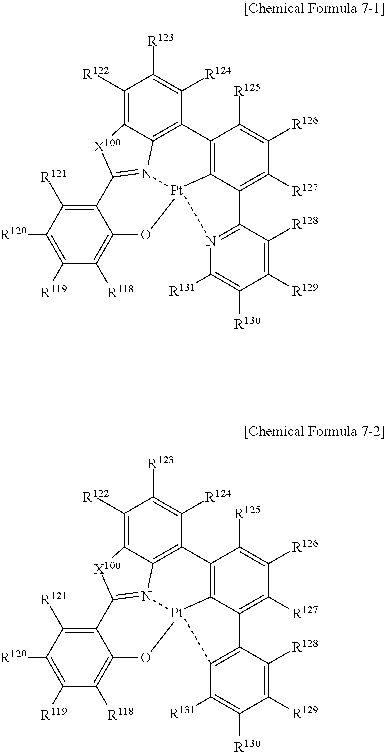

In Chemical Formula 7-1 and Chemical Formula 7-2, X¹⁰⁰ may be, e.g., O, S, or NR¹³².

R¹¹⁸ to R¹³² may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

In an implementation, at least one of R¹¹⁸ to R¹³² may be —SiR¹³³R¹³⁴R¹³⁵ or a tert-butyl group.

R¹³³ to R¹³⁵ may each independently be, e.g., a substituted or unsubstituted C1 to C6 alkyl group.

The organic layer may further include a charge transport region in addition to the light emitting layer.

The charge transport region may be, e.g., the hole transport region 140.

The hole transport region 140 may further increase hole injection and/or hole mobility between the anode 120 and the light emitting layer 130 and block electrons.

In an implementation, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

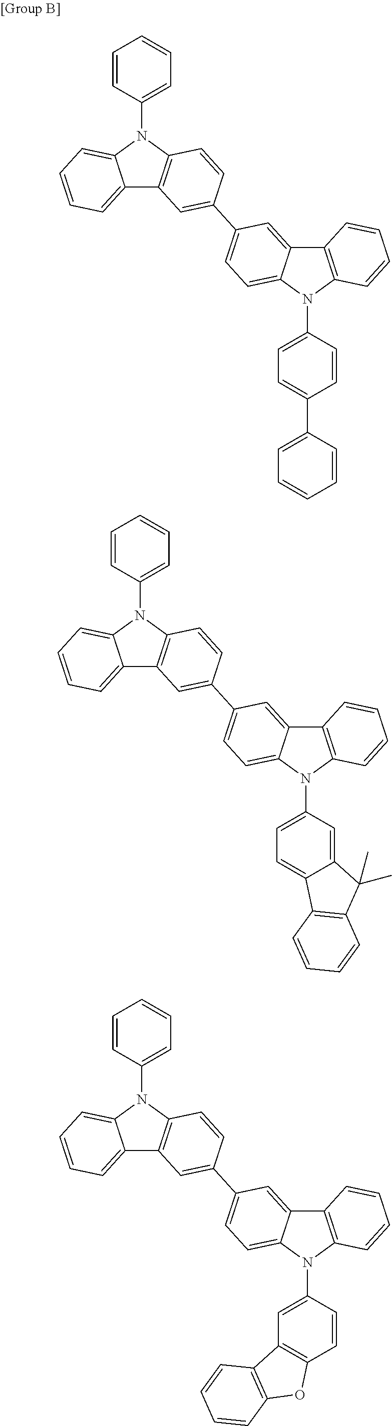 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

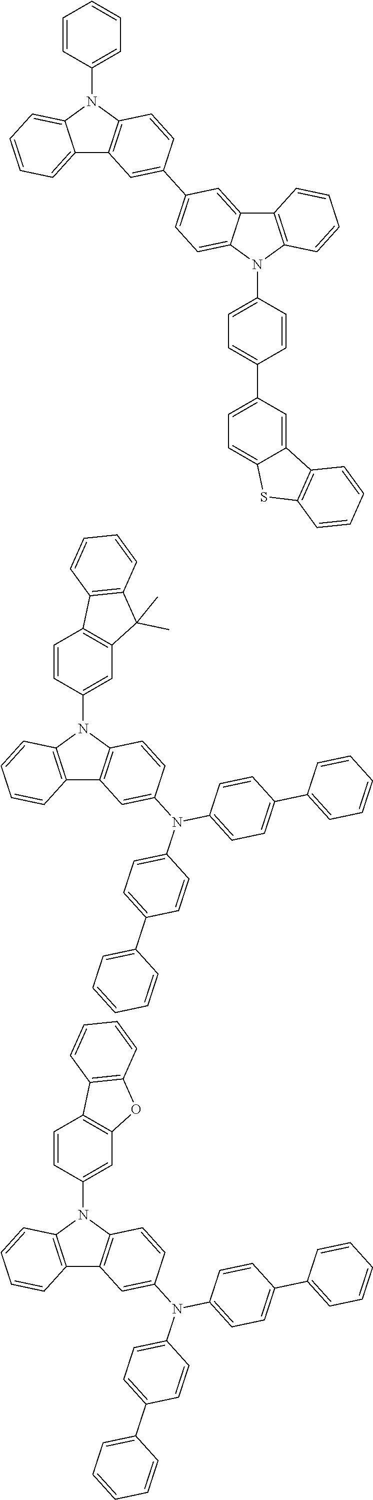

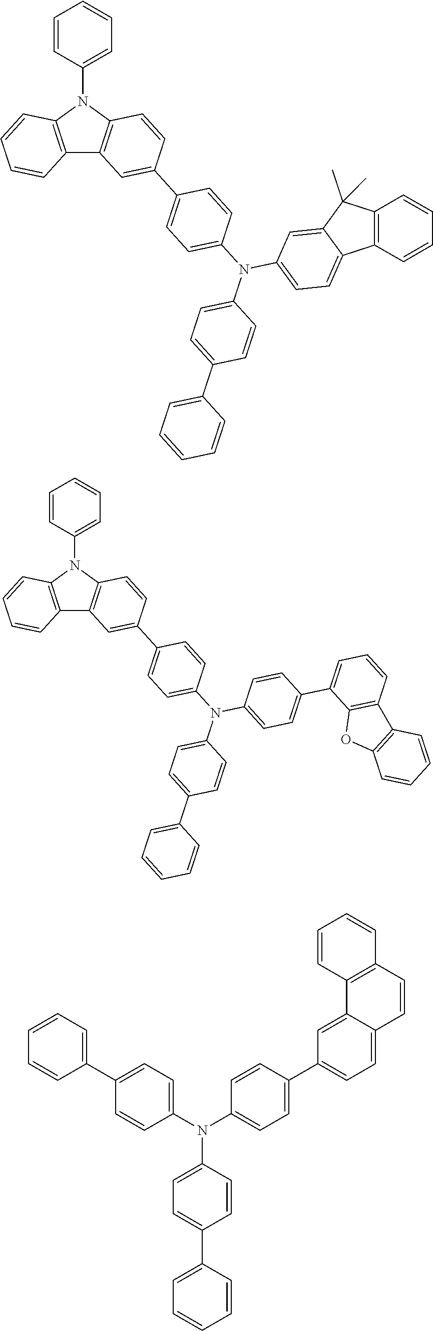 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

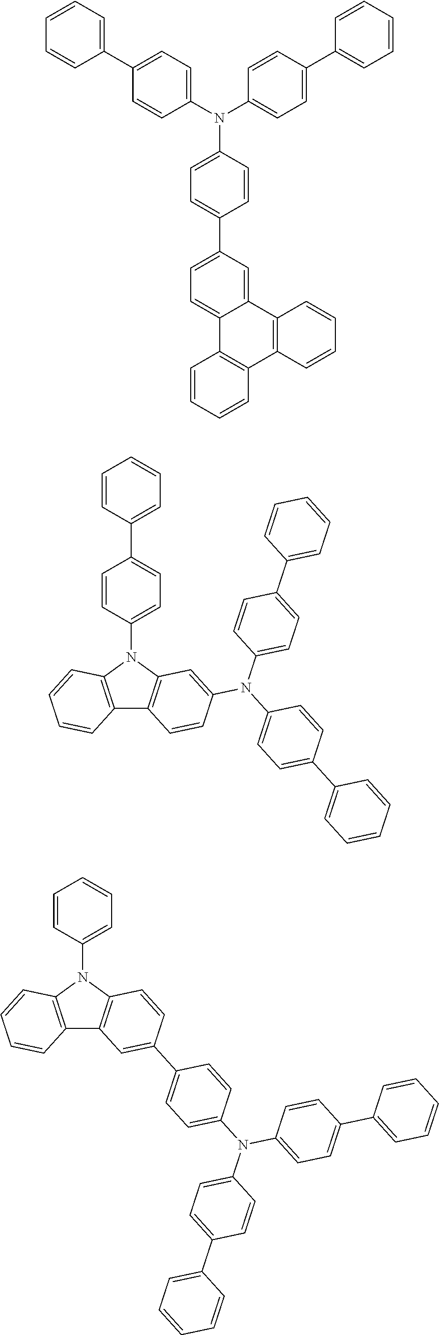 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

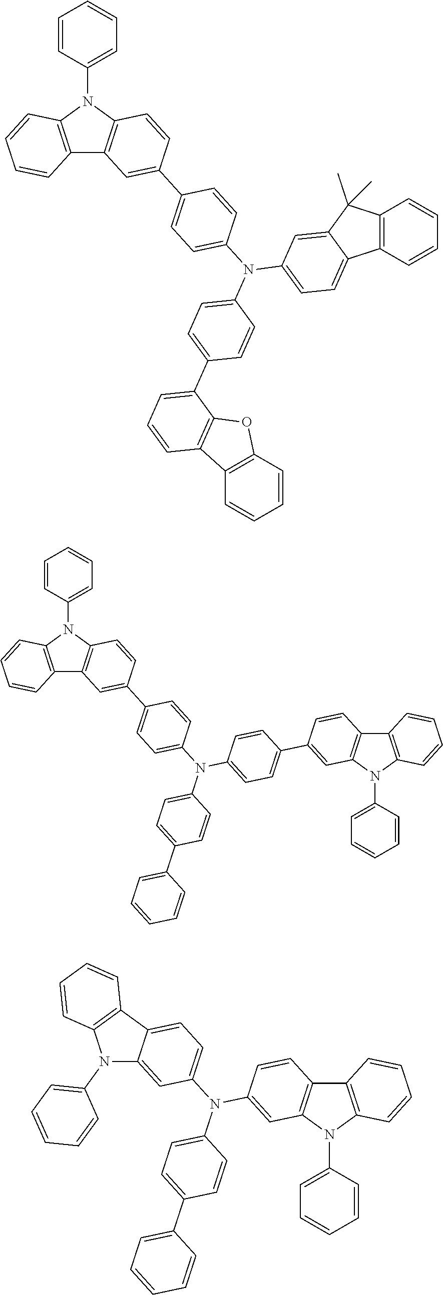

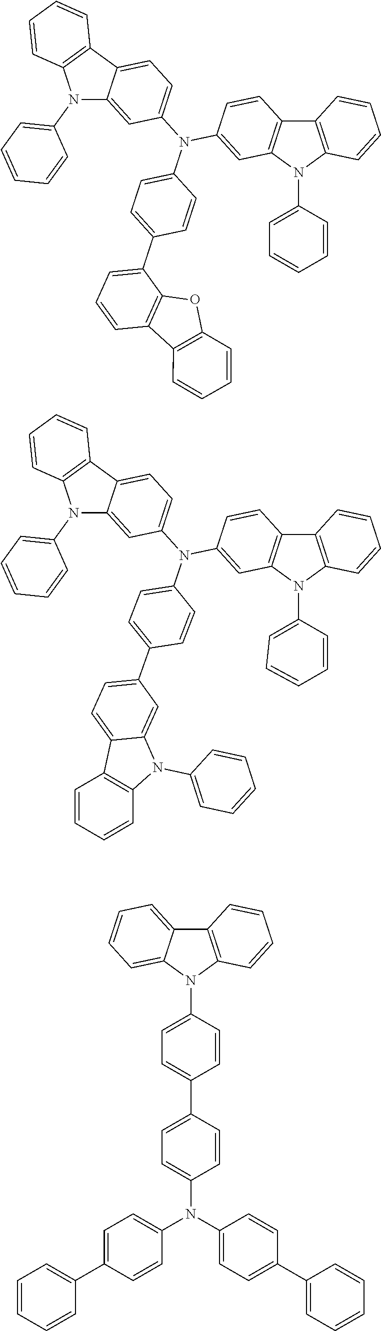

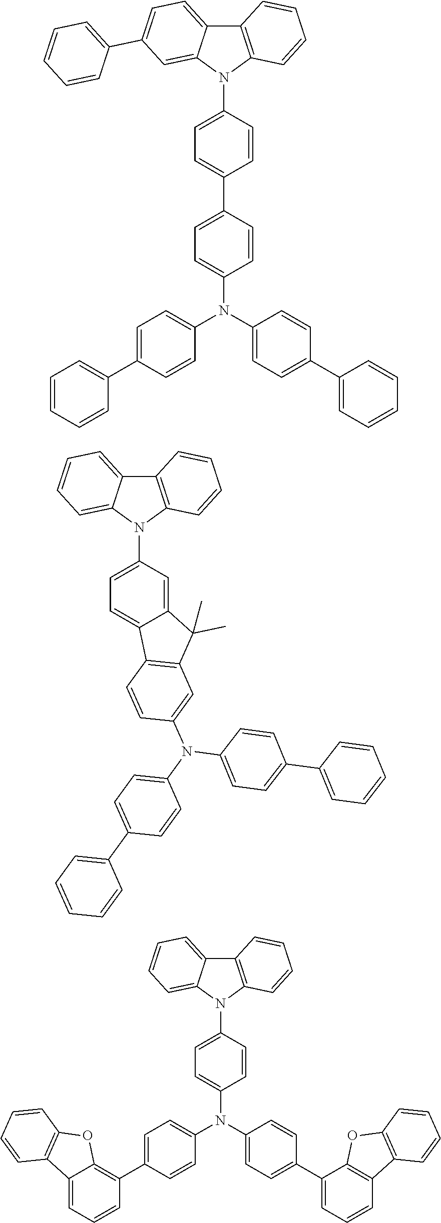

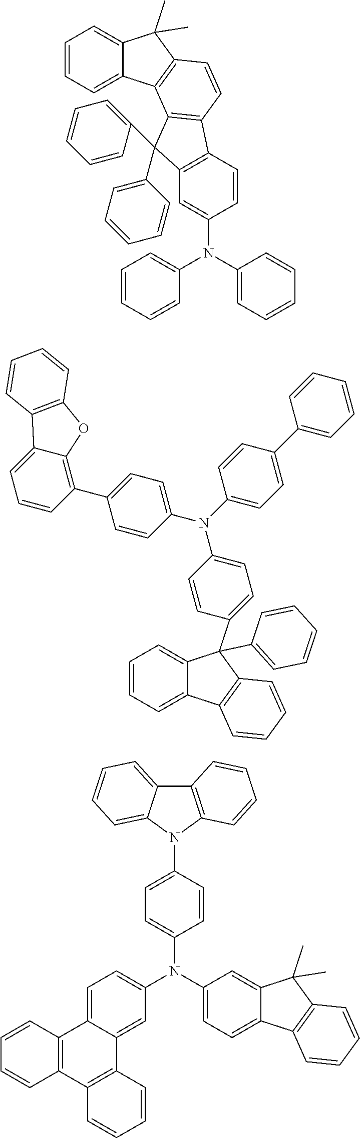

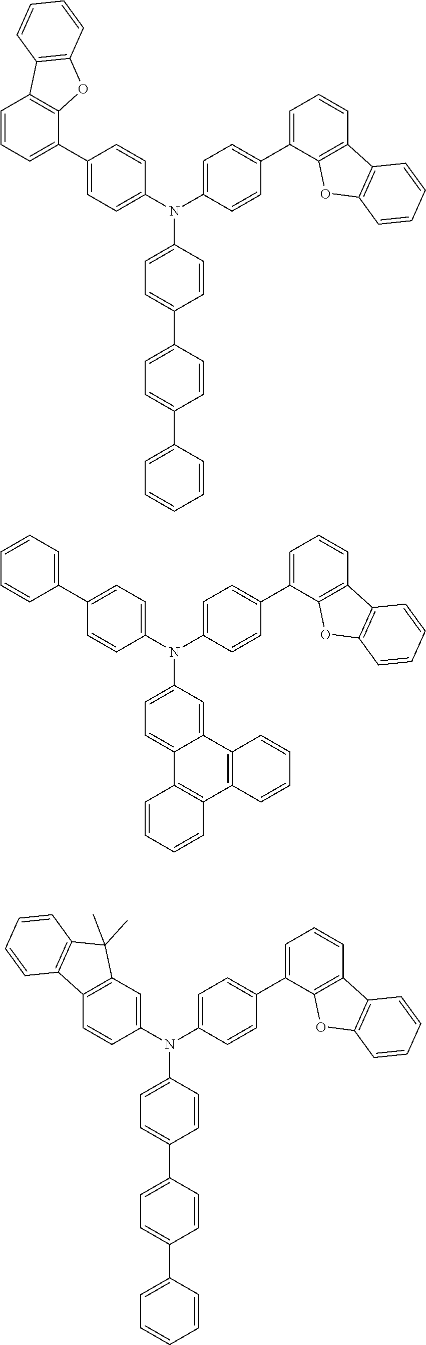 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21]

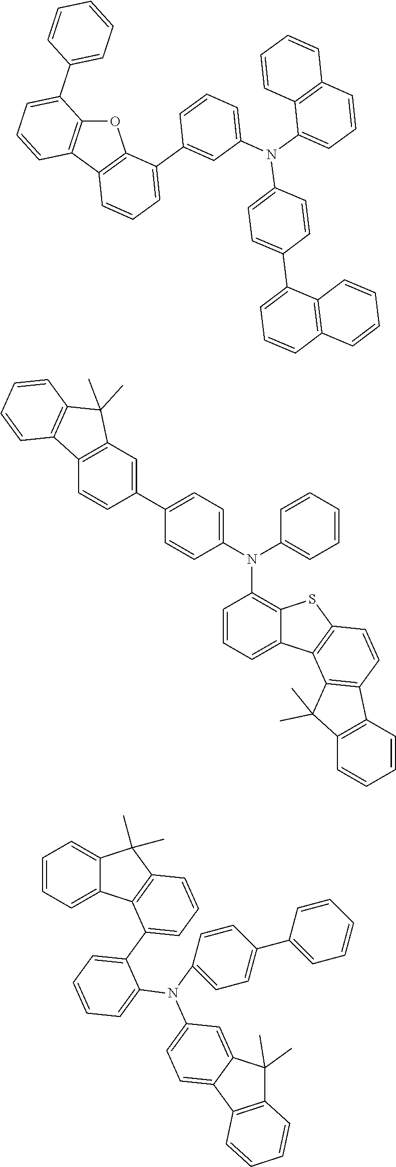 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

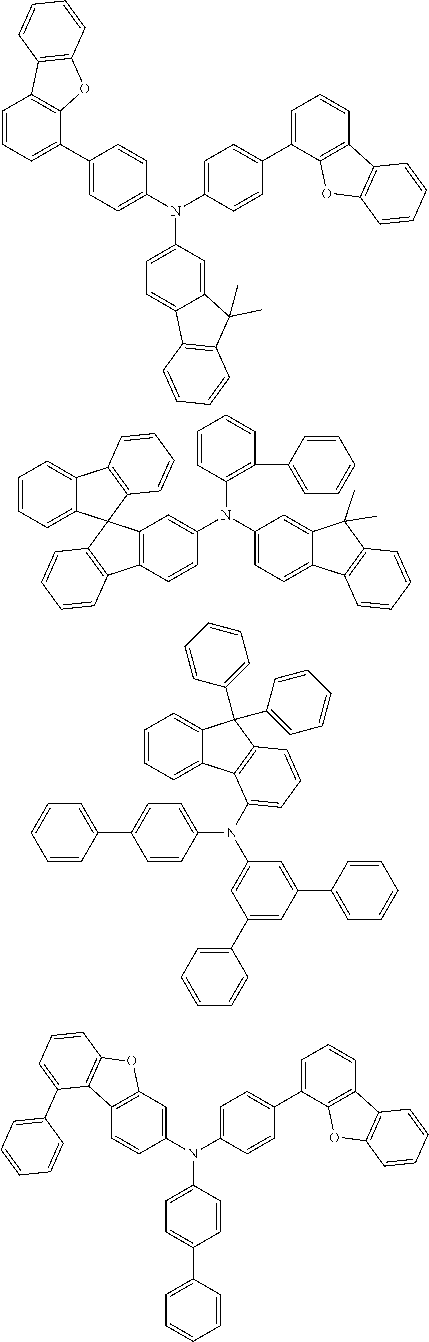

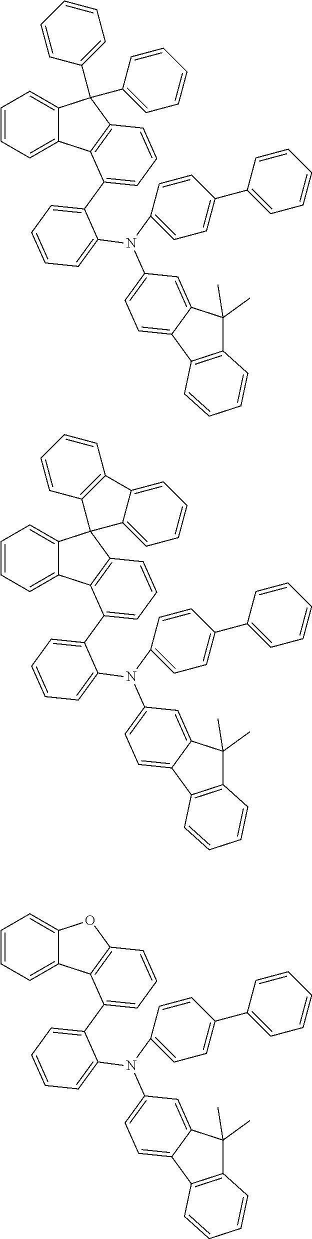 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

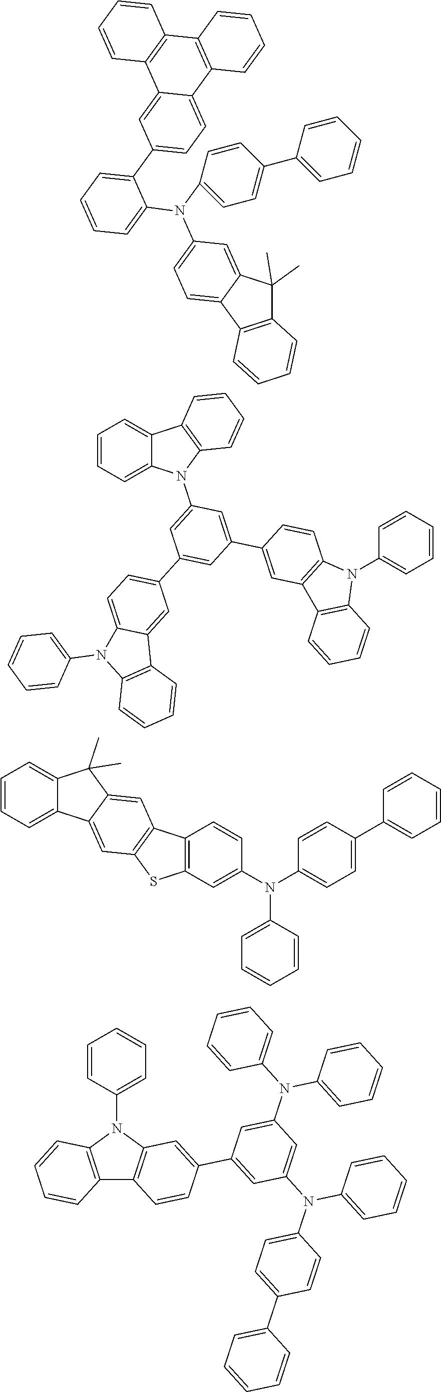

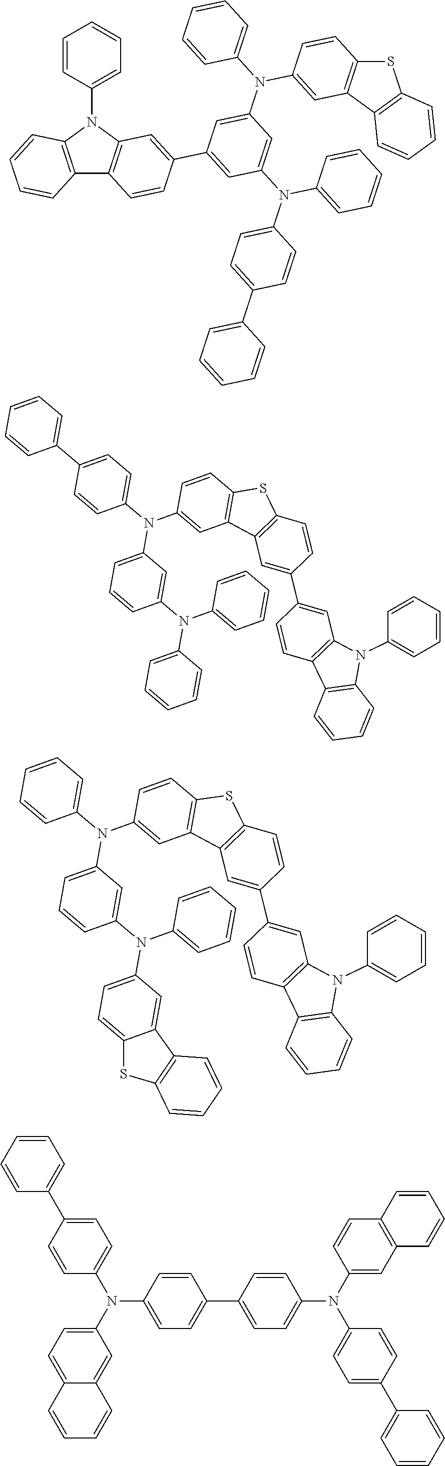 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1]

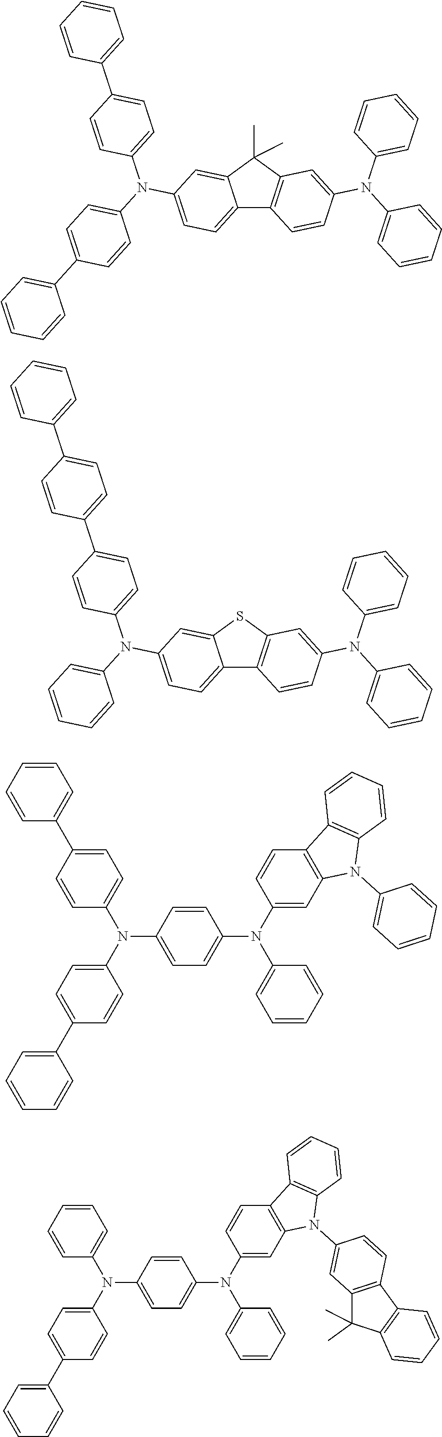

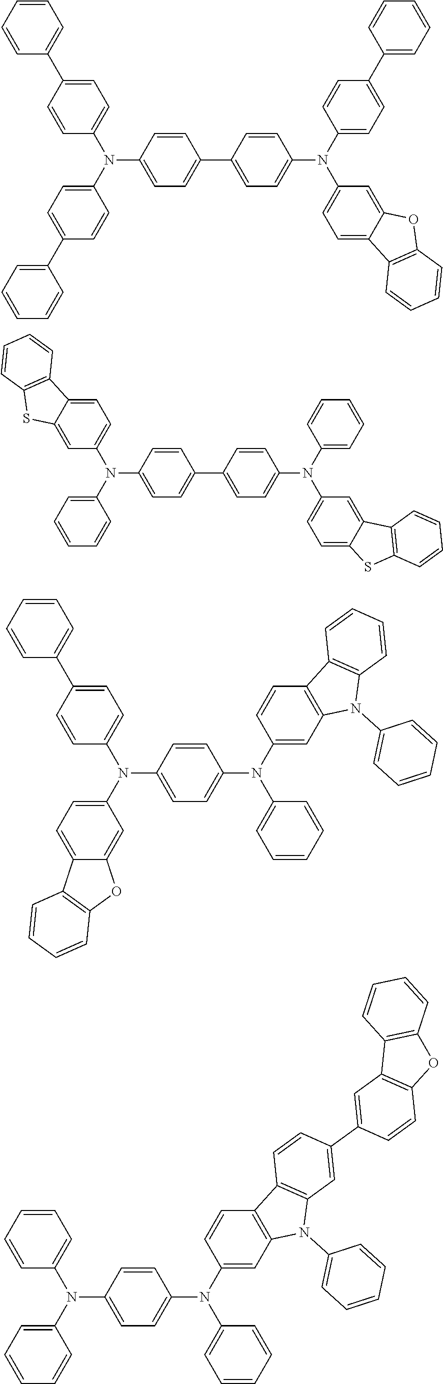

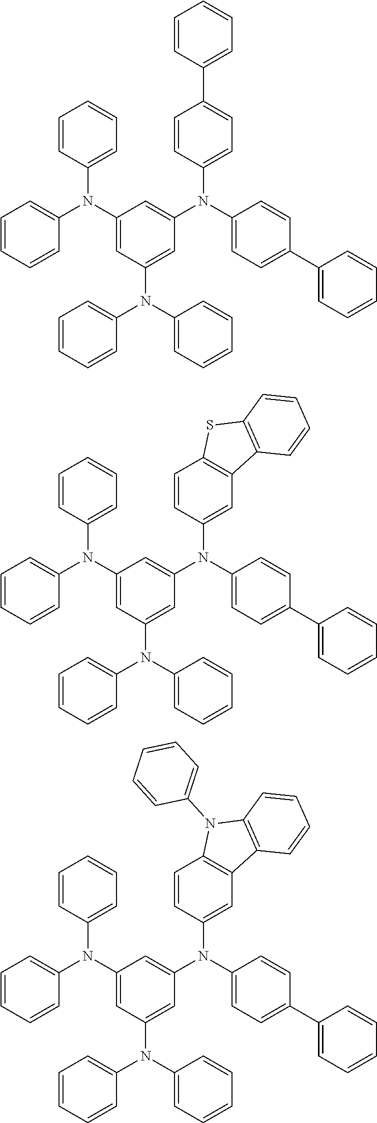

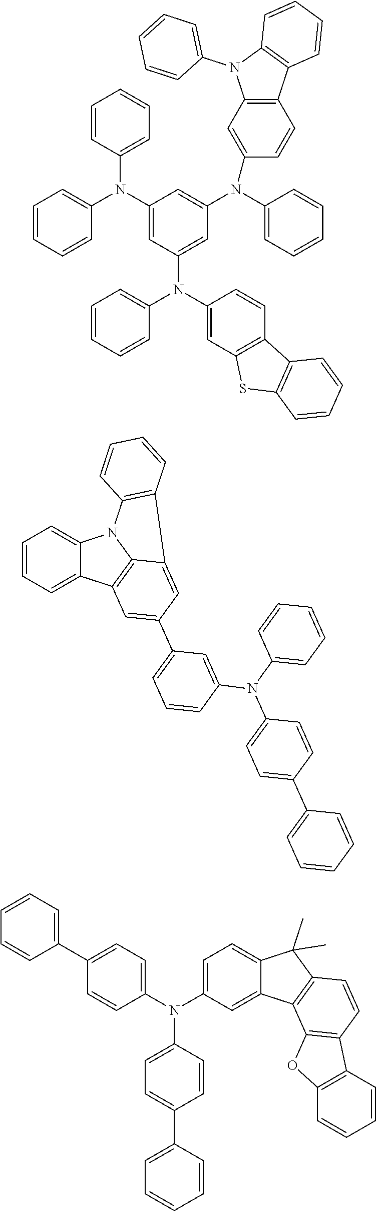 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

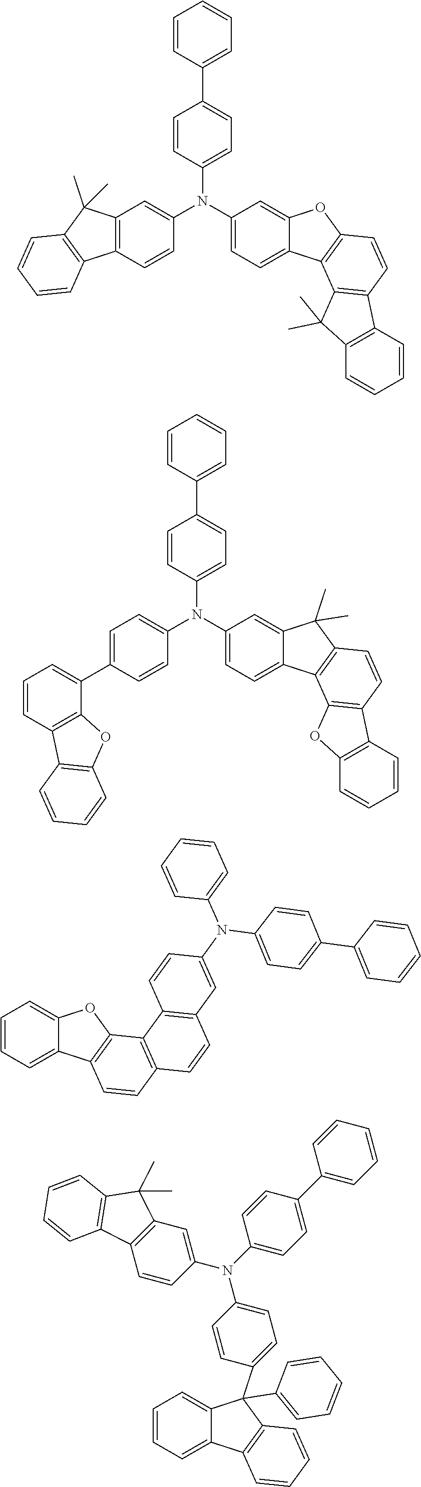 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

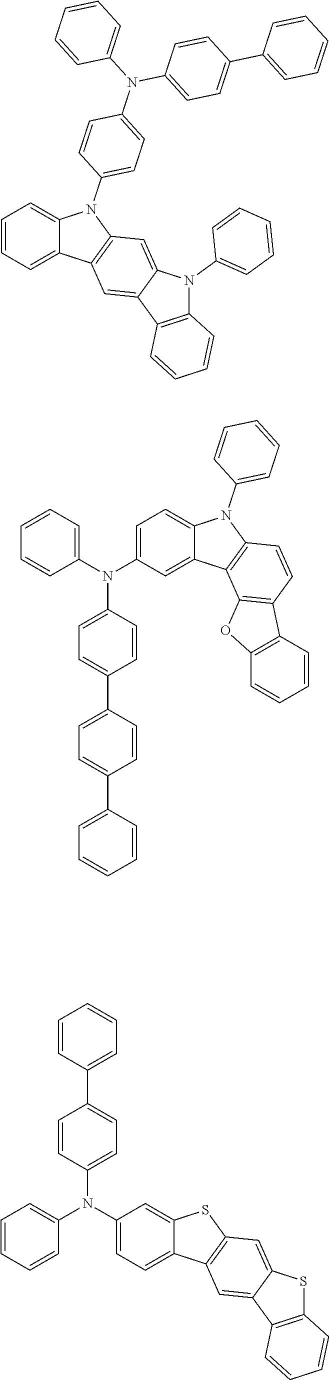 [SMILES: c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1]

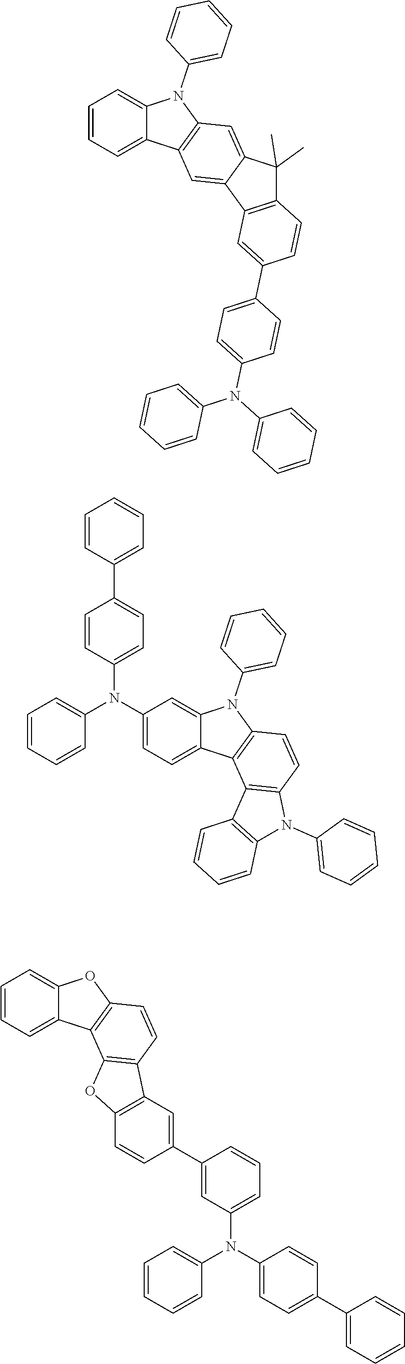 [SMILES: CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1]

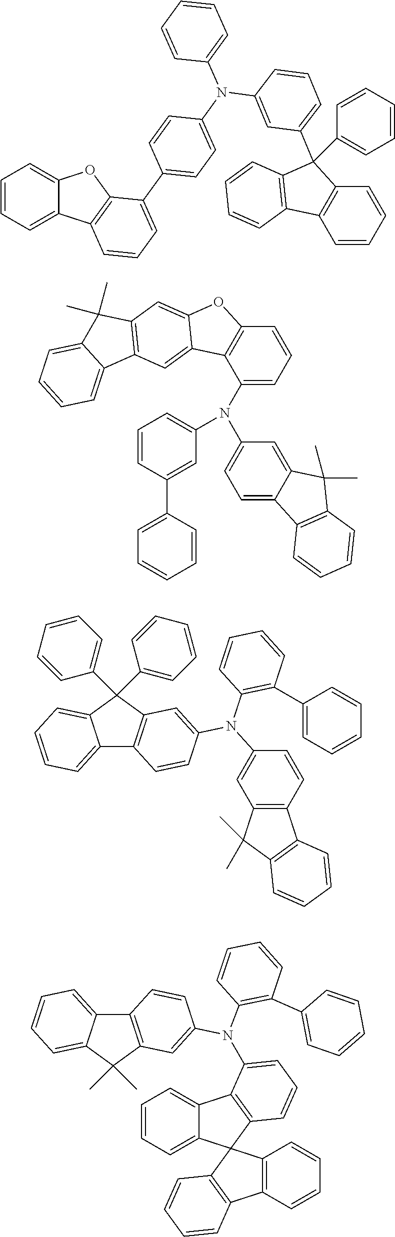

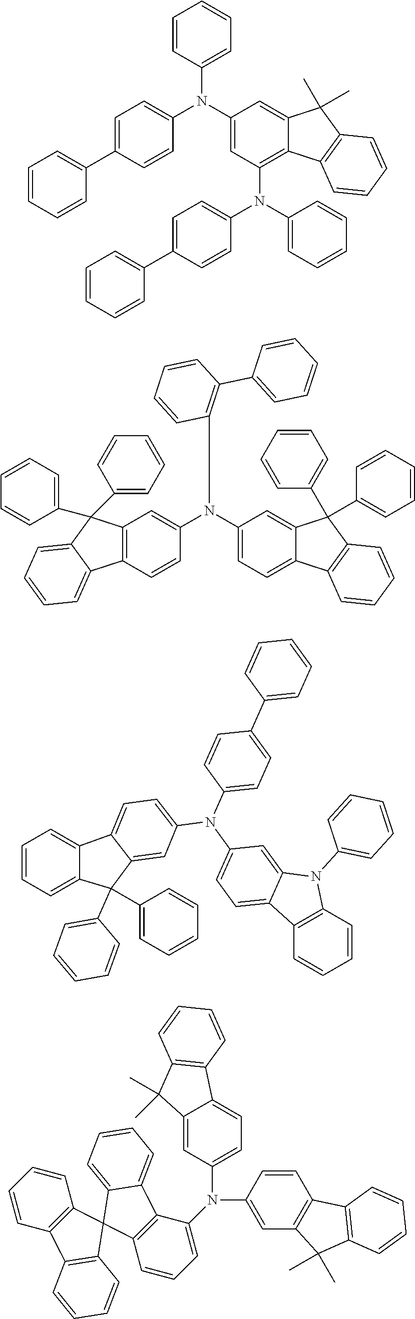 [SMILES: CC1(C)c2ccccc2-c2c(N(c3ccccc3)c3ccc(-c4ccccc4)cc3)cc(N(c3ccccc3)c3ccc(-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21]

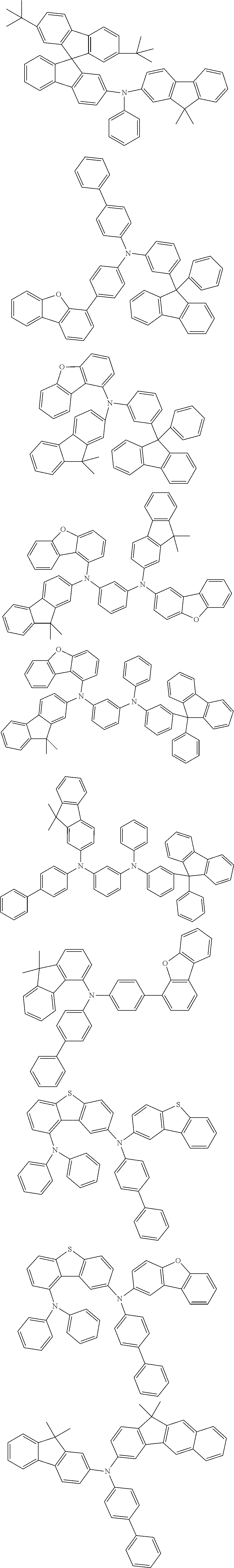

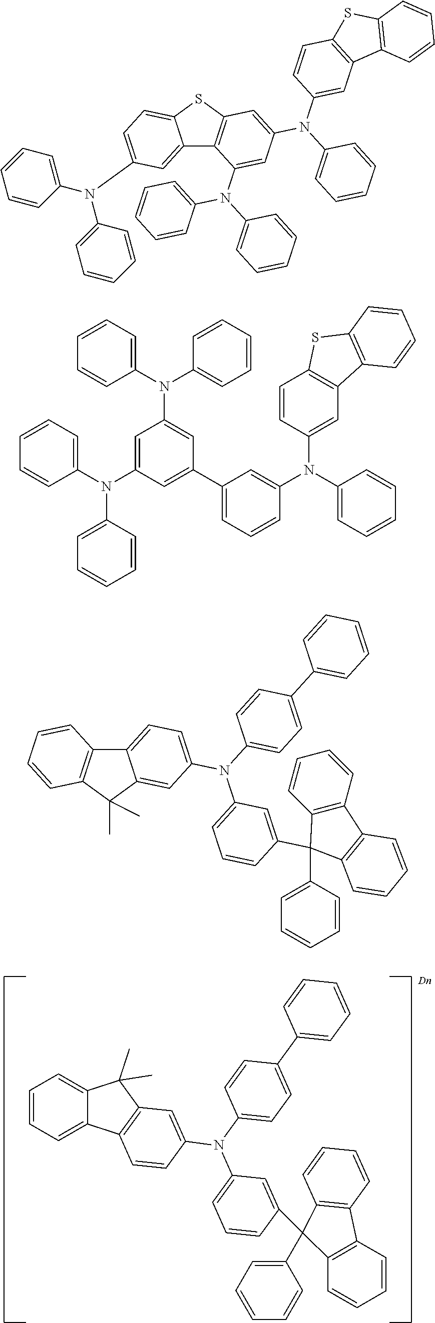

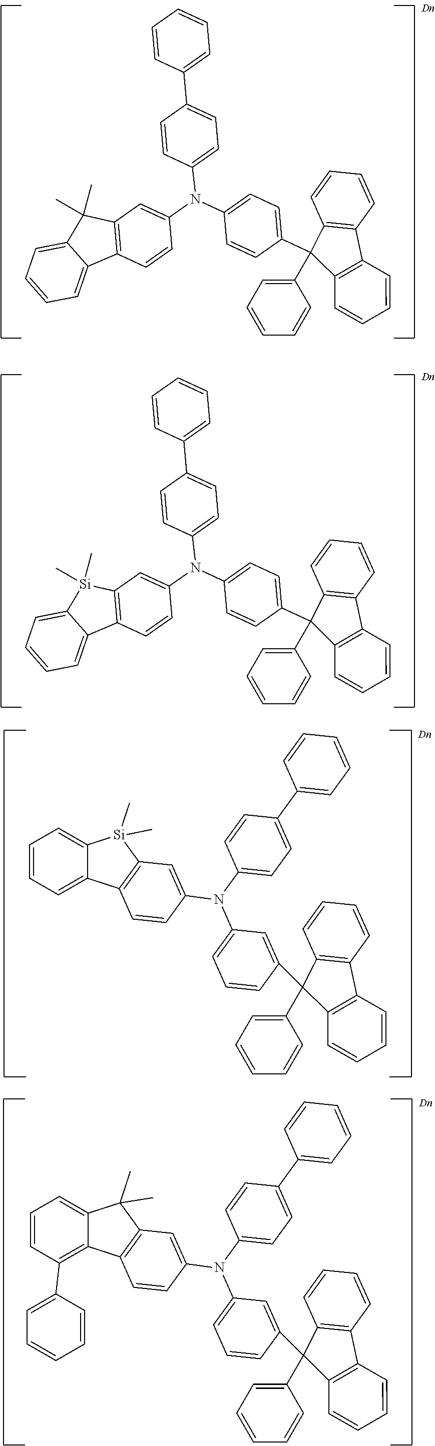

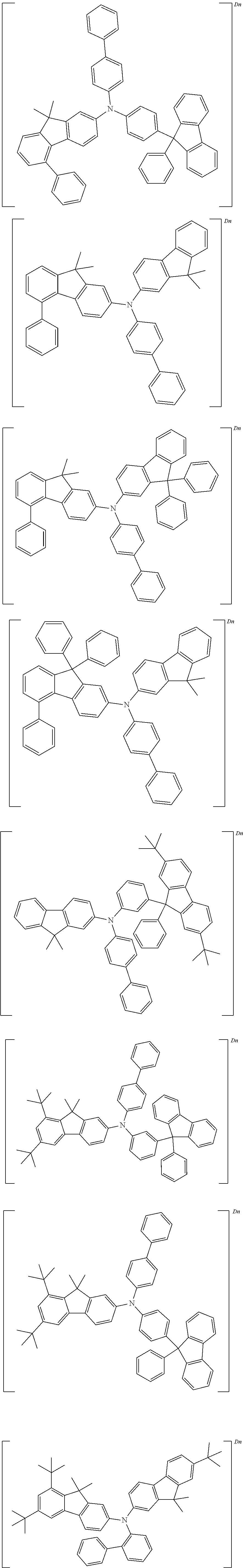

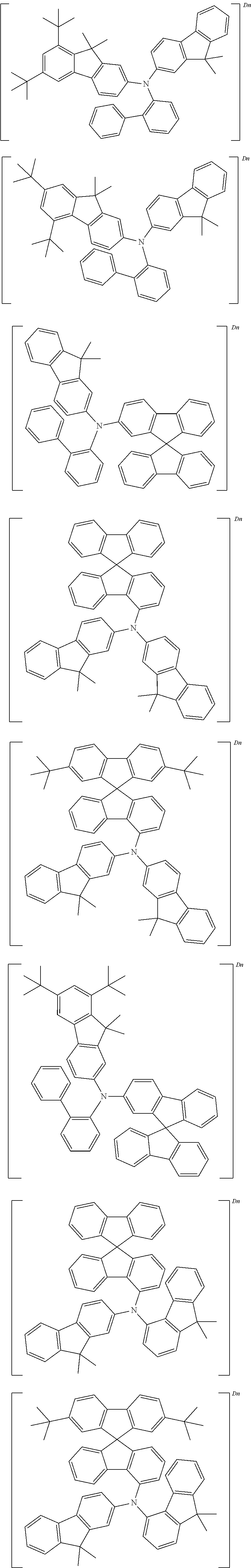

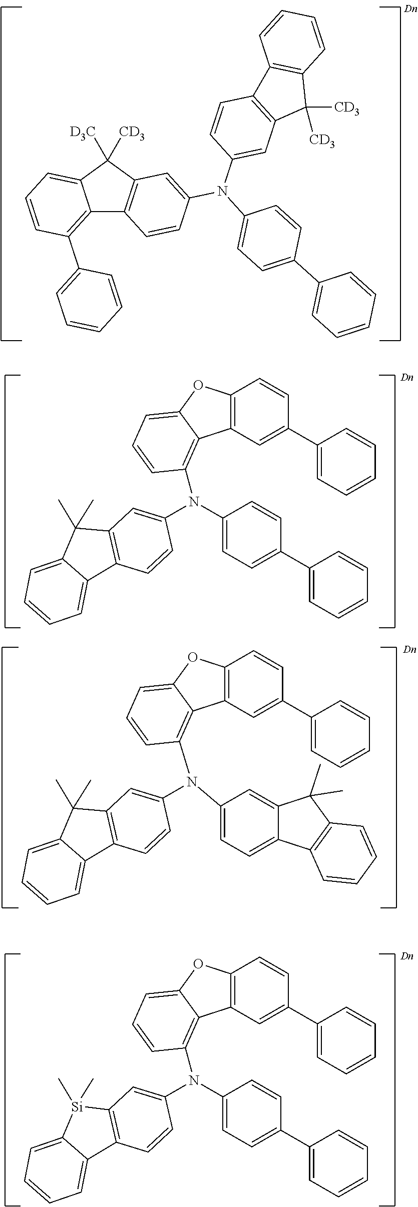

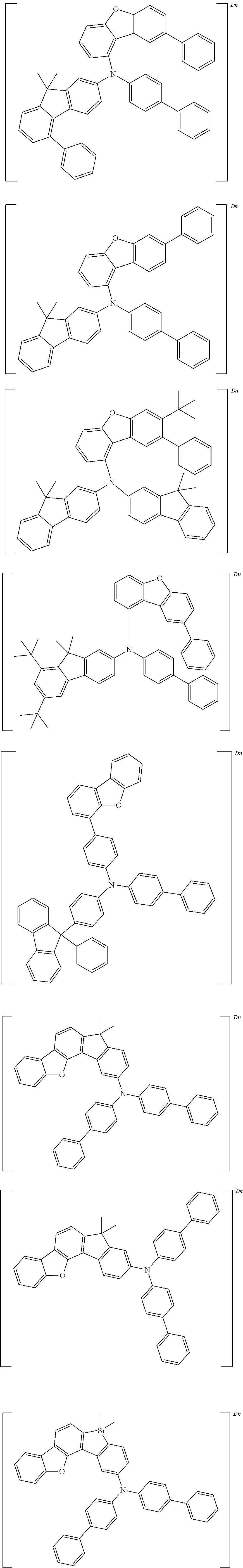

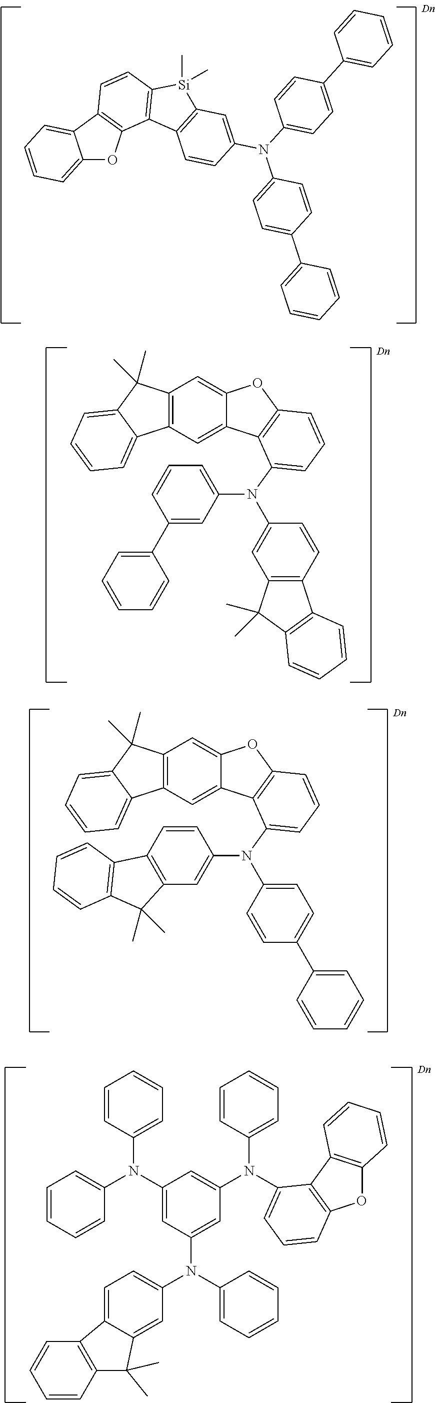

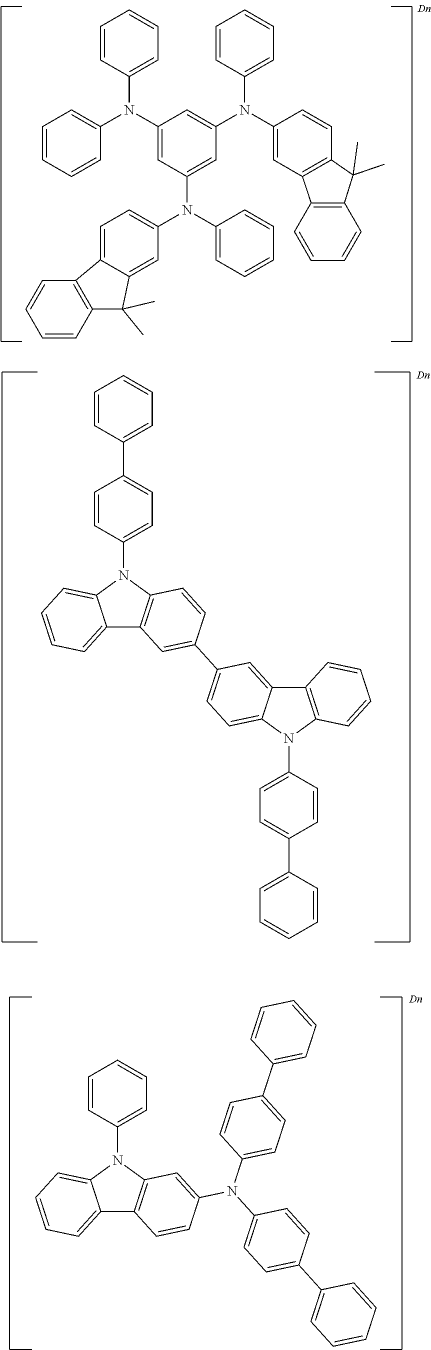

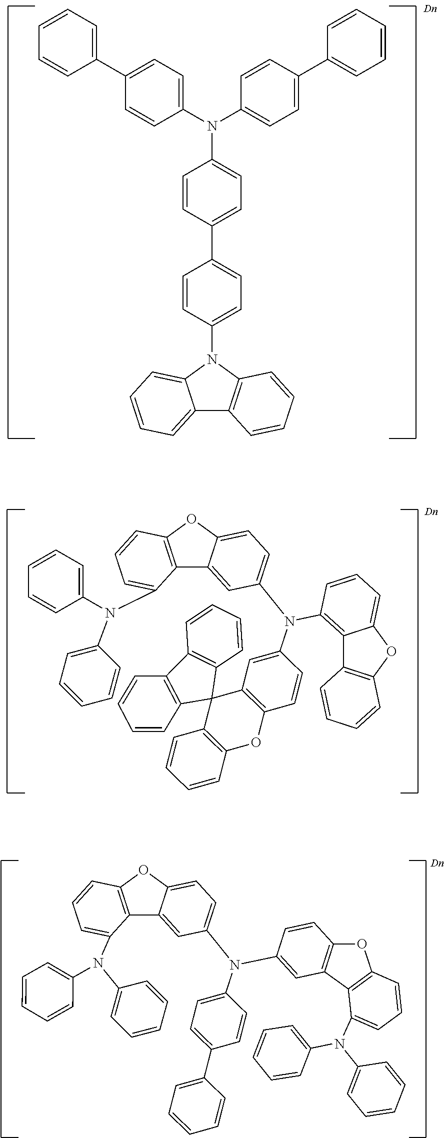

(Dn refers to the number of deuterium substitutions and indicates a structure substituted with one or more deuterium atoms)

In the hole transport region 140, other suitable compounds may also be used.

Also, the charge transport region may be, e.g., the electron transport region 150.

The electron transport region 150 may help further increase electron injection and/or electron mobility between the cathode 110 and the light emitting layer 130 and block holes.

In an implementation, the electron transport region 150 may include an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

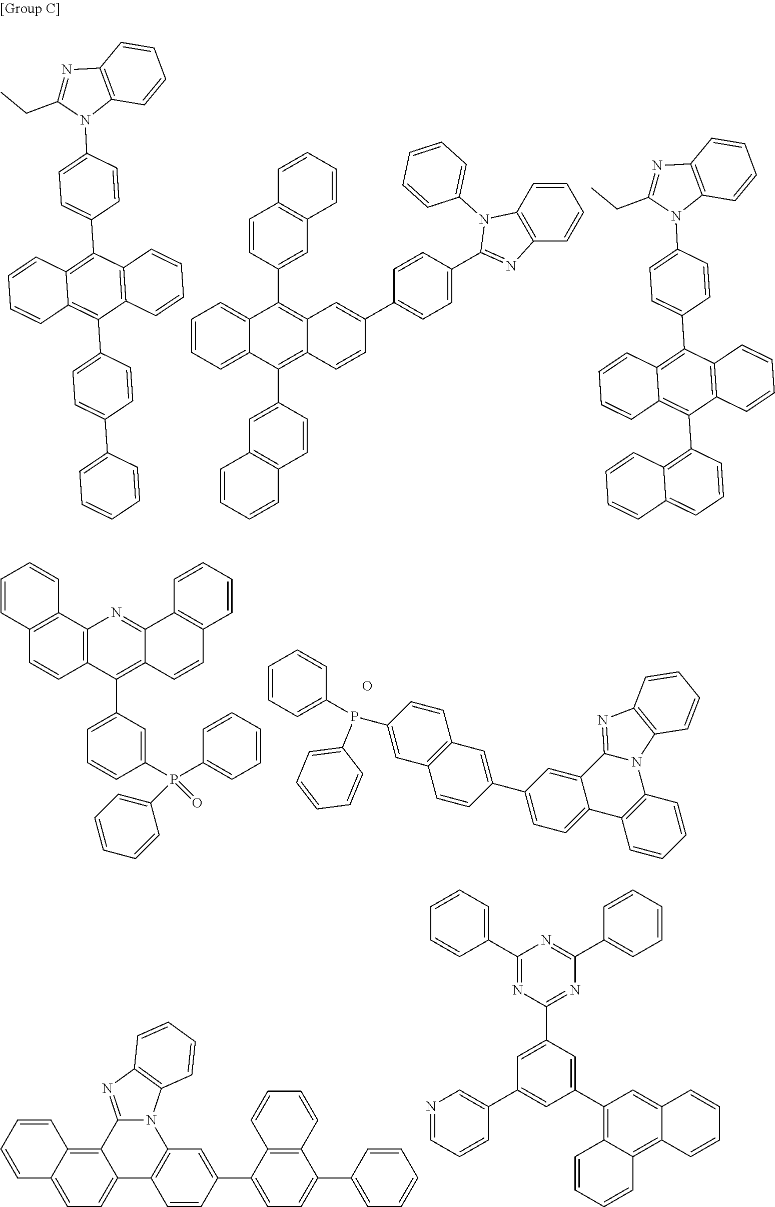

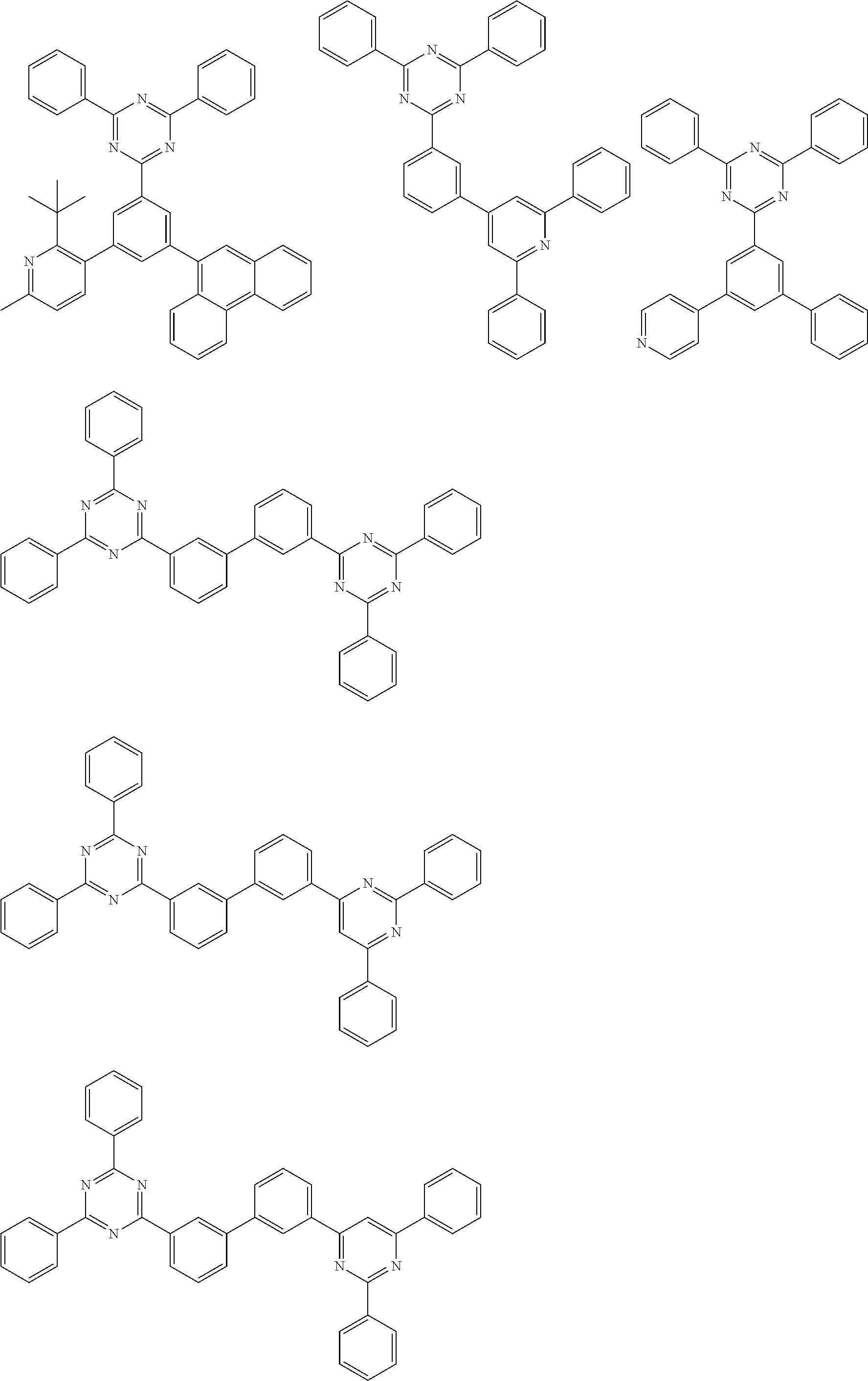

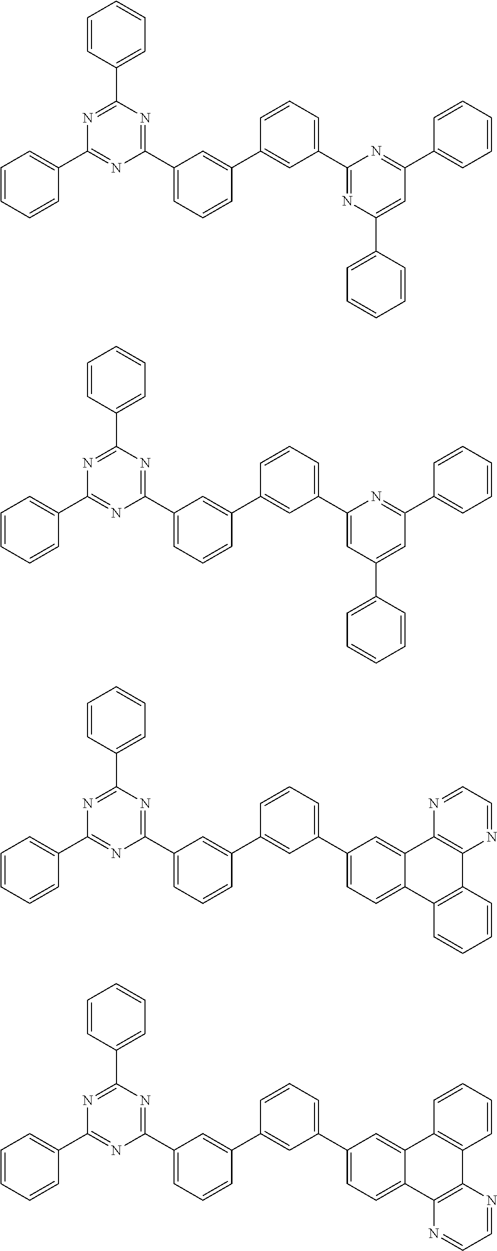 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

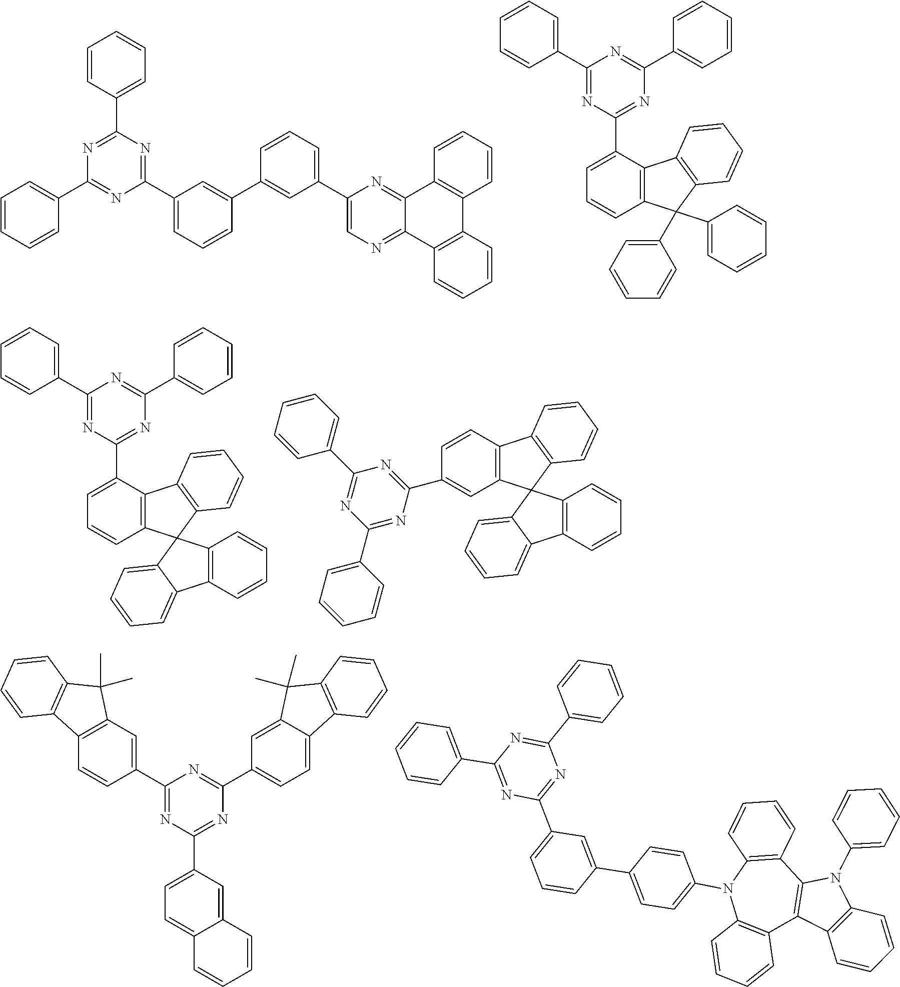

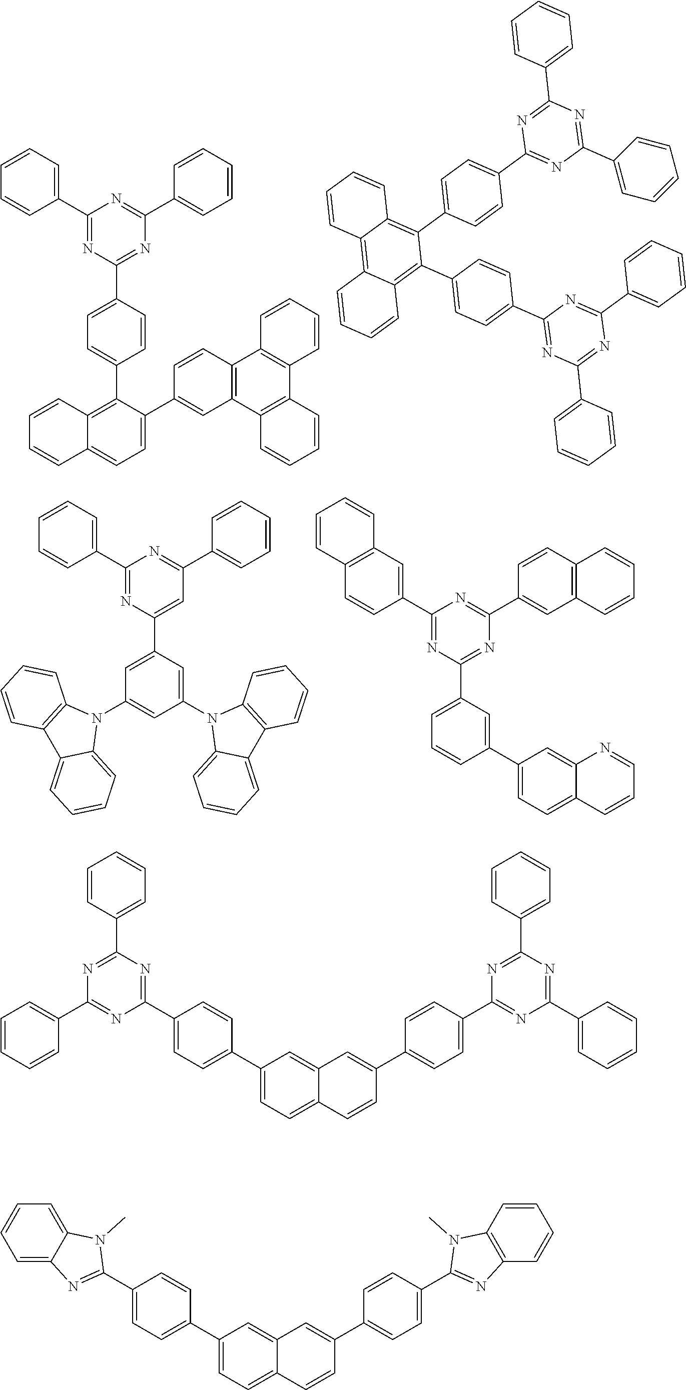

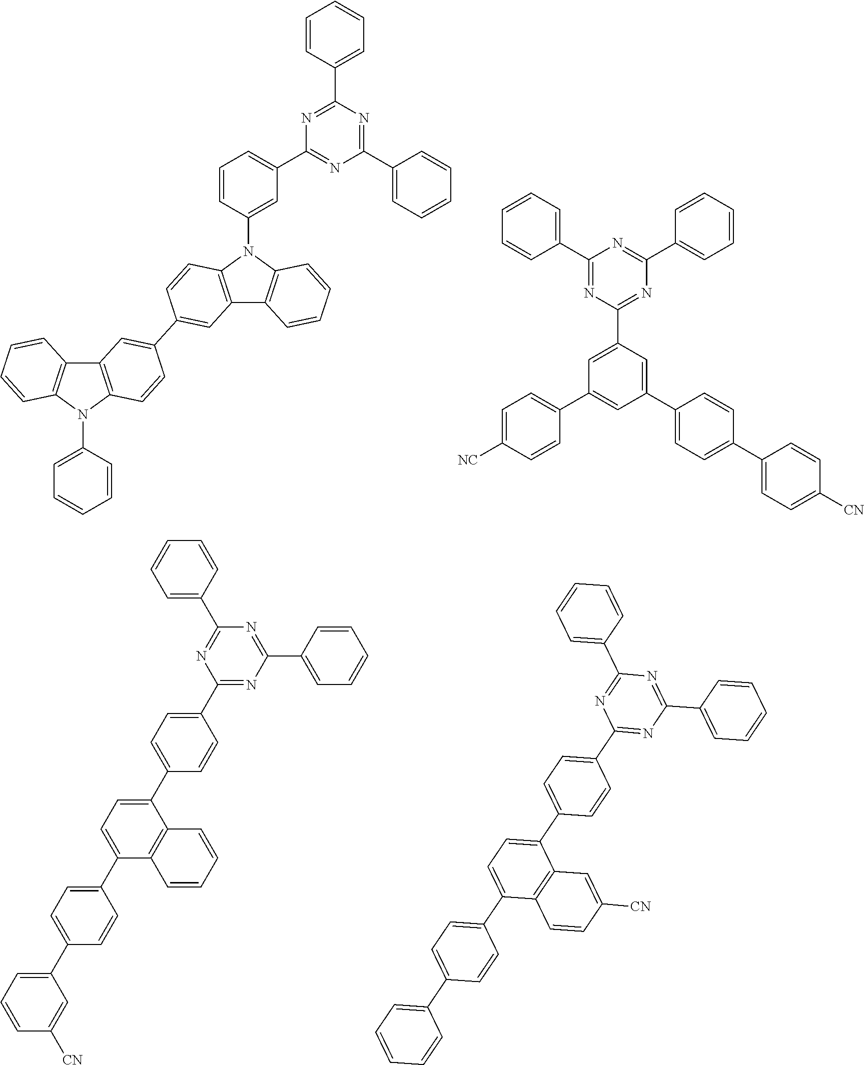 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

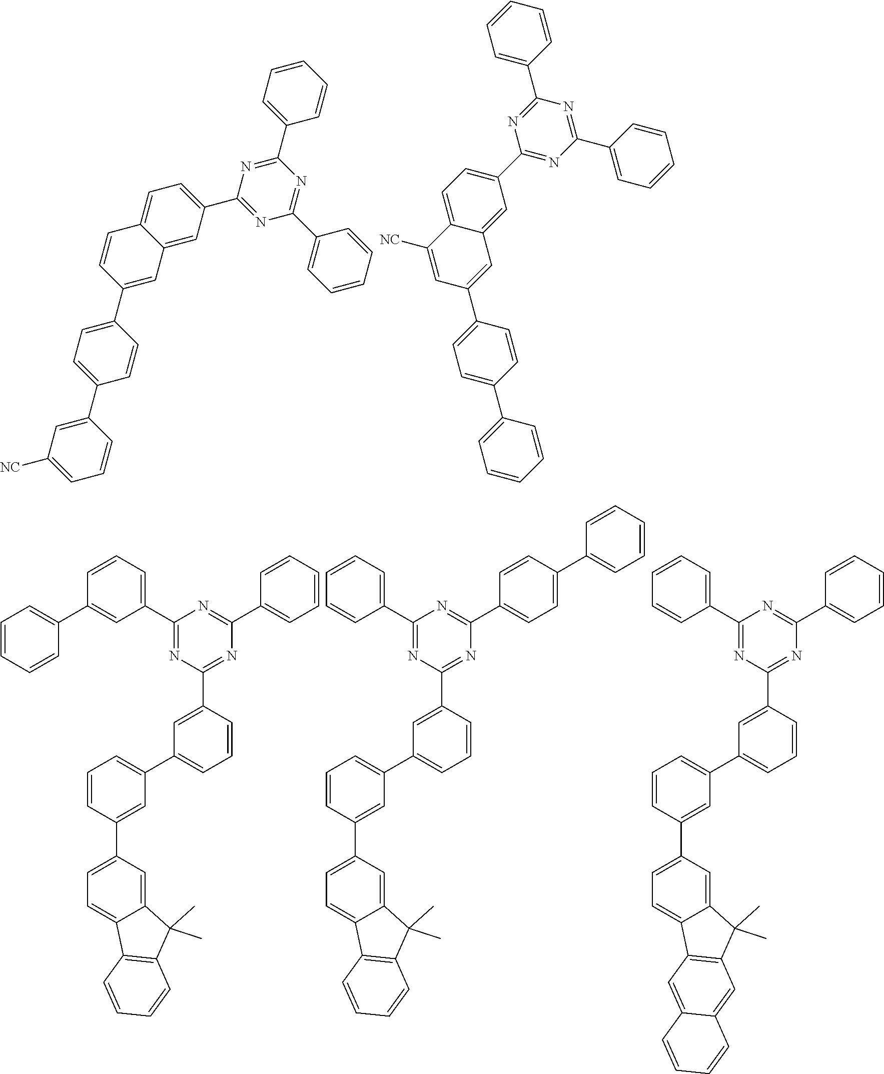

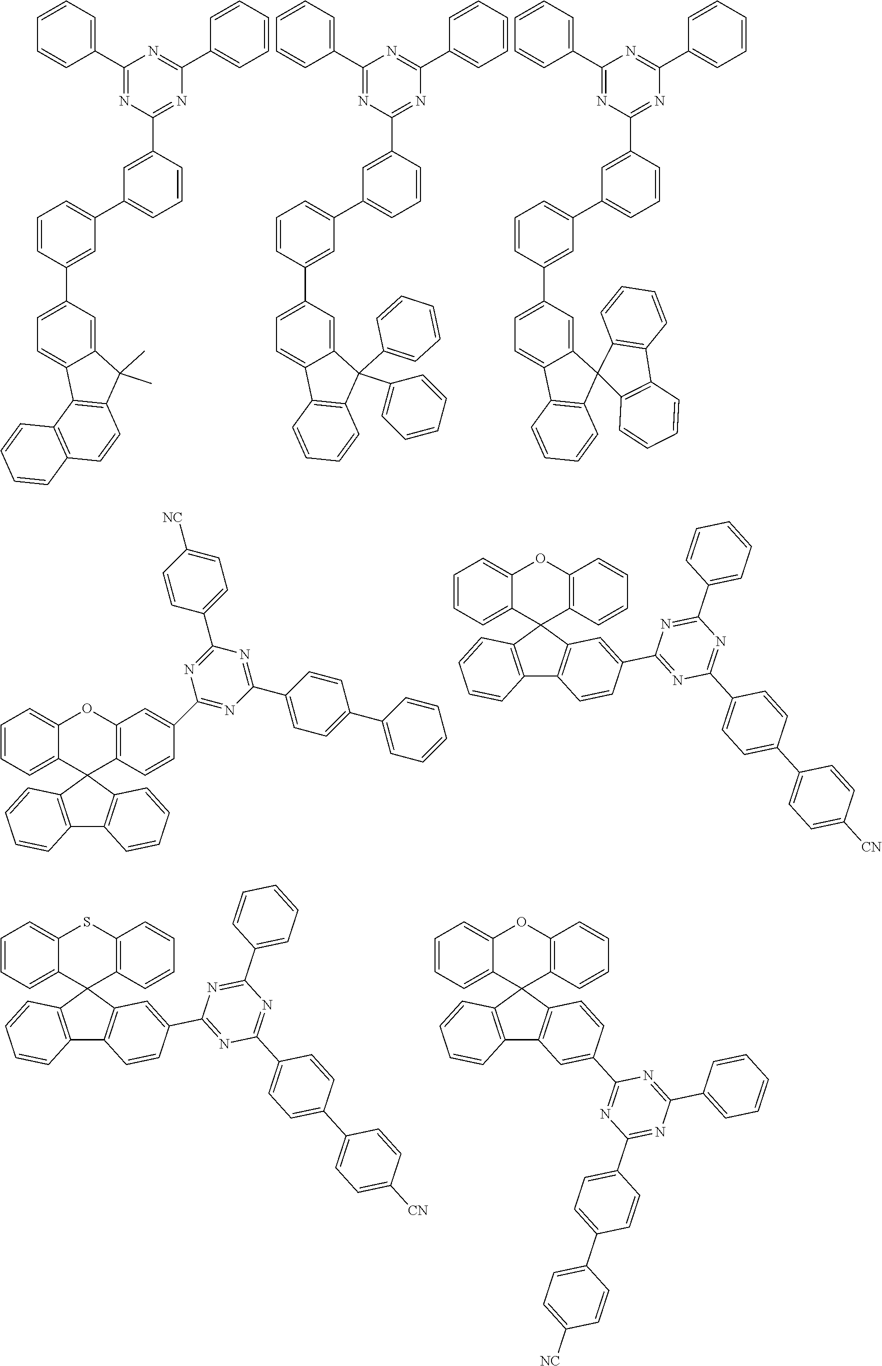

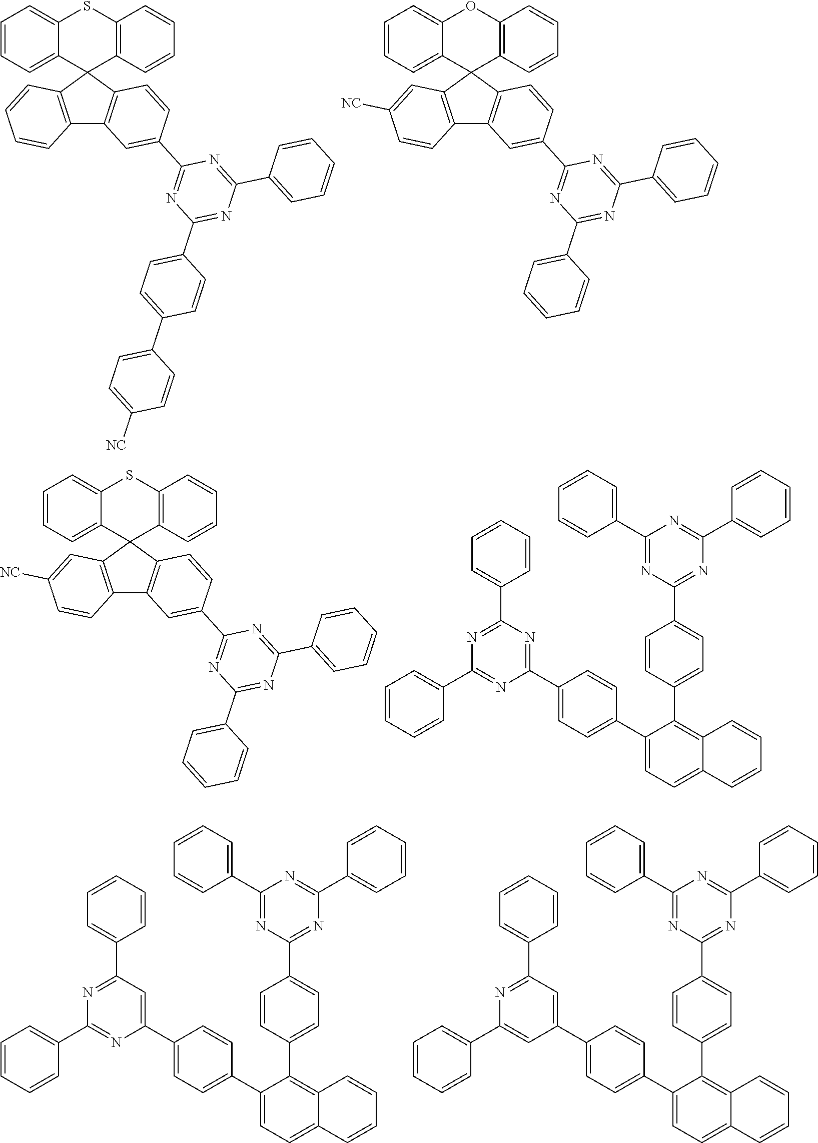

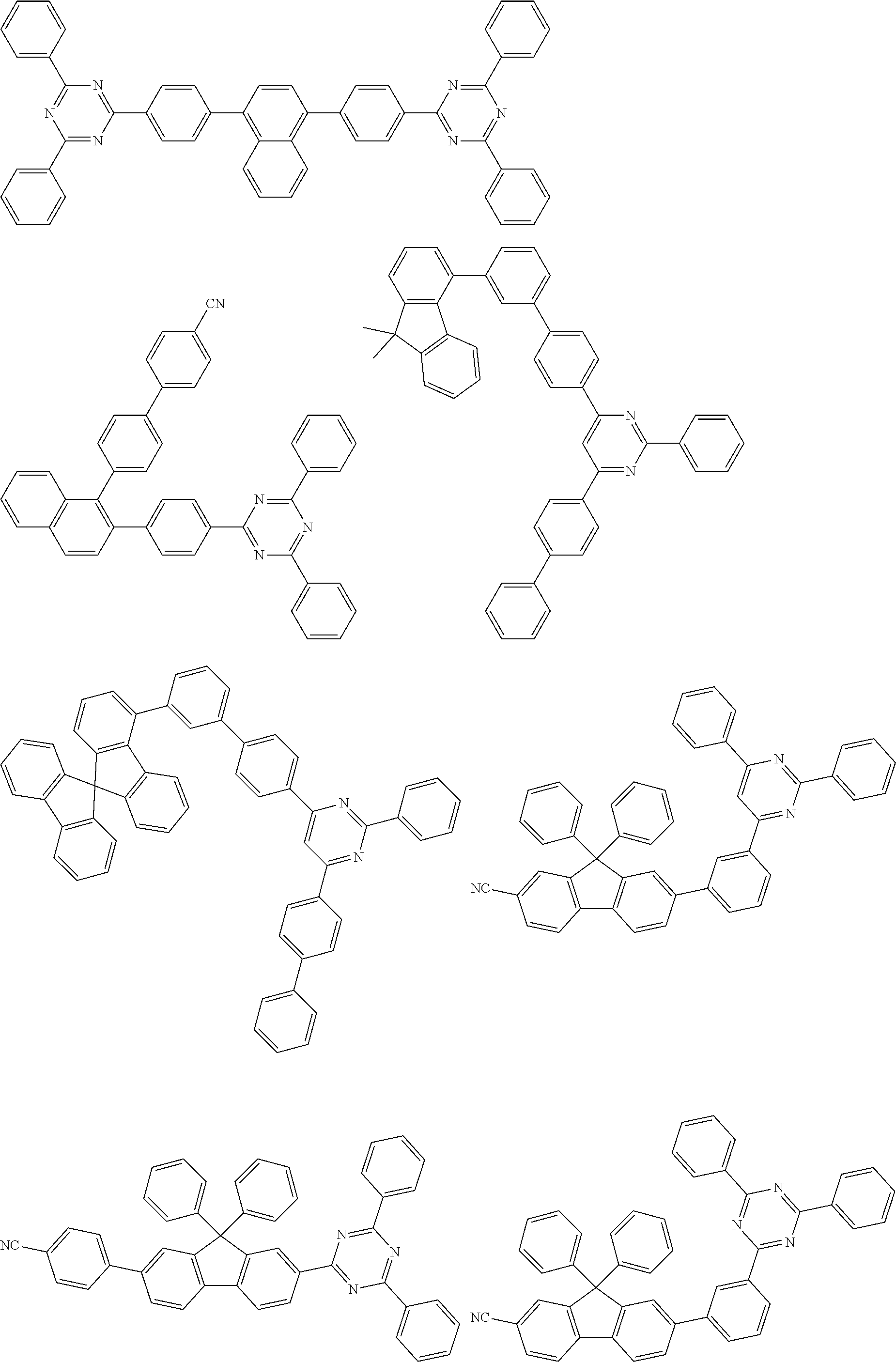

Some embodiments may be an organic light emitting diode including the light emitting layer as the organic layer.

Some embodiments may be an organic light emitting diode including a light emitting layer and a hole transport region as the organic layer.

Some embodiments may be an organic light emitting diode including a light emitting layer and an electron transport region as the organic layer.

An organic light emitting diode according to some embodiments includes a hole transport region 140 and an electron transport region 150 in addition to the light emitting layer 130 as the organic layer 105, as shown in the FIGURE.

In some embodiments, an organic light emitting diode may further include an electron injection layer, a hole injection layer, or the like, in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there in no particular comment or were synthesized by suitable methods.

### (Synthesis of Compound for Organic Optoelectronic Device)

### Synthesis Example 1: Synthesis of Compound A-1

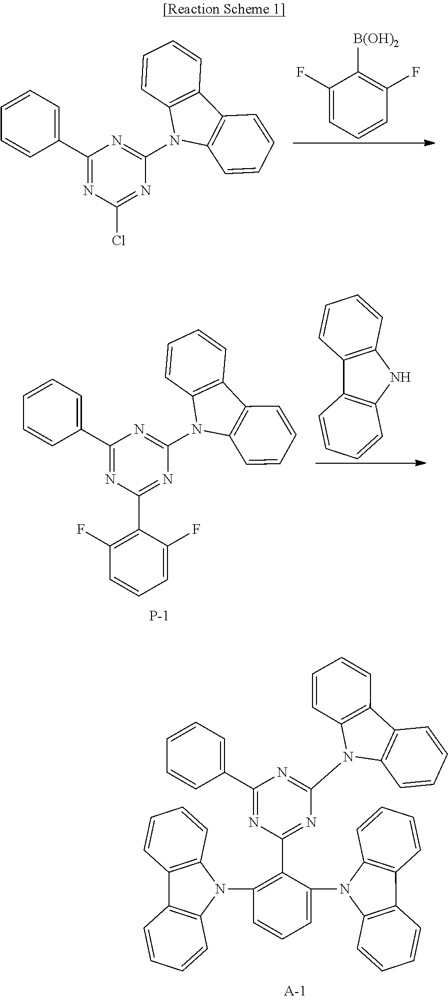

### 1st Step: Synthesis of Intermediate P-1

9-(4-chloro-6-phenyl-1,3,5-triazin-2-yl)-9H-carbazole (100 g/1.0 eq.), (2,6-difluorophenyl)boronic acid (1.1 eq.), Pd(PPh₃)₄ (0.05 eq.), and K₂CO₃ (3.0 eq.) with THF (750 mL) and distilled water (250 mL) were added to a flask and then, refluxed at 80° C. After 12 hours, when the reaction was completed, a resultant therefrom was diluted with dichloromethane (DCM), three times washed with brine, and dried with MgSO₄. Subsequently, 20 g of Intermediate P-1 was obtained through column chromatography.

### 2nd Step: Synthesis of Compound A-1

Intermediate P-1 (10 g/1.0 eq.), 9H-carbazole (2.5 eq.), and K₃PO₄ (3.0 eq.) with dimethyl formamide (DMF) (200 mL) were added to a flask and then, refluxed at 150° C. After 12 hours, when the reaction was completed, a resultant therefrom was diluted with dichloromethane (DCM), three times washed with brine, and dried with MgSO₄. Subsequently, 6 g of Compound A-1 was obtained through column chromatography.

### Synthesis Example 2: Synthesis of Compound A-2

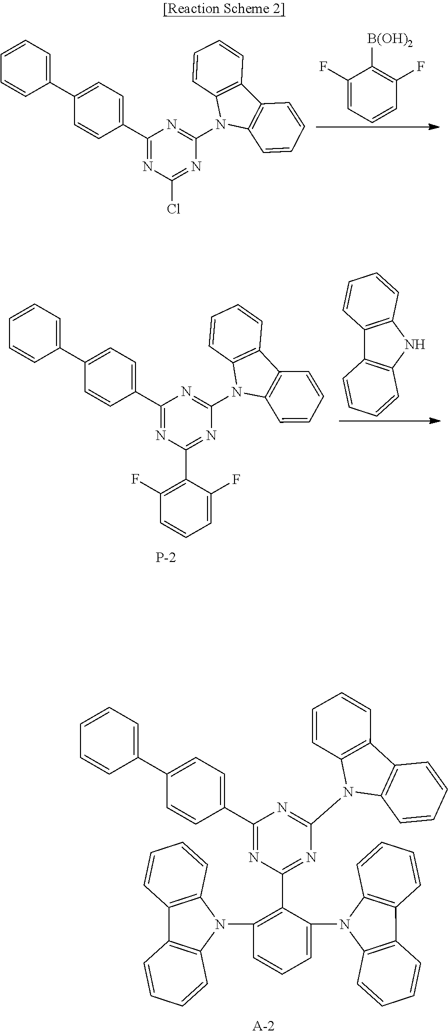

### 1st Step: Synthesis of Intermediate P-2

9-(4-([1,1′-biphenyl]-4-yl)-6-chloro-1,3,5-triazin-2-yl)-9H-carbazole (100 g/1.0 eq.), (2,6-difluorophenyl)boronic acid (1.1 eq.), Pd(PPh₃)₄ (0.05 eq.), and K₂CO₃ (3.0 eq.) with THF (750 mL) and distilled water (250 mL) were added to a flask and then, refluxed at 80° C. After 12 hours, when the reaction was completed, a resultant therefrom was diluted with dichloromethane (DCM), three times washed with brine, and dried with MgSO₄. Subsequently, 28 g of Intermediate P-1 was obtained through column chromatography.

### 2nd Step: Synthesis of Compound A-2

Intermediate P-2 (28 g/1.0 eq.), 9H-carbazole (2.5 eq.), and K₃PO₄ (3.0 eq.) with DMF (200 mL) were added to a flask and then, refluxed at 150° C. After 12 hours, when the reaction was completed, a resultant therefrom was diluted with dichloromethane (DCM), three times washed with brine, and dried with MgSO₄. Subsequently, 20 g of Compound A-2 was obtained through column chromatography.

### Synthesis Example 3: Synthesis of Compound A-15

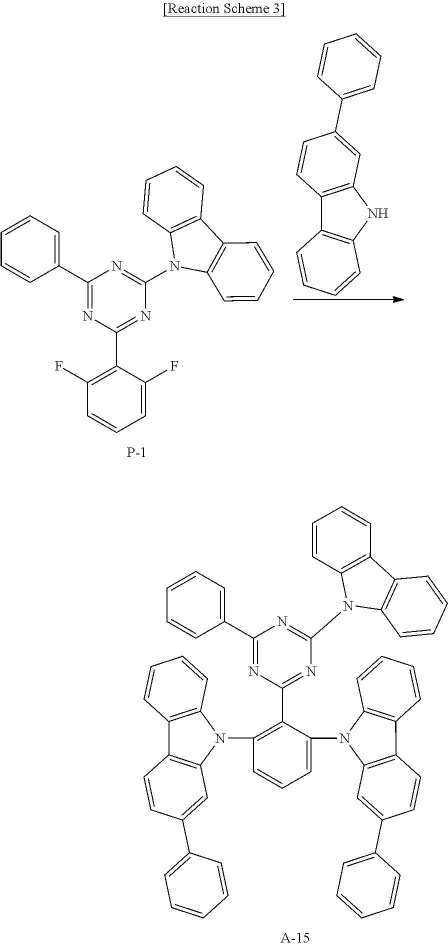

Intermediate P-1 (10 g/1.0 eq.), 2-phenyl-9H-carbazole (2.5 eq.), and K₃PO₄ (3.0 eq.) with DMF (200 mL) were added to a flask and then, refluxed at 150° C. After 12 hours, when the reaction was completed, a resultant therefrom was diluted with dichloromethane (DCM), three times washed with brine, and dried with MgSO₄. Subsequently, 7 g of Compound A-15 was obtained through column chromatography.

### Synthesis Example 4: Synthesis of Compound R-1

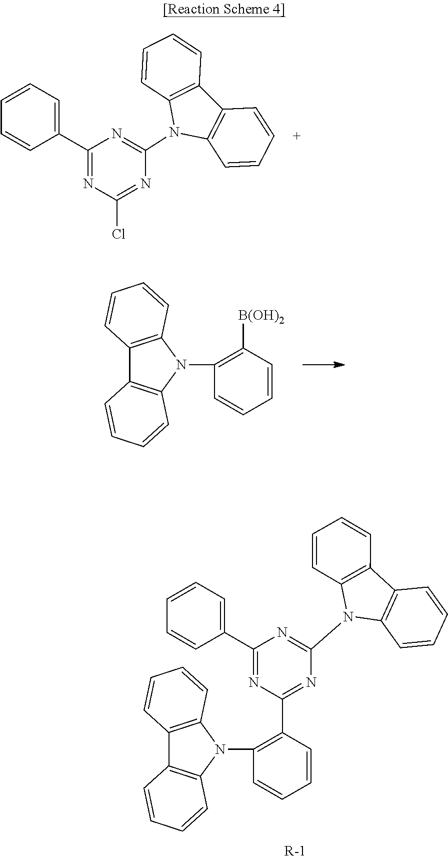 [SMILES: Clc1nc(-c2ccccc2)nc(-n2c3ccccc3c3ccccc32)n1 ; OB(O)c1ccccc1-n1c2ccccc2c2ccccc21 ; c1ccc(-c2nc(-c3ccccc3-n3c4ccccc4c4ccccc43)nc(-n3c4ccccc4c4ccccc43)n2)cc1]

9-(4-chloro-6-phenyl-1,3,5-triazin-2-yl)-9H-carbazole (20 g/1.0 eq.), (2-(9H-carbazol-9-yl)phenyl)boronic acid (1.1 eq.), Pd(PPh₃)₄ (0.05 eq.), and K₂CO₃ (3.0 eq.) with THF (750 mL) and distilled water (250 mL) were added to a flask and then, refluxed at 80° C. After 12 hours, when the reaction was completed, a resultant therefrom was diluted with dichloromethane (DCM), three times washed with brine, and dried with MgSO₄. Subsequently, 15 g of Compound R-1 was obtained through column chromatography.

### Synthesis Example 5: Synthesis of Compound R-2

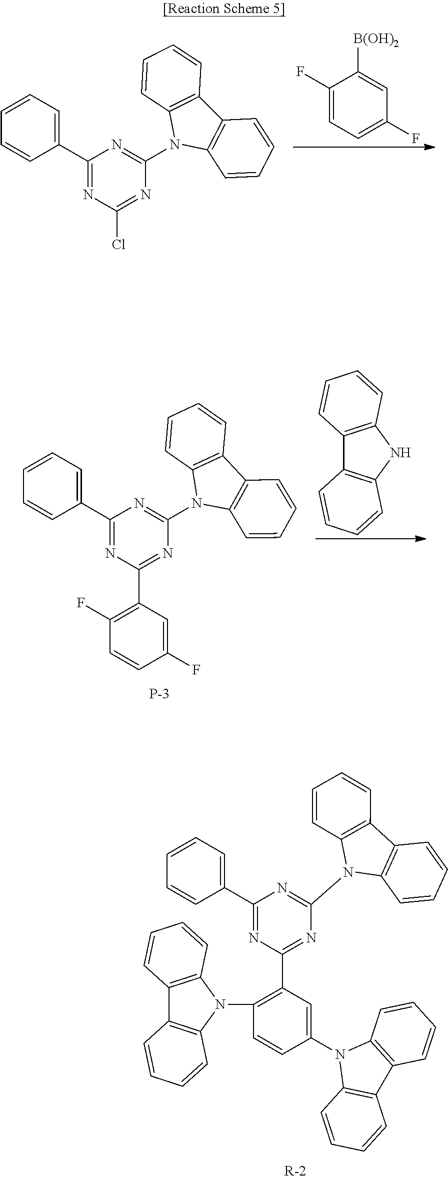

### 1st Step: Synthesis of Intermediate P-3

9-(4-chloro-6-phenyl-1,3,5-triazin-2-yl)-9H-carbazole (20 g/1.0 eq.), (2,5-difluorophenyl)boronic acid (1.1 eq.), Pd(PPh₃)₄ (0.05 eq.), and K₂CO₃ (3.0 eq.) with THF (750 mL) and distilled water (250 mL) were added to a flask and then, refluxed at 80° C. After 12 hours, when the reaction was completed, a resultant therefrom was diluted with dichloromethane (DCM), three times washed with brine, and dried with MgSO₄. Subsequently, 18 g of Intermediate P-3 was obtained through column chromatography.

### 2nd Step: Synthesis of Compound R-2

Intermediate P-3 (18 g/1.0 eq.), 9H-carbazole (2.5 eq.), and K₃PO₄ (3.0 eq.) with DMF (200 mL) were added to a flask and then, refluxed at 150° C. After 12 hours, when the reaction was completed, a resultant therefrom was diluted with dichloromethane (DCM), three times washed with brine, and dried with MgSO₄. Subsequently, 10 g of Compound R-2 was obtained through column chromatography.

### Synthesis Example 6: Synthesis of Compound R-3

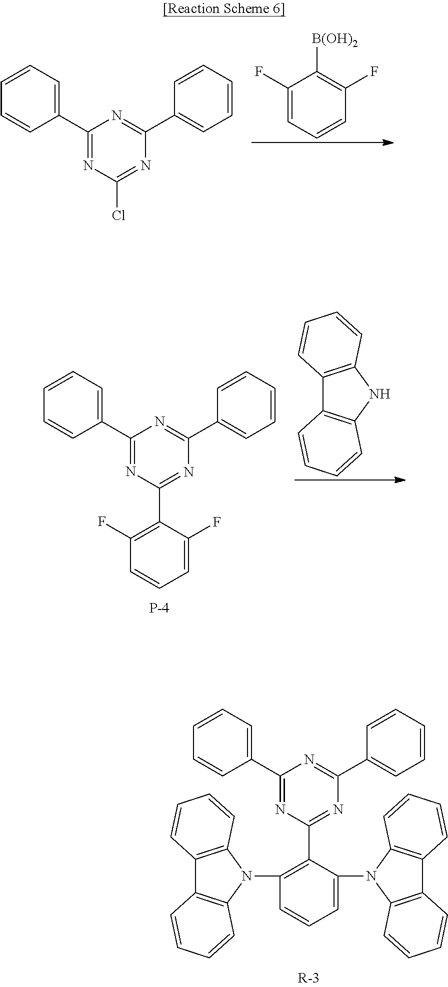

### 1st Step: Synthesis of Intermediate P-4

2-chloro-4,6-diphenyl-1,3,5-triazine (20 g/1.0 eq.), (2,6-difluorophenyl)boronic acid (1.1 eq.), Pd(PPh₃)₄ (0.05 eq.), and K₂CO₃ (3.0 eq.) with THF (750 mL) and distilled water (250 mL) were added to a flask and then, refluxed at 80° C. After 12 hours, when the reaction was completed, a resultant therefrom was diluted with dichloromethane (DCM), three times washed with brine, and dried with MgSO₄. Subsequently, 12 g of Intermediate P-4 was obtained through column chromatography.

### 2nd Step: Synthesis of Compound R-3

Intermediate P-4 (12 g/1.0 eq.), 9H-carbazole (2.5 eq.), and K₃PO₄ (3.0 eq.) with DMF (200 mL) were added to a flask and then, refluxed at 150° C. After 12 hours, when the reaction was completed, a resultant therefrom was diluted with dichloromethane (DCM), three times washed with brine, and dried with MgSO₄. Subsequently, 8 g of Compound R-3 was obtained through column chromatography.

### Synthesis of Second Compound

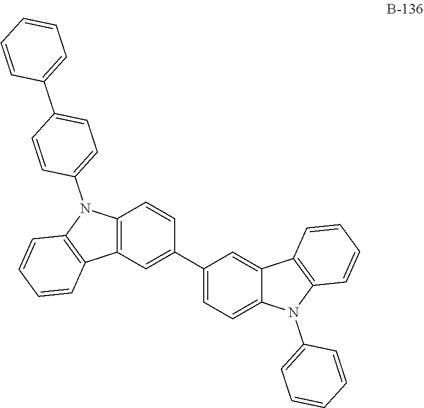 [SMILES: c1ccc(-c2ccc(-n3c4ccccc4c4cc(-c5ccc6c(c5)c5ccccc5n6-c5ccccc5)ccc43)cc2)cc1]

Compound B-136 was purchased from Gemchem Inc.

HRMS (70 eV, EI+): m/z calcd for C42H28N2: 560.2252. found: 560.

Elemental Analysis: C, 90%; H, 5%

### Synthesis Example 7: Synthesis of Compound C-4

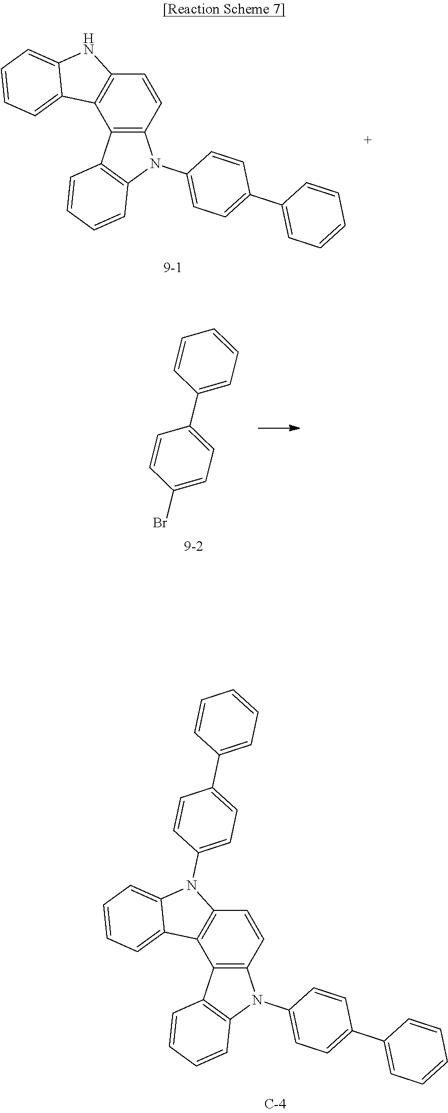 [SMILES: c1ccc(-c2ccc(-n3c4ccccc4c4c5c(ccc43)[nH]c3ccccc35)cc2)cc1 ; Brc1ccc(-c2ccccc2)cc1 ; c1ccc(-c2ccc(-n3c4ccccc4c4c5c6ccccc6n(-c6ccc(-c7ccccc7)cc6)c5ccc43)cc2)cc1]

10.0 g (24.5 mmol) of Intermediate 9-1, 6.3 g (26.9 mmol) of Intermediate 9-2, 1.1 g (1.2 mmol) of Pd₂(dba)₃, 3.5 g (36.7 mmol) of NaOtBu, and 0.7 g (3.7 mmol) of P(t-Bu)₃ were added to a round-bottomed flask, and 122 ml of xylene was added thereto and then, refluxed by stirring at 140° C. for 12 hours. After the reaction was completed, distilled water was added thereto and then, stirred, and after removing an aqueous layer, an organic layer obtained therefrom was filtered with silica gel and recrystallized, obtaining 10.3 g (75%) of Compound C-4.

(LC/MS theoretical value: 560.23 g/mol, measured value: M+=561.54 g/mol)

### Synthesis Example 8: Synthesis of Compound C-5

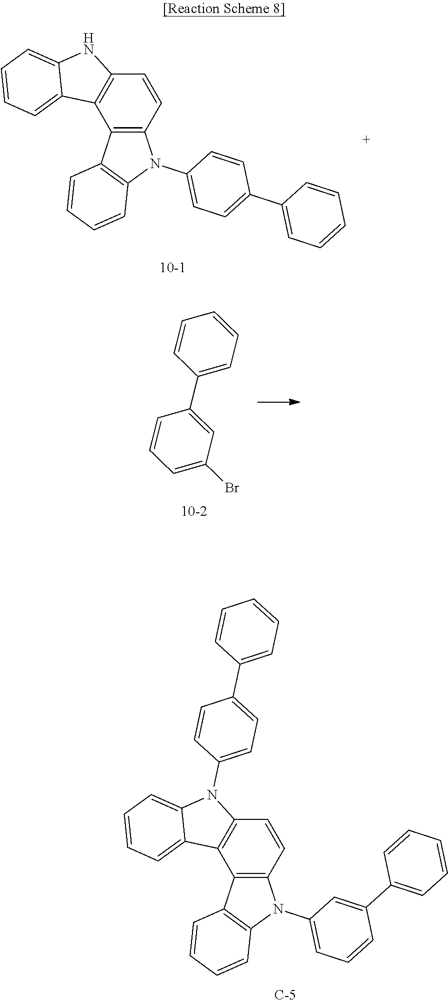 [SMILES: c1ccc(-c2ccc(-n3c4ccccc4c4c5c(ccc43)[nH]c3ccccc35)cc2)cc1 ; Brc1cccc(-c2ccccc2)c1 ; c1ccc(-c2ccc(-n3c4ccccc4c4c5c6ccccc6n(-c6cccc(-c7ccccc7)c6)c5ccc43)cc2)cc1]

10.0 g (24.5 mmol) of Intermediate 10-1, 6.3 g (26.9 mmol) of Intermediate 10-2, 1.1 g (1.2 mmol) of Pd₂(dba)₃, 3.5 g (36.7 mmol) of NaOtBu, and 0.7 g (3.7 mmol) of P(t-Bu)₃ were added to a round-bottomed flask, and 122 ml of xylene was added thereto and then, refluxed at 140° C. for 12 hours. After a reaction was completed, distilled water was added thereto and then, stirred, and after removing an aqueous layer, an organic layer therefrom was filtered with silica gel and recrystallized, obtaining 9.7 g (71%) of Compound C-5.

(LC/MS theoretical value: 560.23 g/mol, measured value: M+=561.57 g/mol)

### Example 1: Manufacturing of Green Organic Light Emitting Diode (Single Host)

A glass substrate coated with a thin film of ITO (indium tin oxide) was ultrasonically cleaned with distilled water. After washing with the distilled water, the glass substrate was ultrasonically washed with isopropyl alcohol, acetone, or methanol and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1,350 Å to form a hole transport layer. Compound B was deposited on the hole transport layer to a thickness of 350 Å to form a hole transport auxiliary layer. On the hole transport auxiliary layer, Compound A-1 was used as a host and PhGD was doped at 7 wt % as a dopant to form a 400 Å-thick light emitting layer by vacuum deposition. Then, Compound C was deposited on the light emitting layer to a thickness of 50 Å to form an electron transport auxiliary layer, and Compound D and LiQ were simultaneously vacuum-deposited in a weight ratio of 1:1 to form a 300 Å-thick electron transport layer. An organic light emitting diode was manufactured by sequentially vacuum-depositing 15 Å of LiQ and 1,200 Å of Al on the electron transport layer to form a cathode.

ITO/Compound A (3% NDP-9 doping, 100 Å)/Compound A (1,350 Å)/Compound B (350 Å)/EML [Host (Compound A-1):PhGD=93 wt %:7 wt %] (400 Å)/Compound C (50 Å)/Compound D: LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-Dibenzofuranyl)phenyl]-N-[4-(9-phenyl-9H-fluoren-9-yl)phenyl][1,1′-biphenyl]-4-amine
Compound C: 2,4-Diphenyl-6-(4′,5′,6′-triphenyl[1,1′:2′,1″:3″,1′″:3′″, 1″″-quinquephenyl]-3″″-yl)-1,3,5-triazine
Compound D: 2-(1,1′-Biphenyl-4-yl)-4-(9,9-diphenylfluoren-4-yl)-6-phenyl-1,3,5-triazine

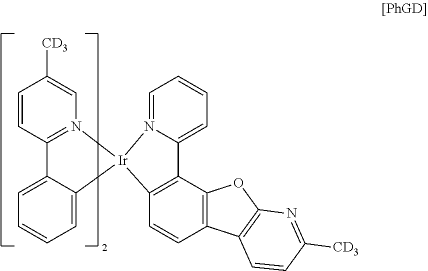

### Example 2 and Example 3 and Comparative Examples 1 to 3

Each organic light emitting diode was manufactured in the same manner as Example 1, except that the compositions were changed to those shown in Table 1.

### Example 4: Manufacturing of Green Organic Light Emitting Diode (Mixed Host)

A glass substrate coated with a thin film of ITO (indium tin oxide) was ultrasonically cleaned with distilled water. After washing with the distilled water, the glass substrate was ultrasonically washed with isopropyl alcohol, acetone, or methanol and dried and then, moved to a plasma cleaner, cleaned by using oxygen plasma for 10 minutes, and moved to a vacuum depositor. This prepared ITO transparent electrode was used as an anode, Compound E doped with 3% NDP-9 (Novaled GmbH) was vacuum-deposited on the ITO substrate to form a 100 Å-thick hole injection layer, and Compound E was deposited on the hole injection layer to a thickness of 1,350 Å to form a hole transport layer. Compound F was deposited on the hole transport layer to a thickness of 270 Å to form a first hole transport auxiliary layer, and Compound G was deposited on the first hole transport layer to a thickness of 50 Å to form a second hole transport auxiliary layer. On the second hole transport auxiliary layer, Compound A-1 and Compound C-4 were simultaneously used as hosts in a weight ratio of 4:6, and IrGD was doped at 8 wt % as a dopant to form a 330 Å-thick light emitting layer by vacuum deposition. Subsequently, Compound H was deposited on the light emitting layer to a thickness of 50 Å to form an electron transport auxiliary layer, and Compound I and LiQ were simultaneously vacuum-deposited in a weight ratio of 1:1 to form a 300 Å-thick electron transport layer. An organic light emitting diode was manufactured by sequentially vacuum-depositing 15 Å of Yb and 1,200 Å of Al on the electron transport layer to form a cathode.

ITO/Compound E (3% NDP-9 doping, 100 Å)/Compound E (1,350 Å)/Compound F (270 Å)/Compound G (50 Å)/EML [Host (Compound A-1: Compound C-4=4:6 wt %/wt %): IrGD=92 wt %: 8 wt %] (380 Å)/Compound H (50 Å)/Compound I: LiQ (300 Å)/Yb (15 Å)/Al (1,200 Å).
Compound E: N-(9,9-diphenyl-9H-fluoren-2-yl)-N,9-diphenyl-9H-carbazol-2-amine
Compound F: 6,8-di-tert-butyl-N-(9,9-dimethyl-9H-fluoren-2-yl)-9,9-dimethyl-N-(2-phenylphenyl)-9H-fluoren-2-amine
Compound G: N-(9,9-dimethyl-9H-fluoren-2-yl)-N-[(9,9-dimethyl-9H-fluoren-4-yl)phenyl]-9,9′-spirobi[fluorene]-4-amine
Compound H: 4-{4-[3-(9,9-dimethyl-9H-fluoren-4-yl)phenyl]phenyl}-2-phenyl-6-(4-phenylphenyl)pyrimidine
Compound I: 2-(4-{1-[4-(diphenyl-1,3,5-triazin-2-yl)phenyl]naphthalen-2-yl}phenyl)-4,6-diphenyl-1,3,5-triazine

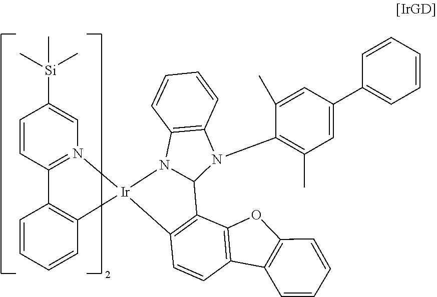

### Examples 5 and 6 and Comparative Examples 4 to 6

Each organic light emitting diode was manufactured in the same manner as Example 4, except that the compositions were changed to those shown in Table 2.

### Evaluation

The driving voltage, luminous efficiency, and life-span characteristics of the organic light emitting diodes according to Examples 1 to 6 and Comparative Examples 1 to 6 were evaluated.

The specific measurement method is as follows, and the results are as shown in Tables 1 to 2.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Luminous efficiency (cd/A) at the same current density (10 mA/cm²) were calculated by using the luminance and current density from (1) and (2) above and voltage.

The luminous efficiency values of Examples 1 to 3 and Comparative Examples 1 to 3 were calculated as relative values based on Comparative Example 1 and are shown in Table 1.

The luminous efficiency values of Examples 4 to 6 and Comparative Examples 4 to 6 were calculated as relative values based on Comparative Example 4 and are shown in Table 2.

### (4) Measurement of Life-Span

Time when each luminous efficiency (cd/A) was reduced to 97%, while maintaining luminance (cd/m²) at 24,000 cd/m², was measured as a life-span.

The life-span values of Examples 1 to 3 and Comparative Examples 1 to 3 were calculated as relative values based on Comparative Example 1 and are shown in Table 1.

The life-span values of Examples 4 to 6 and Comparative Examples 4 to 6 were calculated as relative values based on Comparative Example 4 and are shown in Table 2.

TABLE 1

| | | Luminous | |
|---|---|---|---|
| No. | Host | efficiency (%) | Life-span (%) |
| Example 1 | A-1 | 104 | 124 |
| Example 2 | A-2 | 105 | 155 |
| Example 3 | A-15 | 106 | 190 |
| Comparative Example 1 | R-1 | 100 | 100 |
| Comparative Example 2 | R-2 | 98 | 110 |
| Comparative Example 3 | R-3 | 101 | 65 |

TABLE 2

| | Host | | Luminous | |
|---|---|---|---|---|
| | First | Second | efficiency | Life-span |
| No. | compound | compound | (%) | (%) |
| Example 4 | A-1 | C-4 | 104 | 160 |
| Example 5 | A-2 | C-4 | 105 | 210 |
| Example 6 | A-15 | C-4 | 106 | 250 |
| Comparative Example 4 | R-1 | C-4 | 100 | 100 |
| Comparative Example 5 | R-2 | C-4 | 97 | 115 |
| Comparative Example 6 | R-3 | C-4 | 102 | 52 |

Referring to Tables 1 and 2, the luminous efficiency and life-span characteristics of the organic light emitting diodes according to Examples 1 to 6 are significantly improved compared to the organic light emitting diodes according to Comparative Examples 1 to 6.

One or more embodiments may provide a compound for an organic optoelectronic device that can implement a high efficiency and long life-span organic optoelectronic device.

A high-efficiency and long life-span organic optoelectronic devices may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 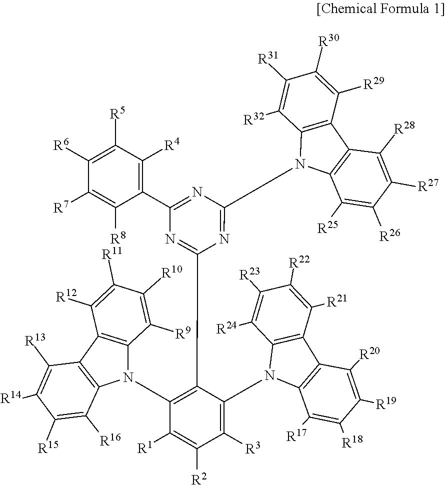
wherein, in Chemical Formula 1,
R¹ to R³ are each independently hydrogen, deuterium, or a substituted or unsubstituted phenyl group, and
R⁴ to R³² are each independently hydrogen, deuterium, a cyano group, a substituted or unsubstituted C1 to C20 alkyl group, a substituted or unsubstituted C1 to C20 alkylsilyl group, or a substituted or unsubstituted C6 to C30 aryl group.

2. The compound for an organic optoelectronic device as claimed in claim 1, wherein:
R⁴ to R⁸ are each independently hydrogen, deuterium, or a substituted or unsubstituted C6 to C20 aryl group, and
R⁹ to R³² are each independently hydrogen, deuterium, a cyano group, a substituted or unsubstituted C1 to C10 alkyl group, a substituted or unsubstituted C1 to C10 alkylsilyl group, or a substituted or unsubstituted C6 to C20 aryl group.

3. The compound for an organic optoelectronic device as claimed in claim 1, wherein:
R⁴ to R⁸ are each independently hydrogen, deuterium, a substituted or unsubstituted phenyl group, or a substituted or unsubstituted biphenyl group, and
R⁹ to R³² are each independently hydrogen, deuterium, a cyano group, a substituted or unsubstituted methyl group, a substituted or unsubstituted ethyl group, a substituted or unsubstituted propyl group, a substituted or unsubstituted trimethylsilyl group, a substituted or unsubstituted phenyl group, or a substituted or unsubstituted biphenyl group.

4. The compound for an organic optoelectronic device as claimed in claim 1, wherein the compound is a compound of Group 1:

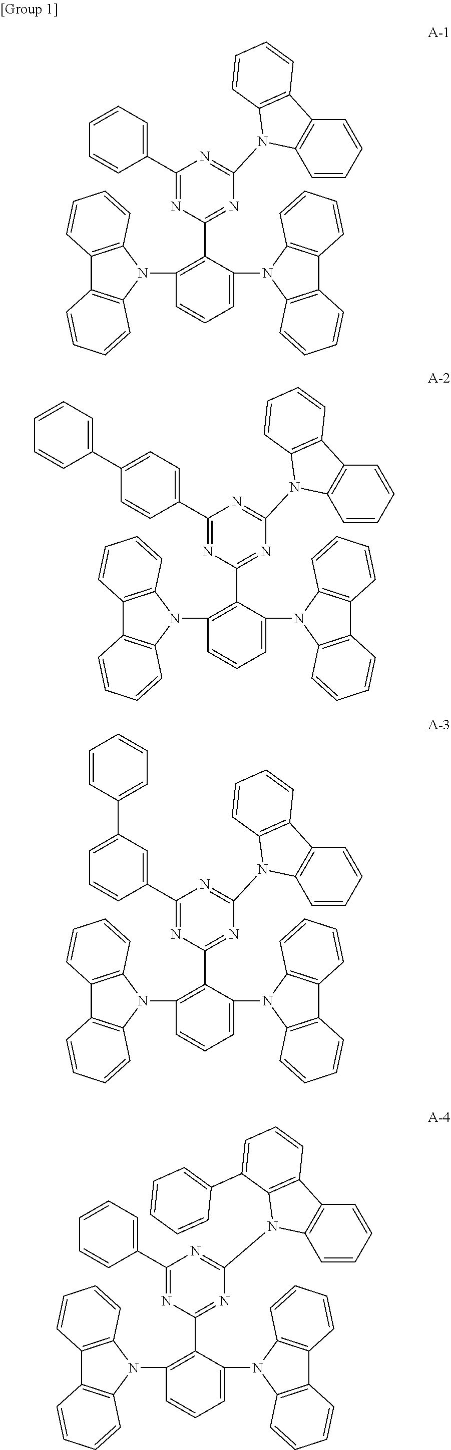

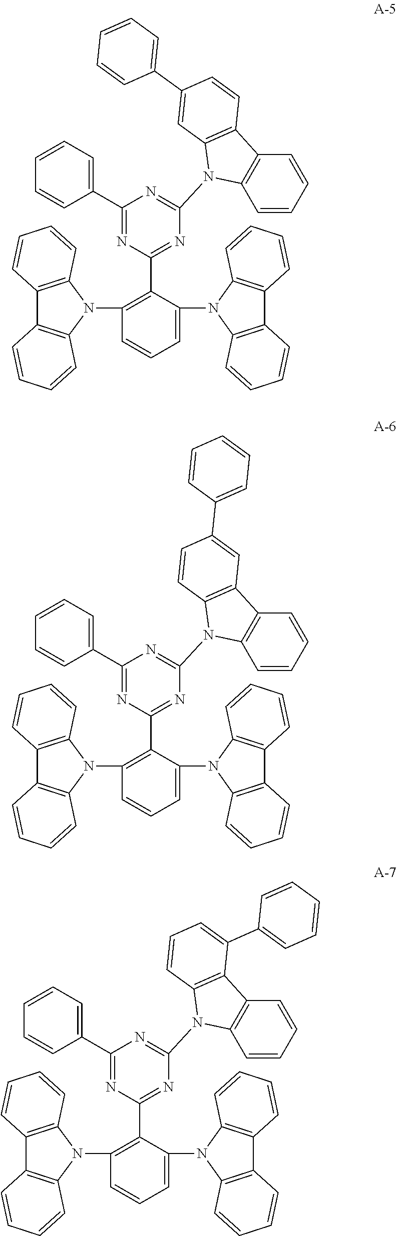

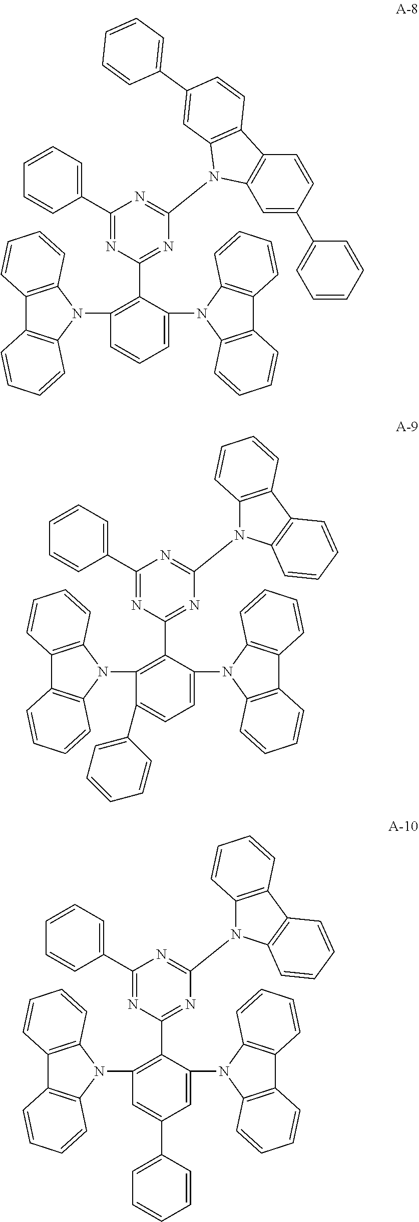

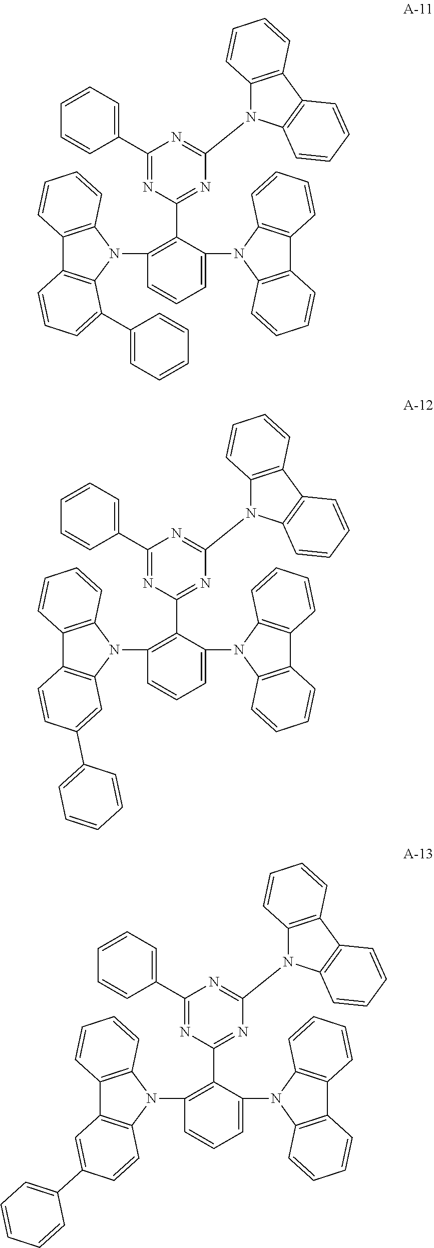 [SMILES: c1ccc(-c2nc(-c3c(-n4c5ccccc5c5ccccc54)cccc3-n3c4ccccc4c4cccc(-c5ccccc5)c43)nc(-n3c4ccccc4c4ccccc43)n2)cc1 ; c1ccc(-c2ccc3c4ccccc4n(-c4cccc(-n5c6ccccc6c6ccccc65)c4-c4nc(-c5ccccc5)nc(-n5c6ccccc6c6ccccc65)n4)c3c2)cc1 ; c1ccc(-c2ccc3c(c2)c2ccccc2n3-c2cccc(-n3c4ccccc4c4ccccc43)c2-c2nc(-c3ccccc3)nc(-n3c4ccccc4c4ccccc43)n2)cc1]

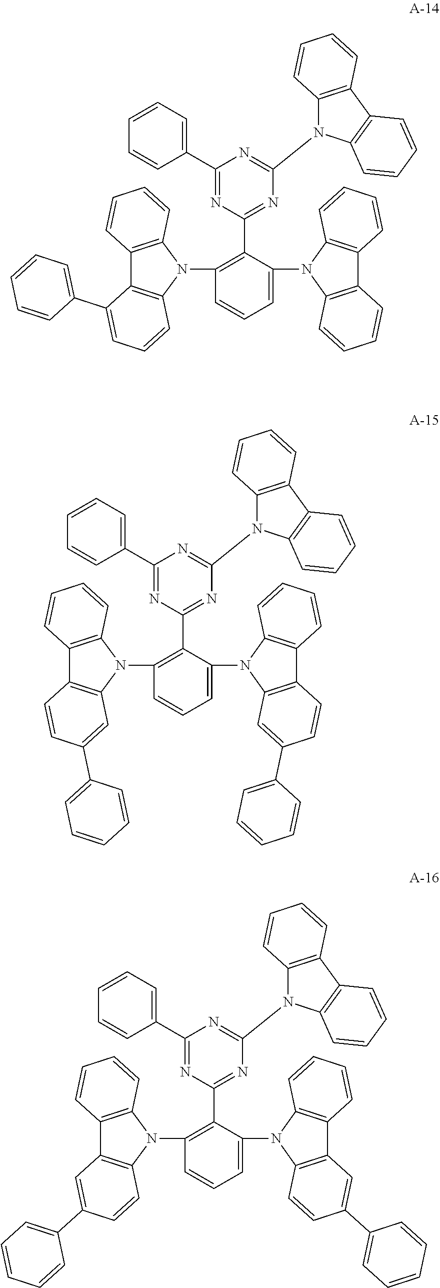 [SMILES: c1ccc(-c2nc(-c3c(-n4c5ccccc5c5ccccc54)cccc3-n3c4ccccc4c4c(-c5ccccc5)cccc43)nc(-n3c4ccccc4c4ccccc43)n2)cc1 ; c1ccc(-c2ccc3c4ccccc4n(-c4cccc(-n5c6ccccc6c6ccc(-c7ccccc7)cc65)c4-c4nc(-c5ccccc5)nc(-n5c6ccccc6c6ccccc65)n4)c3c2)cc1 ; c1ccc(-c2ccc3c(c2)c2ccccc2n3-c2cccc(-n3c4ccccc4c4cc(-c5ccccc5)ccc43)c2-c2nc(-c3ccccc3)nc(-n3c4ccccc4c4ccccc43)n2)cc1]

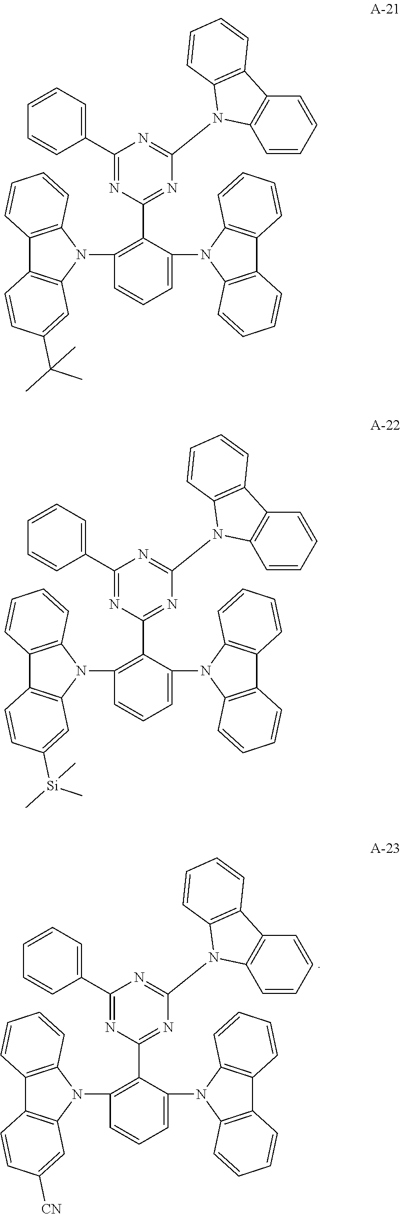 [SMILES: CC(C)(C)c1ccc2c3ccccc3n(-c3cccc(-n4c5ccccc5c5ccccc54)c3-c3nc(-c4ccccc4)nc(-n4c5ccccc5c5ccccc54)n3)c2c1 ; C[Si](C)(C)c1ccc2c3ccccc3n(-c3cccc(-n4c5ccccc5c5ccccc54)c3-c3nc(-c4ccccc4)nc(-n4c5ccccc5c5ccccc54)n3)c2c1 ; N#Cc1ccc2c3ccccc3n(-c3cccc(-n4c5ccccc5c5ccccc54)c3-c3nc(-c4ccccc4)nc(-n4c5ccccc5c5ccccc54)n3)c2c1]

5. A composition for an organic optoelectronic device, the composition comprising:
a first compound; and
a second compound,
wherein:
the first compound is the compound for an organic optoelectronic device as claimed in claim 1, and
the second compound is represented by:
Chemical Formula 2;
a combination of Chemical Formula 3 and Chemical Formula 4; or
Chemical Formula 5, 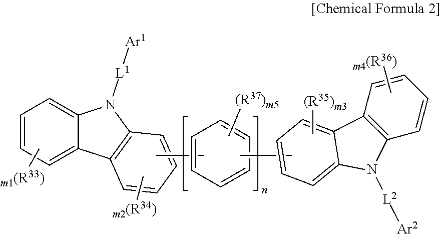
in Chemical Formula 2,
R³³ to R³⁷ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar¹ and Ar² are each independently a substituted or unsubstituted C6 to C20 aryl group or a substituted or unsubstituted C2 to C30 heterocyclic group,
L¹ and L² are each independently a single bond or a substituted or unsubstituted C6 to C20 arylene group,
m1, m4, and m5 are each independently an integer of 1 to 4,
m2 and m3 are each independently an integer of 1 to 3,
when m1 is 2, 3, or 4, each R³³ is the same or different from each other,
when m2 is 2 or 3, each R³⁴ is the same or different from each other,
when m3 is 2 or 3, each R³⁵ is the same or different from each other,
when m4 is 2, 3, or 4, each R³⁶ is the same or different from each other,
when m5 is 2, 3, or 4, each R³⁷ is the same or different from each other, and
n is an integer of 0 to 2; 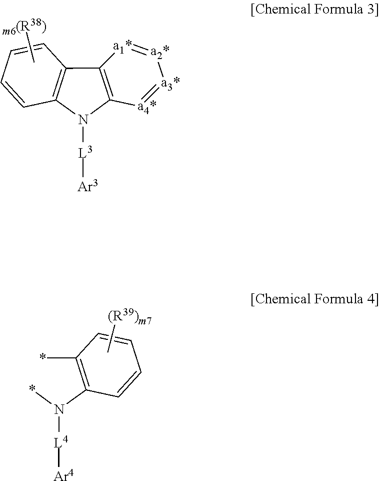
in Chemical Formula 3 and Chemical Formula 4,
two adjacent ones of a₁* to a₄* in Chemical Formula 3 are linking carbons linked at * in Chemical Formula 4, and the remaining two of a₁* to a₄* in Chemical Formula 3, not linked at * of Chemical Formula 4, are C-L^{a}-R^{a},
L^{a}, L³, and L⁴ are each independently a single bond or a substituted or unsubstituted C6 to C20 arylene group,
R^{a}, R³⁸ and R³⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar³ and Ar⁴ are each independently a substituted or unsubstituted C6 to C20 aryl group or a substituted or unsubstituted C2 to C30 heterocyclic group,
m6 and m7 are each independently an integer of 1 to 4,
when m6 is 2, 3, or 4, each R³⁸ is the same or different from each other, and
when m7 is 2, 3, or 4, each R³⁹ is the same or different from each other; 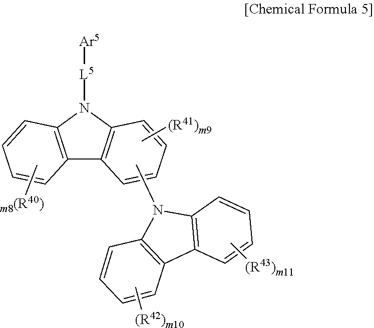
in Chemical Formula 5,
L⁵ is a single bond or a substituted or unsubstituted C6 to C20 arylene group,
R⁴⁰ to R⁴³ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁵ is a substituted or unsubstituted C6 to C20 aryl group or a substituted or unsubstituted C2 to C30 heterocyclic group,
m8, m10 and m11 are each independently an integer of 1 to 4,
m9 is an integer of 1 to 3,
when m8 is 2, 3, or 4, each R⁴⁰ is the same or different from each other,
when m9 is 2 or 3, each R⁴¹ is the same or different from each other,
when m10 is 2, 3, or 4, each R⁴² is the same or different from each other, and
when m11 is 2, 3, or 4, each R⁴³ is the same or different from each other.

6. The composition for an organic optoelectronic device as claimed in claim 5, wherein:
the second compound is represented by Chemical Formula 2,
Chemical Formula 2 is represented by Chemical Formula 2-8, 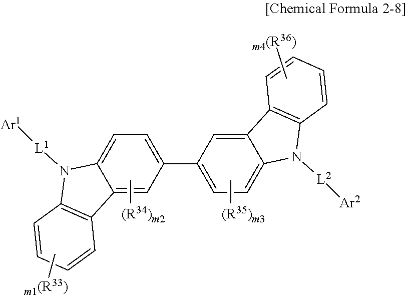
in Chemical Formula 2-8,
R³³ to R³⁶ are each independently hydrogen, deuterium, or a substituted or unsubstituted C6 to C12 aryl group,
m1 and m4 are each independently an integer of 1 to 4,
m2 and m3 are each independently an integer of 1 to 3, and
moieties -L¹-Ar¹ and -L²-Ar² are each independently a moiety of Group I, 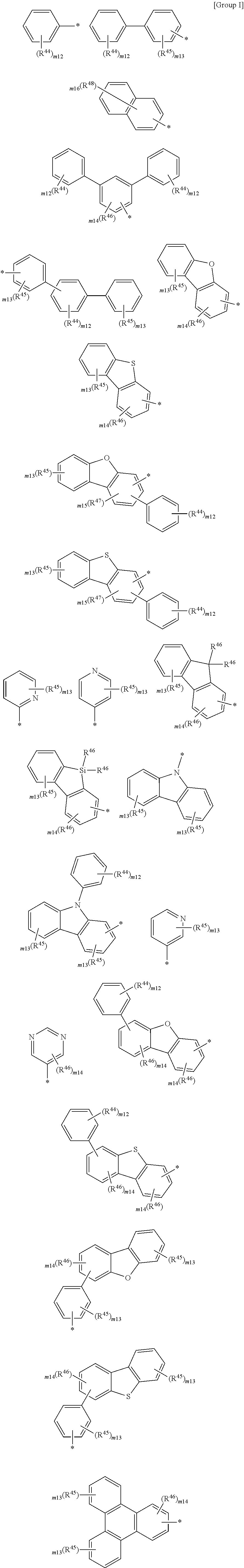 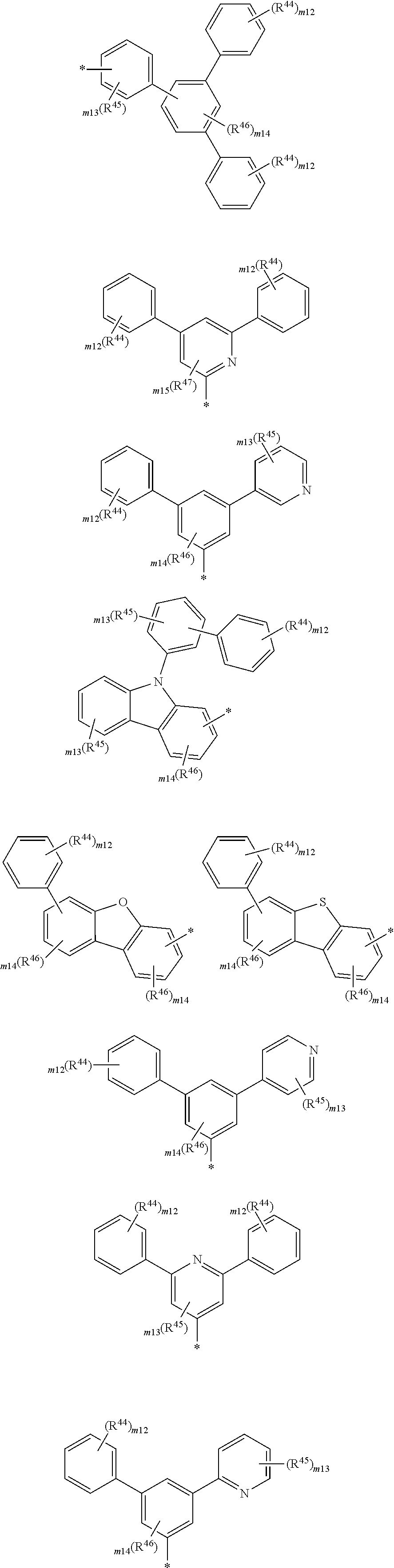
in Group I,
R⁴⁴ to R⁴⁸ are each independently hydrogen, deuterium, a cyano group, a substituted or unsubstituted C1 to C10 alkyl group, or a substituted or unsubstituted C6 to C12 aryl group,
m12 is an integer of 1 to 5,
m13 is an integer of 1 to 4,
m14 is an integer of 1 to 3,
m15 is an integer of 1 or 2,
m16 is an integer of 1 to 7, and
* is a linking point.

7. The composition for an organic optoelectronic device as claimed in claim 5, wherein:
the second compound is represented by a combination of Chemical Formula 3 and Chemical Formula 4,
the combination of Chemical Formula 3 and Chemical Formula 4 is represented by Chemical Formula 3C: 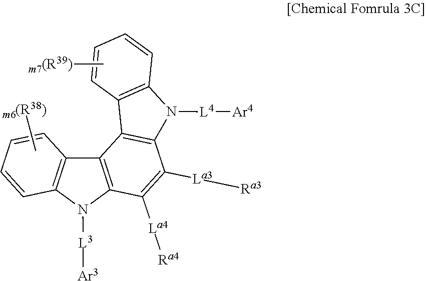
in Chemical Formula 3C,
L^{a3} and L^{a4} are a single bond,
R³⁸, R³⁹, R^{a3}, and R^{a4} are each independently hydrogen, deuterium, or a substituted or unsubstituted C6 to C12 aryl group,
m6 and m7 are each independently an integer of 1 to 4, and
moieties -L³-Ar³ and -L⁴-Ar⁴ are each independently a moiety of Group I, 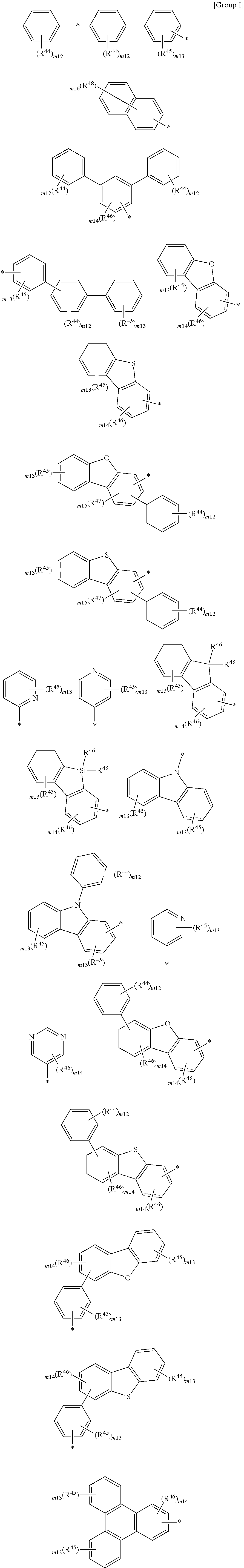 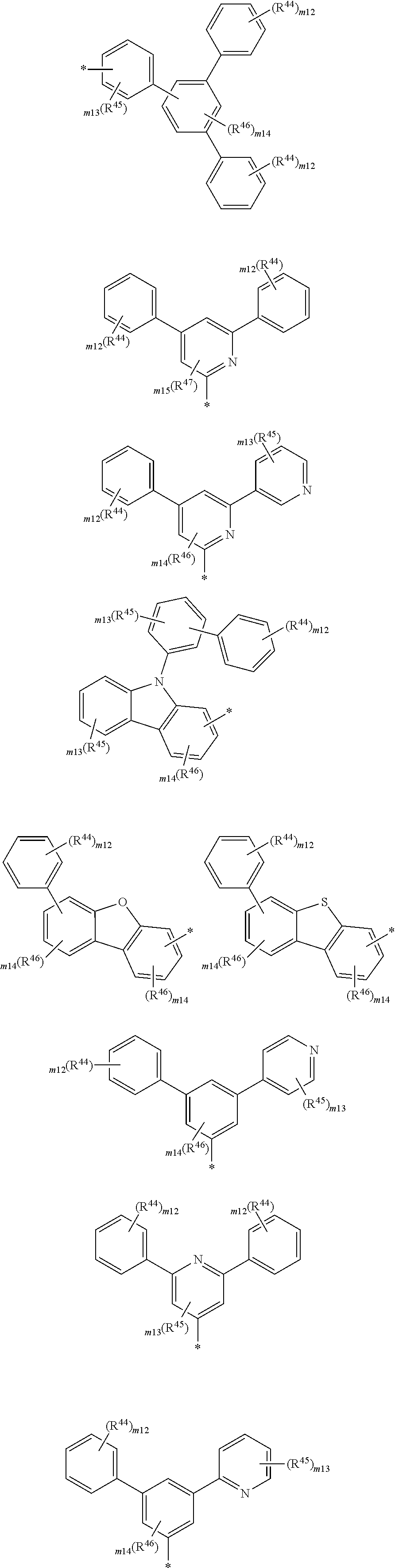
in Group I,
R⁴⁴ to R⁴⁸ are each independently hydrogen, deuterium, a cyano group, a substituted or unsubstituted C1 to C10 alkyl group, or a substituted or unsubstituted C6 to C12 aryl group,
m12 is an integer of 1 to 5,
m13 is an integer of 1 to 4,
m14 is an integer of 1 to 3,
m15 is an integer of 1 or 2,
m16 is an integer of 1 to 7, and
* is a linking point.

8. An organic optoelectronic device, comprising:
an anode and a cathode facing each other, and
at least one organic layer between the anode and the cathode,
wherein the at least one organic layer includes the compound for the organic optoelectronic device as claimed in claim 1.

9. The organic optoelectronic device as claimed in claim 8, wherein:
the at least one organic layer includes a light emitting layer, and
the light emitting layer includes the compound.

10. A display device comprising the organic optoelectronic device as claimed in claim 8.

11. An organic optoelectronic device, comprising:
an anode and a cathode facing each other, and
at least one organic layer between the anode and the cathode,
wherein the at least one organic layer includes the composition for the organic optoelectronic device as claimed in claim 5.

12. The organic optoelectronic device as claimed in claim 11, wherein
the at least one organic layer includes a light emitting layer, and
the light emitting layer includes the composition for an organic optoelectronic device.

13. A display device comprising the organic optoelectronic device as claimed in claim 11.
